SEQUENCE LISTING (449 sequences)

SEQ ID NO: 1 - DNA, 11 bp, Artificial Sequence
  Description of Artificial Sequence Synthetic oligonucleotide
ttagggtacac

SEQ ID NO: 2 - DNA, 20 bp, Artificial Sequence
  Description of Artificial Sequence Synthetic oligonucleotide
ttagggtacaccgtgtacct

SEQ ID NO: 3 - DNA, 19 bp, Artificial Sequence
  Description of Artificial Sequence Synthetic oligonucleotide
acggaccggaaatccggtt

SEQ ID NO: 4 - DNA, 10 bp, Artificial Sequence
  Description of Artificial Sequence Synthetic oligonucleotide
acaggaagtg

SEQ ID NO: 5 - DNA, 18 bp, Artificial Sequence
  Description of Artificial Sequence Synthetic oligonucleotide
tacaaagatcaaagggtt

SEQ ID NO: 6 - DNA, 15 bp, Artificial Sequence
  Description of Artificial Sequence Synthetic oligonucleotide
gggtcagggtgacct

SEQ ID NO: 7 - DNA, 15 bp, Artificial Sequence
  Description of Artificial Sequence Synthetic oligonucleotide
gggtcaaggtgaccc

SEQ ID NO: 8 - DNA, 15 bp, Artificial Sequence
  Description of Artificial Sequence Synthetic oligonucleotide
gggtcatggtgaccc

SEQ ID NO: 9 - DNA, 13 bp, Artificial Sequence
  Description of Artificial Sequence Synthetic oligonucleotide
ggtcacagtgacc

SEQ ID NO: 10 - DNA, 13 bp, Artificial Sequence
  Description of Artificial Sequence Synthetic oligonucleotide
agtcactgtgacc

SEQ ID NO: 11 - DNA, 10 bp, Artificial Sequence
  Description of Artificial Sequence Synthetic oligonucleotide
  modified_base (5)..(5): a, c, t, g, unknown or other
gggrnyyycc

SEQ ID NO: 12 - DNA, 10 bp, Artificial Sequence
  Description of Artificial Sequence Synthetic oligonucleotide
gggactttcc

SEQ ID NO: 13 - DNA, 10 bp, Artificial Sequence
  Description of Artificial Sequence Synthetic oligonucleotide
gggaatttcc

SEQ ID NO: 14 - DNA, 10 bp, Artificial Sequence
  Description of Artificial Sequence Synthetic oligonucleotide
ggggctttcc

SEQ ID NO: 15 - DNA, 8 bp, Artificial Sequence
  Description of Artificial Sequence Synthetic oligonucleotide
tttcccgc

SEQ ID NO: 16 - DNA, 8 bp, Artificial Sequence
  Description of Artificial Sequence Synthetic oligonucleotide
tttggcgc

SEQ ID NO: 17 - DNA, 8 bp, Artificial Sequence
  Description of Artificial Sequence Synthetic oligonucleotide
tttcgcgc

SEQ ID NO: 18 - DNA, 9 bp, Artificial Sequence
  Description of Artificial Sequence Synthetic oligonucleotide
atgagtcat

SEQ ID NO: 19 - DNA, 8 bp, Artificial Sequence
  Description of Artificial Sequence Synthetic oligonucleotide
wggaaaah

SEQ ID NO: 20 - DNA, 9 bp, Artificial Sequence
  Description of Artificial Sequence Synthetic oligonucleotide
atggaaaaa

SEQ ID NO: 21 - DNA, 9 bp, Artificial Sequence
  Description of Artificial Sequence Synthetic oligonucleotide
atggaaaat

SEQ ID NO: 22 - DNA, 9 bp, Artificial Sequence
  Description of Artificial Sequence Synthetic oligonucleotide
atggaaaac

SEQ ID NO: 23 - DNA, 9 bp, Artificial Sequence
  Description of Artificial Sequence Synthetic oligonucleotide
ttggaaaaa

SEQ ID NO: 24 - DNA, 9 bp, Artificial Sequence
  Description of Artificial Sequence Synthetic oligonucleotide
ttggaaaat

SEQ ID NO: 25 - DNA, 9 bp, Artificial Sequence
  Description of Artificial Sequence Synthetic oligonucleotide
ttggaaaac

SEQ ID NO: 26 - DNA, 7 bp, Artificial Sequence
  Description of Artificial Sequence Synthetic oligonucleotide
ygyggty

SEQ ID NO: 27 - DNA, 12 bp, Artificial Sequence
  Description of Artificial Sequence Synthetic oligonucleotide
agctgcggtcat

SEQ ID NO: 28 - DNA, 7 bp, Artificial Sequence
  Description of Artificial Sequence Synthetic oligonucleotide
tgcggtc

SEQ ID NO: 29 - DNA, 7 bp, Artificial Sequence
  Description of Artificial Sequence Synthetic oligonucleotide
cgtggtt

SEQ ID NO: 30 - DNA, 6 bp, Artificial Sequence
  Description of Artificial Sequence Synthetic oligonucleotide
cacgtg

SEQ ID NO: 31 - DNA, 12 bp, Artificial Sequence
  Description of Artificial Sequence Synthetic oligonucleotide
gaccacgtggtc

SEQ ID NO: 32 - DNA, 12 bp, Artificial Sequence
  Description of Artificial Sequence Synthetic oligonucleotide
ggccacgtgacc

SEQ ID NO: 33 - DNA, 6 bp, Artificial Sequence
  Description of Artificial Sequence Synthetic oligonucleotide
cggaag

SEQ ID NO: 34 - DNA, 6 bp, Artificial Sequence
  Description of Artificial Sequence Synthetic oligonucleotide
aggaag

SEQ ID NO: 35 - DNA, 9 bp, Artificial Sequence
  Description of Artificial Sequence Synthetic oligonucleotide
gaccaccca

SEQ ID NO: 36 - DNA, 10 bp, Artificial Sequence
  Description of Artificial Sequence Synthetic oligonucleotide
cggtaagtgg

SEQ ID NO: 37 - DNA, 6 bp, Artificial Sequence
  Description of Artificial Sequence Synthetic oligonucleotide
agatag

SEQ ID NO: 38 - DNA, 10 bp, Artificial Sequence
  Description of Artificial Sequence Synthetic oligonucleotide
aacagatgtt

SEQ ID NO: 39 - DNA, 16 bp, Artificial Sequence
  Description of Artificial Sequence Synthetic oligonucleotide
yaackghhtaacggaa

SEQ ID NO: 40 - DNA, 8 bp, Artificial Sequence
  Description of Artificial Sequence Synthetic oligonucleotide
taacggac

SEQ ID NO: 41 - DNA, 8 bp, Artificial Sequence
  Description of Artificial Sequence Synthetic oligonucleotide
taacggat

SEQ ID NO: 42 - DNA, 8 bp, Artificial Sequence
  Description of Artificial Sequence Synthetic oligonucleotide
taacggca

SEQ ID NO: 43 - DNA, 8 bp, Artificial Sequence
  Description of Artificial Sequence Synthetic oligonucleotide
taacggcc

SEQ ID NO: 44 - DNA, 8 bp, Artificial Sequence
  Description of Artificial Sequence Synthetic oligonucleotide
taacggct

SEQ ID NO: 45 - DNA, 8 bp, Artificial Sequence
  Description of Artificial Sequence Synthetic oligonucleotide
taacggta

SEQ ID NO: 46 - DNA, 8 bp, Artificial Sequence
  Description of Artificial Sequence Synthetic oligonucleotide
taacggtc

SEQ ID NO: 47 - DNA, 8 bp, Artificial Sequence
  Description of Artificial Sequence Synthetic oligonucleotide
taacggtt

SEQ ID NO: 48 - DNA, 16 bp, Artificial Sequence
  Description of Artificial Sequence Synthetic oligonucleotide
gcagccaagcgtgacc

SEQ ID NO: 49 - DNA, 8 bp, Artificial Sequence
  Description of Artificial Sequence Synthetic oligonucleotide
gtctagac

SEQ ID NO: 50 - DNA, 10 bp, Artificial Sequence
  Description of Artificial Sequence Synthetic oligonucleotide
aacagatggt

SEQ ID NO: 51 - DNA, 6 bp, Artificial Sequence
  Description of Artificial Sequence Synthetic oligonucleotide
agaaca

SEQ ID NO: 52 - DNA, 15 bp, Artificial Sequence
  Description of Artificial Sequence Synthetic oligonucleotide
  modified_base (7)..(9): a, c, t, g, unknown or other
agaacannntgttct

SEQ ID NO: 53 - DNA, 19 bp, Artificial Sequence
  Description of Artificial Sequence Synthetic oligonucleotide
ccacagagtgatgtaatca

SEQ ID NO: 54 - DNA, 9 bp, Artificial Sequence
  Description of Artificial Sequence Synthetic oligonucleotide
gcgtgggcg

SEQ ID NO: 55 - DNA, 9 bp, Artificial Sequence
  Description of Artificial Sequence Synthetic oligonucleotide
gcgggggcg

SEQ ID NO: 56 - DNA, 5 bp, Artificial Sequence
  Description of Artificial Sequence Synthetic oligonucleotide
acgtg

SEQ ID NO: 57 - DNA, 5 bp, Artificial Sequence
  Description of Artificial Sequence Synthetic oligonucleotide
gcgtg

SEQ ID NO: 58 - DNA, 9 bp, Artificial Sequence
  Description of Artificial Sequence Synthetic oligonucleotide
ttcgccgaa

SEQ ID NO: 59 - DNA, 9 bp, Artificial Sequence
  Description of Artificial Sequence Synthetic oligonucleotide
ttccccgaa

SEQ ID NO: 60 - DNA, 10 bp, Artificial Sequence
  Description of Artificial Sequence Synthetic oligonucleotide
cttccgggaa

SEQ ID NO: 61 - DNA, 9 bp, Artificial Sequence
  Description of Artificial Sequence Synthetic oligonucleotide
  modified_base (5)..(5): a, c, t, g, unknown or other
ttcynrgaa

SEQ ID NO: 62 - DNA, 9 bp, Artificial Sequence
  Description of Artificial Sequence Synthetic oligonucleotide
ttcttggaa

SEQ ID NO: 63 - DNA, 9 bp, Artificial Sequence
  Description of Artificial Sequence Synthetic oligonucleotide
ttcttagga

SEQ ID NO: 64 - DNA, 9 bp, Artificial Sequence
  Description of Artificial Sequence Synthetic oligonucleotide
ttcctggaa

SEQ ID NO: 65 - DNA, 9 bp, Artificial Sequence
  Description of Artificial Sequence Synthetic oligonucleotide
ttcctagaa

SEQ ID NO: 66 - DNA, 13 bp, Artificial Sequence
  Description of Artificial Sequence Synthetic oligonucleotide
tgctgactcagca

SEQ ID NO: 67 - DNA, 10 bp, Artificial Sequence
  Description of Artificial Sequence Synthetic oligonucleotide
cccattgttc

SEQ ID NO: 68 - DNA, 10 bp, Artificial Sequence
  Description of Artificial Sequence Synthetic oligonucleotide
agaacaatgg

SEQ ID NO: 69 - RNA, 21 nt, Artificial Sequence
  Description of Artificial Sequence Synthetic oligonucleotide
gaucugagccugggagcucuc

SEQ ID NO: 70 - RNA, 27 nt, Artificial Sequence
  Description of Artificial Sequence Synthetic oligonucleotide
gcagaucugagccugggagcucucugc

SEQ ID NO: 71 - DNA, 15 bp, Artificial Sequence
  Description of Artificial Sequence Synthetic oligonucleotide
agcatttcatcacat

SEQ ID NO: 72 - DNA, 18 bp, Artificial Sequence
  Description of Artificial Sequence Synthetic oligonucleotide
tgcagctttctacaaggg

SEQ ID NO: 73 - DNA, 17 bp, Artificial Sequence
  Description of Artificial Sequence Synthetic oligonucleotide
gcgcgagcgcaacaaca

SEQ ID NO: 74 - DNA, 20 bp, Artificial Sequence
  Description of Artificial Sequence Synthetic oligonucleotide
tgcttgaacaagttccgcag

SEQ ID NO: 75 - DNA, 8 bp, Artificial Sequence
  Description of Artificial Sequence Synthetic oligonucleotide
atgataag

SEQ ID NO: 76 - DNA, 20 bp, Artificial Sequence
  Description of Artificial Sequence Synthetic oligonucleotide
tgttactatataaatagaat

SEQ ID NO: 77 - DNA, 13 bp, Artificial Sequence
  Description of Artificial Sequence Synthetic oligonucleotide
atgcaaatgtgct

SEQ ID NO: 78 - DNA, 12 bp, Artificial Sequence
  Description of Artificial Sequence Synthetic oligonucleotide
aattattcatga

SEQ ID NO: 79 - DNA, 5 bp, Artificial Sequence
  Description of Artificial Sequence Synthetic oligonucleotide
cacct

SEQ ID NO: 80 - DNA, 12 bp, Artificial Sequence
  Description of Artificial Sequence Synthetic oligonucleotide
  modified_base (9)..(9): a, c, t, g, unknown or other
  modified_base (11)..(11): a, c, t, g, unknown or other
tgacctttncnt

SEQ ID NO: 81 - DNA, 11 bp, Artificial Sequence
  Description of Artificial Sequence Synthetic oligonucleotide
gtgacgtacag

SEQ ID NO: 82 - DNA, 8 bp, Artificial Sequence
  Description of Artificial Sequence Synthetic oligonucleotide
tgatgcaa

SEQ ID NO: 83 - DNA, 9 bp, Artificial Sequence
  Description of Artificial Sequence Synthetic oligonucleotide
gtgacgtaa

SEQ ID NO: 84 - DNA, 9 bp, Artificial Sequence
  Description of Artificial Sequence Synthetic oligonucleotide
gtgacgtag

SEQ ID NO: 85 - DNA, 9 bp, Artificial Sequence
  Description of Artificial Sequence Synthetic oligonucleotide
gtgacgtca

SEQ ID NO: 86 - DNA, 9 bp, Artificial Sequence
  Description of Artificial Sequence Synthetic oligonucleotide
gtgacgtcg

SEQ ID NO: 87 - DNA, 9 bp, Artificial Sequence
  Description of Artificial Sequence Synthetic oligonucleotide
tgatgaaac

SEQ ID NO: 88 - DNA, 10 bp, Artificial Sequence
  Description of Artificial Sequence Synthetic oligonucleotide
aggtgtgaaa

SEQ ID NO: 89 - DNA, 10 bp, Artificial Sequence
  Description of Artificial Sequence Synthetic oligonucleotide
aggtgtgaac

SEQ ID NO: 90 - DNA, 8 bp, Artificial Sequence
  Description of Artificial Sequence Synthetic oligonucleotide
tcacacct

SEQ ID NO: 91 - DNA, 8 bp, Artificial Sequence
  Description of Artificial Sequence Synthetic oligonucleotide
tcccacct

SEQ ID NO: 92 - DNA, 10 bp, Artificial Sequence
  Description of Artificial Sequence Synthetic oligonucleotide
aaytaggtca

SEQ ID NO: 93 - DNA, 10 bp, Artificial Sequence
  Description of Artificial Sequence Synthetic oligonucleotide
aattaggtca

SEQ ID NO: 94 - DNA, 10 bp, Artificial Sequence
  Description of Artificial Sequence Synthetic oligonucleotide
aactaggtca

SEQ ID NO: 95 - DNA, 6 bp, Artificial Sequence
  Description of Artificial Sequence Synthetic oligonucleotide
aggtca

SEQ ID NO: 96 - DNA, 10 bp, Artificial Sequence
  Description of Artificial Sequence Synthetic oligonucleotide
aaataggtca

SEQ ID NO: 97 - DNA, 17 bp, Artificial Sequence
  Description of Artificial Sequence Synthetic oligonucleotide
  modified_base (12)..(14): a, c, t, g, unknown or other
gatgacgtgtgnnnatt

SEQ ID NO: 98 - DNA, 20 bp, Artificial Sequence
  Description of Artificial Sequence Synthetic oligonucleotide
acacgcttgggaatggacac

SEQ ID NO: 99 - DNA, 20 bp, Artificial Sequence
  Description of Artificial Sequence Synthetic oligonucleotide
ccatgggaagatgttctggg

SEQ ID NO: 100 - DNA, 19 bp, Artificial Sequence
  Description of Artificial Sequence Synthetic oligonucleotide
cacgcttgggaatggacac

SEQ ID NO: 101 - DNA, 18 bp, Artificial Sequence
  Description of Artificial Sequence Synthetic oligonucleotide
gtgtcagagtccatggga

SEQ ID NO: 102 - DNA, 15 bp, Artificial Sequence
  Description of Artificial Sequence Synthetic oligonucleotide
gatgacgtgtagtat

SEQ ID NO: 103 - DNA, 15 bp, Artificial Sequence
  Description of Artificial Sequence Synthetic oligonucleotide
gatgacgtgtagttt

SEQ ID NO: 104 - DNA, 15 bp, Artificial Sequence
  Description of Artificial Sequence Synthetic oligonucleotide
gatgacgtggagttt

SEQ ID NO: 105 - DNA, 15 bp, Artificial Sequence
  Description of Artificial Sequence Synthetic oligonucleotide
gatgacgtggagtat

SEQ ID NO: 106 - DNA, 25 bp, Artificial Sequence
  Description of Artificial Sequence Synthetic oligonucleotide
gccccctcgcgccccgccccttgtc

SEQ ID NO: 107 - DNA, 11 bp, Artificial Sequence
  Description of Artificial Sequence Synthetic oligonucleotide
gtgtaccctaa

SEQ ID NO: 108 - DNA, 20 bp, Artificial Sequence
  Description of Artificial Sequence Synthetic oligonucleotide
aggtacacggtgtaccctaa

SEQ ID NO: 109 - DNA, 19 bp, Artificial Sequence
  Description of Artificial Sequence Synthetic oligonucleotide
aaccggatttccggtccgt

SEQ ID NO: 110 - DNA, 19 bp, Artificial Sequence
  Description of Artificial Sequence Synthetic oligonucleotide
gaccggaaatccggttcgt

SEQ ID NO: 111 - DNA, 19 bp, Artificial Sequence
  Description of Artificial Sequence Synthetic oligonucleotide
acgaaccggatttccggtc

SEQ ID NO: 112 - DNA, 45 bp, Artificial Sequence
  Description of Artificial Sequence Synthetic oligonucleotide
aaccggatttccggtccgtaagggaaacggaccggaaatccggtt

SEQ ID NO: 113 - DNA, 19 bp, Artificial Sequence
  Description of Artificial Sequence Synthetic oligonucleotide
gctccttaagtaaacaaac

SEQ ID NO: 114 - DNA, 19 bp, Artificial Sequence
  Description of Artificial Sequence Synthetic oligonucleotide
gtttgtttacttaaggagc

SEQ ID NO: 115 - DNA, 18 bp, Artificial Sequence
  Description of Artificial Sequence Synthetic oligonucleotide
aaccctttgatctttgta

SEQ ID NO: 116 - DNA, 20 bp, Artificial Sequence
  Description of Artificial Sequence Synthetic primer
gatcaagtgtgacccggact

SEQ ID NO: 117 - DNA, 20 bp, Artificial Sequence
  Description of Artificial Sequence Synthetic primer
cttggggtccatgttctgct

SEQ ID NO: 118 - DNA, 20 bp, Artificial Sequence
  Description of Artificial Sequence Synthetic primer
tacaacacccgagcaaggac

SEQ ID NO: 119 - DNA, 20 bp, Artificial Sequence
  Description of Artificial Sequence Synthetic primer
aggctggttttccactaccc

SEQ ID NO: 120 - DNA, 20 bp, Artificial Sequence
  Description of Artificial Sequence Synthetic primer
agcacggaaagaaagacagc

SEQ ID NO: 121 - DNA, 21 bp, Artificial Sequence
  Description of Artificial Sequence Synthetic primer
tcttggacctgtacctgatgc

SEQ ID NO: 122 - DNA, 20 bp, Artificial Sequence
  Description of Artificial Sequence Synthetic primer
tcggagtcaacggatttggt

SEQ ID NO: 123 - DNA, 20 bp, Artificial Sequence
  Description of Artificial Sequence Synthetic primer
ttcccgttctcagccttgac

SEQ ID NO: 124 - DNA, 25 bp, Artificial Sequence
  Description of Artificial Sequence Synthetic oligonucleotide
tcccacaagctcccatttccctgtc

SEQ ID NO: 125 - DNA, 25 bp, Artificial Sequence
  Description of Artificial Sequence Synthetic oligonucleotide
cccggccccggccccgccccggccc

SEQ ID NO: 126 - DNA, 25 bp, Artificial Sequence
  Description of Artificial Sequence Synthetic oligonucleotide
gccctcaggggcgcctcacactttc

SEQ ID NO: 127 - DNA, 25 bp, Artificial Sequence
  Description of Artificial Sequence Synthetic oligonucleotide
gccgccttccccgccgcccccgggt

SEQ ID NO: 128 - DNA, 25 bp, Artificial Sequence
  Description of Artificial Sequence Synthetic oligonucleotide
gcccacttccgccccgcctgcgtcc

SEQ ID NO: 129 - DNA, 25 bp, Artificial Sequence
  Description of Artificial Sequence Synthetic oligonucleotide
gccgccgtcagtcccgcccccaatc

SEQ ID NO: 130 - DNA, 25 bp, Artificial Sequence
  Description of Artificial Sequence Synthetic oligonucleotide
ccccgcgcactcccgagccctttcc

SEQ ID NO: 131 - DNA, 25 bp, Artificial Sequence
  Description of Artificial Sequence Synthetic oligonucleotide
gcttcctcccgccgcccctccccgc

SEQ ID NO: 132 - DNA, 25 bp, Artificial Sequence
  Description of Artificial Sequence Synthetic oligonucleotide
gccgccgggccgcccgggcctcttc

SEQ ID NO: 133 - DNA, 25 bp, Artificial Sequence
  Description of Artificial Sequence Synthetic oligonucleotide
gtcgccttggccgccgcctccttcc

SEQ ID NO: 134 - DNA, 25 bp, Artificial Sequence
  Description of Artificial Sequence Synthetic oligonucleotide
cccgccggccgccccctcccccgtg

SEQ ID NO: 135 - DNA, 25 bp, Artificial Sequence
  Description of Artificial Sequence Synthetic oligonucleotide
agcccggctggccgcgcccctcccc

SEQ ID NO: 136 - DNA, 25 bp, Artificial Sequence
  Description of Artificial Sequence Synthetic oligonucleotide
gctccctcgggagtggtccttgggc

SEQ ID NO: 137 - DNA, 25 bp, Artificial Sequence
  Description of Artificial Sequence Synthetic oligonucleotide
gaccccgcccaccctcgcgttggtc

SEQ ID NO: 138 - DNA, 25 bp, Artificial Sequence
  Description of Artificial Sequence Synthetic oligonucleotide
gctccctcgcgccggcgccggctcc

SEQ ID NO: 139 - DNA, 25 bp, Artificial Sequence
  Description of Artificial Sequence Synthetic oligonucleotide
gccacctcgcgccgtcaagcgtgat

SEQ ID NO: 140 - DNA, 25 bp, Artificial Sequence
  Description of Artificial Sequence Synthetic oligonucleotide
gcggcctcgggcctccgcctctgac

SEQ ID NO: 141 - DNA, 25 bp, Artificial Sequence
  Description of Artificial Sequence Synthetic oligonucleotide
gccccgcctggcgcagcccccgccc

SEQ ID NO: 142 - DNA, 25 bp, Artificial Sequence
  Description of Artificial Sequence Synthetic oligonucleotide
gagctcgcgcttcccgtcccccgcc

SEQ ID NO: 143 - DNA, 25 bp, Artificial Sequence
  Description of Artificial Sequence Synthetic oligonucleotide
gccccctctcccttcgagccctcct

SEQ ID NO: 144 - DNA, 25 bp, Artificial Sequence
  Description of Artificial Sequence Synthetic oligonucleotide
gcgctttcccgccccgcctcgccta

SEQ ID NO: 145 - DNA, 25 bp, Artificial Sequence
  Description of Artificial Sequence Synthetic oligonucleotide
ccccgcctgagcccttcgacttctc

SEQ ID NO: 146 - DNA, 25 bp, Artificial Sequence
  Description of Artificial Sequence Synthetic oligonucleotide
ccgccctcaggctccgggctttccc

SEQ ID NO: 147 - DNA, 25 bp, Artificial Sequence
  Description of Artificial Sequence Synthetic oligonucleotide
ggccacttccactccgcctcctggc

SEQ ID NO: 148 - DNA, 25 bp, Artificial Sequence
  Description of Artificial Sequence Synthetic oligonucleotide
gcctcctccccgtccccttcctttc

SEQ ID NO: 149 - DNA, 25 bp, Artificial Sequence
  Description of Artificial Sequence Synthetic oligonucleotide
cacccccagcccgccccctcgtctc

SEQ ID NO: 150 - DNA, 25 bp, Artificial Sequence
  Description of Artificial Sequence Synthetic oligonucleotide
cgccgctcccgcccgcccccaaggc

SEQ ID NO: 151 - DNA, 25 bp, Artificial Sequence
  Description of Artificial Sequence Synthetic oligonucleotide
tgcctcttcagccccgaccctgacc

SEQ ID NO: 152 - DNA, 25 bp, Artificial Sequence
  Description of Artificial Sequence Synthetic oligonucleotide
ccctctttcactccctccccttggc

SEQ ID NO: 153 - DNA, 25 bp, Artificial Sequence
  Description of Artificial Sequence Synthetic oligonucleotide
gccccctcccgcgccccctatggcc

SEQ ID NO: 154 - DNA, 25 bp, Artificial Sequence
  Description of Artificial Sequence Synthetic oligonucleotide
gttcccttgcatctctccccagttc

SEQ ID NO: 155 - DNA, 25 bp, Artificial Sequence
  Description of Artificial Sequence Synthetic oligonucleotide
gcccggccgcggcccgctctagacc

SEQ ID NO: 156 - DNA, 25 bp, Artificial Sequence
  Description of Artificial Sequence Synthetic oligonucleotide
gcccccctcctctccgccgcggccc

SEQ ID NO: 157 - DNA, 25 bp, Artificial Sequence
  Description of Artificial Sequence Synthetic oligonucleotide
ccctccttcggccccctctcttgtc

SEQ ID NO: 158 - DNA, 25 bp, Artificial Sequence
  Description of Artificial Sequence Synthetic oligonucleotide
tccgcccgccgcccgggcccacggc

SEQ ID NO: 159 - DNA, 25 bp, Artificial Sequence
  Description of Artificial Sequence Synthetic oligonucleotide
ccacccccgcgttccgaccgctgaa

SEQ ID NO: 160 - DNA, 25 bp, Artificial Sequence
  Description of Artificial Sequence Synthetic oligonucleotide
ccgccaccagccccggctccctgtc

SEQ ID NO: 161 - DNA, 25 bp, Artificial Sequence
  Description of Artificial Sequence Synthetic oligonucleotide
cccctttctccccgggccgcgtttc

SEQ ID NO: 162 - DNA, 25 bp, Artificial Sequence
  Description of Artificial Sequence Synthetic oligonucleotide
gccgccgcgggtcccctccggggtg

SEQ ID NO: 163 - DNA, 25 bp, Artificial Sequence
  Description of Artificial Sequence Synthetic oligonucleotide
tccggctcaagccccgccccctgcc

SEQ ID NO: 164 - DNA, 25 bp, Artificial Sequence
  Description of Artificial Sequence Synthetic oligonucleotide
ccccgcccccgccccgcctcccagg

SEQ ID NO: 165 - DNA, 25 bp, Artificial Sequence
  Description of Artificial Sequence Synthetic oligonucleotide
cccctctgcccagccgcccctactg

SEQ ID NO: 166 - DNA, 25 bp, Artificial Sequence
  Description of Artificial Sequence Synthetic oligonucleotide
gctcccgcgcggccaccggcctctc

SEQ ID NO: 167 - DNA, 25 bp, Artificial Sequence
  Description of Artificial Sequence Synthetic oligonucleotide
gcccagtccagcccagcccaatcta

SEQ ID NO: 168 - DNA, 25 bp, Artificial Sequence
  Description of Artificial Sequence Synthetic oligonucleotide
cggccgtcccgcagcgccccaggtg

SEQ ID NO: 169 - DNA, 25 bp, Artificial Sequence
  Description of Artificial Sequence Synthetic oligonucleotide
gccctcctgggcgctgcccgccgcc

SEQ ID NO: 170 - DNA, 25 bp, Artificial Sequence
  Description of Artificial Sequence Synthetic oligonucleotide
ggcccgtcgccctccggcctgggcc

SEQ ID NO: 171 - DNA, 25 bp, Artificial Sequence
  Description of Artificial Sequence Synthetic oligonucleotide
gccccatcgccccgcccctcccggg

SEQ ID NO: 172 - DNA, 25 bp, Artificial Sequence
  Description of Artificial Sequence Synthetic oligonucleotide
acctccgcgccgccccccacgcgtc

SEQ ID NO: 173 - DNA, 25 bp, Artificial Sequence
  Description of Artificial Sequence Synthetic oligonucleotide
ggctcccgaagccccgcccctggac

SEQ ID NO: 174 - DNA, 25 bp, Artificial Sequence
  Description of Artificial Sequence Synthetic oligonucleotide
cccccctccggctcggcgccggata

SEQ ID NO: 175 - DNA, 25 bp, Artificial Sequence
  Description of Artificial Sequence Synthetic oligonucleotide
gcacctgcgcgaaccaactcctttc

SEQ ID NO: 176 - DNA, 25 bp, Artificial Sequence
  Description of Artificial Sequence Synthetic oligonucleotide
gccccgccccgcccccaggcctgac

SEQ ID NO: 177 - DNA, 25 bp, Artificial Sequence
  Description of Artificial Sequence Synthetic oligonucleotide
tgtccctccgggccggccacgtggc

SEQ ID NO: 178 - DNA, 25 bp, Artificial Sequence
  Description of Artificial Sequence Synthetic oligonucleotide
gctccggccagcccaaccccgcgcc

SEQ ID NO: 179 - DNA, 25 bp, Artificial Sequence
  Description of Artificial Sequence Synthetic oligonucleotide
gcctcttggcgccccctcgatgctc

SEQ ID NO: 180 - DNA, 25 bp, Artificial Sequence
  Description of Artificial Sequence Synthetic oligonucleotide
gccgccccctcccccgggccctgac

SEQ ID NO: 181 - DNA, 25 bp, Artificial Sequence
  Description of Artificial Sequence Synthetic oligonucleotide
acccccttgcgttcaaagcctttcc

SEQ ID NO: 182 - DNA, 25 bp, Artificial Sequence
  Description of Artificial Sequence Synthetic oligonucleotide
acccgcacgcgcacacgcccctgcc

SEQ ID NO: 183 - DNA, 25 bp, Artificial Sequence
  Description of Artificial Sequence Synthetic oligonucleotide
gcctcctctcgcctcctccctggaa

SEQ ID NO: 184 - DNA, 25 bp, Artificial Sequence
  Description of Artificial Sequence Synthetic oligonucleotide
ggtcccgctctccccacccctcagc

SEQ ID NO: 185 - DNA, 25 bp, Artificial Sequence
  Description of Artificial Sequence Synthetic oligonucleotide
gccccaccggggcccgcgcggccac

SEQ ID NO: 186 - DNA, 25 bp, Artificial Sequence
  Description of Artificial Sequence Synthetic oligonucleotide
gccggccgacccccggccacttagc

SEQ ID NO: 187 - DNA, 25 bp, Artificial Sequence
  Description of Artificial Sequence Synthetic oligonucleotide
gcgcccccgcctcccggccgccgtc

SEQ ID NO: 188 - DNA, 25 bp, Artificial Sequence
  Description of Artificial Sequence Synthetic oligonucleotide
gcccccagggtccctcccccgccgc

SEQ ID NO: 189 - DNA, 25 bp, Artificial Sequence
  Description of Artificial Sequence Synthetic oligonucleotide
tcccccccgtgccccgagcctcggt

SEQ ID NO: 190 - DNA, 25 bp, Artificial Sequence
  Description of Artificial Sequence Synthetic oligonucleotide
gcgccacctcgccccgtcccaggct

SEQ ID NO: 191 - DNA, 25 bp, Artificial Sequence
  Description of Artificial Sequence Synthetic oligonucleotide
acgtcatggccccgcgcgcctcctc

SEQ ID NO: 192 - DNA, 25 bp, Artificial Sequence
  Description of Artificial Sequence Synthetic oligonucleotide
gctcctgccctcccggccgcttcct

SEQ ID NO: 193 - DNA, 25 bp, Artificial Sequence
  Description of Artificial Sequence Synthetic oligonucleotide
tcttgctcgcggacagctctttctc

SEQ ID NO: 194 - DNA, 25 bp, Artificial Sequence
  Description of Artificial Sequence Synthetic oligonucleotide
ttccccgcgccgcccacccactccc

SEQ ID NO: 195 - DNA, 25 bp, Artificial Sequence
  Description of Artificial Sequence Synthetic oligonucleotide
gcgcgcactcggctcctccctggcc

SEQ ID NO: 196 - DNA, 25 bp, Artificial Sequence
  Description of Artificial Sequence Synthetic oligonucleotide
gcccaggcgcgacgccccgagtggc

SEQ ID NO: 197 - DNA, 25 bp, Artificial Sequence
  Description of Artificial Sequence Synthetic oligonucleotide
tctccgccccccccccccgcatggc

SEQ ID NO: 198 - DNA, 25 bp, Artificial Sequence
  Description of Artificial Sequence Synthetic oligonucleotide
ttccgcccgcgccgccgccactgcc

SEQ ID NO: 199 - DNA, 25 bp, Artificial Sequence
  Description of Artificial Sequence Synthetic oligonucleotide
ccccccgcgcgctcccccttccgct

SEQ ID NO: 200 - DNA, 25 bp, Artificial Sequence
  Description of Artificial Sequence Synthetic oligonucleotide
gcgccccgcctcccgggcccacttc

SEQ ID NO: 201 - DNA, 25 bp, Artificial Sequence
  Description of Artificial Sequence Synthetic oligonucleotide
gccccctggcccggctctacttctt

SEQ ID NO: 202 - DNA, 25 bp, Artificial Sequence
  Description of Artificial Sequence Synthetic oligonucleotide
tcctccgcgagccgcggcccttgcc

SEQ ID NO: 203 - DNA, 25 bp, Artificial Sequence
  Description of Artificial Sequence Synthetic oligonucleotide
gcccggccgcgcccgcctccccgag

SEQ ID NO: 204 - DNA, 25 bp, Artificial Sequence
  Description of Artificial Sequence Synthetic oligonucleotide
ctctccccttcccccaccccttgcc

SEQ ID NO: 205 - DNA, 25 bp, Artificial Sequence
  Description of Artificial Sequence Synthetic oligonucleotide
ggcctccttccctctcccccttgtc

SEQ ID NO: 206 - DNA, 25 bp, Artificial Sequence
  Description of Artificial Sequence Synthetic oligonucleotide
gcctgctcgctccctccctcccgac

SEQ ID NO: 207 - DNA, 25 bp, Artificial Sequence
  Description of Artificial Sequence Synthetic oligonucleotide
gcctgctcgcgcggggcgtctgagc

SEQ ID NO: 208 - DNA, 25 bp, Artificial Sequence
  Description of Artificial Sequence Synthetic oligonucleotide
tctcccgcgtcccctccgcctcgcc

SEQ ID NO: 209 - DNA, 25 bp, Artificial Sequence
  Description of Artificial Sequence Synthetic oligonucleotide
cagcgctggcgcccggcccccttcc

SEQ ID NO: 210 - DNA, 25 bp, Artificial Sequence
  Description of Artificial Sequence Synthetic oligonucleotide
ggccccacgccccgaggcgcgcggc

SEQ ID NO: 211 - DNA, 25 bp, Artificial Sequence
  Description of Artificial Sequence Synthetic oligonucleotide
tgcacctggcgctgcgccacctgct

SEQ ID NO: 212 - DNA, 25 bp, Artificial Sequence
  Description of Artificial Sequence Synthetic oligonucleotide
gccgtgtcgcgccccaccctgcgcc

SEQ ID NO: 213 - DNA, 25 bp, Artificial Sequence
  Description of Artificial Sequence Synthetic oligonucleotide
gcccccgcgggctcccggccagggc

SEQ ID NO: 214 - DNA, 25 bp, Artificial Sequence
  Description of Artificial Sequence Synthetic oligonucleotide
gccacccccacccccaccgcgactc

SEQ ID NO: 215 - DNA, 25 bp, Artificial Sequence
  Description of Artificial Sequence Synthetic oligonucleotide
cctcctccgcccccagcgcctcata

SEQ ID NO: 216 - DNA, 25 bp, Artificial Sequence
  Description of Artificial Sequence Synthetic oligonucleotide
gaccccccccacctccctcccggac

SEQ ID NO: 217 - DNA, 25 bp, Artificial Sequence
  Description of Artificial Sequence Synthetic oligonucleotide
ggccgcgctctcccctcctccagtc

SEQ ID NO: 218 - DNA, 25 bp, Artificial Sequence
  Description of Artificial Sequence Synthetic oligonucleotide
gaccctggatgcccagcctcaagtc

SEQ ID NO: 219 - DNA, 25 bp, Artificial Sequence
  Description of Artificial Sequence Synthetic oligonucleotide
gcccgcccgcagccccgccccacgc

SEQ ID NO: 220 - DNA, 25 bp, Artificial Sequence
  Description of Artificial Sequence Synthetic oligonucleotide
ggcgccgcgggaccgccctcgtgtc

SEQ ID NO: 221 - DNA, 25 bp, Artificial Sequence
  Description of Artificial Sequence Synthetic oligonucleotide
gctccctcaggacccacgcgcggac

SEQ ID NO: 222 - DNA, 25 bp, Artificial Sequence
  Description of Artificial Sequence Synthetic oligonucleotide
cccccgcctggccagccgcctcgtc

SEQ ID NO: 223 - DNA, 25 bp, Artificial Sequence
  Description of Artificial Sequence Synthetic oligonucleotide
gcccgcgcccctcccgccccgcggc

SEQ ID NO: 224 - DNA, 25 bp, Artificial Sequence
  Description of Artificial Sequence Synthetic oligonucleotide
gctccgagccgcggcgccccctcgc

SEQ ID NO: 225 - DNA, 25 bp, Artificial Sequence
  Description of Artificial Sequence Synthetic oligonucleotide
ggcgccgcgagcttctcctctcctc

SEQ ID NO: 226 - DNA, 25 bp, Artificial Sequence
  Description of Artificial Sequence Synthetic oligonucleotide
cgcagcgcgcgccccctccccggcc

SEQ ID NO: 227 - DNA, 25 bp, Artificial Sequence
  Description of Artificial Sequence Synthetic oligonucleotide
acccagtcggcgcccactcctcgcc

SEQ ID NO: 228 - DNA, 25 bp, Artificial Sequence
  Description of Artificial Sequence Synthetic oligonucleotide
gtctcagagccgccccctccttgtc

SEQ ID NO: 229 - DNA, 25 bp, Artificial Sequence
  Description of Artificial Sequence Synthetic oligonucleotide
gcgcccaggttcccctccccagccc

SEQ ID NO: 230 - DNA, 25 bp, Artificial Sequence
  Description of Artificial Sequence Synthetic oligonucleotide
tccccagctcccgccgcccgtatcc

SEQ ID NO: 231 - DNA, 25 bp, Artificial Sequence
  Description of Artificial Sequence Synthetic oligonucleotide
cgctgcccgggccccgccccgacgc

SEQ ID NO: 232 - DNA, 25 bp, Artificial Sequence
  Description of Artificial Sequence Synthetic oligonucleotide
ttcccagccagccccgcgcctcagc

SEQ ID NO: 233 - DNA, 25 bp, Artificial Sequence
  Description of Artificial Sequence Synthetic oligonucleotide
cctttccccctccccttcccttctc

SEQ ID NO: 234 - DNA, 25 bp, Artificial Sequence
  Description of Artificial Sequence Synthetic oligonucleotide
tccgtcctgcgccctgccgctggcc

SEQ ID NO: 235 - DNA, 25 bp, Artificial Sequence
  Description of Artificial Sequence Synthetic oligonucleotide
acccctccaagcttcccggcttgtc

SEQ ID NO: 236 - DNA, 25 bp, Artificial Sequence
  Description of Artificial Sequence Synthetic oligonucleotide
gccggcgcacgccgcccctccggtg

SEQ ID NO: 237 - DNA, 25 bp, Artificial Sequence
  Description of Artificial Sequence Synthetic oligonucleotide
ccctccccgccccccgcgcccggat

SEQ ID NO: 238 - DNA, 25 bp, Artificial Sequence
  Description of Artificial Sequence Synthetic oligonucleotide
ttccccgcccgccctgctcctacgg

SEQ ID NO: 239 - DNA, 25 bp, Artificial Sequence
  Description of Artificial Sequence Synthetic oligonucleotide
gccccccgcctccctgcccccgggg

SEQ ID NO: 240 - DNA, 25 bp, Artificial Sequence
  Description of Artificial Sequence Synthetic oligonucleotide
ctcccctcgctctccgctcccgggg

SEQ ID NO: 241 - DNA, 25 bp, Artificial Sequence
  Description of Artificial Sequence Synthetic oligonucleotide
gcctcctcgtgtccctccgccccct

SEQ ID NO: 242 - DNA, 25 bp, Artificial Sequence
  Description of Artificial Sequence Synthetic oligonucleotide
atgccctctctcacccctcctattc

SEQ ID NO: 243 - DNA, 25 bp, Artificial Sequence
  Description of Artificial Sequence Synthetic oligonucleotide
cgccagccgcgccgcgcaccgagcc

SEQ ID NO: 244 - DNA, 25 bp, Artificial Sequence
  Description of Artificial Sequence Synthetic oligonucleotide
tccggcttgcgggccgcccttctcc

SEQ ID NO: 245 - DNA, 25 bp, Artificial Sequence
  Description of Artificial Sequence Synthetic oligonucleotide
gccgccgctgccgccgccccgggcg

SEQ ID NO: 246 - DNA, 25 bp, Artificial Sequence
  Description of Artificial Sequence Synthetic oligonucleotide
agcctcgggggccccgggccaggtc

SEQ ID NO: 247 - DNA, 25 bp, Artificial Sequence
  Description of Artificial Sequence Synthetic oligonucleotide
tccccgtcgcacctctcgccgcccc

SEQ ID NO: 248 - DNA, 25 bp, Artificial Sequence
  Description of Artificial Sequence Synthetic oligonucleotide
gcccctgtttccccaaacccttgtt

SEQ ID NO: 249 - DNA, 25 bp, Artificial Sequence
  Description of Artificial Sequence Synthetic oligonucleotide
cgcccctcccccccagctcttgggc

SEQ ID NO: 250 - DNA, 25 bp, Artificial Sequence
  Description of Artificial Sequence Synthetic oligonucleotide
ccccgcggccgctccacccctggcc

SEQ ID NO: 251 - DNA, 25 bp, Artificial Sequence
  Description of Artificial Sequence Synthetic oligonucleotide
cttccctcccgcactcccccgggcc

SEQ ID NO: 252 - DNA, 25 bp, Artificial Sequence
  Description of Artificial Sequence Synthetic oligonucleotide
agccagtgacgccccgcccccacta

SEQ ID NO: 253 - DNA, 25 bp, Artificial Sequence
  Description of Artificial Sequence Synthetic oligonucleotide
ggcctcccgcggccaagtccctgcc

SEQ ID NO: 254 - DNA, 25 bp, Artificial Sequence
  Description of Artificial Sequence Synthetic oligonucleotide
ggccgctcccgcccgcgccgctccc

SEQ ID NO: 255 - DNA, 25 bp, Artificial Sequence
  Description of Artificial Sequence Synthetic oligonucleotide
gccccgccccgccctgccgctaggg

SEQ ID NO: 256 - DNA, 25 bp, Artificial Sequence
  Description of Artificial Sequence Synthetic oligonucleotide
gcccccccgggccgcagctctctcc

SEQ ID NO: 257 - DNA, 25 bp, Artificial Sequence
  Description of Artificial Sequence Synthetic oligonucleotide
gcgcgccccttgccggccgcttgcc

SEQ ID NO: 258 - DNA, 25 bp, Artificial Sequence
  Description of Artificial Sequence Synthetic oligonucleotide
gaaccttcccgcccagcttctgggc

SEQ ID NO: 259 - DNA, 25 bp, Artificial Sequence
  Description of Artificial Sequence Synthetic oligonucleotide
gcctcccctcgccgcccgcccaggc

SEQ ID NO: 260 - DNA, 25 bp, Artificial Sequence
  Description of Artificial Sequence Synthetic oligonucleotide
gcctcttcccggccgccatcacgtc

SEQ ID NO: 261 - DNA, 25 bp, Artificial Sequence
  Description of Artificial Sequence Synthetic oligonucleotide
gcccccgggtggcgcccccccagcc

SEQ ID NO: 262 - DNA, 25 bp, Artificial Sequence
  Description of Artificial Sequence Synthetic oligonucleotide
gccccctggacgcccgcgcttccgg

SEQ ID NO: 263 - DNA, 25 bp, Artificial Sequence
  Description of Artificial Sequence Synthetic oligonucleotide
acccactcccaccctgcacccattg

SEQ ID NO: 264 - DNA, 25 bp, Artificial Sequence
  Description of Artificial Sequence Synthetic oligonucleotide
gcccacttccgccccgcaccgccca

SEQ ID NO: 265 - DNA, 25 bp, Artificial Sequence
  Description of Artificial Sequence Synthetic oligonucleotide
tcccattcggcccccgccccggccg

SEQ ID NO: 266 - DNA, 25 bp, Artificial Sequence
  Description of Artificial Sequence Synthetic oligonucleotide
cccccctgccaccccggcccataac

SEQ ID NO: 267 - DNA, 25 bp, Artificial Sequence
  Description of Artificial Sequence Synthetic oligonucleotide
gggccctcgggcgcagccactgacg

SEQ ID NO: 268 - DNA, 25 bp, Artificial Sequence
  Description of Artificial Sequence Synthetic oligonucleotide
accctcaccggcccagcctggtttc

SEQ ID NO: 269 - DNA, 25 bp, Artificial Sequence
  Description of Artificial Sequence Synthetic oligonucleotide
ggcccccggcgccccccggtgtccc

SEQ ID NO: 270 - DNA, 25 bp, Artificial Sequence
  Description of Artificial Sequence Synthetic oligonucleotide
ccccgctcggcgcacgcccccagcc

SEQ ID NO: 271 - DNA, 21 bp, Artificial Sequence
  Description of Artificial Sequence Synthetic primer
ttgcctgcattaccggtcgat

SEQ ID NO: 272 - DNA, 21 bp, Artificial Sequence
  Description of Artificial Sequence Synthetic primer
gatcctggcaatttcggctat

SEQ ID NO: 273 - DNA, 20 bp, Artificial Sequence
  Description of Artificial Sequence Synthetic primer
aacgagcgcagagttatcgt

SEQ ID NO: 274 - DNA, 24 bp, Artificial Sequence
  Description of Artificial Sequence Synthetic primer
ctaggccacagaattgaaagatct

SEQ ID NO: 275 - DNA, 20 bp, Artificial Sequence
  Description of Artificial Sequence Synthetic primer
gtgagcctctggaagtcgtc

SEQ ID NO: 276 - DNA, 25 bp, Artificial Sequence
  Description of Artificial Sequence Synthetic primer
gtaggtggaaattctagcatcatcc

SEQ ID NO: 277 - DNA, 22 bp, Artificial Sequence
  Description of Artificial Sequence Synthetic primer
cacaaaaacaggttaaacccag

SEQ ID NO: 278 - DNA, 20 bp, Artificial Sequence
  Description of Artificial Sequence Synthetic primer
agcacataggaggcagagac

SEQ ID NO: 279 - DNA, 23 bp, Artificial Sequence
  Description of Artificial Sequence Synthetic primer
gtagtactgttcgttccattccg

SEQ ID NO: 280 - DNA, 31 bp, Artificial Sequence
  Description of Artificial Sequence Synthetic primer
agctagccaccatggcttgagtaagtctgca

SEQ ID NO: 281 - DNA, 21 bp, Artificial Sequence
  Description of Artificial Sequence Synthetic primer
cttggagacatagccacactg

SEQ ID NO: 282 - DNA, 36 bp, Artificial Sequence
  Description of Artificial Sequence Synthetic oligonucleotide
ccggcaacaagatgaagagcaccaactcgagttggt

SEQ ID NO: 283 - DNA, 21 bp, Artificial Sequence
  Description of Artificial Sequence Synthetic oligonucleotide
gctcttcatcttgttgttttt

SEQ ID NO: 284 - DNA, 27 bp, Artificial Sequence
  Description of Artificial Sequence Synthetic oligonucleotide
ccgggcccatcaacagacgttgatact

SEQ ID NO: 285 - DNA, 30 bp, Artificial Sequence
  Description of Artificial Sequence Synthetic oligonucleotide
cgagtatcaacgtctgttgatgggcttttt

SEQ ID NO: 286 - DNA, 34 bp, Artificial Sequence
  Description of Artificial Sequence Synthetic oligonucleotide
ccgggctcatatcaaggaagccttactcgagtaa

SEQ ID NO: 287 - DNA, 23 bp, Artificial Sequence
  Description of Artificial Sequence Synthetic oligonucleotide
ggcttccttgatatgagcttttt

SEQ ID NO: 288 - DNA, 30 bp, Artificial Sequence
  Description of Artificial Sequence Synthetic oligonucleotide
ccggcggcgcacagaggaagagaatctcga

SEQ ID NO: 289 - DNA, 27 bp, Artificial Sequence
  Description of Artificial Sequence Synthetic oligonucleotide
gattctcttcctctgtgcgccgttttt

SEQ ID NO: 290 - DNA, 36 bp, Artificial Sequence
  Description of Artificial Sequence Synthetic oligonucleotide
ccgggtccagatgaagctcccagaactcgagttctg

SEQ ID NO: 291 - DNA, 21 bp, Artificial Sequence
  Description of Artificial Sequence Synthetic oligonucleotide
ggagcttcatctggacttttt

SEQ ID NO: 292 - DNA, 37 bp, Artificial Sequence
  Description of Artificial Sequence Synthetic oligonucleotide
ccggtctaacctcacttgcaataatctcgagattatt

SEQ ID NO: 293 - DNA, 21 bp, Artificial Sequence
  Description of Artificial Sequence Synthetic oligonucleotide
gcaagtgaggttagatttttg

SEQ ID NO: 294 - DNA, 39 bp, Artificial Sequence
  Description of Artificial Sequence Synthetic oligonucleotide
ccggttgttatcagaggactaaatactcgagtatttagt

SEQ ID NO: 295 - DNA, 19 bp, Artificial Sequence
  Description of Artificial Sequence Synthetic oligonucleotide
cctctgataacaatttttg

SEQ ID NO: 296 - DNA, 20 bp, Artificial Sequence
  Description of Artificial Sequence Synthetic primer
gtcttctcagtcaggtcgca

SEQ ID NO: 297 - DNA, 21 bp, Artificial Sequence
  Description of Artificial Sequence Synthetic primer
gagcatgggaatgtgattggc

SEQ ID NO: 298 - DNA, 20 bp, Artificial Sequence
  Description of Artificial Sequence Synthetic primer
gccgagagaggtgctttcat

SEQ ID NO: 299 - DNA, 20 bp, Artificial Sequence
  Description of Artificial Sequence Synthetic primer
aaaccctgcttccaaccgta

SEQ ID NO: 300 - DNA, 20 bp, Artificial Sequence
  Description of Artificial Sequence Synthetic primer
ggattccagctgttttcgcc

SEQ ID NO: 301 - DNA, 20 bp, Artificial Sequence
  Description of Artificial Sequence Synthetic primer
gggcgtgttctccttgtcag

SEQ ID NO: 302 - DNA, 20 bp, Artificial Sequence
  Description of Artificial Sequence Synthetic primer
ttgggattttctgcccgaca

SEQ ID NO: 303 - DNA, 22 bp, Artificial Sequence
  Description of Artificial Sequence Synthetic primer
ctccttgtcccgagtaatctga

SEQ ID NO: 304 - DNA, 23 bp, Artificial Sequence
  Description of Artificial Sequence Synthetic primer
agaagctgtgctatgttgctcta

SEQ ID NO: 305 - DNA, 24 bp, Artificial Sequence
  Description of Artificial Sequence Synthetic primer
acaggattccatacccaagaagga

SEQ ID NO: 306 - DNA, 20 bp, Artificial Sequence
  Description of Artificial Sequence Synthetic primer
aggtctgaggagcagcttca

SEQ ID NO: 307 - DNA, 22 bp, Artificial Sequence
  Description of Artificial Sequence Synthetic primer
caaataccctcaggggaacagg

SEQ ID NO: 308 - DNA, 20 bp, Artificial Sequence
  Description of Artificial Sequence Synthetic primer
aagcgctacgcctacaagtt

SEQ ID NO: 309 - DNA, 20 bp, Artificial Sequence
  Description of Artificial Sequence Synthetic primer
ttcatcttctgtgggtgggc

SEQ ID NO: 310 - DNA, 20 bp, Artificial Sequence
  Description of Artificial Sequence Synthetic primer
tgctcaagactggcgctaaa

SEQ ID NO: 311 - DNA, 19 bp, Artificial Sequence
  Description of Artificial Sequence Synthetic primer
cagtctggctgccaatcca

SEQ ID NO: 312 - DNA, 19 bp, Artificial Sequence
  Description of Artificial Sequence Synthetic primer
gagcagcggttagaatggc

SEQ ID NO: 313 - DNA, 21 bp, Artificial Sequence
  Description of Artificial Sequence Synthetic primer
tcctcctgcttccaactgaac

SEQ ID NO: 314 - DNA, 22 bp, Artificial Sequence
  Description of Artificial Sequence Synthetic primer
tcctgctcagatcaccatgaaa

SEQ ID NO: 315 - DNA, 21 bp, Artificial Sequence
  Description of Artificial Sequence Synthetic primer
ggctgccaggatagcatagtc

SEQ ID NO: 316 - DNA, 17 bp, Artificial Sequence
  Description of Artificial Sequence Synthetic primer
ctggagtgaggggtcgc

SEQ ID NO: 317 - DNA, 18 bp, Artificial Sequence
  Description of Artificial Sequence Synthetic primer
gcggcgtgttctccttgt

SEQ ID NO: 318 - DNA, 20 bp, Artificial Sequence
  Description of Artificial Sequence Synthetic primer
gagctgtcttccaagggagt

SEQ ID NO: 319 - DNA, 25 bp, Artificial Sequence
  Description of Artificial Sequence Synthetic primer
caactccctgtcctgaataatctga

SEQ ID NO: 320 - DNA, 19 bp, Artificial Sequence
  Description of Artificial Sequence Synthetic primer
agcacagagcctcgccttt

SEQ ID NO: 321 - DNA, 17 bp, Artificial Sequence
  Description of Artificial Sequence Synthetic primer
atcacgccctggtgcct

SEQ ID NO: 322 - DNA, 20 bp, Artificial Sequence
  Description of Artificial Sequence Synthetic primer
gactactttccaccgccccc

SEQ ID NO: 323 - DNA, 20 bp, Artificial Sequence
  Description of Artificial Sequence Synthetic primer
taaaatggcgccgcacaagg

SEQ ID NO: 324 - DNA, 22 bp, Artificial Sequence
  Description of Artificial Sequence Synthetic primer
gttgcagcttcgacaaacgtca

SEQ ID NO: 325 - DNA, 23 bp, Artificial Sequence
  Description of Artificial Sequence Synthetic primer
agctatcgattaagcagcctcca

SEQ ID NO: 326 - DNA, 16 bp, Artificial Sequence
  Description of Artificial Sequence Synthetic primer
caccccggggagcgtc

SEQ ID NO: 327 - DNA, 20 bp, Artificial Sequence
  Description of Artificial Sequence Synthetic primer
ggtggaaagtagtccccgcg

SEQ ID NO: 328 - DNA, 15 bp, Artificial Sequence
  Description of Artificial Sequence Synthetic primer
gccccctcgcgcccc

SEQ ID NO: 329 - DNA, 27 bp, Artificial Sequence
  Description of Artificial Sequence Synthetic primer
gagctcttataagtcgcgcagaagccg

SEQ ID NO: 330 - DNA, 20 bp, Artificial Sequence
  Description of Artificial Sequence Synthetic primer
cttgtgcggcgccattttaa

SEQ ID NO: 331 - DNA, 20 bp, Artificial Sequence
  Description of Artificial Sequence Synthetic primer
tcagaccttcctccgtctcc

SEQ ID NO: 332 - DNA, 19 bp, Artificial Sequence
  Description of Artificial Sequence Synthetic primer
caagccgggaaagctgcag

SEQ ID NO: 333 - DNA, 20 bp, Artificial Sequence
  Description of Artificial Sequence Synthetic primer
tgcttcaaattcccaggcgc

SEQ ID NO: 334 - DNA, 19 bp, Artificial Sequence
  Description of Artificial Sequence Synthetic primer
ctgacccagcgggctctag

SEQ ID NO: 335 - DNA, 19 bp, Artificial Sequence
  Description of Artificial Sequence Synthetic primer
atatggacatgcccggcgg

SEQ ID NO: 336 - DNA, 20 bp, Artificial Sequence
  Description of Artificial Sequence Synthetic primer
aagtcgcgctaaccttggcc

SEQ ID NO: 337 - DNA, 20 bp, Artificial Sequence
  Description of Artificial Sequence Synthetic primer
ctcctctgcattcccagcct

SEQ ID NO: 338 - DNA, 18 bp, Artificial Sequence
  Description of Artificial Sequence Synthetic primer
ctcagctgtggccctggg

SEQ ID NO: 339 - DNA, 17 bp, Artificial Sequence
  Description of Artificial Sequence Synthetic primer
tcttcctgctcggcggg

SEQ ID NO: 340 - DNA, 21 bp, Artificial Sequence
  Description of Artificial Sequence Synthetic primer
ccaggagaagcacaaactggc

SEQ ID NO: 341 - DNA, 19 bp, Artificial Sequence
  Description of Artificial Sequence Synthetic primer
gaagtcccgcctcttccgc

SEQ ID NO: 342 - DNA, 23 bp, Artificial Sequence
  Description of Artificial Sequence Synthetic primer
gcaagaggtagagaggtgacctg

SEQ ID NO: 343 - DNA, 23 bp, Artificial Sequence
  Description of Artificial Sequence Synthetic primer
gctgtggttgtgacgccttttag

SEQ ID NO: 344 - DNA, 20 bp, Artificial Sequence
  Description of Artificial Sequence Synthetic primer
atcggaggaccccagaagac

SEQ ID NO: 345 - DNA, 20 bp, Artificial Sequence
  Description of Artificial Sequence Synthetic primer
ggcaccacttactatgcccc

SEQ ID NO: 346 - DNA, 20 bp, Artificial Sequence
  Description of Artificial Sequence Synthetic primer
gcccacattccttcctgacg

SEQ ID NO: 347 - DNA, 18 bp, Artificial Sequence
  Description of Artificial Sequence Synthetic primer
cgggacctgcaggtgacg

SEQ ID NO: 348 - DNA, 21 bp, Artificial Sequence
  Description of Artificial Sequence Synthetic primer
agacagccacagtcctgtctg

SEQ ID NO: 349 - DNA, 18 bp, Artificial Sequence
  Description of Artificial Sequence Synthetic primer
ctgcactccatcctgggc

SEQ ID NO: 350 - DNA, 21 bp, Artificial Sequence
  Description of Artificial Sequence Synthetic primer
ggacaagaccagcggctaatc

SEQ ID NO: 351 - DNA, 19 bp, Artificial Sequence
  Description of Artificial Sequence Synthetic primer
gagcacaccatggctgctg

SEQ ID NO: 352 - DNA, 21 bp, Artificial Sequence
  Description of Artificial Sequence Synthetic primer
ggcgagtatcagaggatggga

SEQ ID NO: 353 - DNA, 23 bp, Artificial Sequence
  Description of Artificial Sequence Synthetic primer
ggtgtggctagttggtaacactt

SEQ ID NO: 354 - DNA, 20 bp, Artificial Sequence
  Description of Artificial Sequence Synthetic primer
ttaggggttgggctggatgc

SEQ ID NO: 355 - DNA, 21 bp, Artificial Sequence
  Description of Artificial Sequence Synthetic primer
catttgccagtggcaacatcc

SEQ ID NO: 356 - DNA, 25 bp, Artificial Sequence
  Description of Artificial Sequence Synthetic probe
gacaaggggcggggcgcgagggggc

SEQ ID NO: 357 - DNA, 25 bp, Artificial Sequence
  Description of Artificial Sequence Synthetic probe
ctcgcgcggcggaacgctccgcgct

SEQ ID NO: 358 - DNA, 25 bp, Artificial Sequence
  Description of Artificial Sequence Synthetic probe
agcgcggagcgttccgccgcgcgag

SEQ ID NO: 359 - DNA, 25 bp, Artificial Sequence
  Description of Artificial Sequence Synthetic probe
gcgccggtggcggcaggatacagcg

SEQ ID NO: 360 - DNA, 25 bp, Artificial Sequence
  Description of Artificial Sequence Synthetic probe
cgctgtatcctgccgccaccggcgc

SEQ ID NO: 361 - DNA, 25 bp, Artificial Sequence
  Description of Artificial Sequence Synthetic probe
gcttctgcgcgacttataagagctc

SEQ ID NO: 362 - DNA, 25 bp, Artificial Sequence
  Description of Artificial Sequence Synthetic probe
gagctcttataagtcgcgcagaagc

SEQ ID NO: 363 - DNA, 25 bp, Artificial Sequence
  Description of Artificial Sequence Synthetic oligonucleotide
caccgccgcagatcgatgtagatgg

SEQ ID NO: 364 - DNA, 25 bp, Artificial Sequence
  Description of Artificial Sequence Synthetic oligonucleotide
aaacccatctacatcgatctgcggc

SEQ ID NO: 365 - DNA, 25 bp, Artificial Sequence
  Description of Artificial Sequence Synthetic oligonucleotide
caccggccccgcagatcgatgtaga

SEQ ID NO: 366 - DNA, 25 bp, Artificial Sequence
  Description of Artificial Sequence Synthetic oligonucleotide
aaactctacatcgatctgcggggcc

SEQ ID NO: 367 - DNA, 25 bp, Artificial Sequence
  Description of Artificial Sequence Synthetic oligonucleotide
caccgccccgcagatcgatgtagat

SEQ ID NO: 368 - DNA, 25 bp, Artificial Sequence
  Description of Artificial Sequence Synthetic oligonucleotide
aaacatctacatcgatctgcggggc

SEQ ID NO: 369 - DNA, 25 bp, Artificial Sequence
  Description of Artificial Sequence Synthetic oligonucleotide
caccggtaatccaaggcttgtacag

SEQ ID NO: 370 - DNA, 25 bp, Artificial Sequence
  Description of Artificial Sequence Synthetic oligonucleotide
aaacctgtacaagccttggattacc

SEQ ID NO: 371 - DNA, 25 bp, Artificial Sequence
  Description of Artificial Sequence Synthetic oligonucleotide
caccggtcagggtgcttagtagtca

SEQ ID NO: 372 - DNA, 25 bp, Artificial Sequence
  Description of Artificial Sequence Synthetic oligonucleotide
aaactgactactaagcaccctgacc

SEQ ID NO: 373 - DNA, 25 bp, Artificial Sequence
  Description of Artificial Sequence Synthetic oligonucleotide
caccgcaagccttggattactttca

SEQ ID NO: 374 - DNA, 25 bp, Artificial Sequence
  Description of Artificial Sequence Synthetic oligonucleotide
aaactgaaagtaatccaaggcttgc

SEQ ID NO: 375 - DNA, 25 bp, Artificial Sequence
  Description of Artificial Sequence Synthetic oligonucleotide
caccgggggctcagcttggtcgaca

SEQ ID NO: 376 - DNA, 25 bp, Artificial Sequence
  Description of Artificial Sequence Synthetic oligonucleotide
aaactgtcgaccaagctgagccccc

SEQ ID NO: 377 - DNA, 25 bp, Artificial Sequence
  Description of Artificial Sequence Synthetic oligonucleotide
caccgcttggtcgacaaggagaaca

SEQ ID NO: 378 - DNA, 25 bp, Artificial Sequence
  Description of Artificial Sequence Synthetic oligonucleotide
aaactgttctccttgtcgaccaagc

SEQ ID NO: 379 - DNA, 25 bp, Artificial Sequence
  Description of Artificial Sequence Synthetic oligonucleotide
caccggccgctgaaggggctcagct

SEQ ID NO: 380 - DNA, 25 bp, Artificial Sequence
  Description of Artificial Sequence Synthetic oligonucleotide
aaacagctgagccccttcagcggcc

SEQ ID NO: 381 - DNA, 24 bp, Artificial Sequence
  Description of Artificial Sequence Synthetic oligonucleotide
atggaggagccgcagtcagatcct

SEQ ID NO: 382 - DNA, 27 bp, Artificial Sequence
  Description of Artificial Sequence Synthetic oligonucleotide
tcagtctgagtcaggcccttctgtctt

SEQ ID NO: 383 - DNA, 20 bp, Artificial Sequence
  Description of Artificial Sequence Synthetic oligonucleotide
ccggacatgcccgggcatgt

SEQ ID NO: 384 - DNA, 20 bp, Artificial Sequence
  Description of Artificial Sequence Synthetic oligonucleotide
ccggacatgcccgggcatgt

SEQ ID NO: 385 - DNA, 20 bp, Artificial Sequence
  Description of Artificial Sequence Synthetic oligonucleotide
ccggacatgcccgggcatgt

SEQ ID NO: 386 - DNA, 20 bp, Artificial Sequence
  Description of Artificial Sequence Synthetic oligonucleotide
ccggacatgcccgggcatgt

SEQ ID NO: 387 - DNA, 20 bp, Artificial Sequence
  Description of Artificial Sequence Synthetic oligonucleotide
acatgcccgggcatgtccgg

SEQ ID NO: 388 - DNA, 25 bp, Artificial Sequence
  Description of Artificial Sequence Synthetic oligonucleotide
gccccctcgcgccccgccccttgtc

SEQ ID NO: 389 - DNA, 25 bp, Artificial Sequence
  Description of Artificial Sequence Synthetic oligonucleotide
gccccctcgcgccccgccccttgtc

SEQ ID NO: 390 - DNA, 25 bp, Artificial Sequence
  Description of Artificial Sequence Synthetic oligonucleotide
gccccctcgcgccccgccccttgtc

SEQ ID NO: 391 - DNA, 25 bp, Artificial Sequence
  Description of Artificial Sequence Synthetic oligonucleotide
gccccctcgcgccccgccccttgtc

SEQ ID NO: 392 - DNA, 25 bp, Artificial Sequence
  Description of Artificial Sequence Synthetic oligonucleotide
gacaaggggcggggcgcgagggggc

SEQ ID NO: 393 - DNA, 25 bp, Artificial Sequence
  Description of Artificial Sequence Synthetic oligonucleotide
gcccgcccgcgccccgcccctcgtc

SEQ ID NO: 394 - DNA, 25 bp, Artificial Sequence
  Description of Artificial Sequence Synthetic oligonucleotide
gcccgcccgcgccccgcccctcgtc

SEQ ID NO: 395 - DNA, 25 bp, Artificial Sequence
  Description of Artificial Sequence Synthetic oligonucleotide
gcccgcccgcgccccgcccctcgtc

SEQ ID NO: 396 - DNA, 25 bp, Artificial Sequence
  Description of Artificial Sequence Synthetic oligonucleotide
gcccgcccgcgccccgcccctcgtc

SEQ ID NO: 397 - DNA, 25 bp, Artificial Sequence
  Description of Artificial Sequence Synthetic oligonucleotide
gacgaggggcggggcgcgggcgggc

SEQ ID NO: 398 - DNA, 25 bp, Artificial Sequence
  Description of Artificial Sequence Synthetic oligonucleotide
ctcccctcgccgcccgcccaggctc

SEQ ID NO: 399 - DNA, 25 bp, Artificial Sequence
  Description of Artificial Sequence Synthetic oligonucleotide
ctcccctcgccgcccgcccaggctc

SEQ ID NO: 400 - DNA, 25 bp, Artificial Sequence
  Description of Artificial Sequence Synthetic oligonucleotide
ctcccctcgccgcccgcccaggctc

SEQ ID NO: 401 - DNA, 25 bp, Artificial Sequence
  Description of Artificial Sequence Synthetic oligonucleotide
ctcccctcgccgcccgcccaggctc

SEQ ID NO: 402 - DNA, 25 bp, Artificial Sequence
  Description of Artificial Sequence Synthetic oligonucleotide
gagcctgggcgggcggcgaggggag

SEQ ID NO: 403 - DNA, 25 bp, Artificial Sequence
  Description of Artificial Sequence Synthetic oligonucleotide
gcggcctcgggcctccgcctctgac

SEQ ID NO: 404 - DNA, 25 bp, Artificial Sequence
  Description of Artificial Sequence Synthetic oligonucleotide
gcggcctcgggcctccgcctctgac

SEQ ID NO: 405 - DNA, 25 bp, Artificial Sequence
  Description of Artificial Sequence Synthetic oligonucleotide
gcggcctcgggcctccgcctctgac

SEQ ID NO: 406 - DNA, 25 bp, Artificial Sequence
  Description of Artificial Sequence Synthetic oligonucleotide
gcggcctcgggcctccgcctctgac

SEQ ID NO: 407 - DNA, 25 bp, Artificial Sequence
  Description of Artificial Sequence Synthetic oligonucleotide
gtcagaggcggaggcccgaggccgc

SEQ ID NO: 408 - DNA, 25 bp, Artificial Sequence
  Description of Artificial Sequence Synthetic oligonucleotide
cccggccccggccccgccccggccc

SEQ ID NO: 409 - DNA, 25 bp, Artificial Sequence
  Description of Artificial Sequence Synthetic oligonucleotide
cccggccccggccccgccccggccc

SEQ ID NO: 410 - DNA, 25 bp, Artificial Sequence
  Description of Artificial Sequence Synthetic oligonucleotide
cccggccccggccccgccccggccc

SEQ ID NO: 411 - DNA, 25 bp, Artificial Sequence
  Description of Artificial Sequence Synthetic oligonucleotide
cccggccccggccccgccccggccc

SEQ ID NO: 412 - DNA, 25 bp, Artificial Sequence
  Description of Artificial Sequence Synthetic oligonucleotide
gggccggggcggggccggggccggg

SEQ ID NO: 413 - DNA, 25 bp, Artificial Sequence
  Description of Artificial Sequence Synthetic oligonucleotide
tccggcttgcgggccgcccttctcc

SEQ ID NO: 414 - DNA, 25 bp, Artificial Sequence
  Description of Artificial Sequence Synthetic oligonucleotide
tccggcttgcgggccgcccttctcc

SEQ ID NO: 415 - DNA, 25 bp, Artificial Sequence
  Description of Artificial Sequence Synthetic oligonucleotide
tccggcttgcgggccgcccttctcc

SEQ ID NO: 416 - DNA, 25 bp, Artificial Sequence
  Description of Artificial Sequence Synthetic oligonucleotide
tccggcttgcgggccgcccttctcc

SEQ ID NO: 417 - DNA, 25 bp, Artificial Sequence
  Description of Artificial Sequence Synthetic oligonucleotide
ggagaagggcggcccgcaagccgga

SEQ ID NO: 418 - DNA, 18 bp, Artificial Sequence
  Description of Artificial Sequence Synthetic oligonucleotide
aaccctttgatctttgta

SEQ ID NO: 419 - DNA, 19 bp, Artificial Sequence
  Description of Artificial Sequence Synthetic oligonucleotide
aaccggatttccggtccgt

SEQ ID NO: 420 - DNA, 19 bp, Artificial Sequence
  Description of Artificial Sequence Synthetic oligonucleotide
tgtgctagctgatgtgcta

SEQ ID NO: 421 - DNA, 19 bp, Artificial Sequence
  Description of Artificial Sequence Synthetic oligonucleotide
tagcacatcagctagcaca

SEQ ID NO: 422 - DNA, 20 bp, Artificial Sequence
  Description of Artificial Sequence Synthetic primer
gcctatgccccctgagagtg

SEQ ID NO: 423 - DNA, 26 bp, Artificial Sequence
  Description of Artificial Sequence Synthetic primer
gcttgagttggcctagtttgttgttc

SEQ ID NO: 424 - DNA, 19 bp, Artificial Sequence
  Description of Artificial Sequence Synthetic primer
gaaggtgaaggtcggagtc

SEQ ID NO: 425 - DNA, 20 bp, Artificial Sequence
  Description of Artificial Sequence Synthetic primer
gaagatggtgatgggatttc

SEQ ID NO: 426 - DNA, 26 bp, Artificial Sequence
  Description of Artificial Sequence Synthetic primer
ttcattttggccatctcttccttcag

SEQ ID NO: 427 - DNA, 26 bp, Artificial Sequence
  Description of Artificial Sequence Synthetic primer
tatccagctcgtacctcatttcctct

SEQ ID NO: 428 - DNA, 21 bp, Artificial Sequence
  Description of Artificial Sequence Synthetic primer
tgcccggaccaaggatacagt

SEQ ID NO: 429 - DNA, 19 bp, Artificial Sequence
  Description of Artificial Sequence Synthetic primer
agcgcgtggccgaactcat

SEQ ID NO: 430 - DNA, 21 bp, Artificial Sequence
  Description of Artificial Sequence Synthetic primer
cactgggcctgggcaaacata

SEQ ID NO: 431 - DNA, 21 bp, Artificial Sequence
  Description of Artificial Sequence Synthetic primer
cacgtcagcctgcggttcttc

SEQ ID NO: 432 - DNA, 26 bp, Artificial Sequence
  Description of Artificial Sequence Synthetic primer
tacggctctgaagtcaccaccaaaat

SEQ ID NO: 433 - DNA, 23 bp, Artificial Sequence
  Description of Artificial Sequence Synthetic primer
ccccagctcacaattccagtcaa

SEQ ID NO: 434 - DNA, 18 bp, Artificial Sequence
  Description of Artificial Sequence Synthetic primer
gagagtgtgcaagagatc

SEQ ID NO: 435 - DNA, 17 bp, Artificial Sequence
  Description of Artificial Sequence Synthetic primer
gcttttggtcaacacgg

SEQ ID NO: 436 - DNA, 18 bp, Artificial Sequence
  Description of Artificial Sequence Synthetic primer
cgcccggaggtgaaagcg

SEQ ID NO: 437 - RNA, 21 nt, Artificial Sequence
  Description of Artificial Sequence Synthetic oligonucleotide
uagcgacuaaacacaucaauu

SEQ ID NO: 438 - RNA, 19 nt, Artificial Sequence
  Description of Artificial Sequence Synthetic oligonucleotide
gaauaaauguacuucccuu

SEQ ID NO: 439 - RNA, 19 nt, Artificial Sequence
  Description of Artificial Sequence Synthetic oligonucleotide
gugcugauauggaagaauu

SEQ ID NO: 440 - RNA, 19 nt, Artificial Sequence
  Description of Artificial Sequence Synthetic oligonucleotide
ccagcaagcuaaagugcaa

SEQ ID NO: 441 - RNA, 19 nt, Artificial Sequence
  Description of Artificial Sequence Synthetic oligonucleotide
agacaaagguucaaagucc

SEQ ID NO: 442 - DNA, 25 bp, Artificial Sequence
  Description of Artificial Sequence Synthetic oligonucleotide
gcccgcccgcgccccgcccctcgtc

SEQ ID NO: 443 - DNA, 30 bp, Artificial Sequence
  Description of Artificial Sequence Synthetic oligonucleotide
ctactttccaccgccccctcgcgccccgcc

SEQ ID NO: 444 - DNA, 30 bp, Artificial Sequence
  Description of Artificial Sequence Synthetic oligonucleotide
ccttgtcctcgcgcggcggaacgctccgcg

SEQ ID NO: 445 - DNA, 11 bp, Artificial Sequence
  Description of Artificial Sequence Synthetic oligonucleotide
gtgtaccctaa

SEQ ID NO: 446 - DNA, 20 bp, Artificial Sequence
  Description of Artificial Sequence Synthetic oligonucleotide
aggtacacggtgtaccctaa

SEQ ID NO: 447 - DNA, 19 bp, Artificial Sequence
  Description of Artificial Sequence Synthetic oligonucleotide
aaccggatttccggyccgt

SEQ ID NO: 448 - DNA, 18 bp, Artificial Sequence
  Description of Artificial Sequence Synthetic oligonucleotide
aaccctttgatctttgta

SEQ ID NO: 449 - DNA, 25 bp, Artificial Sequence
  Description of Artificial Sequence Synthetic oligonucleotide
gccccctcgcgccccgccccttgtc

(19) United States
(12) Patent Application Publication
(10) Pub. No.: US 2024/0190903 A1
(43) Pub. Date: Jun. 13, 2024

(54) COMPOUNDS FOR PROGRAMMABLE PROTEIN DEGRADATION AND METHODS OF USE FOR THE TREATMENT OF DISEASE

(71) Applicants: Mayo Foundation for Medical Education and Research, Rochester, MN (US); BioVentures, LLC, Little Rock, AR (US)

(72) Inventors: Haojie HUANG, Rochester, MN (US); Jingwei SHAO, Little Rock, AR (US); Wei YAN, Little Rock, AR (US); Yuqian YAN, Rochester, MN (US); Hong-yu LI, Little Rock, AR (US)

(21) Appl. No.: 18/277,204

(22) PCT Filed: Feb. 25, 2022
(86) PCT No.: PCT/US2022/017931

§ 371 Date: Aug. 14, 2023

Related U.S. Application Data
(60) Provisional application No. 63/271,534, filed on Oct. 25, 2021.
(60) Provisional application No. 63/158,218, filed on Mar. 8, 2021.
(60) Provisional application No. 63/153,872, filed on Feb. 25, 2021.

(51) Int. Cl. C07F 9/24; A61K 47/54; A61P 35/00; C07F 9/6518; C07F 9/6558

(52) U.S. Cl. CPC C07F 9/2408; A61K 47/549; A61P 35/00; C07F 9/6518; C07F 9/65583

(57) ABSTRACT

Compounds of Formula (IA)

(Targeting Moiety)-(Linker)-(Protease Ligand) (IA),
where the targeting moiety is an oligonucleotide capable of binding a target protein and the protease ligand is a ligand capable of binding a protease, and methods for the use thereof are provided. Also provided are compounds of Formula (IB)

(Targeting Moiety)-(Linker)-(Protease Ligand or E3 Ligase Ligand) (IB),
where the targeting moiety is an oligonucleotide capable of binding a target protein, the protease ligand is a ligand capable of binding a protease, and the E3 ligase ligand is a ligand capable of binding an E3 ligase, and methods for the use thereof are provided.

### CROSS-REFERENCE TO RELATED APPLICATIONS

This application is a National Stage application under 35 U.S.C. § 371 of International Application No. PCT/US2022/017931, having an International Filing Date of Jan. 25, 2022, which claims the benefit of U.S. Patent Application Ser. No. 63/153,872, filed on Feb. 25, 2021, U.S. Patent Application Ser. No. 63/158,218, filed on Mar. 8, 2021, and U.S. Patent Application Ser. No. 63/271,534, filed on Oct. 25, 2021. The disclosures of the prior applications are considered part of (and are incorporated by reference in) the disclosure of this application.

### SEQUENCE LISTING

The instant application contains a Sequence Listing which has been submitted electronically in ASCII format and is hereby incorporated by reference in its entirety. Said ASCII copy, created on Apr. 8, 2022, is named 07039-2085WO1_SL.txt and is 106,211 bytes in size.

### BACKGROUND

### 1. Technical Field

This document relates to the field of double-stranded or single-stranded oligonucleotide-based proteolysis targeting chimera (O′PROTAC) molecules that are useful for degrading target proteins related to all therapeutic areas.

### 2. Background Information

Conventional PROTACs (PROteolysis-TArgeting Chimeras) are hetero-bifunctional small molecules composed of a warhead and an E3 ligase ligand, thus recruiting E3 ligases to a protein of interest (POI) and inducing its degradation through the proteasome pathway. PROTAC technology has been greatly advanced during last decade. It has proven that PROTACs are capable of degrading varieties of proteins, including enzymes and receptors (Burslem et al., J. Am. Chem. Soc., 140(48):16428-16432 (2018); Cromm et al., J. Am. Chem. Soc., 140(49):17019-17026 (2018); Wang et al., Acta Pharmaceutica Sinica B, 10(2): 207-238 (2020); Sakamoto et al., Proc. Natl. Acad. Sci. USA, 98(15):8554-8559 (2001); Khan et al., Nat. Med, 25(12):1938-1947 (2019)). PROTACs offer several advantages over other small molecule inhibitors including expanding target scope, improving selectivity, reducing toxicity and evading inhibitor resistance, suggesting that PROTAC technology is a new promising modality to tackle diseases, in particular for cancer (Pettersson et al., Drug Discov. Today Technol., 31:15-27 (2019)). Despite their intriguing capabilities, PROTACs have some limitations. Most of the reported PROTACs are designed based on the currently existing small molecules targeting POI, which makes it difficult to apply to “undruggable” targets like transcription factors (TFs), which in general lack a ligand binding pocket. Additionally, due to their high molecular weight (˜600-1400 Da), PROTACs often suffer from poor cell permeability, stability, and solubility (Edmondson et al., Bioorg. Med Chem. Lett., 29(13):1555-1564 (2019)). In comparison with classic small molecule drugs, PROTACs are significantly less druggable.

### SUMMARY

Oligonucleotide drug development has become a main stream for new drug hunting in the last decade (Sridharan et al., Br J. Clin. Pharmacol, 82(3):659-672 (2016)). The catalytic advantage of PROTACs (Lai et al., Nat. Rev. Drug Discov., 16(2):101-114 (2017)) incorporated into oligonucleotide drugs could further fuel the field. Moreover, the delivery of oligonucleotide drugs has been advanced significantly in the recent years, notably for mRNA COVID-19 vaccines (Roberts et al., Nat. Rev. Drug. Discov., 19(10):673-694 (2020); and Chung et al., Adv. Drug Deliv. Rev., 170:1-25 (2020)). Therefore, O′PROTACs can be a complementary drug discovery and development platform to conventional PROTACs to derive clinical candidates and accelerate drug discovery.

One aspect of this document features a bifunctional compound (also referred to herein as a “degrader” or “O′PROTAC”), which has a structure represented by Formula (IA):

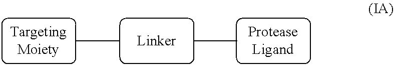

wherein the targeting moiety represents an oligonucleotide that can be recognized by a target protein, the protease ligand represents a ligand that binds a protease, and the linker represents a moiety that links the targeting moiety to the protease ligand, or a pharmaceutically acceptable salt or stereoisomer thereof.

Another aspect of this document features a bifunctional compound (also referred to herein as a “degrader” or “O′PROTAC”), which has a structure represented by Formula (IB):

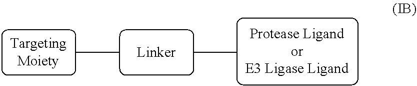

wherein the targeting moiety represents an oligonucleotide that can be recognized by a target protein, the protease ligand represents a ligand that binds a protease, the E3 ligase ligand represents a ligand that binds an E3 ligase, and the linker represents a moiety that links the targeting moiety to the protease ligand or E3 ligase ligand, or a pharmaceutically acceptable salt or stereoisomer thereof.

Another aspect of this document features a pharmaceutical composition containing a therapeutically effective amount of a compound of Formula (IA) or (IB), or a pharmaceutically acceptable salt or stereoisomer thereof, and a pharmaceutically acceptable carrier.

A further aspect of this document features a method of treating a disease or disorder mediated by aberrant (e.g., dysregulated or dysfunctional) protein activity, which includes administering a therapeutically effective amount of a bifunctional compound of Formula (IA) or Formula (IA), or a pharmaceutically acceptable salt or stereoisomer thereof, to a subject in need thereof.

Further aspects of this document feature methods of making the bifunctional compounds.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF THE DRAWINGS

FIG. 1. A working scheme of O′PROTAC (also referred to as OP) according to some embodiments.

FIGS. 2A-2C. ERG O′PROTACs degrade ERG protein in cultured cells. (FIG. 2A) 293T cells were transfected with FITC-labelled ERG O′PROTAC-13 (100 nM and 1,000 nM), and the transfection efficiency was monitored 48 hours post-transfection using a fluorescent microscope. Scale bar: 50 μm. (FIG. 2B) 293T cells were transfected with HA-ERG plasmid and a control or six indicated ERG O′PROTACs (100 nM) and harvested for western blot analysis 48 hours post-transfection. ERK2 was used as a loading control. (FIG. 2C) VCaP cells were transfected with a control or six indicated ERG O′PROTACs (100 nM), and cells were harvested for western blot analysis 48 hours post-transfection. Both endogenous full-length (FL) wild-type and TMPRSS2-ERG (T2-ERG) truncated ERG were detected.

FIG. 3. ERG O′PROTAC promotes ERG degradation via the protostome pathway. VCaP cells were transfected with increasing concentrations of ERG O′PROTAC-13 for 36 hours, followed by treatment of the proteinase inhibitor MG132 (20 μM) for 12 hours and western blot analysis.

FIG. 4. ERG O′PROTACs bind to ERG. 293T cells were transfected with HA-ERG plasmid in combination with control (non-biotin labelled) or six indicated biotin-labelled ERG O′PROTACs (100 nM) and harvested for anti-biotin (streptavidin) pull-down assay 48 hours post-transfection.

FIGS. 5A-5B. ERG O′PROTAC inhibits ERG transcriptional activity. (FIG. 5A) VCaP cells were transfected with 100 nM of biotin-labelled ERG O′PROTAC-13. Cells were harvested at the different time points followed by western blot analysis. (B and C) VCaP cells were transfected with different concentrations of biotin-labelled ERG O′PROTAC-13 and harvested 45 hours post-transfection for western blot analysis (FIG. 5B) and RT-qPCR analysis of mRNA expression of the indicated ERG-targeted genes (ADAM19, MAMP3, AIP9, PLAT and PLAU). P values were calculated using the unpaired two-tailed Student's t-test; * P<0.05; ** P<0.01; *** P<0.001, n.s., not significant.

FIG. 6. LEF-1 O′PROTACs degrade LEF1 protein in cultured cells. PC-3 cells were transfected with a control (500 nM) or six indicated LEF1 O′PROTAC at different concentrations (100 and 500 nM), and cells were harvested for western blot analysis 48 hours post-transfection. ERK2 was used as a loading control.

FIGS. 7A-7F. LEF1 O′PROTAC inhibits LEF1 target gene expression and prostate cancer cell proliferation. (FIGS. 7A-7C) PC-3 cells were transfected with a control (500 nM) or different concentrations of LEF1 O′PROTAC-45. At 48 hours post-transfection, cells were harvested for western blot analysis (Figure A), RT-qPCR analysis of mRNA expression of LEF1 targeted genes (CCND1 and c-MYC) (FIG. 7B), and MTS assay at different days after treatment (FIG. 7C). (FIGS. 7D-7F) DU145 prostate cancer cells were transfected with a control (500 nM) or different concentrations of LEF1 O′PROTAC-45. Transfected cells were subjected to western blot (FIG. 7D), RT-qPCR (FIG. 7E) and MTS assay (FIG. 7F). P values were calculated using the unpaired two-tailed Student's t-test; * P<0.05; ** P<0.01; *** P<0.001, n.s., not significant.

FIGS. 8A-8J. LEF1 OP-V1 inhibits prostate cancer tumor growth in vivo. (FIG. 8A) Photos of PC-3 xenograft tumors from the indicated groups of mice at 18 days after treatment with 1×PBS, control O′PROTACs (OP), or LEF1 OP-V1. (FIG. 8B) PC-3 tumor growth was measured at indicated time points after treatment with 1×PBS, control OP, or LEF1 OP-V1. Data represents means±SD (n=6). P values were determined using the unpaired two-tailed Student's t-test at day 18. n.s., not significant; *** P<0.001. (FIG. 8C) Photos of DU145 xenograft tumors from the indicated groups of mice at 21 days after treatment with 1×PBS, control OP, or LEF1 OP-V1. (FIG. 8D) DU145 tumor growth was measured at indicated time points after treatment with 1×PBS, control OP, or LEF1 OP-V1. Data represents means±SD (n=6). P values were determined using the unpaired two-tailed Student's t-test at day 18. n.s., not significant; *** P<0.001. (FIG. 8E) Body weight of mice was measured at different time points after the indicated treatments in PC-3 or DU145 xenograft. Data represents means±SD (n=6). (FIG. 8F) PC-3 and DU145 xenograft tumors were harvested from mice at day 18 or day 21, respectively, and their weight was measured. Data represents means±SD (n=6). P values were determined using the unpaired two-tailed Student's t-test. n.s., not significant; *** P<0.001. (FIG. 8G) Western blot analysis of expression of LEF1, cyclin D1, and c-MYC protein in PC-3 xenograft tumors. (FIG. 8H) RT-qPCR analysis of mRNA level of LEF1, CCND1, and c-MYC genes in PC-3 xenograft tumors. Data represents means±SD (n=6). P values were determined using the unpaired two-tailed Student's t-test. n.s., not significant; *** P<0.001. (FIG. 8I) Representative images of IHC of LEF1, Ki67, and cleaved caspase-3 in PC-3 xenograft tumors harvested from mice at 18 days after treatment with 1×PBS, control OP, or LEF1 OP-V1. (FIG. 8J) The quantification data of the LEF1, Ki67, and cleaved caspase-3 IHC. Data represents means±SD (n=6). P values were determined using the unpaired two-tailed Student's t-test. n.s., not significant; *** P<0.001.

FIGS. 9A-9M. ERG O′PROTAC induces ERG protein degradation. (FIG. 9A) A schematic diagram for ERG O′PROTACs that includes an ERG binding consensus sequence as identified from a forward ERG O′PROTAC sequence (SEQ ID NO:3) and a reverse ERG O′PROTAC sequence (SEQ ID NO:419). (FIG. 9B) VCaP cells were transfected with control or seven indicated ERG O′PROTACs (100 nM) for 36 hours and harvested for western blot analysis. ERK2 was used as a loading control. (FIG. 9C) A schematic diagram for ERG OP-C-N1 structure. (FIG. 9D) Biotin-labeled ERG OP-C-N1 (100 nM) was incubated with VCaP nuclear extract in the presence of an increasing amount of the unlabeled counterparts (1-, 10-, and 100-fold higher than the concentration of biotin-labeled probe) followed by EMSA. (FIG. 9E) Biotin-labeled ERG OP-C-N1 was incubated with VCaP nuclear extract and an increasing amount of ERG antibody, followed by EMSA. (FIG. 9F) VCaP cells were transfected with control OP, ERG OP-C-N1 (100 nM), or OP-C-A1 (100 nM) for 36 hours, followed by treatment of the proteasome inhibitor MG132 (20 μM) for 12 hours and western blot analysis. (FIG. 9G) VCaP cells were transfected with control OP or ERG OP-C-N1 at a final concentration of 100 nM for 36 hours and incubated with 1-, 25- or 50-fold of CRBN ligand pomalidomide, followed by western blot analysis of ERG expression. (FIGS. 9H and 91) 293T (H) and VCaP cells (FIG. 91) were treated with ERG OP-C-N1 (100 nM) for 36 hours and the proteasome inhibitor MG132 (20 μM) for 12 hours before harvested for western blot analysis of ERG ubiquitination. (FIGS. 9J and 9K) VCaP cells were cultured in Matrigel for five days followed by the treatment of 200 nM of ERG OP-C-N1 for another five days. The representative images with 3D spheres are shown in (FIG. 9J), and the quantification data of the diameters of the 3D spheres are shown in (FIG. 9K). Data represents means±SD (n=50). P values were determined using the unpaired two-tailed Student's t-test; *** P<0.001. (FIGS. 9L and 9M). 22Rv1 cells transfected with ERG expression plasmid and 100 nM of ERG OP-C-N1 were plated onto Matrigel-coated transwells for 48 hours. The invaded cells were stained with crystal violet. Photos are shown in (FIG. 9L) and the quantification data are shown in (FIG. 9M). Data represents means±SD (n=4). P values were determined using the unpaired two-tailed Student's t-test. *** P<0.001.

FIGS. 10A-10D. ERG OP-C-N1 degrades ERG protein in a time- and dose-dependent manner. (FIG. 10A) VCaP cells were transfected with a final concentration of 100 nM and harvested at different time points, followed by Western blot to detect ERG expression. (FIG. 10B) VCaP cells were transfected with increasing concentrations of ERG OP-C-N1 for 36 hours, followed by western blot to detect ERG expression. (FIGS. 10C and 10D) VCaP cells were transfected with increasing concentrations of ERG OP-C-N1 for 24 hours and treated with 20 μg/mL cycloheximide (CHX) for another 12 hours, followed by Western blot to detect ERG expression (FIG. 10C). The remaining ERG protein (%) was calculated by normalizing the values to that in the group without ERG OP-C-N1 treatment, and DC₅₀ was determined (FIG. 10D). This experiment was repeated once and similar results were obtained.

FIGS. 11A-11E. ERG O′PROTACs degrade ERG oncoprotein. (FIG. 11A) The 293T cells were transfected with pCMV-HA-ERG plasmid and control or six indicated ERG O′PROTACs (100 nM) and harvested for western blot analysis 48 hours post-transfection. ERK2 was used as a loading control. C stands for CRBN-based OP, while V stands for VHL. (FIG. 11B) The VCaP cells were transfected with control or six indicated ERG O′PROTACs (100 nM) and harvested for western blot analysis 48 hours post-transfection. Both endogenous full-length (FL) (wild-type) and TMPRSS2-ERG (T1/E4, truncated) were detected. (FIG. 11C) The HPLC spectrum of ERG OP-C-P1 detected by UV at 260 nm. (FIG. 11D) Deconvoluted mass spectrum of ERG OP-C-P1. (FIG. 11E) The line drawn structure of ERG OP-C-P1 where the forward (F) sequence includes SEQ ID NO: 3 and the reverse (R) sequence includes SEQ ID NO:419.

FIGS. 12A-12H. Phthalic acid-based ERG O′PROTAC degrades ERG oncoprotein. (FIGS. 12A and 12B) FITC-labeled ERG O′PROTACs, including ERG OP-C-P1, ERG OP-C1, OP-C-A1 and C-N1 were individually transfected into 293T (FIG. 12A) and VCaP cells (FIG. 12B) at a final concentration of 100 nM with Lipofectamine 2000. ERG OP-C-A1 and C-N1 served as positive controls. Parental cells without any transfection were used as a negative control. Representative images of bright (upper) and fluorescent (lower) fields for indicated O′PROTACs are shown. Scale bar: 50 μm. (FIG. 12C) The 293T cells were transfected with control or four indicated ERG O′PROTACs at a final concentration of 100 nm and harvested for western blot analysis. (FIGS. 12D and 12E) The VCaP cells were transfected with control or four indicated ERG O′PROTACs at a final concentration of 100 nm and harvested for western blot analysis (FIG. 12D) or RT-qPCR to detect the mRNA level of ERG FL and T1/E4 (FIG. 12E) after 48 hours post-transfection. Data represents means±SD (n=3). The P values were determined using the unpaired two-tailed Student's t-test. n.s. represents not significant by comparing the values in ERG O′PROTAC-treated groups to the control OP-treated group. (FIG. 12F) The VCaP cells were transfected with ERG OP-C-P1 at a final concentration of 100 nM and harvested at different time points, followed by western blot analysis of ERG protein expression. (FIGS. 12G and 12H) The VCaP cells were transfected with increasing concentrations of ERG OP-C-P1 for 36 hours and treated with 20 μg/mL of cycloheximide (CHX) for another 12 hours. (FIG. 12G) Cells were harvested for western blot analysis of ERG protein expression. The remaining ERG protein (%) was calculated by normalizing the value of each group to that of the group without ERG OP-C-P1 treatment. (FIG. 12H) The concentration of ERG OP-C-P1 degrading 50% of ERG protein (DC₅₀) was calculated with Prism software.

FIGS. 13A-13G. Phthalic acid-based ERG O′PROTAC degrades ERG via CRBN and the proteasome pathway. (FIG. 13A) The VCaP cells were transfected with a final concentration of 100 nM of control OP or ERG OP-C-P1 for 36 hours and treated with or without MG132 (20 μM) for another 12 hours before harvested for western blot analysis. (FIGS. 13B and 13C) The 293T (FIG. 13B) and VCaP cells (FIG. 13C) were transfected with the indicated plasmids and ERG OP-C-P1 at a final concentration of 100 nM for 36 hours and treated with the proteasome inhibitor MG132 (20 μM) for 12 hours before harvested for protein extraction. ERG protein was immunoprecipitated with HA (FIG. 13B) or ERG antibody (FIG. 13C) by protein A/G beads to detect its ubiquitination level by western blot analysis. (FIG. 13D) Biotin-labeled ERG OP-C-P1 (100 nM) was incubated with VCaP nuclear extract in the presence of an increasing amount of the unlabeled counterparts (1-, 10-, and 100-fold higher than the concentration of biotin-labeled probe) followed by electrophoretic mobility shift assay (EMSA). DPC stands for DNA-protein complex. (FIG. 13E) Biotin-labeled ERG OP-C-P1 was incubated with VCaP nuclear extract in the presence of increasing amounts of ERG antibody (0.5 and 1 μg), followed by EMSA. (FIG. 13F) The VCaP cells were transfected with a final concentration of 100 nM of control OP or ERG OP-C-P1 and siRNA control (siNS) or siCRBN for 48 hours before harvested for western blot analysis. (FIG. 13G) The VCaP cells were transfected with control OP or ERG OP-C-P1 at a final concentration of 100 nM and incubated with 1-, 10-, or 50-fold of CRBN ligand pomalidomide for 36 hours, followed by western blot analysis of ERG protein level.

FIGS. 14A-14F. Phthalic acid-based ERG OP inhibits ERG target gene expression and prostate cancer cell growth and invasion. (FIGS. 14A and 14B) The VCaP cells were transfected with control OP or ERG OP-C-P1 a final concentration of 100 nM for 48 hours and harvested for western blot analysis (FIG. 14A) and RT-qPCR for the indicated ERG target genes (FIG. 14B). Data represents means±SD (n=3). P values were determined using the unpaired two-tailed Student's t-test; * P<0.05; ** P<0.01. (FIGS. 14C and 14D) The VCaP cells were embedded in matrigel and cultured for 5 days, followed by the treatment of 200 nM of control OP or ERG OP-C-P1 for another 5 days. The representative images with three-dimension (3D) spheres are shown in (FIG. 14C) and the quantified diameters of 3D spheres are shown in (FIG. 14D). Data are demonstrated with box and whiskers; whiskers represent min to max, and each point is one value of an individual 3D sphere (n=50). The P value was determined using the unpaired two-tailed Student's t-test; *** P<0.001. (FIGS. 14E and 14F) The 22Rv1 cells were transfected with pCMV-HA-ERG and 100 nM of control OP or ERG OP—C-P1, followed by plating on matrigel-coated chambers and incubating for 48 hours in 37° C. incubator. The invaded cells were stained with 0.5% of crystal violet. The representative fields are shown in (FIG. 14E) and the quantification data are shown in (FIG. 14F). Data represents means SD (n=4). The P value was determined using the unpaired two-tailed Student's t-test. ** P<0.01.

FIG. 15. MEME-ChIP DNA motif analysis in 416 gain of function (GOF) p53 ChIP-seq peaks in VCaP cells. Motif sequences shown, from top to bottom.

FIGS. 16A-16I. Transcriptional regulation of CTNNB1 by GOF p53 mutants. (FIG. 16A) p53 ChIP-seq data showing the distribution of p53 R248W mutant binding peaks in VCaP cells. (FIG. 16B) KEGG pathway analysis of p53-occupied genes revealed by ChIP-seq in VCaP cells. (FIG. 16C) UCSC Genome Browser screenshots showing the occupancy of p53 R248W mutant in the CTNNB1 promoter in VCaP cells. (FIG. 16D) ChIP-qPCR analysis of p53 R248 binding at the CTNNB1 promoter in VCaP cells. **, p<0.01. (FIG. 16E) Scheme showing the locations of p53 ChIP-qPCR amplicons and EMSA DNA probes in the CTNNB1 promoter region. (FIG. 16F) ChIP-qPCR analysis of p53 R248 binding at the CTNNB1 promoter in VCaP cells using three sequential pairs of primers shown in (FIG. 16E). (FIG. 16G) EMSA assay using DNA probes from the CTNNB1 promoter indicated in (FIG. 16E) and nuclear extract from VCaP cells. DPC, DNA-protein complex. (FIG. 16H) EMSA assay using biotin-labeled and unlabeled DNA probe 1 shown in (FIG. 16E) and nuclear extract from VCaP cells. (FIG. 16I) Top, scheme showing the p53 missense mutants used in experiments. Bottom, results of EMSA assay using DNA probe 1 shown in (FIG. 16E, showing a MP53BS sequence of SEQ ID NO:106) and GST recombinant proteins for p53WT or indicated mutants purified from bacteria.

FIGS. 17A-17C. GOF p53 mutants bind CTNNB1 gene promoter and regulate gene expression. (FIG. 17A) UCSC Genome Browser screenshots showing ChIP-seq results of p53 WT and DNA binding domain (DBD) mutants (R273H, R249S, R248Q) from the indicated breast cancer cell lines, showing a MP53BS sequence of SEQ ID NO:106. (FIG. 17B) EMSA assay using DNA probe 1 from the CTNNB1 promoter and nuclear extract from VCaP cells in the presence or absence of anti-p53 antibody. DPC, DNA-protein complex. Supershift indicates the DNA-protein-antibody complex. (FIG. 17C) DNA sequence alignment among WT p53 binding consensus motif (SEQ ID NO: 383), MP53BS in human (SEQ ID NO:106) and mouse (SEQ ID NO: 442) CTNNB1 gene promoter and the MP53BS-like sequences in other GOF p53 mutant (R248W) binding targets KAT6A (SEQ ID NO:398), KMT2A (SEQ ID NO:403), MCLI (SEQ ID NO:408), andMED23 (SEQ ID NO:413) (MUT p53 CTNNB1 sequence disclosed as SEQ ID NO: 449).

FIGS. 18A-18N. LEF1/TCF O′PROTAC inhibits pyrimidine synthesis gene (PSG) expression and PCa patient-derived xenograft (PDX) growth in vivo. (FIG. 18A) DNA oligonucleotides used for LEF1/TCF O′PROTAC (OP; SEQ ID NO:5) and its sequence alignment with the consensus sequence of the DNA motif element of the members of LEF/TCF family, LEF1, TCF1, TCF3, and TCF4. (FIG. 18B) Western blot analysis of indicated proteins in VCaP cells treated with control or LEF1/TCF O′PROTAC for 48 hours. (FIG. 18C) MTS assay in VCaP cells treated with control or LEF1/TCF OP. (FIG. 18D) Sanger sequencing confirmation of C238Y mutation in LuCaP 23.1 PDX tumor samples. (FIG. 18E) Western blot analysis of indicated proteins in organoids derived from LuCaP 23.1 PDXs (PDXO). (FIGS. 18F-18H) LuCaP 23.1 PDXOs were treated with indicated O′PROTAC and/or deoxynucleotides and harvested for Western blot analysis 48 hours after treatment (FIG. 18F) or cultured for 3 days followed by photographing (FIG. 18G) and quantification of the diameters of organoids (FIG. 18H). Data shown as means±S.D. (n=60 organoids from three independent experiments/group). Two-tailed Students' t test was performed. ***, p<0.001. n.s., not significant. (FIG. 18I) Representative images of LuCaP 23.1 PDX tumors in mice at 21 days after treatment with vehicle or indicated OP. (FIG. 18J) Growth curve of LuCaP 23.1 PDX in mice treated with vehicle or indicated OP. Data shown as means±S.D. (n=6). ***, p<0.001. n.s., not significant. (FIG. 18K) Weight of LuCaP 23.1 PDX tumors in mice at 21 days after treatment with vehicle or indicated OP. Data shown as means±S.D. (n=6). ***, p<0.001. (FIG. 18L) Body weight of mice at 21 days after treatment with vehicle or indicated OP. Data shown as means±S.D. (n=6). n.s., not significant. (FIG. 18M) Representative IHC images of indicated proteins from tumors shown in (FIG. 18I). (FIG. 18N) Quantification of IHC staining of indicated proteins. See details of staining scoring and index in Example 13. Data shown as means±S.D. (n=3 sections/group). ***, p<0.001.

FIG. 19. A scheme of a synthesis route of O′PROTAC according to some embodiments.

FIG. 20. A scheme of a synthesis route of O′PROTAC according to some embodiments.

FIGS. 21A-21D. HPLC and mass spectrum of oligonucleotides. (FIG. 21A) The HPLC spectrum of ERG-R-OP-C1 detected by UV at 260 nm. (FIG. 21B) The mass spectrum of ERG OP-R-C1. The deconvoluted mass is shown in the upper right corner. (FIG. 21C) The HPLC spectrum of ERG-F-FITC detected by UV at 260 nm. (FIG. 21D) The deconvoluted mass spectrum mass spectrum of ERG-F-FITC.

FIGS. 22A-22B. Docking model of CRBN bound by thalidomide (FIG. 22A) and 3-N-substituted-aminophthalic acid (FIG. 22B). Dotted black lines represent hydrogen bond and dotted cyan lines represent pi-pi interaction.

FIGS. 23A-23G. Clinically relevant co-expression of TMPRSS2-ERG and p53 mutant induces prostate tumorigenesis in mice. (FIG. 23A) OncoPrint image from cBioPortal showing the percentage of genetic alterations in the ERG and TP53 genes in PCa patients from TCGA (top) and SU2C (low) cohorts. (FIG. 23B) Fisher exact test (two-tailed) of the association between TMPRRS2-ERG fusion and TP53 alteration in TCGA (left) and SU2C (right) PCa patient samples. (FIG. 23C) Representative images of H&E and IHC of ERG, AR and Ki67 proteins in prostate tissues from mice with the indicated genotypes at 15 months of age. (FIG. 23D) Quantification of incidences of PIN and/or cancer in mice with indicated genotypes shown in (FIG. 23C). ***, p<0.001. (FIG. 23E) Quantification of Ki67 positive cells from tissue sections in (FIG. 23C). ***, p<0.001. (FIG. 23F) Western blot analysis of indicated proteins in VCaP cells stably expressing the indicated shRNAs. ERK2 was used as a loading control. (FIG. 23G) MTS assay in VCaP cells stably expressing the indicated shRNAs. ***, p<0.001. n.s., nonsignificant.

FIGS. 24A-24I. Expression of pyrimidine synthesis genes (PSGs) is co-regulated by ERG and GOF p53 mutants in murine prostate tumors and human PCa cells. (FIG. 24A) Venn diagram showing the overlap between the genes uniquely upregulated in prostate tissues from Pb-ERG; Trp53^{R172H/-} mice (n=3, 15 months) and those from Pb-ERG; Trp53^{-/-} mice (n=3, 15 months) revealed by RNA-seq data. (FIG. 24B) Venn diagram showing the overlap of the genes uniquely upregulated in the prostate tissues from Pb-ERG; Trp53^{R172H/-} mice (n=3, 15 months) with ERG bound target genes revealed by ChIP-seq in murine PCa (GSM1145303). (FIG. 24C) Heatmap of RNA-seq data showing a subset of genes (n=531) differentially expressed in the prostate tissues of mice (15 months) with the indicated genotypes (n=3 except Trp53^{pcR172H/-} group). (FIG. 24D) KEGG pathway analysis of 531 ERG target genes uniquely upregulated in prostate tissues from Pb-ERG; Trp53^{R172H/-} mice shown in (FIG. 24C). (FIG. 24E) Diagram elucidating key pyrimidine synthesis enzymes including UMPS, RRM1, RRM2 and TYMS. (FIG. 24F) UCSC Genome Browser screenshots showing the results in the Umps gene locus of RNA-seq in the prostate tissues from Pb-ERG; Trp53^{R172H/-} mice shown in (FIG. 24C) and ERG ChIP-seq (GSM1145303). (FIG. 24G) RT-qPCR analysis of expression of PSGs in prostate tissues of the indicated mouse types (n=3, 15 months). **, p<0.01. (FIGS. 24H and 241) Western blot (FIG. 24H) and RT-qPCR (FIG. 24I) analyses of indicated proteins and PSG gene mRNAs in VCaP cells stably expressing control or gene-specific shRNAs. ***, p<0.01, **, p<0.01.

FIGS. 25A-25L. Promoter binding and CTNNB1 gene expression regulation by GOF p53 mutants. (FIG. 25A) p53 ChIP-seq data showing the distribution of p53 R248W mutant binding peaks in VCaP cells. (FIG. 25B) KEGG pathway analysis of p53-occupied target genes revealed by ChIP-seq in VCaP cells. (FIG. 25C) UCSC Genome Browser screenshots showing the occupancy of p53 R248W mutant in the CTNNB1 gene promoter in VCaP cells. (FIG. 25D) ChTP-qPCR analysis of p53 R248 binding at the CTNNB1 promoter in VCaP cells. **, p<0.01. n.s., not significant. (FIG. 25E) Scheme showing the locations of p53 ChIP-qPCR amplicons and EMSA DNA probes in the CTNNB1 promoter region. (FIG. 25F) ChIP-qPCR analysis of p53 R248 binding at the CTNNB1 promoter in VCaP cells using three sequential pairs of primers shown in (FIG. 25E). **, p<0.01. n.s., not significant. (FIG. 25G) EMSA assay using double-stranded (ds) DNA probes from the CTNNB1 promoter indicated in (FIG. 25E) and nuclear extract from VCaP cells. DPC, DNA-protein complex. (FIG. 25H) EMSA assay using biotin-labeled and unlabeled ds DNA probe 1 shown in (FIG. 25E) and nuclear extract from VCaP cells. (FIG. 25I) Top, scheme showing the p53 missense mutants used in experiments. Bottom, results of EMSA assay using ds DNA probe 1 shown in (FIG. 25E, showing a MP53BS sequence of SEQ ID NO:106) and GST recombinant proteins for p53 WT or indicated mutants purified from bacteria. (FIGS. 25J and 25K) Western blot (FIG. 25J) and RT-qPCR (FIG. 25K) analyses of indicated proteins and mRNAs in VCaP cells stably expressing the indicated shRNAs. **, p<0.01. ***, p<0.001. (FIG. 25L) Meta-analysis of RNA-seq data showing the expression of CTNNB1, MDM2 (p53 canonical target, positive control) and ACTB (non-specific internal control) mRNA levels in PCa patient samples of the SU2C cohort with p53 wild-type (WT), loss (null) and mutation (Mut) in the DBD domain. **, p<0.01. *, p<0.05. n.s., not significant.

FIGS. 26A-260. Co-regulation of PSG expression by ERG and β-Catenin. (FIGS. 26A and 26B) Western blot (FIG. 26A) and RT-qPCR (FIG. 26B) analysis of indicated proteins and mRNAs in VCaP cells stably expressing indicated shRNAs. ***, p<0.001. **, p<0.01. *, p<0.05. (FIG. 26C) UCSC Genome Browser screenshots showing occupancy of ERG and β-Catenin in UMPS and RRM2 gene loci revealed by ERG ChIP-seq in VCaP cells and β-Catenin ChIP-seq (GSE53927). (FIGS. 26D and 26E) ChIP-qPCR analysis of occupancy of ERG (FIG. 26D) and β-Catenin (FIG. 26E) at UMPS, RRM1, RRM2 and TYMS gene loci in VCaP cells. ***, p<0.001. **, p<0.01. (FIG. 26F) ChIP-qPCR analysis of ERG and β-Catenin co-occupancy at the UMPS gene promoter. ***, p<0.001. (FIGS. 26G and 26H) Western blot (FIG. 26G) and RT-qPCR (FIG. 26H) analysis of indicated proteins and mRNAs in p53 KO DU145 cells expressing indicated plasmids and/or shRNAs. **, p<0.01. (FIG. 26I) Chromosome Conformation Capture (3C) assay for analysis of chromatin interaction between ERG- and β-Catenin-occupied sites in the RRM2 locus in p53 KO DU145 cells expressing indicated plasmids and/or shRNAs. **, p<0.01. (FIG. 26J) A hypothetical model depicting the probable spacial interaction in PSG loci. (FIG. 26K) Western blot analysis of indicated proteins in VCaP cells expressing indicated shRNAs. (FIGS. 26L and 26M) Representative chromatograms (FIG. 26L) and quantitative data (FIG. 26M) showing the levels of UDP and dTDP measured by LC-MS in VCaP cells with co-depletion of ERG and p53 proteins as in (FIG. 26K). *, p<0.05; **, p<0.01. (FIG. 26N) Western blot analysis of UMPS, RRM1 and RRM2 proteins in VCaP cells expressing indicated sgRNAs. (FIG. 26O) MTS assay in VCaP cells with depletion of indicated proteins as in (FIG. 26N). Two-way ANOVA was performed. ***, p<0.001.

FIGS. 27A-27M. CBP PROTAC inhibits PSG expression and PCa xenograft growth in mice. (FIG. 27A) Meta-analysis of RNA-seq data showing the association of increased expression of UMPS, RRM1 and RRM2 with high level of CTNNB1 mRNA in TMPRRS2-ERG fusion-positive PCa samples of the TCGA cohort. (FIG. 27B) Kaplan-Meier Survival curve showing the association of high mRNA expression of three PSGs (UMPS, RRM1 and RRM2) with poor survival of TMPRRS2-ERG fusion-positive PCa samples of the TCGA cohort. Log-rank (Mantel-Cox) was used. (FIG. 27C) Strategy of inhibition of β-Catenin's transcriptional activity via CBP PROTACs. (FIG. 27D) The linear structures of CBP PROTACs (CP1 to CP4) used in the study. (FIG. 27E) Western blot analysis of CBP and β-Catenin proteins in VCaP cells treated with ICG-001 or CBP PROTACs. (FIG. 27F) VCaP cells were treated with CP2 for 36 hours and MG132 for 8 hours and harvested for IP and Western blots with indicated antibodies. (FIG. 27G) VCaP cells were treated with CP2 for 36 hours and MG132 for 8 hours followed by Western blots with indicated antibodies. (FIGS. 27H and 271) VCaP cells were treated with vehicle or two doses of CP2 for 48 hours and harvested for RT-qPCR (FIG. 27H) and Western blot (FIG. 27I) analysis of indicated genes or proteins. ***, p<0.001. **, p<0.01. *, p<0.05. (FIG. 27J) MTS assay in VCaP cells treated with CP2 at different doses. Two-way ANOVA was performed. ***, p<0.001. (FIG. 27K) MTS assay in VCaP cells treated with CP2 and/or indicated deoxynucleotides. *, p<0.05; ***, p<0.001; n.s., not significant. (FIG. 27L) Representative images of tumors isolated from mice at 23 days after the indicated treatment. (FIG. 27M) Tumor growth curve in mice treated with vehicle, ICG-001 and CP2. Data shown as means±S.D. (n=5 tumors/group). Two-way ANOVA was performed. ***, p<0.001. **, p<0.01.

FIGS. 28A-28N. LEF1/TCF O′PROTAC inhibits PSG expression and PCa PDX growth. (FIG. 28A) Sequence of the DNA oligonucleotide used in LEF1/TCF O′PROTAC (OP; SEQ ID NO:5) and its alignment with the consensus DNA binding elements of the members of LEF/TCF family, LEF1, TCF1, TCF3, and TCF4. (FIG. 28B) Western blot analysis of indicated proteins in VCaP cells treated with control or LEF1/TCF OP for 48 hours. (FIG. 28C) MTS assay in VCaP cells treated with control or LEF1/TCF OP. (FIG. 28D) Sanger sequencing confirmation of C238Y mutation in LuCaP 23.1 PDX tumor samples. (FIG. 28E) Western blot analysis of indicated proteins in organoids derived from LuCaP 23.1 PDXs (PDXO). (FIGS. 28F-28H) LuCaP 23.1 PDXOs were treated with indicated OP and/or deoxynucleotides and harvested for Western blot analysis 48 hours after treatment (FIG. 28F) or cultured for 3 days followed by photographing (FIG. 28G) and quantification of the diameters of organoids (FIG. 28H). Data shown as means±S.D. (n=60 organoids from three independent experiments/group). Two-tailed Students' t test was performed. ***, p<0.001. n.s., not significant. (FIG. 28I) Representative images of LuCaP 23.1 PDX tumors in mice at 21 days after treatment with vehicle or indicated OP. (FIG. 28J) Growth curve of LuCaP 23.1 PDX in mice treated with vehicle or indicated OP. Data shown as means±S.D. (n=6). ***, p<0.001. n.s., not significant. (FIG. 28K) Weight of LuCaP 23.1 PDX tumors in mice at 21 days after treatment with vehicle or indicated OP. Data shown as means±S.D. (n=6). ***, p<0.001. (FIG. 28L) Body weight of mice at 21 days after treatment with vehicle or indicated OP. Data shown as means±S.D. (n=6). n.s., not significant. (FIG. 28M) Representative IHC images of indicated proteins from tumors shown in (FIG. 28I). (FIG. 28N) Quantification of IHC staining of indicated proteins. See details of staining scoring and index in Methods. Data shown as means±S.D. (n=3 sections/group). ***, p<0.001.

FIG. 29. A hypothetical model deciphering the cooperativity of TMPRSS2-ERG and GOF p53 mutants in PCa development and progression. Co-expression of TMPRSS2-ERG and GOF p53 mutants drives pyrimidine synthesis gene (PSG) expression and PCa growth and progression via p53 mutant-dependent upregulation of CTNNB1 gene expression and the functional interaction of β-Catenin with ERG on chromatin at genomic loci of PSGs and other cancer related genes. The β-Catenin dependency can be pharmacologically targeted by CBP PROTAC and LEF1/TCF O′PROTAC for the treatment of ERG/GOF p53 mutant PCa.

FIGS. 30A-30E. Co-occurrence of TMPRSS2-ERG and p53 alteration in PCa patient samples and co-expression of ERG and GOF p53 mutant induces early onset of prostate tumors in mice, related to FIG. 23. (FIG. 30A) OncoPrint image from cBioPortal showing the percentage of genetic alterations in the ERG and TP53 genes in PCa patients from the MSKCC cohort. (FIG. 30B) Fisher exact test (two-tailed) of the association between TMPRRS2-ERG fusion and TP53 alteration in MSKCC PCa patient samples. (FIG. 30C) Representative images of H&E and IHC of ERG, AR and Ki67 proteins in prostate tissues from mice with the indicated genotypes at 10 months of age. (FIG. 30D) Quantification of incidences of PIN and/or cancer in mice with indicated genotypes shown in (FIG. 30C). **, p<0.01. (FIG. 30E) Quantification of Ki67 positive cells in prostate tissues from mice shown in (FIG. 30C). ** p<0.01.

FIGS. 31A-31F. Comparison of the genes uniquely upregulated in Pb-ERG; Trp53^{R172H/-}, Pb-ERG; Trp53^{-/-} and other genotypic mice, related to FIG. 2. (FIGS. 31A and 31B) Venn diagram showing the genes uniquely expressed in prostate tissues from indicated genotypic mice at 15 months of age revealed by RNA-seq data (n=3/group except Trp53^{R172H/-} group for which the data from one mouse were excluded from analysis due to poor quality). (FIGS. 31C-31E) UCSC Genome Browser screenshots showing the RNA-seq and ERG ChIP-seq (GSM1145303) data in RRM1 (FIG. 31C), RRM2 (FIG. 31D), and TYMS (FIG. 31E) gene loci. (FIG. 31F) MEME-ChIP DNA motif analysis in 416 p53 ChIP-seq peaks obtained from VCaP cells. Motif sequences shown, from top to bottom.

FIGS. 32A-32H. GOF p53 mutants bind CTNNB1 gene promoter and regulate (3-Catenin expression in different cancer cell lines, related to FIG. 25. (FIG. 32A) UCSC Genome Browser screenshots showing ChIP-seq results of p53 WT and GOF DBD mutants (R273H, R249S, R248Q) from the indicated breast cancer cell lines, showing a MP53BS sequence of SEQ ID NO:106. (FIG. 32B) Agarose gel (4%) electrophoresis of single-strand (ss) sense (S) and antisense (AS) oligos and annealed double-stranded (ds) DNA Probes used for EMSA. (FIG. 32C) EMSA assay using ds DNA probe 1 from the CTNNB1 promoter as shown in FIG. 3E and nuclear extract from VCaP cells in the presence or absence of anti-p53 antibody. DPC, DNA-protein complex. Supershift indicates the DNA-protein-antibody complex. (FIG. 32D) DNA sequence alignment among WT p53 binding consensus element (SEQ ID NO: 383), MP53BS in human (SEQ ID NO:106) and mouse (SEQ ID NO: 442) CTNNB1 gene promoter and the MP53BS-like sequences in other GOF p53 mutant (R248W) binding targets KAT6A (SEQ ID NO:398), KMT2A (SEQ ID NO:403), MCLI (SEQ ID NO:408), andMED23 (SEQ ID NO:413) (MUT p53 CTNNB1 sequence disclosed as SEQ ID NO: 449). (FIGS. 32E-32H) UCSC Genome Browser screenshots showing the occupation of p53 R248W mutant in the promoter ofKAT6A (FIG. 32E (SEQ ID NO: 398)), KMT2A (FIG. 32F (SEQ ID NO: 140)), MCLI (FIG. 32G (SEQ ID NO: 125)) and MED23 (FIG. 32H (SEQ ID NO: 244)) gene in VCaP cells.

FIGS. 33A-33I. Regulation of CTNNB1 mRNA expression by GOF p53 mutants in human PCa cell lines and mouse PCa tissues, related to FIG. 25. (FIGS. 33A and 33B) Western blot (FIG. 33A) and RT-qPCR (FIG. 33B) analysis of β-Catenin protein and mRNA in p53 mutated DU145 cells stably expressing control or p53-specific sgRNAs. ***, p<0.001. (FIGS. 33C and 33D) Western blot (FIG. 33C) and RT-qPCR (FIG. 33D) analysis of β-Catenin protein and mRNA in p53 WT LNCaP cells stably expressing control or p53-specific sgRNAs. n.s., not significant. (FIGS. 33E and 33F) p53 knockout (KO) DU145 cells were infected with lentivirus expressing empty vector (EV), WT p53 or the indicated mutants. Cells were harvested for Western blot analysis (FIG. 33E) and nuclear extract preparation for EMSA using ds DNA probe 1 from the CTNNB1 promoter as indicated in FIG. 25E (FIG. 33F). H3 was used as a loading control. (FIG. 33G) UCSC Genome Browser screenshots showing the Ctnnb1 mRNA level revealed by RNA-seq in different groups of the indicated genotypic mice at 15 months of age. (FIG. 33H) Quantitative data showing the RNA-seq reads of Ctnnb1 mRNA in prostate tumor tissues from WT and Pb-ERG; Trp53^{R172H/-} mice at 15 months of age (n=3/group). Log₁₀ (FPKM) was calculated for the expression of Ctnnb1 mRNA. Student's t-test was used to assess the significance. * p<0.05. (FIG. 33I) Top, UCSC Genome Browser screenshots showing the occupancy of ERG in the CTNNB1 gene promoter in VCaP cells. Bottom, two core elements of ERG binding sequence (ERGBS; SEQ ID NO:443) in red and MP53BS (SEQ ID NO:444) in blue are indicated.

FIGS. 34A-34E. Assessment of chromatin looping between ERG and β-catenin binding sites at PSG loci, related to FIG. 26. (FIGS. 34A-34B) UCSC Genome Browser screenshots showing the occupancy of ERG and β-catenin proteins at RRM1 (FIG. 34A) and TYMS (FIG. 34B) gene loci as revealed by ChIP-seq data. (FIGS. 34C and 34D) Chromosome Conformation Capture (3C) assay for analysis of chromatin interaction between the ERG- and β-catenin-occupied sites in the RRM2 (FIG. 34C) and TYMS (FIG. 34D) loci in p53 KO DU145 cells expressing indicated plasmids and/or shRNAs. **, p<0.01. (FIGS. 34E and 34F) p53 KO DU145 cells were transfected with indicated plasmids and/or infected lentivirus expression indicted shRNAs and cells were harvested for ChIP-qPCR analysis of the levels of H3K27ac (FIG. 34E) and Pol II-S2-p (FIG. 34F) at the indicated PSG loci. ***, p<0.001. **, p<0.01. *, p<0.05. n.s., not significant.

FIGS. 35A-35L. β-Catenin/CBP complex inhibitor effectively decreases PSG expression and TMPRSS2-ERG/p53 mutant-positive PCa cell growth, related to FIG. 27. (FIG. 35A) MTS assay in VCaP cells infected lentivirus expressing control (shCon) or O-Catenin-specific shRNAs. ERK2 was used as a loading control. ***, p<0.001. (FIGS. 35B and 35C) RT-qPCR (FIG. 35B) and Western blot (FIG. 35C) analysis of expression of indicated mRNAs and proteins in VCaP cells treated with vehicle or different doses of ICG-001. ***, p<0.001. **, p<0.01. *, p<0.05. (FIG. 35D) MTS assay in VCaP cells treated with vehicle or different doses of ICG-001. ***, p<0.001. (FIGS. 35E and 35F) RT-qPCR (FIG. 35E) and Western blot (FIG. 35F) analysis of expression of indicated mRNAs and proteins in VCaP cells treated with vehicle or different doses of PRI-724. ***, p<0.001. **, p<0.01. *, p<0.05. (FIG. 35G) MTS assay in VCaP cells treated with vehicle or different doses of PRI-724. ***, p<0.001. (FIG. 35H) Comparison of the weight of tumors obtained from mice at 23 days after treatment with vehicle, ICG-001 or CP2. ***, p<0.001. **, p<0.01. (FIG. 35I) MTS assay in VCaP cells treated with different doses of ICG-001 and CP2 for IC₅₀ determination. **, p<0.001. (FIG. 35J) Body weight of mice at 23 days after treatment with vehicle, ICG-001 or CP2. n.s., not significant. (FIG. 35K) Left, Representative IHC images of indicated proteins in tumors shown in FIG. 27L and, right, quantitative data of IHC intensity of each protein. See details in Methods for the calculation of staining index. ***, p<0.001. **, p<0.01. *, p<0.05. (FIG. 35L) Western blot analysis of indicated proteins in PDX tumors obtained from mice with indicated treatments (n=3 tumors/treatment). ERK2 was used as a loading control.

### DETAILED DESCRIPTION

In general, the bifunctional compounds described herein can have a structure represented by Formula (IA):

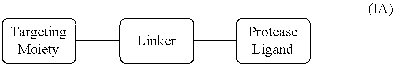

wherein the targeting moiety represents an oligonucleotide that can bind to a target protein, the protease ligand represents a ligand that binds to a protease, and the linker represents a moiety that connects the targeting moiety and the protease ligand, or a pharmaceutically acceptable salt or stereoisomer thereof.

In some cases, the bifunctional compound described herein can have a structure represented by Formula (IB):

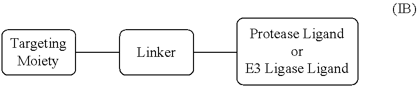

wherein the targeting moiety represents an oligonucleotide that can bind to a target protein, the protease ligand represents a ligand that binds to a protease, the E3 ligase ligand represents a ligand that binds an E3 ligase, and the linker represents a moiety that links the targeting moiety to the protease ligand or the E3 ligase ligand, or a pharmaceutically acceptable salt or stereoisomer thereof.

### Targeting Moiety

As described herein, a targeting moiety is an oligonucleotide capable of binding a protein. The term “oligonucleotide” refers to a molecule consisting of DNA, RNA, or DNA/RNA hybrids.

In some embodiments, the targeting moiety is a double-stranded nucleotide molecule that can bind to a target protein. The targeting moiety may be a double-stranded nucleotide that is comprised of two nucleotide strands that are sufficiently complementary to hybridize to form a duplex structure. In other embodiments, the targeting moiety is a single nucleotide strand that is self-complementary capable of forming a double-strand like structure. A target protein can be any protein that can bind to double-stranded nucleotides directly or indirectly. In some embodiments, a double-stranded oligonucleotide comprises a first non-protein recruiting region having between 0 and about 30 nucleotides, a protein recruiting region having between 3 and about 50 nucleotides, and a second protein recruiting region having between 0 and about 30 nucleotides. Each strand of a double-stranded oligonucleotide is generally between 3 and 100 nucleotides in length. Each strand of the duplex can be the same length or of different lengths.

In some embodiments, a target protein is a disease related protein (e.g., a protein for which changes in its function or activity cause disease, or whose function is considered important to the propagation of the disease state).

In some embodiments, a target protein targeted by a bifunctional compound described herein (e.g., an O′PROTAC provided herein) can be a protein related to cancer (e.g., prostate cancer, neuroendocrine prostate cancer, breast cancer, colorectal cancer, chronic lymphocytic leukemia (CLL), lymphoma, glioblastoma, myeloid leukemia, acute myeloid leukemia (AML), acute T-cell lymphoma, T-cell lymphoma, leukemia, lympho-plasmacytoid B-cell lymphoma, glioma, small cell lung cancer, neuroplastoma, angiosarcoma, chondrosarcoma, Ewing's sarcoma, fibroblastic sarcoma, gynecological sarcoma, liposarcoma, osteosarcoma, rhabdomyosarcoma, soft tissue sarcoma, synovial sarcoma, PRAD (prostate adenocarcinoma), BRCA (breast invasive carcinoma), BLCA (bladder urothelial carcinoma), LUAD (lung adenocarcinoma), LIHC (liver hepatocellular carcinoma), CESC (cervical squamous cell carcinoma and endocervical adenocarcinoma), CHOL (cholangiocarcinoma), LUSC (lung squamous cell carcinoma), COAD (colon adenocarcinoma), READ (rectum adenocarcinoma), PAAD (pancreatic adenocarcinoma), UCEC (uterine corpus endometrial carcinoma), UCS (uterine carcinosarcoma), HNSC (head and neck squamous cell carcinoma), MESO (mesothelioma), TGCT (testicular germ cell tumors), OV (ovarian serous cystadenocarcinoma), THCA (thyroid carcinoma), SARC (sarcoma), SKCM (skin cutaneous melanoma), ACC (adrenocortical carcinoma), KIRC (kidney renal clear cell carcinoma), PCPG (pheochromocytoma and paraganglioma), KIRP (kidney renal papillary cell carcinoma), DLBC (lymphoid neoplasm diffuse large B-cell lymphoma), THYM (thymoma), LGG (brain lower grade glioma), KICH (kidney chromophobe), GBM (glioblastoma multiforme), LAML (acute myeloid leukemia) and UVM (uveal melanoma). In some embodiments, a target protein targeted by a bifunctional compound described herein (e.g., an O′PROTAC provided herein) can be a protein related to a carcinoma or a hematological cancer (e.g., a lymphoma, leukemia, or lymphoid malignancy). In some embodiments, a target protein targeted by a bifunctional compound described herein (e.g., an O′PROTAC provided herein) can be a protein related to a cancer associated with Fos or a cancer associated with Jun. In some embodiments, a target protein targeted by a bifunctional compound described herein (e.g., an O′PROTAC provided herein) can be a protein related to a metastatic cancer (e.g., a metastatic cancer of any of the cancers described herein).

In some embodiments, a target protein targeted by a bifunctional compound described herein (e.g., an O′PROTAC provided herein) can be a protein related to an autoimmune disease (e.g., HIV/AIDS, diabetes, or multiple sclerosis).

In some embodiments, a target protein targeted by a bifunctional compound described herein (e.g., an O′PROTAC provided herein) can be a protein related to an inflammatory disease (e.g., rheumatoid arthritis, fatty liver disease, or inflammatory bowel disease) or ischemia.

In some embodiments, a target protein targeted by a bifunctional compound described herein (e.g., an O′PROTAC provided herein) can be a protein related to a neurodegenerative disease (e.g., Parkinson's disease, Huntington's disease, Alzheimer's disease, frontal temporal dementia, amyotrophic lateral sclerosis, or multiple sclerosis).

In some embodiments, a target protein targeted by a bifunctional compound described herein (e.g., an O′PROTAC provided herein) can be a protein related to a developmental disease, Müller-Weiss disease (MWD), campomelic dysplasia, a cardiovascular disease, a rare disease, a kidney disease, or a brain disease (e.g., adrenoleukodystrophy). In some embodiments, a target protein targeted by a bifunctional compound described herein (e.g., an O′PROTAC provided herein) can be a protein related to a fibrotic disease or condition including, without limitation, scars, idiopathic pulmonary fibrosis, non-alcoholic steatohepatitis, and fibrosis of the liver, eye, kidney or cardiac tissues. Examples of target proteins that can be targeted by a bifunctional compound described herein (e.g., an O′PROTAC provided herein) include, without limitation, DNA-binding proteins, such as transcription factors, transcription co-regulators, polymerases, nucleases, and histones as well as RNA-binding proteins. Examples of transcription factors that can be targeted by a bifunctional compound described herein (e.g., an O′PROTAC provided herein) include, without limitation, androgen receptor (AR), ERG, forkhead box A1 (FOXA1), LEF1, estrogen receptor (ER), NF-κB, E2 factor (E2F) (e.g., E2F1, E2F2, E2F3a, E2F3b, E2F4, E2F5, E2F6, E2F7, or E2F8), c-Myc, transactivator of transcription (TAT), Jun proto-oncogene (Jun/c-Jun), Fos proto-oncogene (Fos/c-Fos), nuclear factor of activated T cell (NFAT) (e.g., NFATc1, NFATC2, NFATC3, or NFATC4), Runt-related transcription factor 1 (RUNX1/AML1), Myc proto-oncogene (Myc/c-Myc), ETS proto-oncogene (ETS1), glioma-associated oncogene (GL1), ERG/FUS fusion, T-cell leukemia homeobox 1 (TLX1), LIM domain only 1 (LMO1), LIM domain only 2 (LMO2), lymphoblastic leukemia associated hematopoiesis regulator 1 (LYL1/E2a heterodimer), MYB proto-oncogene (MYB), paired box 5 (PAX-5), SKI proto-oncogene (SKI), T-cell acute lymphocytic leukemia protein 1 (TAL1), T-cell acute lymphocytic leukemia protein 2 (TAL2), glucocorticoid receptor, nuclear factor for IL-6 expression (NF-IL6), early growth response protein 1 (EGR-1), hypoxia-inducible factor 1-alpha (HIF-1a), signal transducer and activator of transcription 1 (STAT1), signal transducer and activator of transcription 3 (STAT3), signal transducer and activator of transcription 5 (STAT5), V-Maf avian musculoaponeurotic fibrosarcoma oncogene homolog-A (MAFA), SRY-box transcription factor 2 (SOX2), SRY-box transcription factor 9 (SOX9), CAAT/enhancer-binding protein alpha (CEBPA), CAAT/enhancer-binding protein beta (CEBPB), Globin transcription factor (GATA) (e.g., GATA1, GATA2, GATA3), myocyte enhancer factor 2 (MEF2) (e.g., MEF2A, MEF2B, MEF2C, MEF2D), POU class 3 homeobox 2 (BRN2), zinc finger E-box binding homeobox 2 (ZEB2), nuclear receptor subfamily 4 group A member 1 (NR4A1), activating transcription factor 4 (ATF4), T-box transcription factor 21 (TBX21), RAR related orphan receptor C (RORC), and X-box binding protein (XBP-1s). Nucleotides that recognize and bind to a target protein are well known or readily available to one skilled in the art. Table A provides a list of target proteins (e.g., transcription factors) that can be targeted by a bifunctional compound described herein (e.g., an O′PROTAC provided herein). Table A also provides one or more exemplary nucleotide sequences that can be used to create a targeting moiety of a bifunctional compound described herein (e.g., an O′PROTAC provided herein). In some cases, a bifunctional compound described herein (e.g., an O′PROTAC provided herein) having a targeting moiety containing a double stranded nucleic acid that includes the sequence provided in Table A can be used to treat the indicated disease(s) as set forth in Table A.

TABLE A
Exemplary Transcription Factors and Sequences for a Targeting Moiety

| | | | SEQ |
|---|---|---|---|
| Transcription Factor | Disease(s) | DNA Sequence(s) | ID NO |
| AR | Prostate cancer | TTAGGGTACAC | 1 |
| | | TTAGGGTACACCGTGTACCT | 2 |
| ERG | Metastatic cancer | ACGGACCGGAAATCCGGTT | 3 |
| | | ACAGGAAGTG | 4 |
| FOXA1 | Prostate cancer, breast | TTAGGGTACACCGTGTACCT | 2 |
| | cancer | | |
| LEF1 | Colorectal cancer, CLL | TACAAAGATCAAAGGGTT | 5 |
| ER | | GGGTCAGGGTGACCT | 6 |
| | | GGGTCAAGGTGACCC | 7 |
| | | GGGTCATGGTGACCC | 8 |
| | | GGTCACAGTGACC | 9 |
| | | AGTCACTGTGACC | 10 |
| NF-κB | Cancer | GGGRNYYYCC, wherein R is A or G; | 11 |
| | | and Y is T or C; and N is any | |
| | | nucleotide | |
| | | GGGACTTTCC | 12 |
| | | GGGAATTTCC | 13 |
| | | GGGGCTTTCC | 14 |
| E2F: | Cancer | TTTCCCGC | 15 |
| (E2F1, E2F2, E2F3a, | | TTTGGCGC | 16 |
| E2F3b, E2F4, E2F5, | | TTTCGCGC | 17 |
| E2F6, E2F7, E2F8) | | | |
| JUN/c-Jun | Cancer with Fos | ATGAGTCAT | 18 |
| | Fibrotic disorders | | |
| Fos/c-Fos | Cancer with Jun | ATGAGTCAT | 18 |
| NFAT | Cancer | (A/T)GGAAAA(A/T/C) | 19 |
| (NFATc1, NFATc2, | | ATGGAAAAA | 20 |
| NFATc3, NFATc4) | | ATGGAAAAT | 21 |
| | | ATGGAAAAC | 22 |
| | | TTGGAAAAA | 23 |
| | | TTGGAAAAT | 24 |
| | | TTGGAAAAC | 25 |
| RUNX1/AML1 | AML | YGYGGTY; wherein Y is T or C | 26 |
| | | AGCTGCGGTCAT | 27 |
| | | TGCGGTC | 28 |
| | | CGTGGTT | 29 |
| MYC/c-MYC | Cancer | CACGTG | 30 |
| | | GACCACGTGGTC | 31 |
| | | GGCCACGTGACC | 32 |
| ETS1 | Lymphoma | CGGAAG | 33 |
| | | AGGAAG | 34 |
| GLI1 | Glioblastoma | GACCACCCA | 35 |
| ERG/FUS fusion | Myeloid leukemia | ACAGGAAGTG | 4 |
| (ERG transcription) | | | |
| TLX1 | Acute T-cell Lymphoma | CGGTAAGTGG | 36 |
| | | CGGTAAGTGG | 36 |
| LMO1/LMO2 | T-cell lymphoma | AGATAG | 37 |
| LYL1/E2a | Acute T-cell lymphoma | AACAGATGTT | 38 |
| hetrodimer | | | |
| MYB | Leukemias | (T/C)AAC(G/T)G(A/C/T)(A/C/T) | 39 |
| | | TAACGGAA | |
| | | TAACGGAC | 40 |
| | | TAACGGAT | 41 |
| | | TAACGGCA | 42 |
| | | TAACGGCC | 43 |
| | | TAACGGCT | 44 |
| | | TAACGGTA | 45 |
| | | TAACGGTC | 46 |
| | | TAACGGTT | 47 |
| PAX-5 | Lympho-plasmacytoid | GCAGCCAAGCGTGACC | 48 |
| | B-cell lymphoma | | |
| SKI | Carcinoma | GTCTAGAC | 49 |
| TAL1/TAL2 | Acute T-cell leukemia | AACAGATGGT | 50 |
| SCL | Leukemia | CACGTG | 30 |
| GR | Cancer | AGAACA | 51 |
| | | AGAACANNNTGTTCT; for homodimer | 52 |
| | | binding, N is any nucleotide | |
| NF-IL6 | Inflammatory Diseases | CCACAGAGTGATGTAATCA | 53 |
| EGR-1 | Cancer, Ischemia | GCGTGGGCG | 54 |
| | | GCGGGGGCG | 55 |
| HIF-1a | glioma | ACGTG | 56 |
| | | GCGTG | 57 |
| STAT1 | Autoimmune | TTCGCCGAA | 58 |
| | | TTCCCCGAA | 59 |
| STAT3 | Autoimmune | CTTCCGGGAA | 60 |
| STAT5 | Autoimmune | TTC(T/C)N(G/A)GAA; N is any | 61 |
| | | nucleotide | |
| | | TTCTTGGAA | 62 |
| | | TTCTTAGGA | 63 |
| | | TTCCTGGAA | 64 |
| | | TTCCTAGAA | 65 |
| MAFA | Diabetes, cancer | TGCTGACTCAGCA | 66 |
| SOX2 | Cancer, Developmental | CCCATTGTTC | 67 |
| | disorders | | |
| SOX9 | campomelic dysplasia | AGAACAATGG | 68 |
| TAT | HIV | TAR RNA: GAUCUGAGCCUGGGAGCUCUC | 69 |
| | | GCAGAUCUGAGCCUGGGAGCUCUCUGC | 70 |
| CEBPA | Huntington′s disease, | AGCATTTCATCACAT | 71 |
| | cancer (AML, CML) | TGCAGCTTTCTACAAGGG | 72 |
| CEBPB | Parkinson′s disease, | GCGCGAGCGCAACAACA | 73 |
| | HIV | TGCTTGAACAAGTTCCGCAG | 74 |
| GATA (GATA1, | Heart Disease, | ATGATAAG | 75 |
| GATA2, GATA3) | Developmental | | |
| | disorders, Hematological | | |
| | cancers and disorders | | |
| MEF2 (MEF2A, | Cardiovascular disease, | TGTTACTATATAAATAGAAT | 76 |
| MEF2B, MEF2C, | neurodegeneration, | | |
| MEF2D) | cancer (B Cell | | |
| | Lymphoma) | | |
| BRN2 | Glioblastoma, | ATGCAAATGTGCT | 77 |
| | neuroblastoma, small | | |
| | cell lung cancer, and | | |
| | neuroendocrine prostate | AATTATTCATGA | 78 |
| | cancer | | |
| ZEB2 | Rare disease, ALD, | CACCT | 79 |
| | Kidney Disease, Cancer | | |
| NR4A1 | Inflammatory diseases | TGACCTTTNCNT | 80 |
| ATF4 | MWD | consensus: 5′-GTGACGT[AC][AG]-3′ | 81 |
| | | TGATGCAA | 82 |
| | | GTGACGTAA | 83 |
| | | GTGACGTAG | 84 |
| | | GTGACGTCA | 85 |
| | | GTGACGTCG | 86 |
| | | TGATGAAAC | 87 |
| TBX21 | Inflammation/Rheumatoid | AGGTGTGAAA | 88 |
| | arthritis | AGGTGTGAAC | 89 |
| | | TCACACCT | 90 |
| | | TCCCACCT | 91 |
| RORC | Inflammation/Rheumatoid | Consensus sequence: AAYTAGGTCA; | 92 |
| | arthritis | where Y is T or C | |
| | | AATTAGGTCA | 93 |
| | | AACTAGGTCA | 94 |
| | | AGGTCA | 95 |
| | | AATTAGGTCA | 93 |
| | | AAATAGGTCA | 96 |
| XBP-1s | Inflammation/Rheumatoid | consensus 5′-GATGACGTG[TG]NNN[AT]T-3′ | 97 |
| | arthritis | ACACGCTTGGGAATGGACAC | 98 |
| | | CCATGGGAAGATGTTCTGGG | 99 |
| | | CACGCTTGGGAATGGACAC | 100 |
| | | GTGTCAGAGTCCATGGGA | 101 |
| | | GATGACGTGTAGTAT | 102 |
| | | GATGACGTGTAGTTT | 103 |
| | | GATGACGTGGAGTTT | 104 |
| | | GATGACGTGGAGTAT | 105 |
| Gain of Function | Cancer | GCCCCCTCGCGCCCCGCCCCTTGTC | 106 |
| Mutant p53 | | | |

### Modifications

In some embodiments, the nucleotide is chemically modified to enhance stability. Nucleotides synthesis is well known in the art, as is synthesis of nucleotides containing modified bases and backbone linkages. The synthesis and/or modification by methods well established in the art, such as those described in “Current protocols in nucleic acid chemistry,” Beaucage et al. (Eds.), John Wiley & Sons, Inc., New York, NY, USA, which is hereby incorporated herein by reference.

Modified backbones include those that retain a phosphorus atom in the backbone and those that do not have a phosphorus atom in the backbone. For the purposes of this specification, and as sometimes referenced in the art, modified nucleosides that do not have a phosphorus atom in their internucleoside backbone can also be considered as nucleosides.

Modified backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linkages, and those having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference.

Modified oligonucleotide backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other suitable nucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, a nucleotide mimetic that has been shown to have excellent hybridization properties, is referred to as a peptide nucleic acid (PNA). In PNA compounds, the sugar backbone of a nucleotide is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 254:1497-1500 (1991).

Other embodiments of the invention are nucleotides with phosphorodiamidate morpholino (PMO) backbones (Heasman, Developmental Biology 243(2):209-214 (2002); and Nan et al., Front. Microbiol. 9: 750 (2018)), phosphorothioate backbones and nucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—, and —N(CH₃)—CH₂—CH₂—[wherein the native phosphodiester backbone is represented as-O—PO—CH₂—] of the above-referenced U.S. Pat. No. 5,489,677, and the amide backbones of the above-referenced U.S. Pat. No. 5,602,240. Also preferred are nucleotides having morpholino backbone structures of the above-referenced U.S. Pat. No. 5,034,506.

Modified dsRNAs may also contain one or more substituted sugar moieties. Preferred dsRNAs comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C1 to C10 alkenyl and alkynyl. Other preferred dsRNAs comprise one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃,OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. Similar modifications may also be made at other positions on the dsRNA, particularly the 3′ position of the sugar on the 3′ terminal nucleotide or in 2′-5′ linked dsRNAs and the 5′ position of 5′ terminal nucleotide. DsRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

### Conjugates

Another modification of the nucleotides involves chemically linking to the nucleotides one or more moieties or conjugates that enhance the activity, cellular distribution, or cellular uptake of the nucleotides. Such moieties include, but are not limited to, lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-Stritylthiol (Manoharan et al., Ann. N.Y. Acad. Sci., 1992, 660:306-309; Manoharan et al., Biorg. Med Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behrnoaras et al., EMBO J, 1991, 10:1 111-1 118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Then, 1996, 277:923-937). Representative U.S. patents that teach the preparation of such dsRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

Typical conjugation protocols involve the synthesis of nucleotides bearing an amino linker at one or more positions of the sequence. The amino group is then reacted with the molecule being conjugated using appropriate coupling or activating reagents. The conjugation reaction may be performed either with the nucleotides still bound to the solid support or following cleavage of the nucleotides in solution phase. Purification of the nucleotides conjugate by HPLC typically affords the pure conjugate.

In some embodiments, the targeting moiety is dsDNA. A dsDNA includes two DNA strands that are sufficiently complementary to hybridize to form a duplex structure or one DNA strand that is self-complementary to form a double-strand like structure. A dsDNA can comprise a first non-protein recruiting region having between 0 and about 30 bases, a protein recruiting region having between 3 and about 50 bases, and a second protein recruiting region having between 0 and about 30 bases. Each strand of a dsDNA is generally between 5 and 100 bases in length. Each strand of the duplex can be the same length or of different lengths.

In some embodiments, the dsDNA can be a dsDNA represented by any one of the following sequences targeting AR (A and B disclose SEQ ID NOS 1 and 445 and 2 and 446, respectively, in order of appearance), ERG (C discloses SEQ ID NOS 3 and 447, respectively, in order of appearance), FOXA1 (D discloses SEQ ID NOS 2 and 446, respectively, in order of appearance), or LEF (E discloses SEQ ID NOS 5 and 448, respectively, in order of appearance):

A:
TTAGGGTACAC
AATCCCATGTG
B:
TTAGGGTACACCGTGTACCT
AATCCCATGTGGCACATGGA
C:
ACGGACCGGAAATCCGGTT
TGCCYGGCCTTTAGGCCAA
D:
TTAGGGTACACCGTGTACCT
AATCCCATGTGGCACATGGA
E:
TACAAAGATCAAAGGGTT
ATGTTTCTAGTTTCCCAA

### Linkers

The Linker (L) provides a covalent attachment of the targeting moiety to the protease ligand or the E3 ligase ligand (e.g., an E3 ubiquitin ligase ligand).

In some embodiments, the linker may be attached to the terminal nucleotide or the nucleotide in the middle of the sequence.

In some embodiments, the linker may be attached to the 5′ or 3′ or 2′ sugar moiety of a terminal nucleotide or the nucleotide in the middle of the sequence.

In some embodiments, the linker may be attached to the sugar mimetics of a terminal nucleotide or the nucleotide in the middle of the sequence.

In some embodiments, the linker may be attached to the modified nucleobase of a terminal nucleotide or the nucleotide in the middle of the sequence.

In some embodiments, the linker group L is a group comprises one or more covalently connected structural units of A (e.g. -A₁ . . . A_{q}-), wherein A₁ is coupled to a targeting moiety, and q is an integer greater than or equal to 0. In certain embodiments, q is an integer greater than or equal to 1.

In certain embodiments, e.g., wherein q is greater than 2, A_{q} is a group that is connected to a protease ligand or an E3 ligase ligand, and A₁ and A_{q} are connected via structural units of A (number of such structural units of A: q-2).

In certain embodiments, e.g., wherein q is 2, A_{q} is a group that is connected to A₁, and to a protease ligand or an E3 ligase ligand.

In certain embodiments, e.g., wherein q is 1, the structure of the linker group L is -A₁-, and A₁ is a group that is connected to a protease ligand or an E3 ligase ligand and an targeting moiety.

In additional embodiments, q is an integer from 1 to 100, 1 to 90, 1 to 80, 1 to 70, 1 to 60, 1 to 50, 1 to 40, 1 to 30, 1 to 20, or 1 to 10.

In certain embodiments, A₁ to A_{q} are, each independently, a bond, CR^{L}1R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C-C, SiR^{L1}CR^{L2}, P(O)OR^{L1}, P(O)OR^{L1}, NR^{L3}C(═NCN)NR^{L4}, NR^{L3}C(═NCN), NR^{L3}C (═CNO)NR^{L4}, C₃₋₁₁ cycloalkyl optionally substituted with 0-6 R^{L1} and/or R^{L2} groups, C₃₋₁₁ heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, wherein R^{L1} or R^{L2}, each independently, can be linked to other A groups to form cycloalkyl and/or heterocyclyl moeity which can be further substituted with 0-4 R^{L5} groups. In some cases, R^{L1}, R^{L2}, R^{L3}, R^{L4} and R^{L5} are, each independently, H, halo, C₁₋₈alkyl, OC₁₋₈ alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, S C₁₋₈cycloalkyl, NH C₁₋₈cycloalkyl, N(C₁₋₈cycloalkyl)₂, N (C₁₋₈cycloalkyl) (C₁₋₈alkyl), OH, NH₂, SH, SO₂ C₁₋₈alkyl, P (O) (OC₁₋₈alkyl) (C₁₋₈alkyl), P(O) (O C₁₋₈alkyl)₂, CC—C₁₋₈alkyl, CCH, CH═CH (C₁₋₈alkyl), C (C₁₋₈alkyl)═CH (C₁₋₈alkyl), C(C₁₋₈alkyl)═C (C₁₋₈ alkyl)₂, Si(OH)₃, Si (C₁₋₈alkyl)₃, Si (OH) (C₁₋₈alkyl)₂, CO C₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON (C₁₋₈alkyl)₂, NHCONH₂, N(C₁₋₈alkyl)SONH(C₁₋₈alkyl), N(C₁₋₈alkyl) SO₂N(C₁₋₈alkyl)₂, NHSONH(C₁₋₈alkyl), NHSON(C₁₋₈alkyl)₂, or NHSO₂NH₂.

In some embodiments, the linker may be an alkylene chain or a bivalent alkylene chain, either of which may be interrupted by, and/or terminate (at either or both termini) in —P(O)(OH)O—, —O—PO(OH)—O—, —O—, —S—, —N(R′)—, —C(O)—, —C(O)O—, —OC(O)—, —OC(O)O—, —C(NOR′)—, C(O)N(R′)—, —C(O)N(R′)C(O)—, —C(O)N(R)C(O)N(R′)—, —N(R)C(O)—, —N(R)C(O)N(R)—, —N(R)C(O)O—, —OC(O)N(R)—, —C(NR)—, —N(R′)C(NR′)—, —C(NR′)N(R)—, —N(R′)C(NR)N(R′)—, —S(O)₂— —OS(O)—, —S(O)₂— —S(O)—, —OS(O)₂—, —S(O)₂O—, —N(R)S(O)₂—, —S(O)₂N(R)—, —N(R′)S(O)—, —S(O)N(R′)—, —N(R)S(O)₂N(R′)—, —N(R)S(O)N(R)—, C1-C12 carbocyclene, 3- to 12-membered heterocyclene, 5- to 12-membered heteroarylene or any combination thereof, wherein R is H or C1-C12 alkyl, wherein the interrupting and the one or both terminating groups may be the same or different.

In some embodiments, the linker may be a polyethylene glycol chain which may terminate (at either or both termini) in —P(O)(OH)O—, —O—PO(OH)—O—, —S—, —N(R′)—, —C(O)—, —C(O)O—, —OC(O)—, —OC(O)O—, —C(NOR)—, —C(O)N(R′)—, —C(O)N(R)C(O)—, —C(O)N(R)C(O)N(R′)—, —N(R)C(O)—, —N(R′)C(O)N(R)—, —N(R)C(O)O—, —OC(O)N(R)—, —C(NR′)—, —N(R)C(NR′)—, —C(NR′)N(R)—, —N(R)C(NR′)N(R)—, —S(O)₂—, —OS(O)—, —S(O)O—, —S(O)—, —OS(O)₂—, —S(O)₂O—, —N(R)S(O)₂—, —S(O)₂N(R)—, —N(R′)S(O)—, —S(O)N(R)—, —N(R)S(O)₂N(R′)—, —N(R′)S(O)N(R′)—, C3-12 carbocyclene, 3 to 12-membered heterocyclene, 5 to 12-membered heteroarylene or any combination thereof, wherein R is H or C1-C6 alkyl, wherein the one or both terminating groups may be the same or different.

In some embodiments, the linker is an alkylene chain having 1-20 alkylene units and interrupted by or terminating in —O—, —NMe- —PO(OH)—O—, —O—PO(OH)—O—,

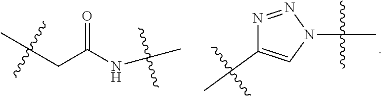 [SMILES: *CC(=O)N* ; *c1cn(*)nn1]

In some embodiments, the linker is a polyethylene glycol linker having 2-20 PEG units and interrupted by or and terminating in —O—, —NMe-, —PO(OH)—O—, —O—PO(OH)—O—,

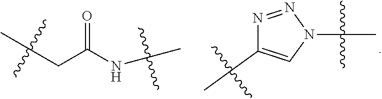 [SMILES: *CC(=O)N* ; *c1cn(*)nn1]

Thus, in some embodiments, a linker of a bifunctional compound described herein (e.g., an O′PROTAC provided herein) can be represented by any of the following structures:

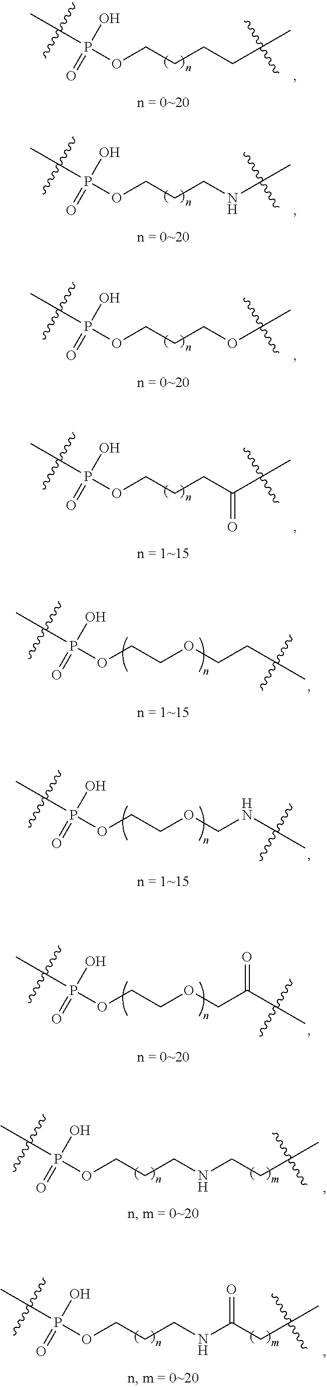

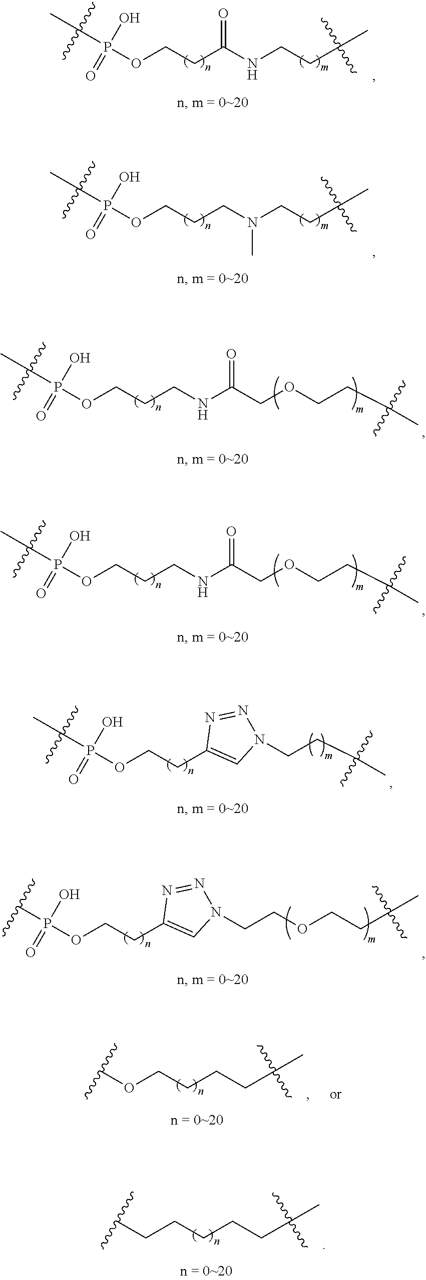

In some embodiments, a linker of a bifunctional compound described herein (e.g., an O′PROTAC provided herein) can be represented by any of the following linker structures shown in the context of an LEF1 OP-V1:

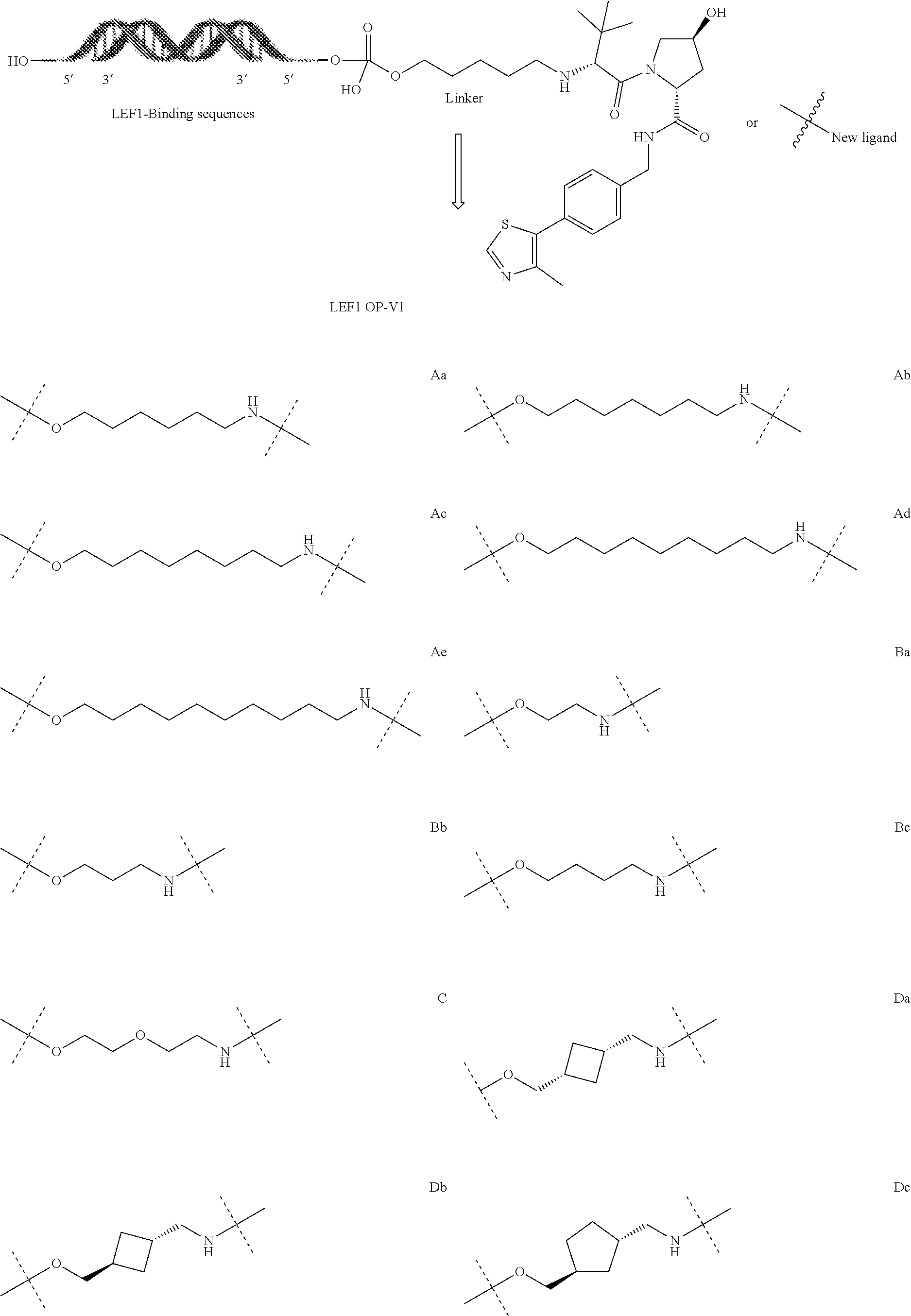

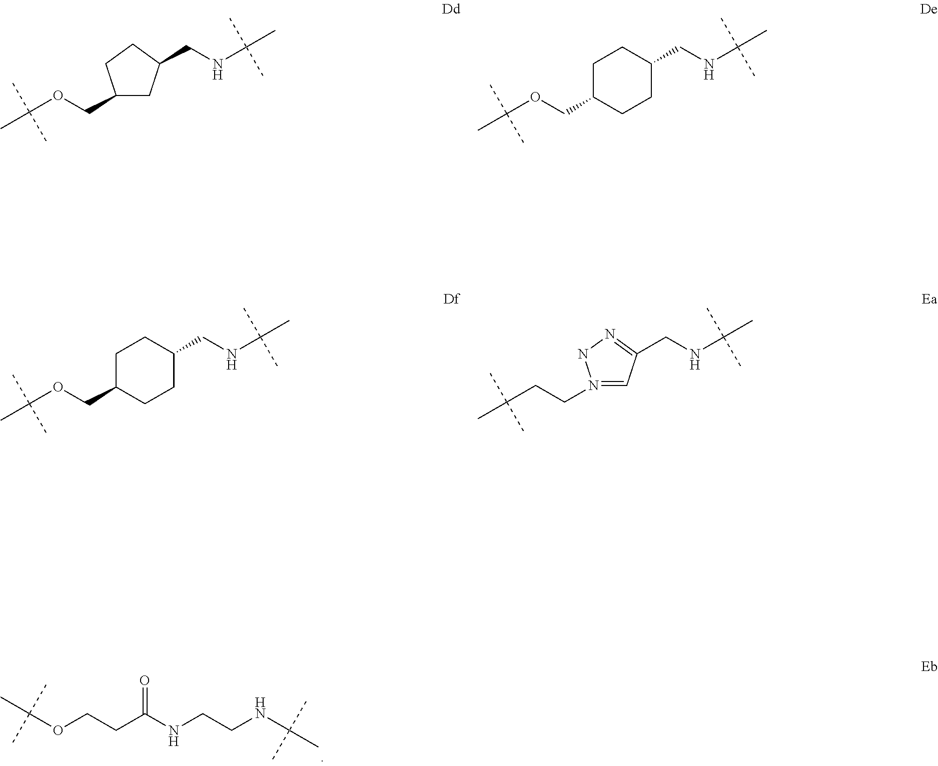

### Protease Ligands and E3 Ligase Ligands

The protease ligand is a functional moiety that binds a protease. The protease ligand is a functional moiety capable of binding with a protease, allowing for the protease to be brought into proximity with the POI such that the POI may be degraded. In some embodiments, the protease ligand is a peptide or small molecule. As used herein, small molecule means that the protease ligand has a molecular weight of less than about 900 D and, suitably, less than about 800 D, 700 D, or 600 D.

The E3 ligase ligand is a functional moiety that binds an E3 ligase. The E3 ligase ligand is a functional moiety capable of binding with an E3 ligase, allowing for the E3 ligase to be brought into proximity with the POI such that the POI may be degraded. In some embodiments, the E3 ligase ligand is a peptide or small molecule. As used herein, small molecule means that the E3 ligase ligand has a molecular weight of less than about 900 D and, suitably, less than about 800 D, 700 D, or 600 D.

In some embodiments, the ligand component of a compound provided herein is an E3 ligase ligand. The E3 ligase ligand is a functional moiety that binds an E3 ubiquitin ligase. E3 ubiquitin ligases (of which over 600 are known in humans) confer substrate specificity for ubiquitination. There are known ligands which bind to these ligases. As described herein, an E3 ubiquitin ligase binding group is a peptide or small molecule that can bind an E3 ubiquitin ligase. Specific E3 ubiquitin ligases include: von Hippel-Lindau (VHL); cereblon; XIAP; E3A; MDM2; Anaphase-promoting complex (APC); UBR5 (EDD1); SOCS/BC-box/eloBC/CUL5/RING; LNXp80; CBX4; CBLLl; HACE1; HECTD1; HECTD2; HECTD3; HECW1; HECW2; HERC1; HERC2; HERC3; HERC4; HUWE1; ITCH; EDD4; NEDD4L; PPIL2; PRPF19; PIASI; PIAS2; PIAS3; PIAS4; RANBP2; R4; RBX1; SMURFI; SMURF2; STUB1; TOPORS; TRIP12; UBE3A; UBE3B; UBE3C; UBE4A; UBE4B; UBOX5; UBR5; WWPl; WWP2; Parkin; A20/TNFAIP3; AMFR/gp78; ARA54; beta-TrCP1/BTRC; BRCA1; CBL; CHIP/STUB1; E6; E6AP/UBE3A; F-box protein 15/FBX015; FBXW7/Cdc4; GRAIL/RNF 128; HOIP/RNF31; cIAP-1/HIAP-2; cIAP-2/HIAP-1; cIAP (pan); ITCH/AIP4; KAPl; MARCH8; Mind Bomb 1/MIB1; Mind Bomb 2/MIB2; MuRF1/TRFM63; DFIP 1; EDD4; NleL; Parkin; R F2; R F4; RNF8; R F 168; R F43; SART1; Skp2; SMURF2; TRAF-1; TRAF-2; TRAF-3; TRAF-4; TRAF-5; TRAF-6; TRFM5; TRFM21; TRFM32; UBR5; and ZRF3.

In some embodiments, the bifunctional compound of Formula (IB) includes an E3 ligase ligand that binds cereblon. Representative examples of ligands that bind cereblon and which may be suitable for use as a protease ligand or E3 ligase ligand as described herein are described in U.S. Patent Application Publication 2018/0015085 or U.S. Patent Application Publication 2018/0215731.

In some embodiments, the bifunctional compound of Formula (IB) includes an E3 ligase ligand that binds cereblon and is represented by any one of the following structures:

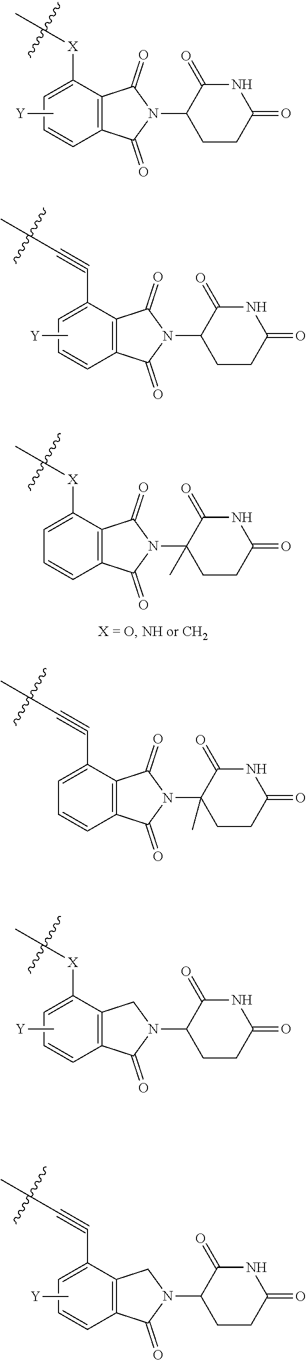

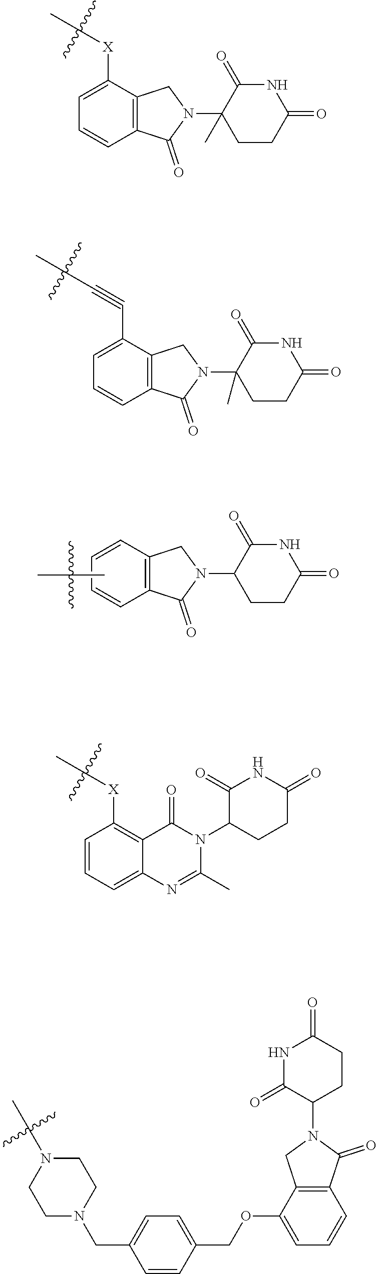

wherein X is a bond, NH, O or CH₂, Y is halo, alkyl, CN, CF₃, OCF₃ or OCHF₂.

In some embodiments, the E3 ligase ligand binds a Von Hippel-Lindau (VHL) tumor suppressor. Representative examples of E3 ligase ligands that bind VHL are as follows:

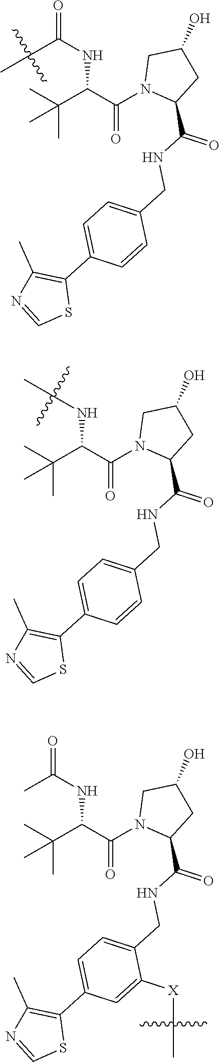

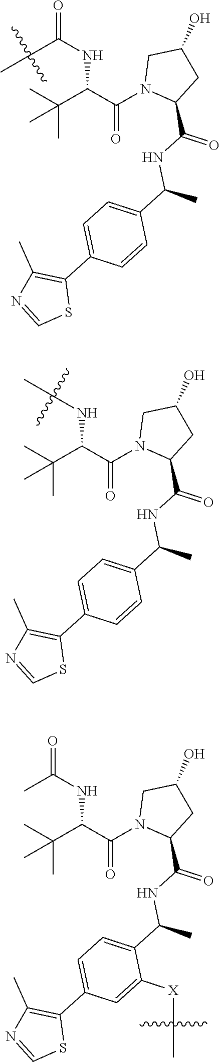

wherein X is a bond, N, O or C.

Yet other E3 ligase ligands that bind VHL and which may be suitable for use as an E3 ligase ligand of a bifunctional compound described herein (e.g., an O′PROTAC provided herein) are disclosed in WO2013/106643, U.S. Patent Application Publication No. 2016/0045607, WO2014/187777, U.S. Patent Application Publication No. 2014/0356322, and U.S. Pat. No. 9,249,153.

In some embodiments, the E3 ligase ligand binds an inhibitor of apoptosis protein (IAP) and is represented by any one of the following structures:

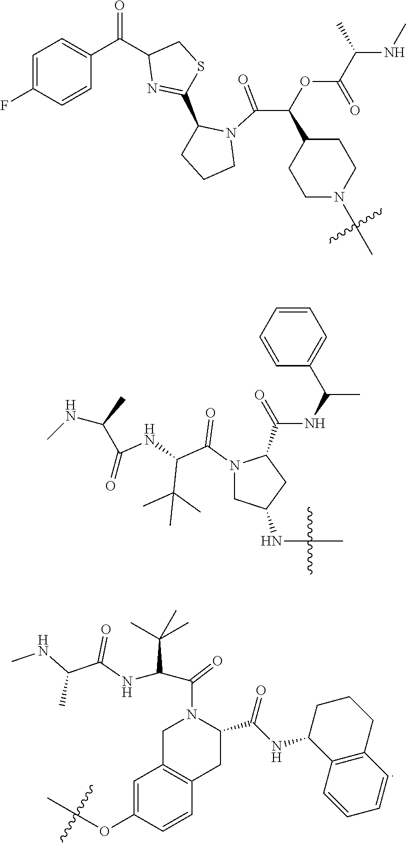

Yet other E3 ligase ligands that bind IAPs and which may be suitable for use as an E3 ligase ligand of a bifunctional compound described herein (e.g., an O′PROTAC provided herein) are disclosed in International Patent Application Publications WO2008/128171, WO2008/016893, WO2014/060768, WO2014/060767, and WO2015092420. IAPs are known in the art to function as ubiquitin-E3 ligases.

In some embodiments, the bifunctional compound of Formula (IB) includes an E3 ligase ligand that binds murine double minute 2 (MDM2) and is represented by any one of the following structures:

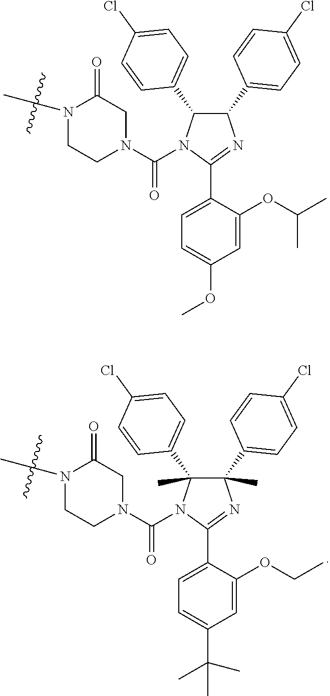 [SMILES: *N1CCN(C(=O)N2C(c3ccc(OC)cc3OC(C)C)=N[C@@H](c3ccc(Cl)cc3)[C@H]2c2ccc(Cl)cc2)CC1=O ; *N1CCN(C(=O)N2C(c3ccc(C(C)(C)C)cc3OCC)=N[C@@](C)(c3ccc(Cl)cc3)[C@@]2(C)c2ccc(Cl)cc2)CC1=O]

Yet other E3 ligase ligands that bind MDM2 and which may be suitable for use as an E3 ligase ligand of a bifunctional compound described herein (e.g., an O′PROTAC provided herein) are disclosed in WO2012/121361; WO2014/038606; WO2010/082612; WO2014/044401; WO2009/151069; WO2008/072655; WO2014/100065; WO2014/100071; WO2014/123882; WO2014/120748; WO2013/096150; WO2015/161032; WO2012/155066; WO2012/065022; WO2011/060049; WO2008/036168; WO2006/091646; WO2012/155066; WO2012/065022; WO2011/153509; WO2013/049250; WO2014/151863; WO2014/130470; WO2014/134207; WO2014/200937; WO2015/070224; WO2015/158648; WO2014/082889; WO2013/178570; WO2013/135648; WO2012/116989; WO2012/076513; WO2012/038307; WO2012/034954; WO2012/022707; WO2012/007409; WO2011/134925; WO2011/098398; WO2011/101297; WO2011/067185; WO2011/061139; WO2011/045257; WO2010/121995; WO2010/091979; WO2010/094622; WO2010/084097; WO2009/115425; WO2009/080488; WO2009/077357; WO2009/047161; WO2008/141975; WO2008/141917; WO2008/125487; WO2008/034736; WO2008/055812; WO2007/104714; WO2007/104664; WO2007/082805; WO2007/063013; WO2006/136606; WO2006/097261; WO2005/123691; WO2005/110996; WO2005/003097; WO2005/002575; WO2004/080460; WO2003/051360; WO2003/051359; WO1998/001467; WO2011/023677; WO2011/076786; WO2012/066095; WO2012/175487; WO2012/175520; WO2012/176123; WO2013/080141; WO2013/111105; WO2013/175417; WO2014/115080; WO2014/115077; WO2014/191896; WO2014/198266; WO2016/028391; WO2016/028391; WO2016/026937; WO2016/001376; WO2015/189799; WO2015/155332; WO2015/004610; WO2013/105037; WO2012/155066; WO2012/155066; WO2012/033525; WO2012/047587; WO2012/033525; WO2011/106650; WO2011/106650; WO2011/005219; WO2010/058819; WO2010/028862; WO2009/037343; WO2009/037308; WO2008/130614; WO2009/019274; WO2008/130614; WO2008/106507; WO2008/106507; WO2007/107545; WO2007/107543; WO2006032631; WO2000/015657; WO1998/001467; WO1997/009343; WO1997/009343; WO1996/002642; US2007/0129416; Med. Chem. Lett, 2013, 4, 466-469; J. Med. Chem., 2015, 58, 1038-1052; Bioorg. Med. Chem. Lett. 25 (2015) 3621-3625; or Bioorg. Med. Chem. Lett. 16 (2006) 3310-3314. Further specific examples of small molecular binding compounds for MDM2 contemplated for use as described herein include RG71 12, RG7388, MI 773/SAR 405838, AMG 232, DS-3032b, R06839921, R05045337, R05503781, Idasanutlin, CGM-097, and MK-8242. MDM2 is known in the art to function as a ubiquitin-E3 ligase.

In some embodiments, the E3 ligase ligand of a bifunctional compound described herein (e.g., an O′PROTAC provided herein) is represented by any of the following structures:

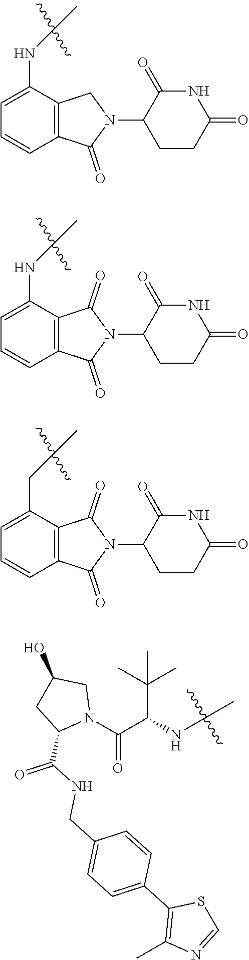 [SMILES: *Nc1cccc2c1CN(C1CCC(=O)NC1=O)C2=O ; *Nc1cccc2c1C(=O)N(C1CCC(=O)NC1=O)C2=O ; *Cc1cccc2c1C(=O)N(C1CCC(=O)NC1=O)C2=O ; *N[C@H](C(=O)N1C[C@H](O)C[C@H]1C(=O)NCc1ccc(-c2scnc2C)cc1)C(C)(C)C]

### Pharmaceutical Compositions

In some embodiments, pharmaceutical compositions contain a compound of Formula (IA) or (IB), as described herein, pharmaceutically acceptable salt or stereoisomer thereof, and a pharmaceutically acceptable carrier. The pharmaceutical compositions containing a compound of Formula (IA) or (IB) are useful for treating a disease or disorder associated with the expression or activity of a protein. Such pharmaceutical compositions can be formulated based on the mode of delivery.

The pharmaceutical compositions provided herein may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g., intraparenchymal, intrathecal or intraventricular, administration. A bifunctional compound described herein (e.g., an O′PROTAC provided herein) can be delivered in a manner to target a particular tissue, such as the liver (e.g., the hepatocytes of the liver).

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Suitable topical formulations include those in which a compound of Formula (IA) or (IB) described herein (e.g., an O′PROTAC provided herein) are in admixture with a topical delivery agent such as lipids, liposomes, polymeric nanoparticles fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine. DOTMA). A bifunctional compound described herein (e.g., an O′PROTAC provided herein) may be encapsulated within liposomes or may form complexes thereto, in particular to cationic liposomes. Alternatively, a bifunctional compound described herein (e.g., an O′PROTAC provided herein) may be complexed to lipids, in particular to cationic lipids. Suitable fatty acids and esters include, but are not limited to, arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C Oalkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

### Pharmaceutically Acceptable Salts

In some embodiments, a salt of a compound of Formula (IA) or (IB) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of Formula (IA) or (IB) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, (3-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (IA) or (IB) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of Formula (IA) or (IB), or pharmaceutically acceptable salts thereof, are substantially pure.

### Methods of Use

In some aspects, the bifunctional compound of Formula (IA) or (IB) may be useful in the treatment of diseases and disorders mediated by aberrant (e.g., dysregulated such as upregulated) protein activity. The diseases or disorders may be said to be characterized or mediated by dysfunctional protein activity (e.g., elevated levels of protein relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The bifunctional compounds of Formula (IA) or (IB) may be useful in the treatment of cancers, autoimmune diseases, central nervous system (CNS) diseases, and metabolic diseases, and infection diseases.

Examples of cancer to be treated herein include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Autoimmune diseases for which a bifunctional compound described herein (e.g., an O′PROTAC provided herein) may be used in treatment include rheumatologic disorders (such as, for example, rheumatoid arthritis, Sjogren's syndrome, scleroderma, lupus such as systemic lupus erythematosus (SLE) and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA associated vasculitis, including Churg-Strauss vasculitis, Wegener's granulomatosis, and polyarteritis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulindependent diabetes mellitus (IDDM), Addison's disease, and autoimmune thyroid disease (e.g., Graves' disease and thyroiditis)). More preferred such diseases include, for example, rheumatoid arthritis, ulcerative colitis, ANCA-associated vasculitis, lupus, multiple sclerosis, Sjogren's syndrome, Graves' disease, IDDM, pernicious anemia, thyroiditis, and glomerulonephritis.

Central nervous system (CNS) diseases include psychiatric disorders (e.g., panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence (e.g., alcohol, psychostimulants (e.g., crack, cocaine, speed, and meth), opioids, and nicotine), epilepsy, headache, acute pain, chronic pain, neuropathies, cereborischemia, dementia (including Alzheimer's type), movement disorders, and multiple sclerosis.

Metabolic diseases refer to disorders of metabolic processes and may be accompanied by one or more of the following symptoms: an increase in visceral obesity, serum glucose, and insulin levels, along with hypertension and dyslipidemia. It can be congenital due to inherited enzyme abnormality or acquired due to disease of an endocrine organ or failure of a metabolically important organ such as the pancreas. Within the term metabolic disease, the term “metabolic syndrome” is a name for a group of symptoms that occur together and are associated with the increased risk of developing coronary artery disease, stroke, and T2D. The symptoms of metabolic syndrome include central or abdominal obesity, high blood pressure, high triglycerides, insulin resistance, low HIDL cholesterol, and tissue damage caused by high glucose.

The infectious disease is caused by one or more bacteria, one or more viruses, one or more protozoa, one or more fungi, or one or more parasites, or a combination thereof.

In another aspect, the bifunctional compound of Formula (IA) or (IB) may be useful in a methods for assaying or diagnosing diseases and disorders mediated by aberrant protein activity. In some embodiments, such methods may be practiced in vitro or ex vivo. In other embodiments, such methods may be practice in vivo.

### Synthesis

A bifunctional compound described herein (e.g., an O′PROTAC provided herein) can be synthesized by synthetic routes that include processes analogous to those well-known in the chemical arts. Starting materials are generally available from commercial sources such as Aldrich Chemicals or are readily prepared using methods well known to those skilled in the art.

The general procedures and Examples provide exemplary methods for preparing bifunctional compounds described herein (e.g., O′PROTACs described herein). Those skilled in the art will appreciate that other synthetic routes may be used to synthesize the bifunctional compounds described herein (e.g., O′PROTACs described herein). Although specific starting materials and reagents are depicted and discussed in the Schemes, general procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

Generally, the preparation consists of synthesizing the two single strand nucleotides or modified nucleotides of the duplex by conventional solid phase oligonucleotide synthesis. After purification, the two nucleotides are annealed into the duplex.

In some embodiments, a modified nucleotide may be prepared by reacting a nucleotide with a phosphoramidite reagent according to the well-known procedures. The following synthetic routes describe exemplary methods of preparing modified nucleotides, the linker is as described before, not limited to this synthetic example.
a. Modified nucleotide can be prepared using phosphoramidite 1 for linking the nucleotide to the linker and E3 ligase ligand.
b. Nucleotide can be reacted with phosphoramidite 2 first, then coupling with compound 6 by amide condensation.
c. Nucleotide can be reacted with phosphoramidite 3 first, then coupling with compound 5 by amide condensation.
d. Nucleotide can be reacted with phosphoramidite 7 first, then coupling with compound 5 by click reaction.
e. Modified nucleotide (e.g., 8, 9) can be added to the oligonucleotide sequence directly.

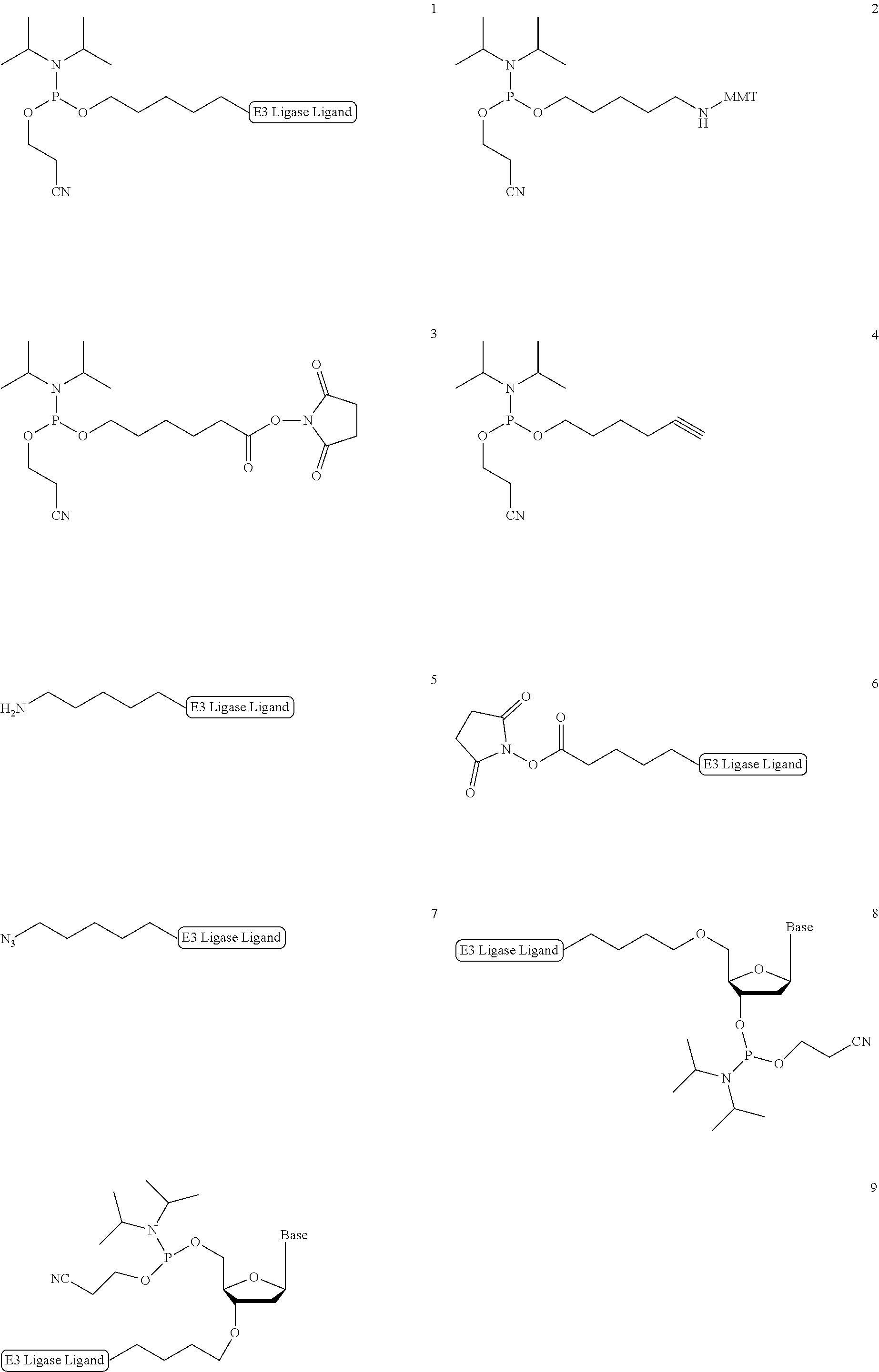

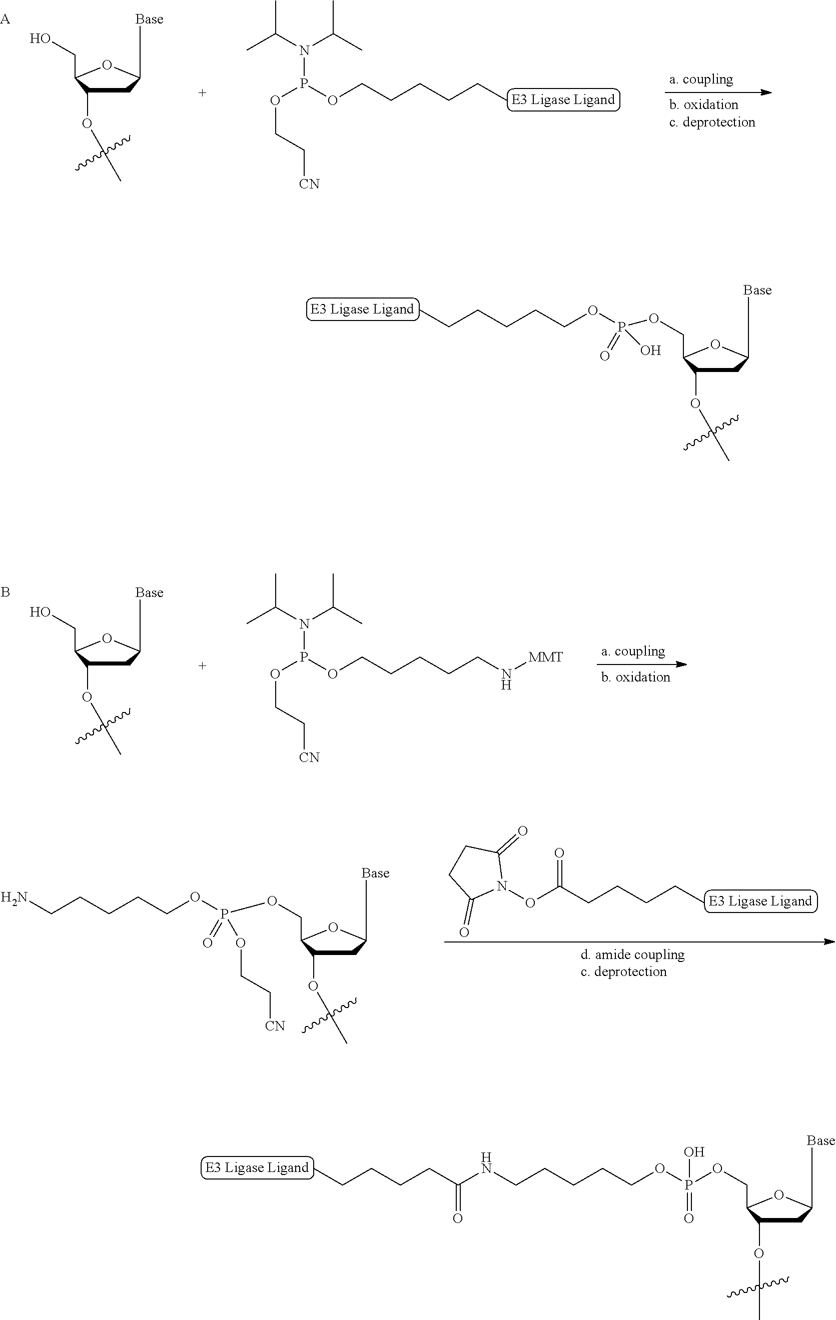

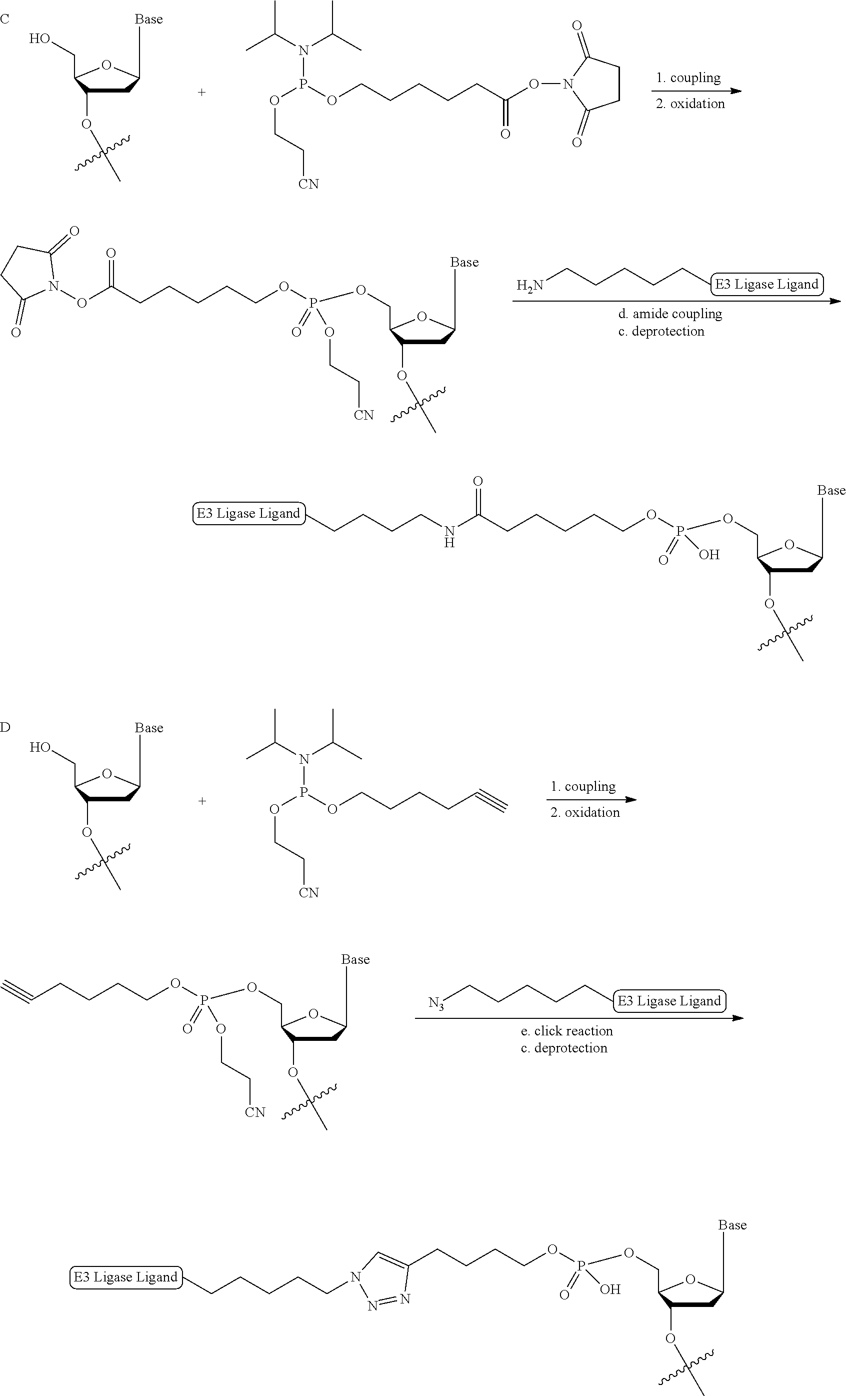

Referring to FIG. 19, the targeting moiety (i) (e.g., an oligonucleotide or a peptide capable of binding a target protein) may comprise a hydroxyl group. As such, the targeting moiety may be reacted with a reagent (ii) comprising a phosphine moiety that is reactive with a hydroxyl group. The reaction of the compounds (i) and (ii) may be carried out, for example, in an assembly buffer. For example, compound (ii) may be mixed with of 5-(ethylthio)-1H-tetrazole (EET) in acetonitrile to protonate the tertiary amine and subsequently added in excess to compound (i) to produce compound (iii). The resultant protected phosphate compound can be further deprotected to yield the compound (A). For example, compound (iii) can be mixed with concentrated aqueous ammonia and heated to produce compound (A), deprotecting compound (iii).

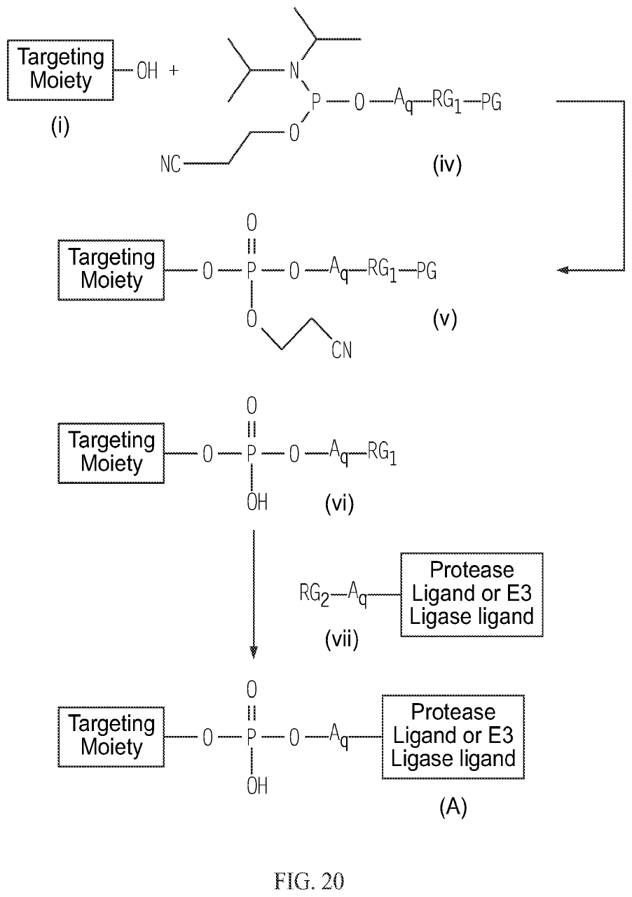

Referring to FIG. 20, a reagent (iv) may be prepared from a compound having general formula OH-A_{q}-RG₁, for example, by reacting this formula with Cl-PO(cyanoethyl)N Pr₂ to obtain the phosphine compound, followed by protecting the RG₁ group with a suitable protecting group. The compound of Formula (iv) may then be coupled with the compound of Formula (i) to obtain the phosphate compound (v). The phosphate compound (v) may be further deprotected. The deprotection reaction may simultaneously remove cyanoethyl protecting group from the phosphate and the PG group from the RG₁. For example, deprotection may be carried out by adding concentrated, aqueous ammonia and subsequently heating the reaction. In some cases, the deprotection conditions may be selected such that cyanoethyl group is removed first, followed by removal of the PG group, or such that the PG group is removed first, followed by removing the cyanoethyl group from the phosphate. The deprotected, reactive compound (vi) may then be coupled with the protease ligand-containing reagent or the E3 ligase ligand-containing reagent (vii) to obtain the final compound A. In this coupling reaction, the RG₁ group and the RG₂ group react to form an A group. For example, when RG₁ is an amino group and the RG₂ group react, an A group is formed which is C(O)NH. In another example, when RG₁ group is an alkyne and RG₂ group is an azide, an A group is formed which is a triazole.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds described herein that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated. Cis and trans geometric isomers of the compounds described herein may be isolated as a mixture of isomers or as separated isomeric forms. In some embodiments, a compound provided herein has the (R)-configuration. In some embodiments, a compound provided herein has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, without limitation, ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

### Phthalic Acid-Based PROTACs

In some cases, a compound provided herein can be designed such that the protease ligand of Formula (IA) or the protease ligand or E3 ligase ligand of Formula (IB) is based on phthalic acid or 3-aminophthalic acid. For example, the E3 ligase ligand of Formula (IB) can be:

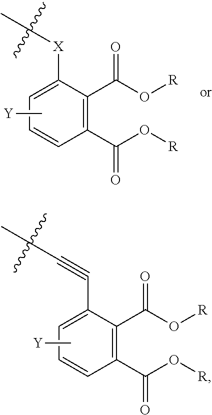

wherein each X is independently selected from a bond, NH, O and CH₂; wherein each Y is independently selected from halo, alkyl, CN, CF₃, OCF₃, and OCHF₂; and wherein each R is independently selected from H and C₁₋₈ alkyl. Such E3 ligase ligands can have the ability to bind to cereblon.

When the protease ligand of Formula (IA) or the protease ligand or E3 ligase ligand of Formula (IB) is based on phthalic acid or 3-aminophthalic acid, the targeting moiety can be an oligonucleotide that binds to a target protein as described herein or can be any other appropriate molecule (e.g., a molecule that lacks nucleotides) that binds to a target protein. In some cases when the protease ligand of Formula (IA) or the protease ligand or E3 ligase ligand of Formula (IB) is based on phthalic acid or 3-aminophthalic acid, the targeting moiety can have any structure that recognizes and binds to a target protein. In some cases when the protease ligand of Formula (IA) or the protease ligand or E3 ligase ligand of Formula (IB) is based on phthalic acid or 3-aminophthalic acid, the targeting moiety can be a binding domain of a polypeptide or protein that recognizes and binds to a target protein. In some cases when the protease ligand of Formula (IA) or the protease ligand or E3 ligase ligand of Formula (IB) is based on phthalic acid or 3-aminophthalic acid, the targeting moiety can be an inhibitor of the activity of a target protein (e.g., a kinase inhibitor, a HDAC inhibitor, or an angiogenesis inhibitor). In some cases when the protease ligand of Formula (IA) or the protease ligand or E3 ligase ligand of Formula (IB) is based on phthalic acid or 3-aminophthalic acid, the targeting moiety can be a small molecule that is capable of binding to a target protein. In some cases when the protease ligand of Formula (IA) or the protease ligand or E3 ligase ligand of Formula (IB) is based on phthalic acid or 3-aminophthalic acid, the targeting moiety can be an immunosuppressive compound. In some cases when the protease ligand of Formula (IA) or the protease ligand or E3 ligase ligand of Formula (IB) is based on phthalic acid or 3-aminophthalic acid, the targeting moiety can be a small molecule that binds a target protein.

The invention will be further described in the following examples, which do not limit the scope of the invention described in the claims.

### EXAMPLES

### Example 1: Synthesis of Phosphoramidites P1-6

TABLE 1
The structures of phosphoramidites P1-6.

| Compound | Structure |
|---|---|
| P1 | 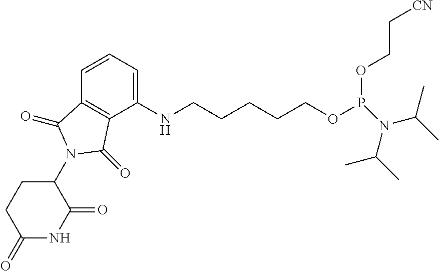 [SMILES: CC(C)N(C(C)C)P(OCCC#N)OCCCCCNc1cccc2c1C(=O)N(C1CCC(=O)NC1=O)C2=O] |
| P2 | 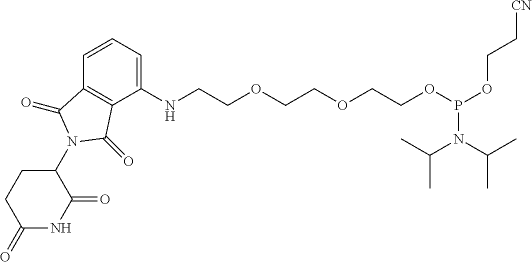 [SMILES: CC(C)N(C(C)C)P(OCCC#N)OCCOCCOCCNc1cccc2c1C(=O)N(C1CCC(=O)NC1=O)C2=O] |
| P3 | 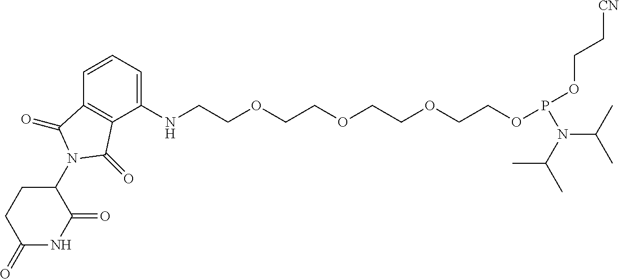 [SMILES: CC(C)N(C(C)C)P(OCCC#N)OCCOCCOCCOCCNc1cccc2c1C(=O)N(C1CCC(=O)NC1=O)C2=O] |
| P4 | 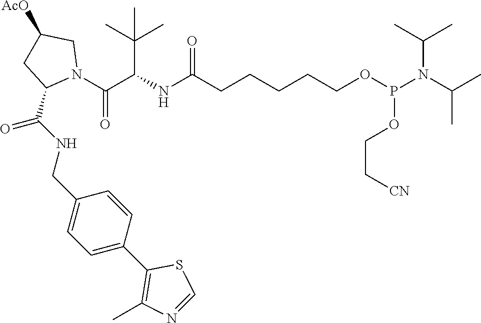 [SMILES: CC(=O)O[C@@H]1C[C@@H](C(=O)NCc2ccc(-c3scnc3C)cc2)N(C(=O)[C@@H](NC(=O)CCCCCOP(OCCC#N)N(C(C)C)C(C)C)C(C)(C)C)C1] |
| P5 | 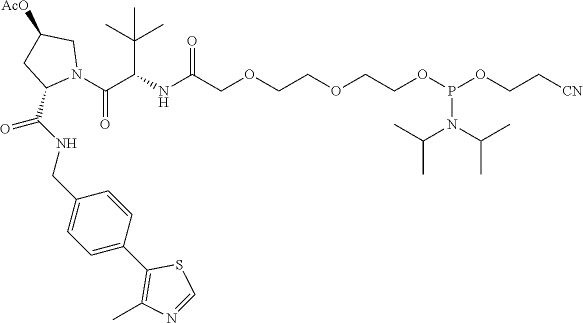 [SMILES: CC(=O)O[C@@H]1C[C@@H](C(=O)NCc2ccc(-c3scnc3C)cc2)N(C(=O)[C@@H](NC(=O)COCCOCCOP(OCCC#N)N(C(C)C)C(C)C)C(C)(C)C)C1] |
| P6 | 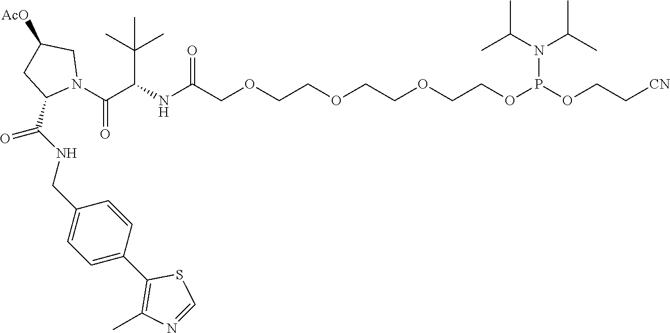 [SMILES: CC(=O)O[C@@H]1C[C@@H](C(=O)NCc2ccc(-c3scnc3C)cc2)N(C(=O)[C@@H](NC(=O)COCCOCCOCCOP(OCCC#N)N(C(C)C)C(C)C)C(C)(C)C)C1] |

### Synthesis of Phosphoramidites 1-6

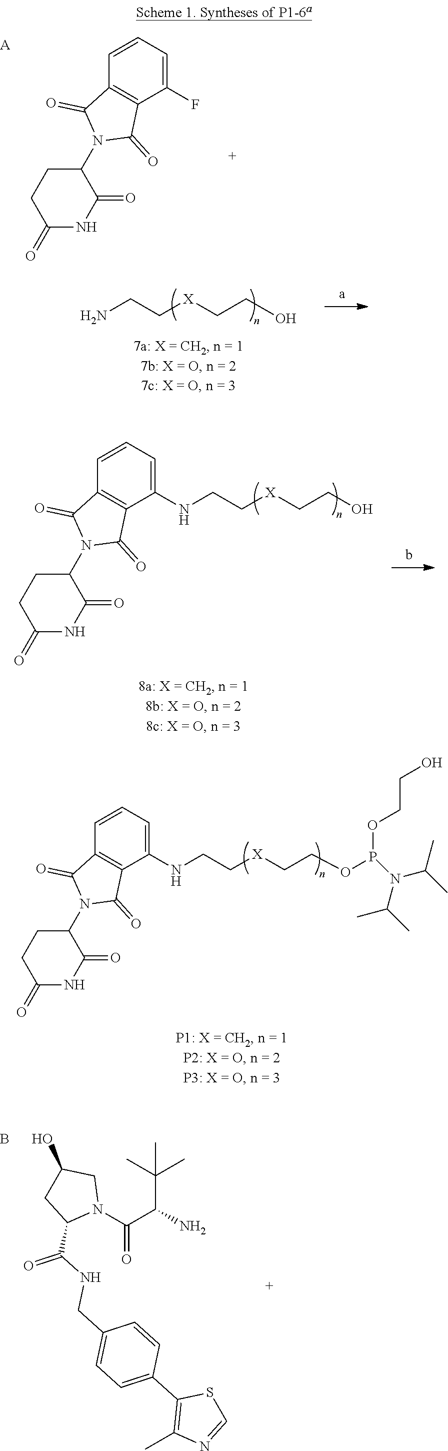

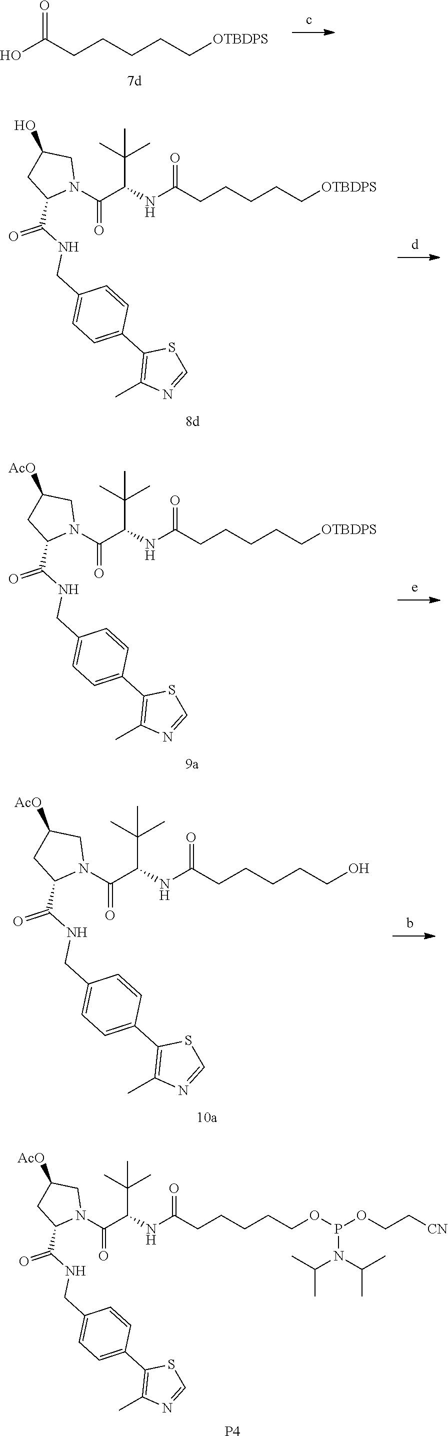

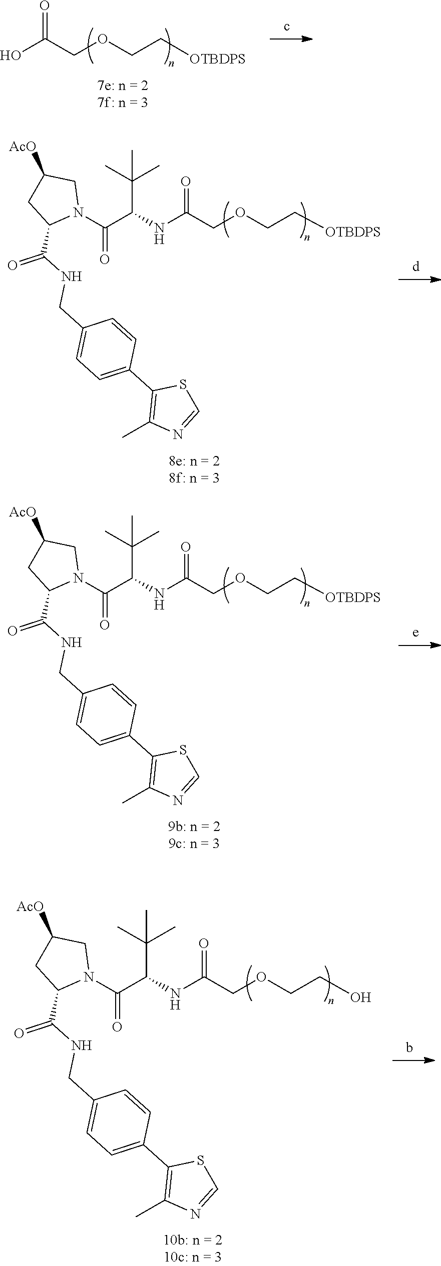

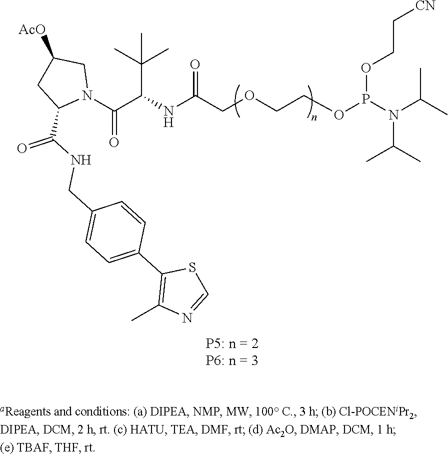

Synthesis of compound 8a-c: Compound 4-fluoro-thalidomide (1.0 equiv) was dissolved in NMP, DIPEA (2.0 equiv) and 7a-c (1.5 equiv) were added, the mixture was heated to 100° C. under microwave condition for 3 hours. then the mixture was absorbed on diatomite and purified by reversed-phase flash chromatography (H₂O:MeOH=90:10 to 50:50), giving compounds 8a-c.

2-(2,6-dioxopiperidin-3-yl)-4-((5-hydroxypentyl)amino)isoindoline-1,3-dione (8a): Yellow solid, 65%.1H NMR (400 MHz, CDCl₃) δ 8.16 (s, 1H), 7.49 (dd, J=8.5, 7.1 Hz, 1H), 7.09 (d, J=7.1 Hz, 1H), 6.88 (d, J=8.5 Hz, 1H), 4.91 (dd, J=12.1, 5.4 Hz, 1H), 3.66 (q, J=6.3 Hz, 2H), 3.28 (t, J=7.0 Hz, 2H), 2.93-2.67 (m, 3H), 2.12 (ddd, J=9.6, 5.8, 2.9 Hz, 1H), 1.75-1.66 (m, 2H), 1.64-1.59 (m, 2H), 1.54-1.46 (m, 2H).

2-(2,6-dioxopiperidin-3-yl)-4-((2-(2-(2-hydroxyethoxy)ethoxy)ethyl)amino)isoindoline-1,3-dione (8b): Yellow oil, 40%. ¹H NMR (400 MHz, CDCl₃) δ 8.31 (s, 1H), 7.48 (dd, J=8.5, 7.2 Hz, 1H), 7.10 (d, J=7.1 Hz, 1H), 6.90 (d, J=8.5 Hz, 1H), 4.91 (dd, J=11.9, 5.3 Hz, 1H), 3.76-3.70 (m, 4H), 3.69-3.64 (m, 4H), 3.62-3.58 (m, 2H), 3.47 (t, J=5.3 Hz, 2H), 2.90-2.65 (m, 3H), 2.15-2.07 (m, 1H).

2-(2,6-dioxopiperidin-3-yl)-4-((2-(2-(2-(2-hydroxyethoxy)ethoxy)ethoxy)ethyl)amino)isoindoline-1,3-dione (8c): Yellow oil, 30%. ¹H NMR (400 MHz, DMSO-d₆) δ 11.08 (s, 1H), 7.63-7.55 (dd, J=8.5, 7.0 Hz, 1H), 7.15 (d, J=8.5 Hz, 1H), 7.04 (d, J=7.0 Hz, 1H), 6.60 (t, J=5.9 Hz, 1H), 5.05 (dd, J=13.0, 5.4 Hz, 1H), 4.55 (t, J=5.4 Hz, 1H), 3.62 (t, J=5.3 Hz, 2H), 3.59-3.43 (m, 12H), 3.39 (t, J=5.2 Hz, 2H), 2.94-2.82 (m, 1H), 2.56 (dd, J=19.8, 10.4 Hz, 2H), 2.08-1.96 (m, 1H).

Synthesis of compound P1-3: compound 8a-c (1.0 equiv) was dissolved in anhydrous DCM, DIPEA (2.0 equiv) and Cl-POCEN′Pr₂ (1.5 equiv) was added. The mixture was stirred at room temperature for 1 hour. Solvent was removed, and the residue was purified with flash chromatography (Hexane:Actone (5% TEA)=100:0 to 75:25), giving product P1-3.

2-cyanoethyl (5-((2-(2,6-dioxopiperidin-3-yl)-1,3-dioxoisoindolin-4-yl)amino)pentyl) diisopropylphosphoramidite (P1): Yellow oil, 65%. ¹H NMR (400 MHz, DMSO-d₆) δ 11.08 (s, 1H), 7.57 (t, J=7.9 Hz, 1H), 7.09 (d, J=8.5 Hz, 1H), 7.01 (d, J=6.2 Hz, 1H), 6.54 (s, 1H), 5.04 (dd, J=12.4, 4.5 Hz, 1H), 3.78-3.65 (m, 2H), 3.64-3.45 (m, 4H), 2.95-2.82 (m, 1H), 2.74 (t, J=5.4 Hz, 2H), 2.63-2.52 (m, 2H), 2.02 (d, J=12.2 Hz, 1H), 1.59 (s, 4H), 1.42 (d, J=6.3 Hz, 2H), 1.15 (dt, J=13.9, 7.3 Hz, 12H).

2-cyanoethyl (2-(2-(2-((2-(2,6-dioxopiperidin-3-yl)-1,3-dioxoisoindolin-4-yl)amino)ethoxy)ethoxy)ethyl) diisopropylphosphoramidite (P2): Yellow oil, 68%. ¹H NMR (400 MHz, DMSO-d₆) δ 11.08 (s, 1H), 7.61-7.54 (dd, J=8.6, 7.1 Hz, 1H), 7.14 (d, J=8.6 Hz, 1H), 7.04 (d, J=7.1 Hz, 1H), 6.60 (t, J=5.7 Hz, 1H), 5.05 (dd, J=12.9, 5.4 Hz, 1H), 3.79-3.66 (m, 2H), 3.61 (m, 2H), 3.59-3.50 (m, 10H), 3.47 (dd, J=11.0, 5.4 Hz, 2H), 2.88 (m, 1H), 2.75 (t, J=6.0 Hz, 2H), 2.63-2.52 (m, 2H), 2.06-1.99 (m, 1H), 1.12 (dd, J=6.7, 3.7 Hz, 12H).

2-cyanoethyl (2-(2-(2-(2-((2-(2,6-dioxopiperidin-3-yl)-1,3-dioxoisoindolin-4-yl)amino)ethoxy)ethoxy)ethoxy)ethyl) diisopropylphosphoramidite (P3): Yellow oil, 48%. ¹H NMR (400 MHz, DMSO-d₆) δ 11.08 (s, 1H), 7.58 (dd, J=8.5, 7.2 Hz, 1H), 7.14 (d, J=8.6 Hz, 1H), 7.04 (d, J=7.0 Hz, 1H), 6.60 (t, J=5.7 Hz, 1H), 5.05 (dd, J=12.9, 5.4 Hz, 1H), 4.03 (m, 2H), 3.76-3.67 (m, 3H), 3.66-3.59 (m, 3H), 3.59-3.50 (m, 8H), 3.50-3.37 (m, 4H), 2.94-2.82 (m, 1H), 2.75 (t, J=6.0 Hz, 2H), 2.63-2.53 (m, 2H), 2.06-1.98 (m, 1H), 1.15-1.07 (m, 12H).

Synthesis of compound 8d-f: Compound VHL-032 (1.0 equiv) was dissolved in DCM and DMF (1:1), and TEA (3.0 equiv), 7d-f (1.5 equiv), and HATU (1.5 equiv) was added. The mixture was stirred at rt overnight. The reaction solution was diluted with DCM, washed with NaHCO₃ solution. The organic phase was concentrated and purified with flash chromatography (DCM:MeOH=100:0 to 98:2), giving compound 8d-f.

(2S,4R)—1-((S)-2-(6-((tert-butyldiphenylsilyl)oxy)hexanamido)-3,3-dimethylbutanoyl)-4-hydroxy-N-(4-(4-methylthiazol-5-yl)benzyl)pyrrolidine-2-carboxamide (8d): White foam solid, 70%. ¹H NMR (400 MHz, CDCl₃) δ 8.72 (s, 1H), 7.64 (dd, J=7.9, 1.6 Hz, 4H), 7.44-7.32 (m, 10H), 6.08 (d, J=8.7 Hz, 1H), 4.70 (t, J=7.9 Hz, 1H), 4.56 (dd, J=15.0, 6.6 Hz, 1H), 4.49 (d, J=8.8 Hz, 2H), 4.33 (dd, J=15.0, 5.2 Hz, 1H), 4.11-4.05 (m, 1H), 3.61 (m, 3H), 2.57-2.49 (m, 4H), 2.16 (t, J=7.6 Hz, 2H), 2.13-2.03 (m, 1H), 1.63-1.50 (m, 4H), 1.41-1.30 (m, 2H), 1.05-1.00 (m, 9H), 0.92 (s, 9H).

(2S,4R)—1-((S)-14-(tert-butyl)-2,2-dimethyl-12-oxo-3,3-diphenyl-4,7,10-trioxa-13-aza-3-silapentadecan-15-oyl)-4-hydroxy-N-(4-(4-methylthiazol-5-yl)benzyl)pyrrolidine-2-carboxamide (8e): Colorless oil, 62%. ¹H NMR (400 MHz, CDCl₃) δ 8.72 (s, 1H), 7.69-7.63 (m, 4H), 7.44-7.28 (m, 10H), 4.73 (t, J=7.9 Hz, 1H), 4.54 (m, 2H), 4.43 (d, J=8.3 Hz, 1H), 4.32 (dd, J=15.0, 5.3 Hz, 1H), 4.12 (d, J=11.4 Hz, 1H), 3.99 (q, J=15.8 Hz, 2H), 3.80 (dd, J=7.8, 3.3 Hz, 2H), 3.71-3.54 (m, 7H), 2.56 (m, 1H), 2.51 (s, 3H), 2.14-2.06 (m, 1H), 1.06-1.00 (m, 9H), 0.92 (s, 9H).

(2S,4R)—1-((S)-17-(tert-butyl)-2,2-dimethyl-15-oxo-3,3-diphenyl-4,7,10,13-tetraoxa-16-aza-3-silaoctadecan-18-oyl)-4-hydroxy-N-(4-(4-methylthiazol-5-yl)benzyl)pyrrolidine-2-carboxamide (8f): Colorless oil, 60%. ¹H NMR (400 MHz, CDCl₃) δ 8.70 (s, 1H), 7.69-7.64 (m, 4H), 7.43-7.32 (m, 10H), 4.72 (t, J=7.9 Hz, 1H), 4.53 (m, 2H), 4.47 (d, J=8.5 Hz, 1H), 4.33 (dd, J=15.0, 5.3 Hz, 1H), 4.08 (d, J=10.2 Hz, 1H), 4.03-3.91 (m, 2H), 3.79 (t, J=5.3 Hz, 2H), 3.68-3.55 (m, 11H), 2.56-2.48 (m, 4H), 2.15-2.06 (m, 1H), 1.03 (d, J=2.9 Hz, 9H), 0.94 (s, 9H).

Synthesis of compound 9a-c: Compound 8d-f (1.0 equiv) was dissolved in DCM and cooled to 0° C., then TEA (1.5 equiv) and DMAP (0.01 equiv) was added. The mixture was stirred and Ac₂O (1.5 equiv) was added slowly. The reaction was stirred at 0° C. for 1h. the reaction solution was washed with water, and the organic phase was dried with Na₂SO₄, filtered and concentrated. The residue was purified with flash chromatography (DCM:MeOH=100:0 to 98:2), giving compound 9a-c.

(3R,5S)-1-((S)-2-(6-((tert-butyldiphenylsilyl)oxy)hexanamido)-3,3-dimethylbutanoyl)-5-((4-(4-methylthiazol-5-yl)benzyl)carbamoyl)pyrrolidin-3-yl acetate (9a): White foam solid, 90%. ¹H NMR (400 MHz, CDCl₃) δ 8.89 (d, J=3.7 Hz, 1H), 7.64 (dd, J=7.6, 1.3 Hz, 4H), 7.43-7.32 (m, 10H), 7.18-7.13 (m, 1H), 6.04 (d, J=9.1 Hz, 1H), 5.37 (s, 1H), 4.70-4.65 (m, 1H), 4.62-4.50 (m, 2H), 4.34 (dd, J=14.9, 5.3 Hz, 1H), 4.05 (d, J=12.7 Hz, 1H), 3.84-3.76 (m, 1H), 3.63 (t, J=6.4 Hz, 2H), 2.71 (m, 1H), 2.54 (s, 3H), 2.17 (m, 3H), 2.03 (s, 3H), 1.57 (m, 4H), 1.36 (m, 2H), 1.03 (s, 9H), 0.89 (s, 9H).

(3R,5S)-1-((S)-14-(tert-butyl)-2,2-dimethyl-12-oxo-3,3-diphenyl-4,7,10-trioxa-13-aza-3-silapentadecan-15-oyl)-5-((4-(4-methylthiazol-5-yl)benzyl)carbamoyl)pyrrolidin-3-yl acetate (9b): Colorless oil, 92%. ¹H NMR (400 MHz, CDCl₃) δ 8.76 (s, 1H), 7.66 (dd, J=7.8, 1.5 Hz, 4H), 7.43-7.30 (m, 10H), 7.22 (d, J=8.4 Hz, 2H), 5.36 (s, 1H), 4.73-4.67 (m, 1H), 4.56-4.47 (m, 2H), 4.33 (dd, J=14.9, 5.4 Hz, 1H), 4.05 (d, J=11.9 Hz, 1H), 3.99 (d, J=4.9 Hz, 2H), 3.84-3.75 (m, 3H), 3.70-3.56 (m, 7H), 2.77-2.69 (m, 1H), 2.52 (s, 3H), 2.15 (m, 1H), 2.03 (s, 3H), 1.03 (s, 9H), 0.90 (s, 9H).

(3R,5S)-1-((S)-17-(tert-butyl)-2,2-dimethyl-15-oxo-3,3-diphenyl-4,7,10,13-tetraoxa-16-aza-3-silaoctadecan-18-oyl)-5-((4-(4-methylthiazol-5-yl)benzyl)carbamoyl)pyrrolidin-3-yl acetate (9c): Colorless oil, 87%. ¹H NMR (400 MHz, CDCl₃) δ 8.75 (s, 1H), 7.69-7.64 (m, 4H), 7.43-7.31 (m, 10H), 7.23 (dd, J=14.1, 7.4 Hz, 2H), 5.36 (s, 1H), 4.71 (dd, J=8.2, 6.6 Hz, 1H), 4.57-4.49 (m, 2H), 4.34 (dd, J=14.9, 5.4 Hz, 1H), 4.05 (d, J=13.7 Hz, 1H), 3.98 (d, J=4.3 Hz, 2H), 3.80 (dd, J=11.0, 5.8 Hz, 3H), 3.70-3.61 (m, 8H), 3.57 (t, J=5.3 Hz, 2H), 2.77-2.68 (m, 1H), 2.52 (s, 3H), 2.20-2.13 (m, 1H), 2.04 (s, 3H), 1.06-1.01 (s, 9H), 0.91 (s, 9H).

Synthesis of compound 10a-c: Compound 9a-c (1.0 equiv) was dissolved in THE and TBAF (1M in THF, 2.0 equiv) was added. The mixture was stirred at rt overnight. The solvent was removed and the residue was purified with flash chromatography (DCM:MeOH=100:0 to 97:3), giving compound 10a-c.

(3R,5S)-1-((S)-2-(6-hydroxyhexanamido)-3,3-dimethylbutanoyl)-5-((4-(4-methylthiazol-5-yl)benzyl)carbamoyl)pyrrolidin-3-yl acetate (10a): White solid, 60%. ¹H NMR (400 MHz, CDCl₃) δ 8.75 (s, 1H), 7.40-7.32 (m, 4H), 7.20 (t, J=6.0 Hz, 1H), 6.03 (d, J=9.2 Hz, 1H), 5.37 (m, 1H), 4.73-4.65 (m, 1H), 4.57 (dd, J=14.9, 6.6 Hz, 1H), 4.51 (d, J=9.2 Hz, 1H), 4.34 (dd, J=14.9, 5.2 Hz, 1H), 4.07 (d, J=11.7 Hz, 1H), 3.79 (dd, J=11.6, 4.6 Hz, 1H), 3.66-3.57 (m, 2H), 2.75-2.66 (m, 1H), 2.54 (s, 3H), 2.19 (m, 3H), 2.05 (s, 3H), 1.64 (m, 2H), 1.60-1.51 (m, 2H), 1.47 (m, 2H), 0.90 (s, 9H).

(3R,5S)-1-((S)-2-(2-(2-(2-hydroxyethoxy)ethoxy)acetamido)-3,3-dimethylbutanoyl)-5-((4-(4-methylthiazol-5-yl)benzyl)carbamoyl)pyrrolidin-3-yl acetate (10b): White solid, 68%. ¹H NMR (400 MHz, CDCl₃) δ 8.72 (s, 1H), 7.54 (d, J=9.5 Hz, 1H), 7.37 (s, 4H), 7.16 (t, J=5.8 Hz, 1H), 5.40 (m, 1H), 4.66 (dd, J=8.2, 6.7 Hz, 2H), 4.57 (dd, J=14.8, 6.6 Hz, 1H), 4.34 (dd, J=14.8, 5.4 Hz, 1H), 4.05 (dd, J=16.1, 5.5 Hz, 1H), 3.98-3.90 (m, 2H), 3.83 (dd, J=11.8, 4.7 Hz, 1H), 3.78-3.56 (m, 9H), 2.75-2.67 (m, 1H), 2.53 (d, J=3.3 Hz, 3H), 2.18 (m, 1H), 2.04 (d, J=2.5 Hz, 3H), 0.92 (s, 9H).

(3R,5S)-1-((S)-2-(tert-butyl)-14-hydroxy-4-oxo-6,9,12-trioxa-3-azatetradecanoyl)-5-((4-(4-methylthiazol-5-yl)benzyl)carbamoyl)pyrrolidin-3-yl acetate (10c): Colorless oil, 52%. ¹H NMR (400 MHz, CDCl₃) δ 8.72 (s, 1H), 7.50 (dd, J=11.1, 5.3 Hz, 1H), 7.39-7.28 (m, 5H), 5.39 (m, 1H), 4.69 (dd, J=8.1, 6.4 Hz, 1H), 4.57 (m, 2H), 4.33 (dd, J=14.9, 5.3 Hz, 1H), 4.02 (d, J=8.6 Hz, 2H), 3.84 (dd, J=11.6, 4.9 Hz, 1H), 3.72-3.62 (m, 10H), 3.60 (m, 1H), 3.56 (m, 1H), 3.54-3.48 (m, 1H), 3.47 (d, J=1.4 Hz, 2H), 2.74-2.65 (m, 1H), 2.53 (d, J=4.0 Hz, 3H), 2.21-2.12 (m, 1H), 2.04 (s, 3H), 0.93 (s, 9H).

Synthesis of compound P4-6: compound 10a-c (1.0 equiv) was dissolved in anhydrous DCM, DIPEA (2.0 equiv) and Cl-POCEN′Pr₂ (1.5 equiv) was added. The mixture was stirred at room temperature for 1 hour. Solvent was removed, and the residue was purified with flash chromatography (Hexane:Actone (5% TEA)=100:0 to 60:40), giving product as colorless oil.

(3R,5S)-1-((2S)-2-(6-(((2-cyanoethoxy)(diisopropylamino)phosphaneyl)oxy)hexanamido)-3,3-dimethylbutanoyl)-5-((4-(4-methylthiazol-5-yl)benzyl)carbamoyl)pyrrolidin-3-yl acetate (P4): Colorless oil, 60%. ¹H NMR (400 MHz, CDCl₃) δ 8.68 (s, 1H), 7.36 (q, J=8.1 Hz, 4H), 7.19 (t, J=5.7 Hz, 1H), 6.01 (d, J=9.1 Hz, 1H), 5.37 (m, 1H), 4.74-4.68 (m, 1H), 4.60-4.49 (m, 2H), 4.34 (dd, J=14.7, 5.1 Hz, 1H), 4.04 (d, J=12.1 Hz, 1H), 3.87-3.73 (m, 3H), 3.69-3.53 (m, 4H), 2.74 (m, 1H), 2.63 (t, J=6.5 Hz, 2H), 2.52 (d, J=0.6 Hz, 3H), 2.19 (m, 3H), 2.05 (s, 3H), 1.60 (m, 4H), 1.42-1.35 (m, 2H), 1.16 (q, J=6.0 Hz, 12H), 0.89 (s, 9H).

(3R,5S)-1-((2S)-2-(2-(2-(2-(((2-cyanoethoxy)(diisopropylamino)phosphaneyl)oxy)ethoxy)ethoxy)acetamido)-3,3-dimethylbutanoyl)-5-((4-(4-methylthiazol-5-yl)benzyl)carbamoyl)pyrrolidin-3-yl acetate (P5): Colorless oil, 67%. ¹H NMR (400 MHz, CDCl₃) δ 8.67 (s, 1H), 7.36 (q, J=8.2 Hz, 4H), 7.26-7.22 (m, 1H), 7.19 (d, J=9.2 Hz, 1H), 5.37 (m, 1H), 4.72 (dd, J=8.0, 6.7 Hz, 1H), 4.59-4.48 (m, 2H), 4.35 (dd, J=14.9, 5.3 Hz, 1H), 4.07-4.02 (m, 1H), 4.00 (d, J=3.5 Hz, 2H), 3.91-3.76 (m, 4H), 3.75-3.64 (m, 7H), 3.59 (1m, 2H), 2.79-2.70 (m, 1H), 2.66-2.61 (m, 2H), 2.52 (s, 3H), 2.21-2.12 (m, 1H), 2.04 (s, 3H), 1.19-1.14 (m, 12H), 0.91 (s, 9H).

(3R,5S)-1-((2S)-2-(tert-butyl)-14-(((2-cyanoethoxy)(diisopropylamino)phosphaneyl)oxy)-4-oxo-6,9,12-trioxa-3-azatetradecanoyl)-5-((4-(4-methylthiazol-5-yl)benzyl)carbamoyl)pyrrolidin-3-yl acetate (P6): Colorless oil, 40%. ¹H NMR (400 MHz, CDCl₃) δ 8.68 (s, 1H), 7.36 (q, J=8.1 Hz, 4H), 7.25-7.17 (m, 2H), 5.37 (m, 1H), 4.75-4.69 (m, 1H), 4.59-4.49 (m, 2H), 4.36 (dd, J=14.9, 5.3 Hz, 1H), 4.07-4.02 (m, 1H), 4.00 (d, J=4.7 Hz, 2H), 3.90-3.75 (m, 4H), 3.75-3.53 (m, 13H), 2.80-2.71 (m, 1H), 2.64 (t, J=6.5 Hz, 2H), 2.52 (s, 3H), 2.16 (m, 1H), 2.04 (s, 3H), 1.21-1.14 (m, 12H), 0.92 (s, 9H).

### Synthesis of Modifiers

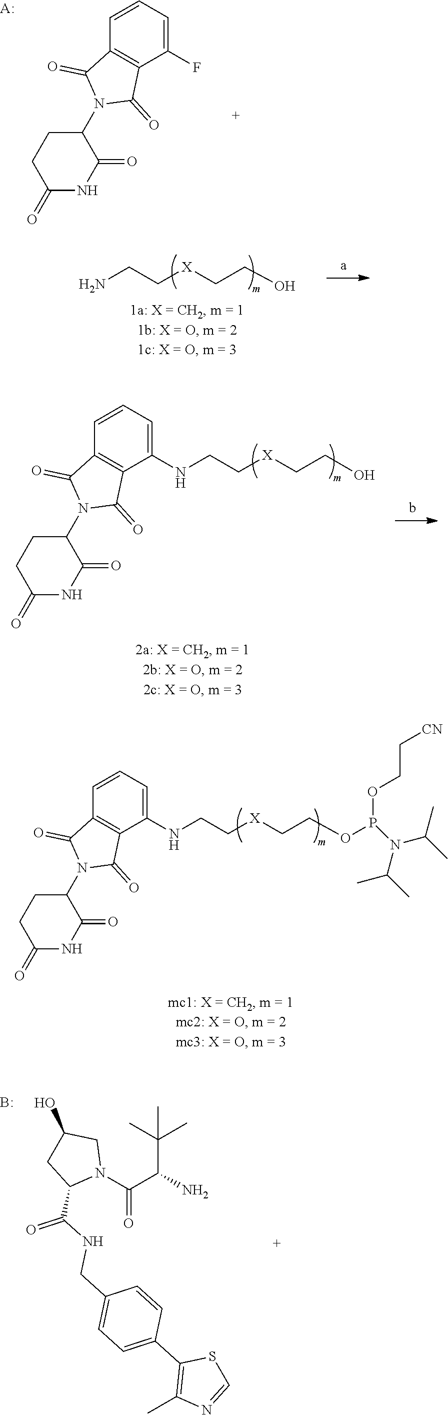

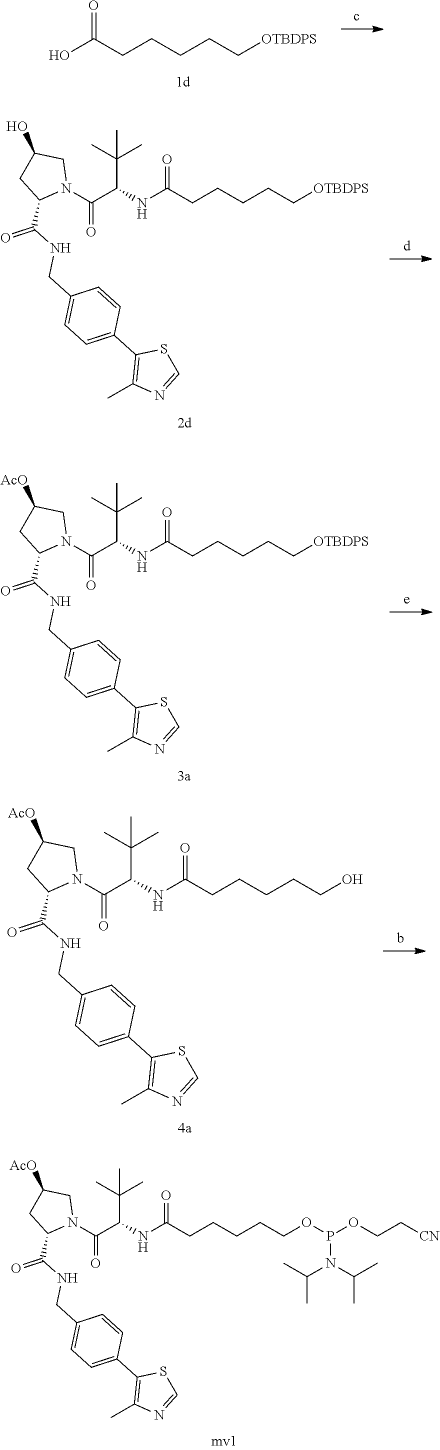

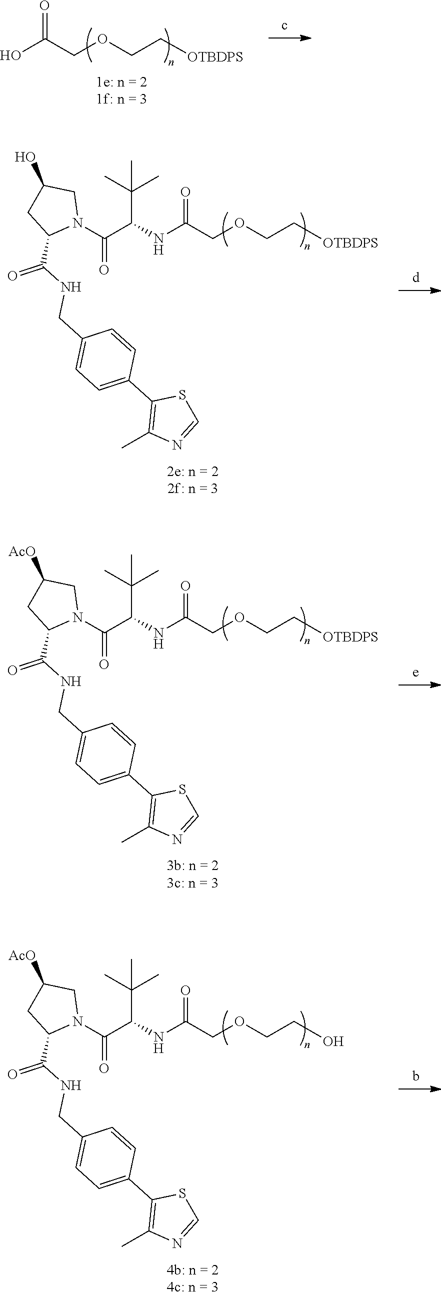

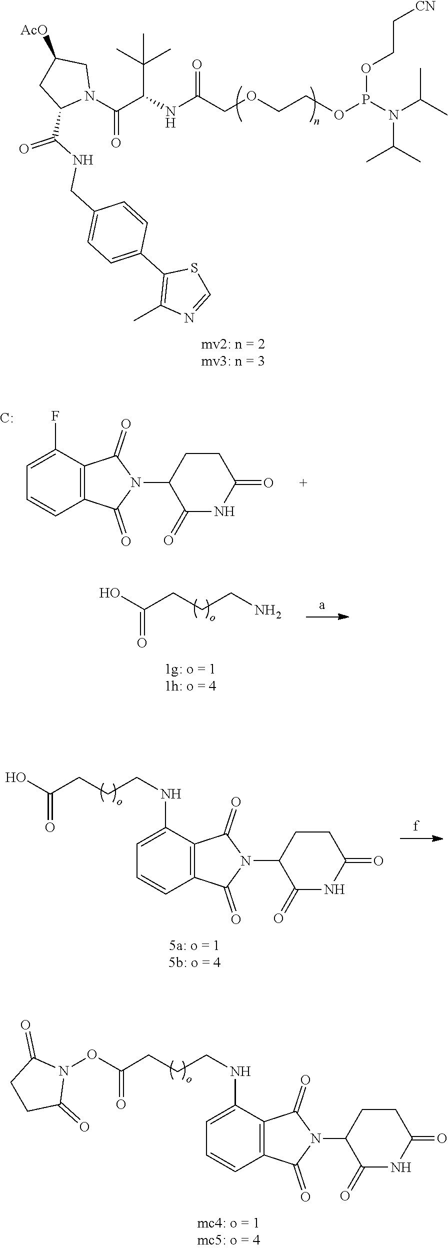

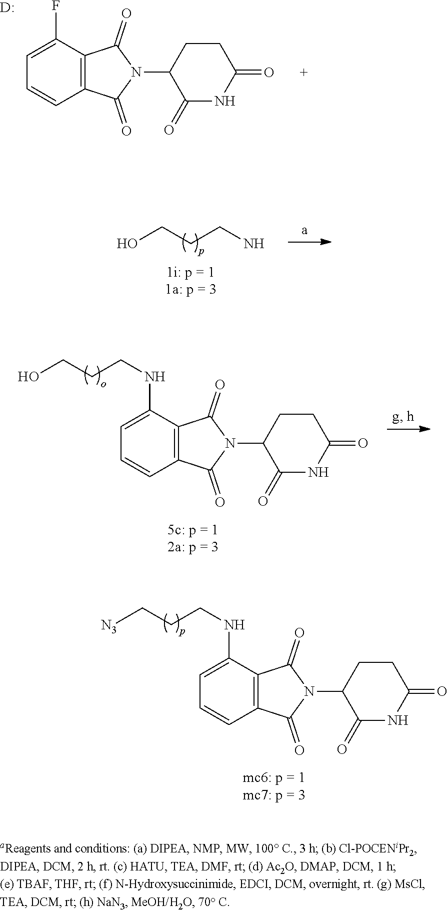

Synthesis of compounds 5a-5c: Compound 4-fluoro-thalidomide (1.0 equiv) was dissolved in DMA, DIPEA (2.0 equiv) and compound 1g-i (1.5 equiv) were added, the mixture was heated to 100° C. in sealed tube overnight. then the mixture was concentrated and purified by reverse phase flash chromatography (H2O:MeOH=100:0 to 50:50), giving compounds 5a-5c.

4-((2-(2,6-dioxopiperidin-3-yl)-1,3-dioxoisoindolin-4-yl)amino)butanoic acid (5a): ¹H NMR (400 MHz, DMSO-d₆) δ 11.10 (s, 1H), 7.58 (t, J=7.8 Hz, 1H), 7.13 (d, J=8.6 Hz, 1H), 7.02 (d, J=7.1 Hz, 1H), 6.66 (t, J=5.8 Hz, 1H), 5.05 (dd, J=12.8, 5.1 Hz, 1H), 3.31 (m, 2H), 2.94-2.81 (m, 1H), 2.64-2.51 (m, 2H), 2.30 (t, J=7.1 Hz, 2H), 2.02 (d, J=6.8 Hz, 1H), 1.78 (m, 2H).

7-((2-(2,6-dioxopiperidin-3-yl)-1,3-dioxoisoindolin-4-yl)amino)heptanoic acid (5b): ¹H NMR (400 MHz, DMSO-d₆) δ 12.00 (s, 1H), 11.10 (s, 1H), 7.58 (t, J=7.8 Hz, 1H), 7.09 (d, J=8.6 Hz, 1H), 7.02 (d, J=7.0 Hz, 1H), 6.54 (t, J=5.7 Hz, 1H), 5.05 (dd, J=12.9, 5.2 Hz, 1H), 3.31-3.24 (m, 2H), 2.88 (m, 1H), 2.55 (m, 2H), 2.20 (t, J=7.3 Hz, 2H), 2.07-1.97 (m, 1H), 1.61-1.44 (m, 4H), 1.32 (m, 4H).

2-(2,6-dioxopiperidin-3-yl)-4-((3-hydroxypropyl)amino)isoindoline-1,3-dione (5c):Yellow solid, 60%. ¹HNMR (400 MHz, CDCl₃) δ 8.15 (s, 1H), 7.50 (t, J=7.8 Hz, 1H), 7.09 (d, J=7.1 Hz, 1H), 6.93 (d, J=8.5 Hz, 1H), 4.92 (dd, J=11.9, 5.1 Hz, 1H), 3.82 (t, J=5.7 Hz, 2H), 3.44 (t, J=6.6 Hz, 2H), 2.93-2.66 (m, 3H), 2.16-2.07 (m, 1H), 1.96-1.87 (m, 2H).

Synthesis of compounds mc4 and mc5: Compound 5a or 5b (1.0 equiv) and N-Hydroxysuccinimide (1.5 equiv) were mixed in DCM, cool to 0° C., then EDCI (1.3 equiv) was added slowly. The mixture was stirred at RT overnight. The reaction was diluted with DCM and washed, with H₂O and brine. The organic phase was dried with Na₂SO₄, filtered and concentrated, giving mc4 and mc5 as yellow solid.

2,5-dioxopyrrolidin-1-yl 4-((2-(2,6-dioxopiperidin-3-yl)-1,3-dioxoisoindolin-4-yl)amino)butanoate (mc4): 88%; LC-MS (ESI⁺): m/z 457.2 [M+H⁺]

2,5-dioxopyrrolidin-1-yl 7-((2-(2,6-dioxopiperidin-3-yl)-1,3-dioxoisoindolin-4-yl)amino)heptanoate (mc5): 85%; LC-MS (ESI⁺): m/z 499.3 [M+H⁺]

Synthesis of compounds mc6 and mc7: Compound 5c or 8 was dissolved in DCM, TEA (2.0 equiv) and MsCl (1.2 equiv) were added, the mixture was stirred at RT for 2h. The reaction was added water, then extracted with DCM, the organic phase was dried and concentrated. The residue was dissolved in DCM MeOH/H₂O and NaN₃ was added, then the mixture was heated to 70° C. overnight. Solvent was removed, to the residue was added water, then extracted with EA twice. The organic phase was concentrated and purified by flash chromatography (DCM:EA=100:0 to 85:15), giving compounds mc6 and mc7.

4-((3-azidopropyl)amino)-2-(2,6-dioxopiperidin-3-yl)isoindoline-1,3-dione (mc6):Yellow solid, 30%. ¹H NMR (400 MHz, CDCl₃) δ 8.07 (s, 1H), 7.52 (t, J=7.8 Hz, 1H), 7.12 (d, J=7.1 Hz, 1H), 6.92 (d, J=8.6 Hz, 1H), 6.29 (s, 1H), 4.92 (dd, J=11.9, 5.2 Hz, 1H), 3.47 (t, J=6.3 Hz, 2H), 3.41 (t, J=6.7 Hz, 2H), 2.80 (m, 3H), 2.19-2.08 (m, 1H), 1.92 (m, 2H).

4-((5-azidopentyl)amino)-2-(2,6-dioxopiperidin-3-yl)isoindoline-1,3-dione (mc7):Yellow solid, 46%. ¹H NMR (400 MHz, CDCl₃) δ 8.17 (s, 1H), 7.50 (t, J=7.8 Hz, 1H), 7.09 (d, J=7.1 Hz, 1H), 6.88 (d, J=8.5 Hz, 1H), 6.24 (s, 1H), 4.91 (dd, J=12.0, 5.3 Hz, 1H), 3.30 (m, 4H), 2.93-2.67 (m, 3H), 2.17-2.08 (m, 1H), 1.68 (m, 4H), 1.50 (m, 2H).

### Synthesis of Modified Reverse Strand

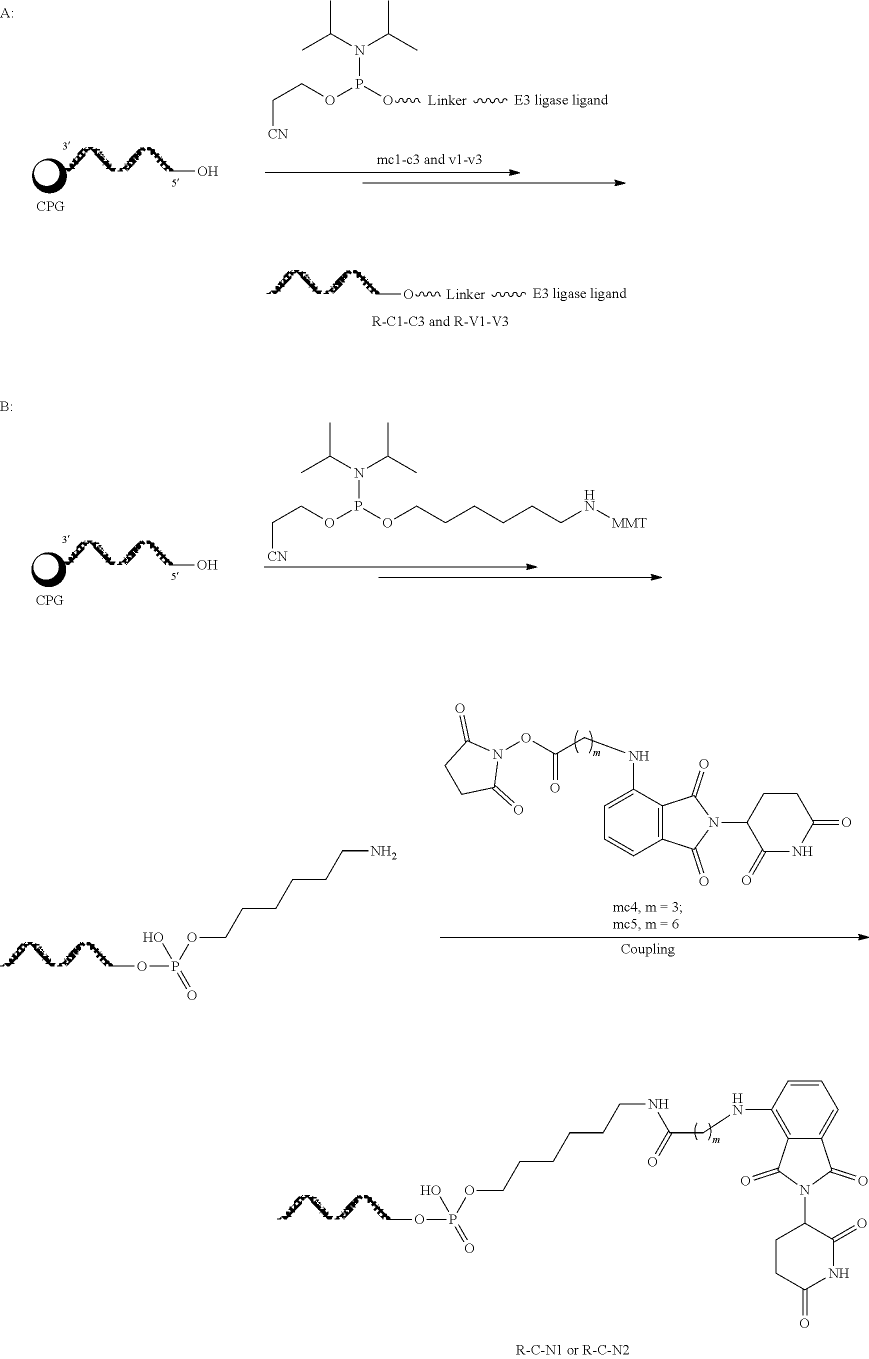

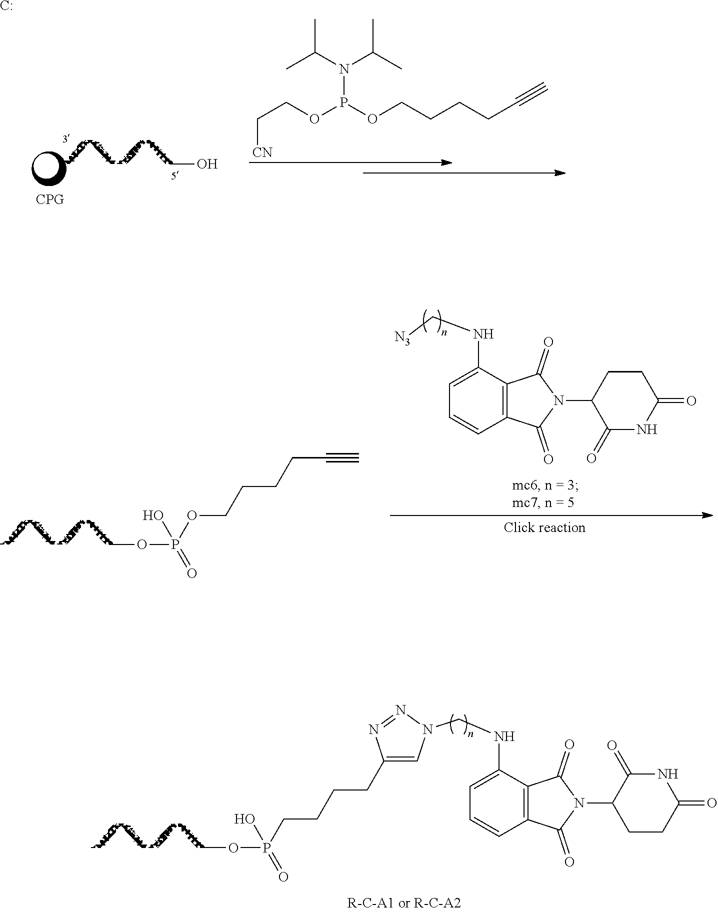

### Synthesis of Oligonucleotides

All oligonucleotides used in this work were synthesized and reverse phase-HPLC purified by ExonanoRNA company (Columbus, OH). Mass and purity (>95%) was confirmed by LC-MS from Novatia, LLC company with Xcalibur system.

### Annealing Reaction

Single stranded and reverse oligonucleotides were mixed in an assembly buffer (10 mM Tris-HCl [pH7.5], 100 mM NaCl, 1 mM EDTA), and heated to 90° C. for 5 minutes, then slowly cool down to 37° C. within 1 hour. Double stranded O′PROTACs were mixed well, aliquoted and stored at −20° C. for the future use.

### Exemplary O′PROTACs

TABLE 2
Exemplary O′PROTACs.

| O′PROTAC | O′PROTAC | Sequence | SEQ ID NO |
|---|---|---|---|
| AR-11 | AR OP-C1 | Forward: 5′-TTAGGGTACAC-3′ | 1 |
| | | Reverse: 5′-P1-GTGTACCCTAA-3′ | 107 |
| AR-12 | AR OP-C2 | Forward: 5′-TTAGGGTACAC-3′ | 1 |
| | | Reverse: 5′-P2-GTGTACCCTAA-3′ | 107 |
| AR-13 | AR OP-C3 | Forward: 5′-TTAGGGTACAC-3′ | 1 |
| | | Reverse: 5′-P3-GTGTACCCTAA-3′ | 107 |
| AR-14 | AR OP-V1 | Forward: 5′-TTAGGGTACAC-3′ | 1 |
| | | Reverse: 5′-P4-GTGTACCCTAA-3′ | 107 |
| AR-15 | AR OP-V2 | Forward: 5′-TTAGGGTACAC-3′ | 1 |
| | | Reverse: 5′-P5-GTGTACCCTAA-3′ | 107 |
| AR-16 | AR OP-V3 | Forward: 5′-TTAGGGTACAC-3′ | 1 |
| | | Reverse: 5′-P6-GTGTACCCTAA-3′ | 107 |
| AR-21 | AR-2 OP-C1 | Forward: 5′-TTAGGGTACACCGTGTACCT-3′ | 2 |
| | | Reverse: 5′-P1-AGGTACACGGTGTACCCTAA-3′ | 108 |
| AR-22 | AR-2 OP-C2 | Forward: 5′-TTAGGGTACACCGTGTACCT-3′ | 2 |
| | | Reverse: 5′-P2-AGGTACACGGTGTACCCTAA-3′ | 108 |
| AR-3 | AR-2 OP-C3 | Forward: 5′-TTAGGGTACACCGTGTACCT-3′ | 2 |
| | | Reverse: 5′-P3-AGGTACACGGTGTACCCTAA-3′ | 108 |
| AR-24 | AR-2 OP-V1 | Forward: 5′-TTAGGGTACACCGTGTACCT-3′ | 2 |
| | | Reverse: 5′-P4-AGGTACACGGTGTACCCTAA-3′ | 108 |
| AR-25 | AR-2 OP-V2 | Forward: 5′-TTAGGGTACACCGTGTACCT-3′ | 2 |
| | | Reverse: 5′-P5-AGGTACACGGTGTACCCTAA-3′ | 108 |
| AR-26 | AR-2 OP-V3 | Forward: 5′-TTAGGGTACACCGTGTACCT-3′ | 2 |
| | | Reverse: 5′-P6-AGGTACACGGTGTACCCTAA-3′ | 108 |
| ERG-31 | ERG OP-C1 | Forward: 5′-ACGGACCGGAAATCCGGTT-3′ | 3 |
| | | Reverse: 5′-P1-AACCGGATTTCCGGTCCGT-3′ | 109 |
| ERG-32 | ERG OP-C2 | Forward: 5′-ACGGACCGGAAATCCGGTT-3′ | 3 |
| | | Reverse: 5′-P2-AACCGGATTTCCGGTCCGT-3′ | 109 |
| ERG-33 | ERG OP-C3 | Forward: 5′-ACGGACCGGAAATCCGGTT-3′ | 3 |
| | | Reverse: 5′-P3-AACCGGATTTCCGGTCCGT-3′ | 109 |
| ERG-34 | ERG OP-V1 | Forward: 5′-ACGGACCGGAAATCCGGTT-3′ | 3 |
| | | Reverse: 5′-P4-AACCGGATTTCCGGTCCGT-3′ | 109 |
| ERG-35 | ERG OP-V2 | Forward: 5′-ACGGACCGGAAATCCGGTT-3′ | 3 |
| | | Reverse: 5′-P5-AACCGGATTTCCGGTCCGT-3′ | 109 |
| ERG-36 | ERG OP-V3 | Forward: 5′-ACGGACCGGAAATCCGGTT-3′ | 3 |
| | | Reverse: 5′-P6-AACCGGATTTCCGGTCCGT-3′ | 109 |
| ERG-37 | ERG OP-C4 | Forward: 5′-P1-GACCGGAAATCCGGTTCGT-3′ | 110 |
| | | Reverse: 5′-ACGAACCGGATTTCCGGTC-3′ | 111 |
| ERG-38 | ERG OP-C5 | 5′-P1-AACCGGATTTCCGGTCCGTAAGGGAAACGGAC | 112 |
| | | CGGAAATCCGGTT-3′ | |
| FITC-ERG 3-13 | FITC-ERG OP-C1 | Forward: 5′-FITC-ACGGACCGGAAATCCGGTT-3′ | 3 |
| | | Reverse: 5′-P1-AACCGGATTTCCGGTCCGT-3′ | 109 |
| Biotin-ERG 3-13 | Biotin-ERG OP-C1 | Forward: 5′-Biotin-ACGGACCGGAAATCCGGTT-3′ | 3 |
| | | Reverse: 5′-P1-AACCGGATTTCCGGTCCGT-3′ | 109 |
| FOXA1 4-14 | FOXAI OP-C1 | Forward: 5′-GCTCCTTAAGTAAACAAAC-3′ | 113 |
| | | Reverse: 5′-P1-GTTTGTTTACTTAAGGAGC-3 | 114 |
| FOXA1 4-24 | FOXA1 OP-C2 | Forward: 5′-GCTCCTTAAGTAAACAAAC-3′ | 113 |
| | | Reverse: 5′-P2-GTTTGTTTACTTAAGGAGC-3 | 114 |
| FOXA1 4-34 | FOXA1 OP-C3 | Forward: 5′-GCTCCTTAAGTAAACAAAC-3′ | 113 |
| | | Reverse: 5′-P3-GTTTGTTTACTTAAGGAGC-3 | 114 |
| FOXA1 4-44 | FOXA1 OP-V1 | Forward: 5′-GCTCCTTAAGTAAACAAAC-3′ | 113 |
| | | Reverse: 5′-P4-GTTTGTTTACTTAAGGAGC-3 | 114 |
| FOXA1 4-54 | FOXA1 OP-V2 | Forward: 5′-GCTCCTTAAGTAAACAAAC-3′ | 113 |
| | | Reverse: 5′-P5-GTTTGTTTACTTAAGGAGC-3 | 114 |
| FOXA1 4-64 | FOXA1 OP-V3 | Forward: 5′-GCTCCTTAAGTAAACAAAC-3′ | 113 |
| | | Reverse: 5′-P6-GTTTGTTTACTTAAGGAGC-3 | 114 |
| LEF1-51 | LEF1 OP-C1 | Forward: 5′-TACAAAGATCAAAGGGTT-3′ | 5 |
| | | Reverse: 5′-P1-AACCCTTTGATCTTTGTA-3′ | 115 |
| LEF1-52 | LEF1 OP-C2 | Forward: 5′-TACAAAGATCAAAGGGTT-3′ | 5 |
| | | Reverse: 5′-P2-AACCCTTTGATCTTTGTA-3′ | 115 |
| LEF1-53 | LEF1 OP-C3 | Forward: 5′-TACAAAGATCAAAGGGTT-3′ | 5 |
| | | Reverse: 5′-P3-AACCCTTTGATCTTTGTA-3′ | 115 |
| LEF1-54 | LEF1 OP-V1 | Forward: 5′-TACAAAGATCAAAGGGTT-3′ | 5 |
| | | Reverse: 5′-P4-AACCCTTTGATCTTTGTA-3′ | 115 |
| LEF1-55 | LEF1 OP-V2 | Forward: 5′-TACAAAGATCAAAGGGTT-3′ | 5 |
| | | Reverse: 5′-P5-AACCCTTTGATCTTTGTA-3′ | 115 |
| LEF1-56 | LEF1 OP-V3 | Forward: 5′-TACAAAGATCAAAGGGTT-3′ | 5 |
| | | Reverse: 5′-P6-AACCCTTTGATCTTTGTA-3′ | 115 |

### Cell Culture and Transfection

PC-3, DU145, VCaP and 293T cells were obtained from the American Type Culture Collection (ATCC). 293T cells were maintained in DMEM medium with 10% FBS, and PC-3 and DU145 cells were maintained in RPMI medium with 10% FBS, while VCaP cells were maintained in RPMI medium with 15% FBS. Cells were transiently transfected using Lipofectamine 2000 f mixed with O′PROTAC according to the manufacturer's instructions.

### Western Blot

Cell lysates were subjected to SDS-PAGE, and proteins were transferred to nitrocellulose membranes (GE Healthcare Sciences). The membranes were blocked in Tris-buffered saline (TBS, pH 7.4) containing 5% non-fat milk and 0.1% Tween-20, washed twice in TBS containing 0.1% Tween-20, and incubated with primary antibody overnight at 4° C., followed by secondary antibody for 1 h at room temperature. The proteins of interest were visualized using ECL chemiluminescence system (Thermo Fisher).

### Example 2: ERG O′PROTACs

ERG transcription factor belongs to the ETS family and is involved in bone development, hematopoiesis, angiogenesis, vasculogenesis, inflammation, migration and invasion (Oncogene 2016; 35:403-14). Notably, ERG protein is overexpressed in approximately 50% of all human prostate cancer cases including both primary and metastatic prostate cancer, most due to the fusion of ERG gene with the androgen-responsive TMPRSS2 gene promoter. TMPRSS2-ERG fusion gene results in aberrant overexpression of truncated ERG which contain the intact DNA binding domain and transactivation, implying that increased expression of truncated but fully functional ERG is a key factor to drive prostate cancer progression (Am J Surg Pathol. 2007; 31:882-8). Therefore, therapeutic targeting ERG is urgently needed to effectively treat prostate cancer patients.

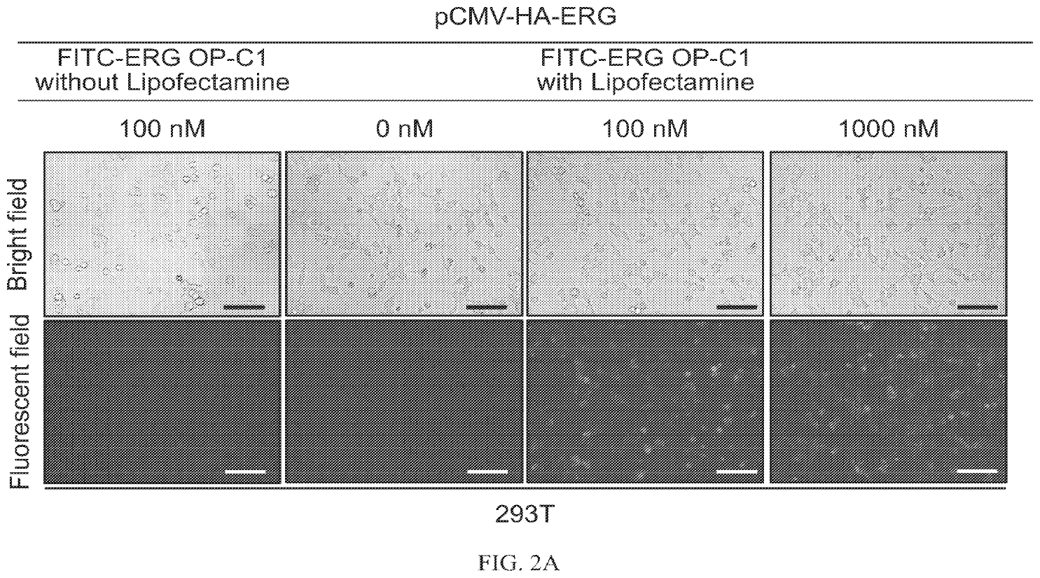

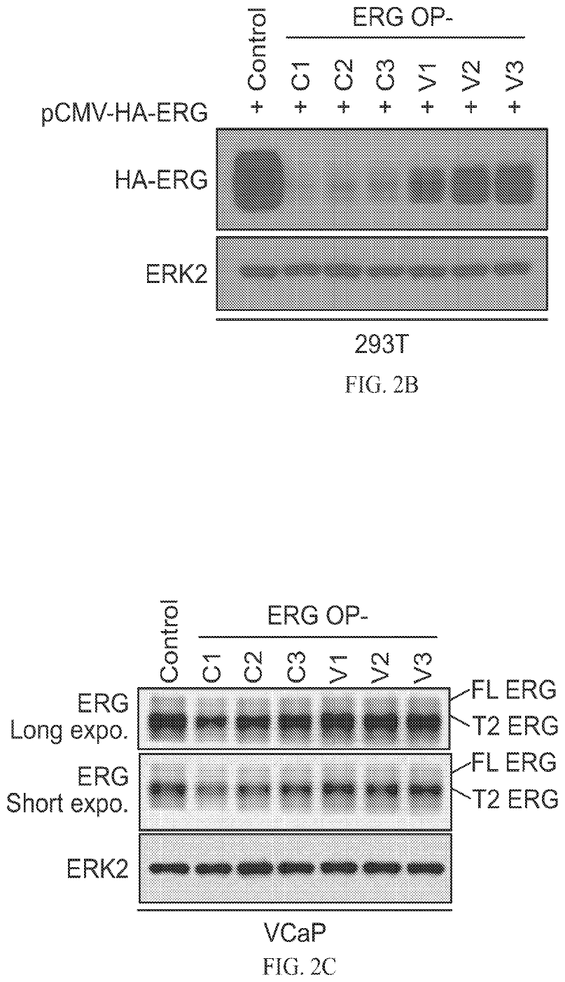

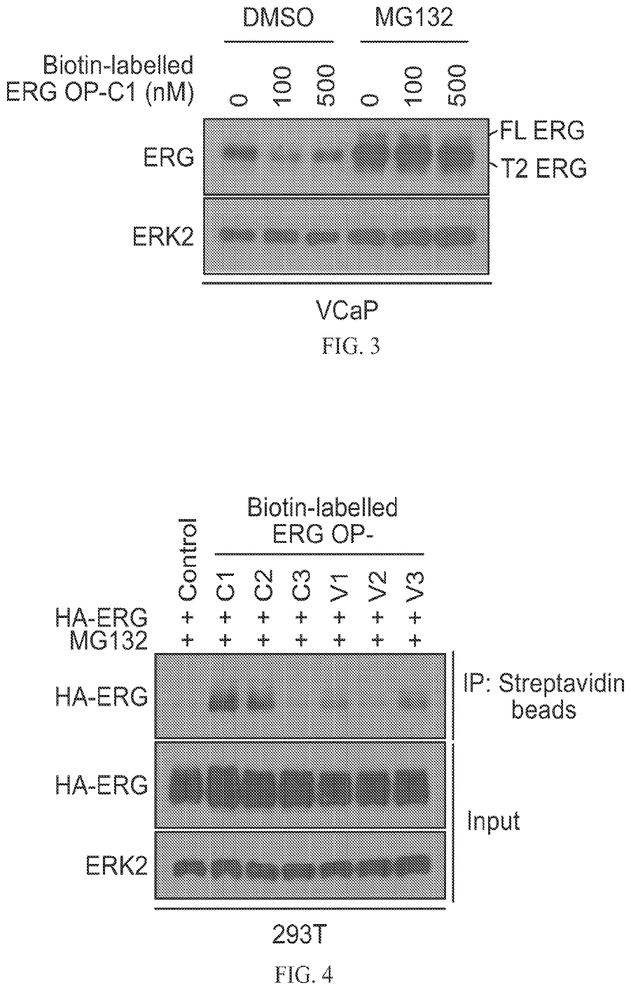

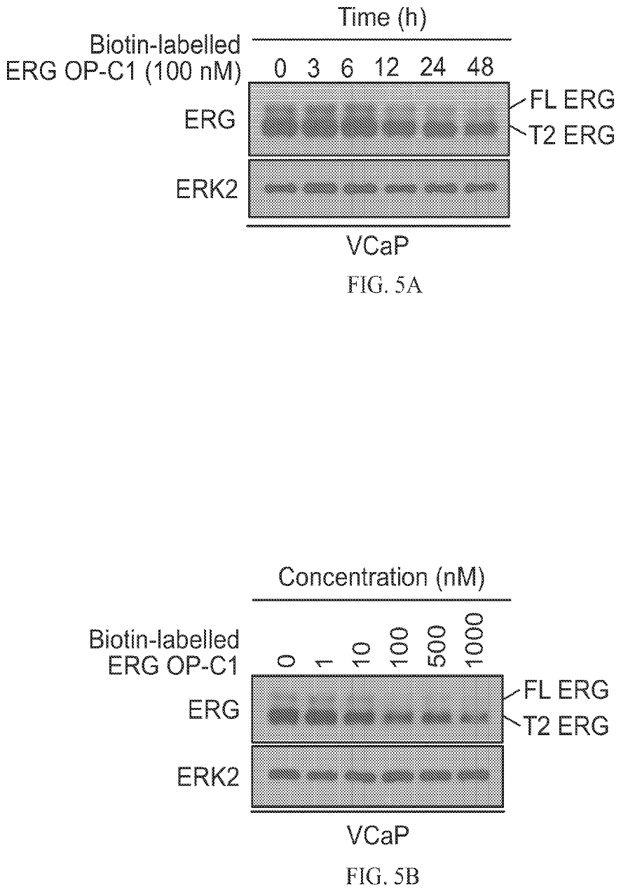

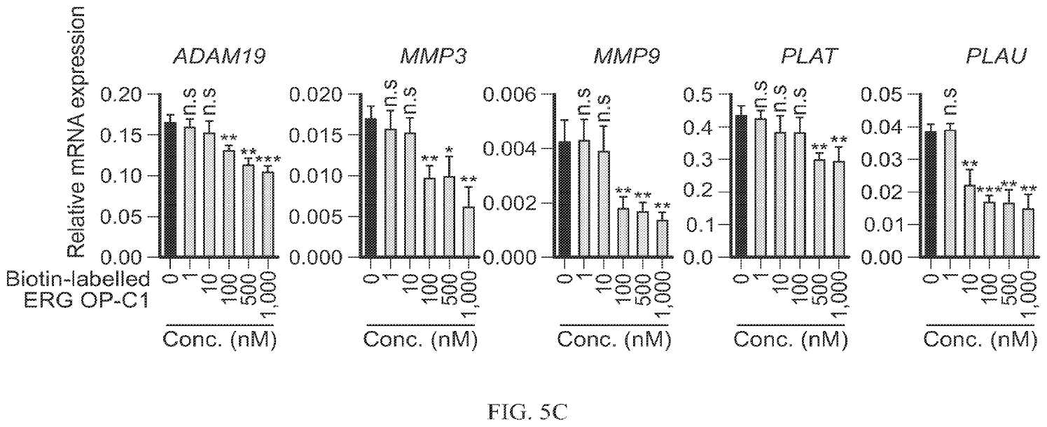

To assess the effects of ERG O′PROTACs on the protein level of ERG in cells, 293T cells were transfected with HA-ERG plasmid and biotin-labelled O′PROTAC at 100 nM for 48 hours. Then ERG protein level was measured by western blotting. Strikingly, a significant downregulation of ERG protein level was observed upon treatment with ERG O′PROTAC-31, 32 and 33 attached with pomalidomide at quite low concentration while it was not effectively detected in cells transfected with ERG O′PROTAC 34, 35 and 36 conjugated with VH 032 (FIG. 2B). Furthermore, using biotin-pulldown assay, a significant amount of ectopically expressed HA-ERG was effectively pulled down by both biotin-labelled ERG O′PROTAC 31 and 32, but no or less effectively by other ERG O′PROTACs (FIG. 4), indicating that these two O′PROTACs can effectively bind to ERG proteins as anticipated. This might provide a plausible explanation for the observation that ERG O′PROTACs 31 and 32 had greater effect on ERG protein degradation compared to other ERG O′PROTACs examined. Intriguingly, a shorter linker such as five carbon atoms was favored for the more stable ternary complex. To further investigate the cellular effect on endogenous ERG protein level, ERG O′PROTACs were tested in ERG-overexpressed human prostate cancer cell line (VCaP cell) that harbors TMPRSS2-ERG fusion. Similar to the effects in 293T cells, treatment of VCaP cells with ERG O′PROTACs 31 and 32 effectively decreased the level of endogenous full-length (FL) ERG and TMPRSS2-ERG (T2-ERG) proteins (FIG. 2C). Importantly, ERG O′PROTAC-induced downregulation of ERG proteins was completely blocked by treatment of cells with the proteasome inhibitor MG132 (FIG. 3), suggesting that ERG O′PROTAC induces proteasomal degradation of ERG proteins. Further time-course results have shown that ERG O′PROTACs were effective starting from 12 hrs to 48 hrs examined (FIG. 5A). Consistent with the results shown in FIG. 3, the dose-course experiment revealed that 100 nM of ERG O′PROTAC 31 resulted in a significant inhibition of ERG protein level and this effect was not obviously improved by higher concentrations such as 500 nM and 1,000 nM, indicating that the effect of ERG 31 can be saturated in a high concentration (FIG. 5B F). Additionally, treatment of VCaP cells with ERG O′PROTAC 31 inhibited mRNA expression of ERG target genes including ADAM19, MMP3, MMP9, PLAT and PLAU (FIG. 5C), suggesting that ERG O′PROTAC inhibits ERG transcriptional activity in VCaP prostate cancer cells.

### Example 3: LEF-1 O′PROTACs

LEF1 belongs to a family of transcriptional factors, namely lymphoid enhancer factor/T cell factor (LEF/TCF) which is regarded as an important transcriptional complex with 0-catenin (Nature, 1996, 382(6592): p. 638-42). LEF1 is implicated in the development of prostate cancer particularly in regulating prostate cancer growth and invasion capabilities (Oncogene, 2006, 25(24): p. 3436-44; Cancer Res, 2009, 69(8): p. 3332-8). Therefore, the inhibition of LEF1 is becoming an important target for therapy of cancer such as prostate cancer.

The degradation capability of each LEF1 O′PROTACs in PC-3 prostate cancer cell line was evaluated. Western blot assay was utilized to detect the expression of LEF1 protein. Expression of LEF1 was decreased in PC-3 cells transfected with LEF1 O′PROTAC 54 (FIG. 6), suggesting that LEF1 O′PROTAC 54 is effective in degrading LEF1 protein.

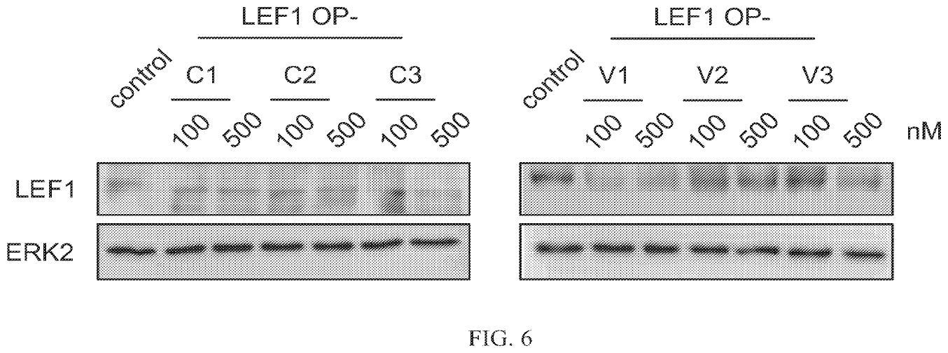

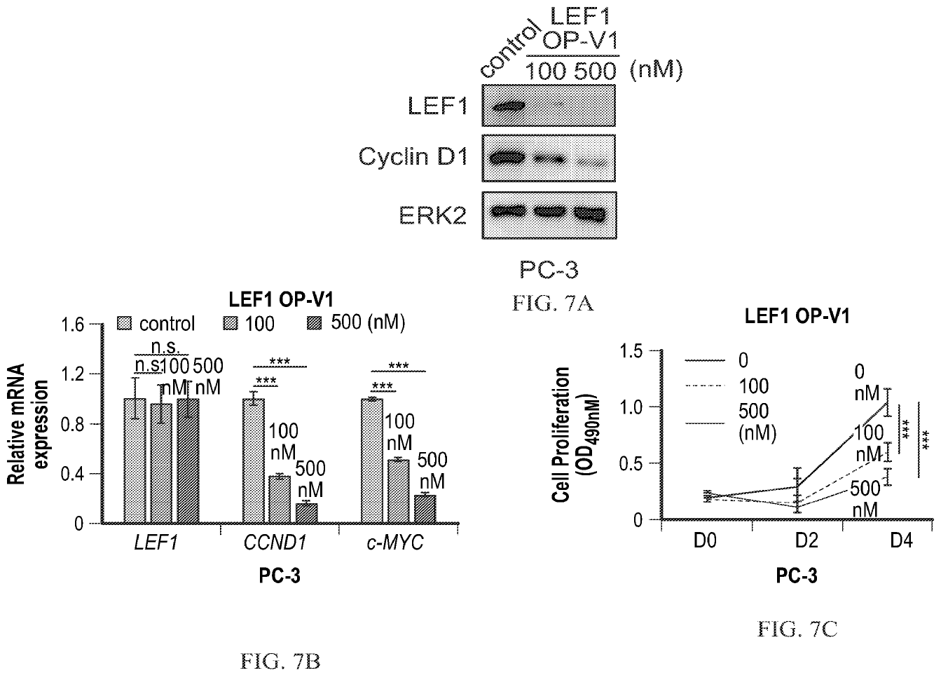

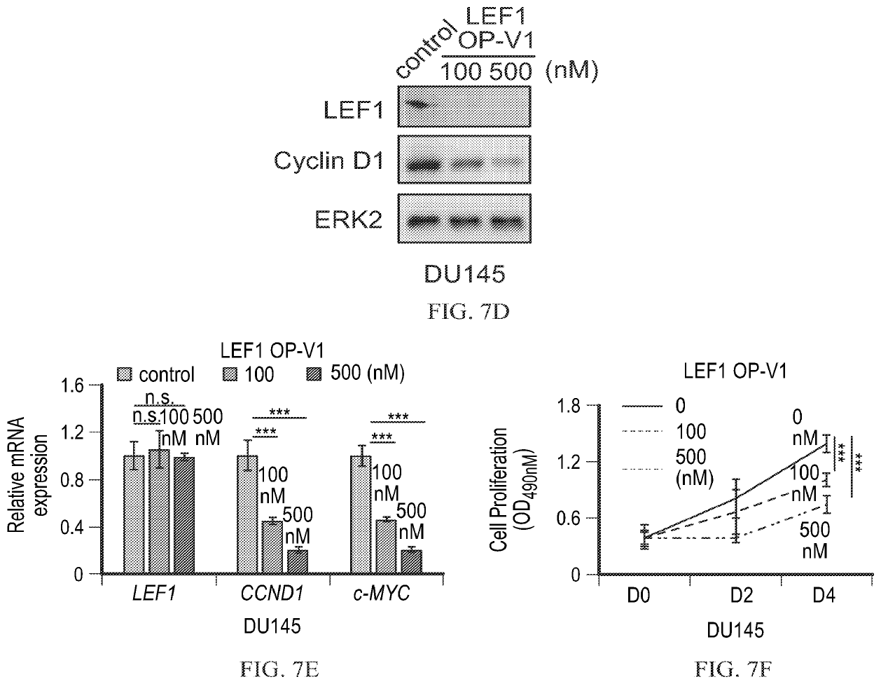

Next, the effect of LEF1 O′PROTAC on the transcriptional activity of Catenin/LEF1 was examined. Treatment of PC-3 prostate cancer cells with LEF1 O′PROTAC 54 downregulated mRNA expression of CCND1 and c-MYC, two target genes of Catenin/LEF1 in a dose-dependent manner (FIG. 7A, B). While LEF1 O′PROTAC 54 treatment did not affect mRNA expression of LEF1, it markedly decreased expression of LEF1 protein and its target protein Cyclin D1 in PC-3 (FIG. 7A). Importantly, LEF1 O′PROTAC 54 treatment significantly inhibited growth of PC-3 cells in a time- and dose-dependent fashion (FIG. 7A, C). Similar results were obtained in another prostate cancer cell line DU145 (FIG. 7D-F).

### Example 4: LEF1 OP-VJ Inhibits Prostate Cancer Tumor Growth In Vivo

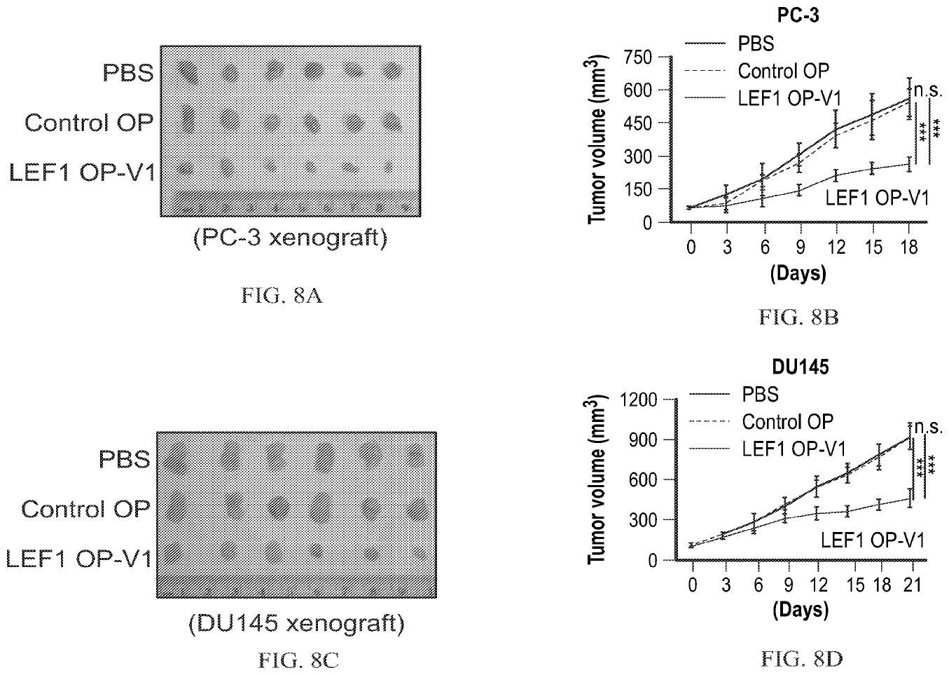

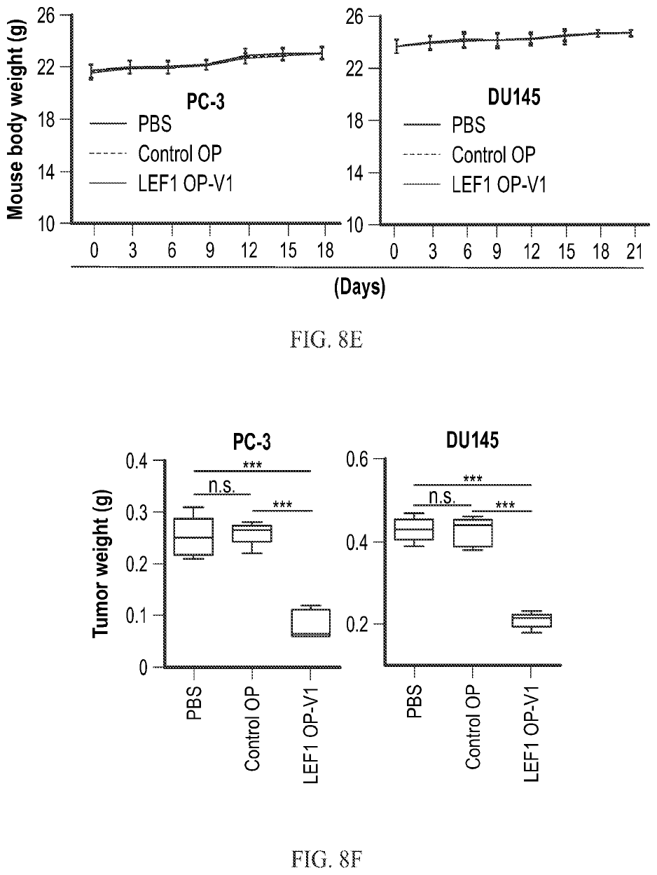

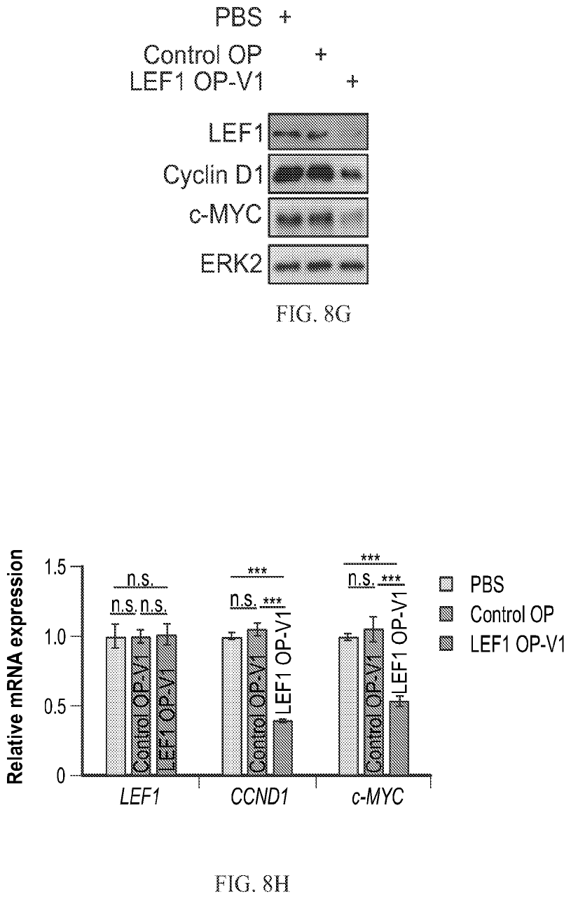

The effect of LEF OP-V1 was further investigated in vivo. PC-3 and DU145 xenograft tumors were generated by subcutaneous injection of PC-3 and DU145 cells into SCID mice. By treating mice with positively charged polyethylenimine (PEI)-condensed DNA oligo-based O′PROTAC, it was demonstrated that LEF1 OP-V1 effectively inhibited PC-3 and DU145 tumor growth in mice compared to the treatment of phosphate-buffered saline (PBS) or control OP (FIGS. 8A-8D). Little or no pronounced effect was observed on the weight loss of mice after administration of LEF1 OP-V1 (FIG. 8E). On the contrary, the tumor weight was largely decreased by the treatment of LEF1 OP-V1 (FIG. 8F), implying the inhibitory effect of LEF1 OP-V1 on tumor growth was not caused by the general toxicity of the O′PROTAC in mice. Consistent with the effect of LEF1 OP-V1 on tumor growth, LEF1 OP-V1 treatment decreased LEF1 protein and inhibited LEF1/O-Catenin target gene expression in tumors (FIGS. 8G and 8H). Importantly, LEF1 OP-V1 treatment also significantly impeded Ki67 expression in PC-3 tumors we examined, and little or no noticeable effect of LEF1 OP-V1 on cell death was observed (FIGS. 81 and 8J). These results suggest that LEF1 O′PROTAC can effectively deplete LEF1 protein and inhibit prostate cancer cell growth in vivo.

### Example 5: ERG O′PROTAC Inhibits Prostate Cancer Cell Growth In Vitro and Decreases Cell Invasion

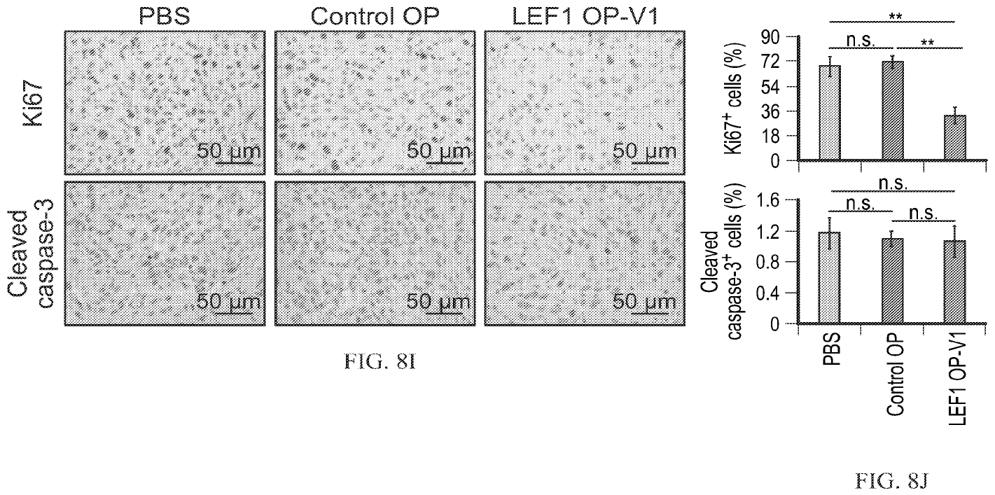

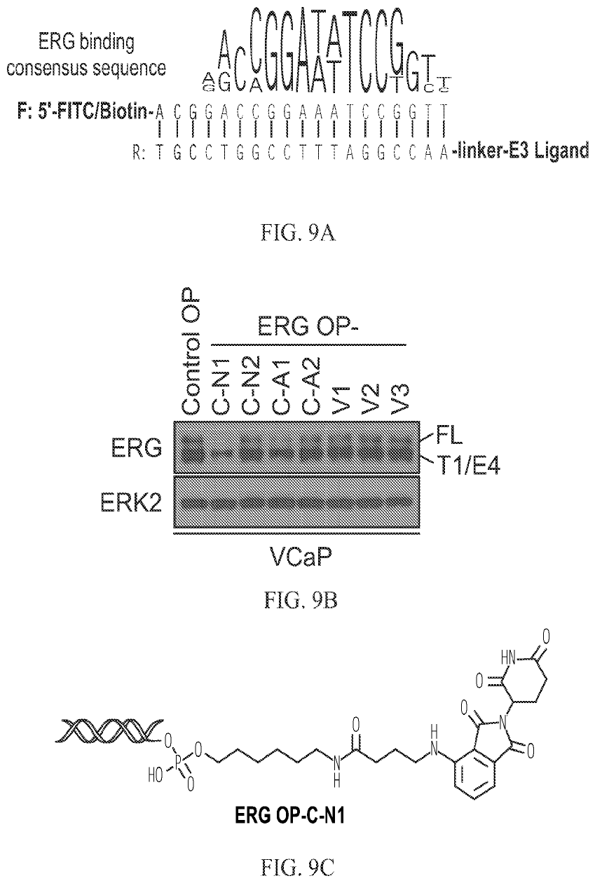

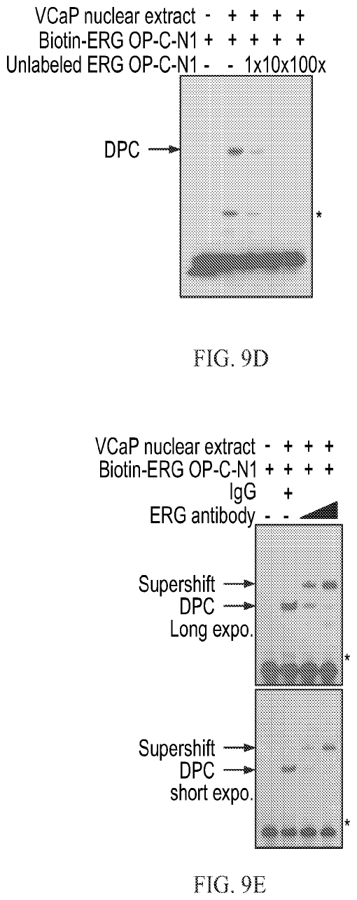

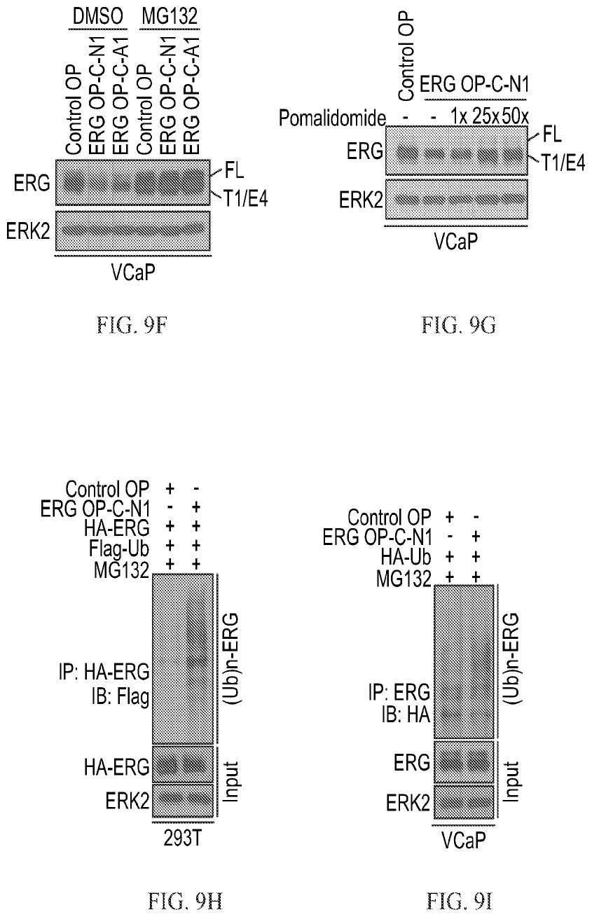

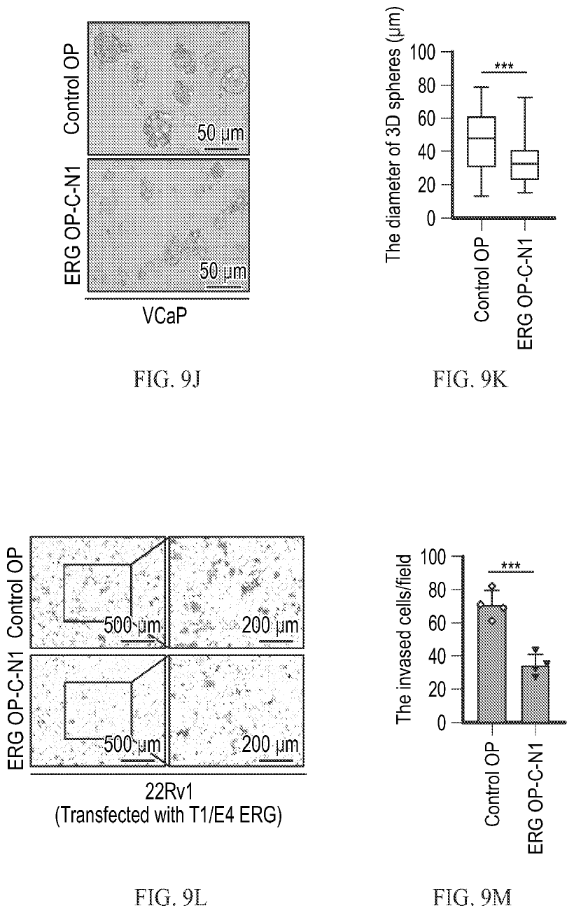

Four ERG pomalidomide-based PROTACs (termed OP-C-N1, OP-C-N2, OP-C-A1, and OP-C-A2) were generated following synthesis of NHS-ester and azide intermediates and incorporation of oligonucleotides through NHS-ester modification and click reaction, respectively (FIG. 9A and Table 3).

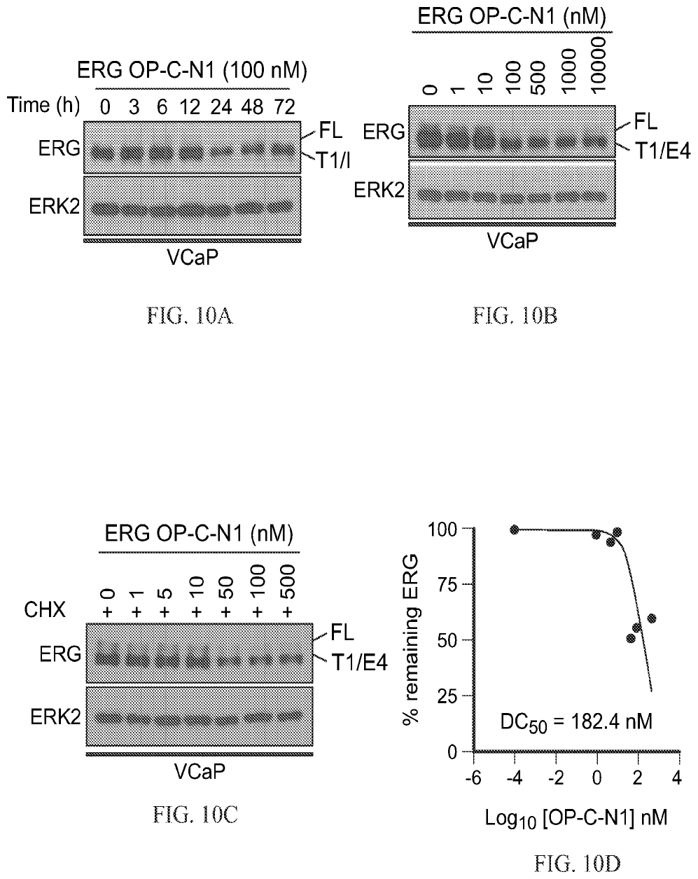

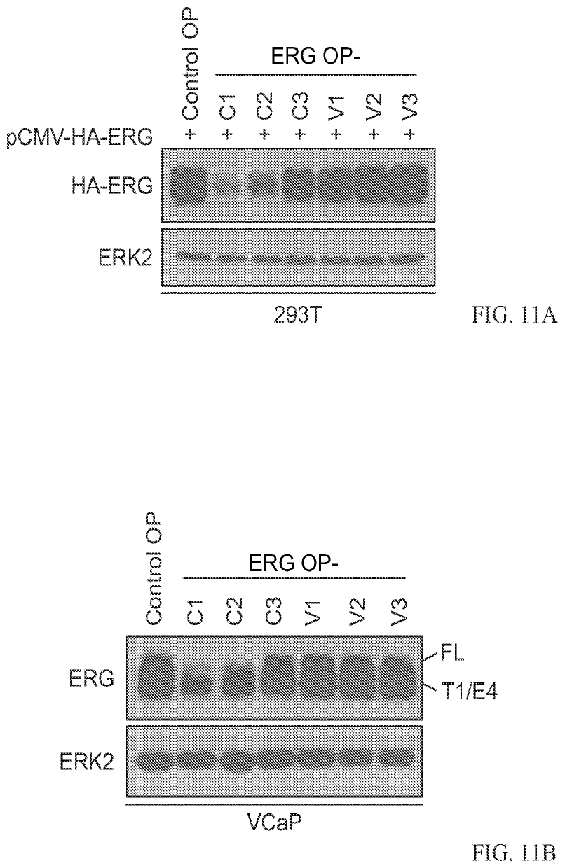

ERG OP-C-N1 and ERG OP-C-A1 degraded ERG protein in VCaP cells (FIG. 9B). ERG OP-C-N1 was selected for further investigation (FIG. 9C). The kinetics experiment showed that ERG OP-C-N1 effectively degraded ERG protein in a time- and dose-dependent manner (FIGS. 10A and 10B). Moreover, the DC₅o of ERG OP-C-N1 was 182.4 nM (FIGS. 10C and 10D). To examine whether ERG OP-C-N1 can bind to ERG in vitro, EMSA was performed using nuclear extract of VCaP cells. It was demonstrated that biotin-labeled ERG OP-C-N1 formed a DNA-protein complex (DPC) after incubation with VCaP nuclear extract. This binding was abolished by the addition of competitive non-biotin-labelled ERG OP-C-N1 (FIG. 9D). Addition of ERG antibody resulted in a supershift of DPC (FIG. 9E), suggesting that the detected DPC contains ERG protein. Furthermore, the destabilization of ERG protein by ERG OP-C-N1 was abolished by pretreatment with MG132 (FIG. 9F) and pomalidomide (FIG. 9G). Next, it was demonstrated that ERG OP-C-N1 treatment increased poly-ubiquitination of ERG protein (FIGS. 9H and 9l).

To determine the anti-cellular effect of ERG OP-C-N1, 3D culture for VCaP cells after the treatment of ERG OP-C-N1 was performed. The quantification of 3D culture diameter showed that ERG OP-C-N1 inhibited VCaP cell growth in vitro (FIGS. 10J and 10K). Moreover, a cell invasion assay showed that the treatment of ERG OP-C-N1 decreased the invasion ability of VCaP cells (FIGS. 9L and 9M). Thus, a bioactive ERG O′PROTAC was identified that can degrade ERG protein and inhibit cancer cell growth in vitro.

TABLE 3
Design and Composition of Exemplary O′PROTACs.

| | | | Reverse (3′ to 5′) + Linker + E3 ligase ligand | | | |
|---|---|---|---|---|---|---|
| O′PRO- | Forward (5′ to 3′) | | | Linker | | |
| TAC | Label | Sequence | Sequence | Name | Structure | E3 ligase ligand |
| LEF1 OP-C1 | N/A | -LEF1-F- | -LEF1-R- | L1 | 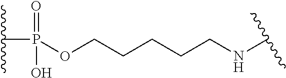 [SMILES: *NCCCCCOP(*)(=O)O] | 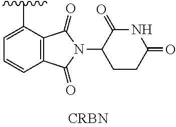 [SMILES: *c1cccc2c1C(=O)N(C1CCC(=O)NC1=O)C2=O] |
| LEF1 OP-C2 | N/A | -LEF1-F- | -LEF1-R- | L2 | 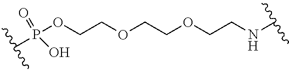 [SMILES: *NCCOCCOCCOP(*)(=O)O] | |
| LEF1 OP-C3 | N/A | -LEF1-F- | -LEF1-R- | L3 | 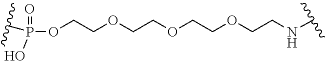 [SMILES: *NCCOCCOCCOCCOP(*)(=O)O] | |
| LEF1 OP-V1 | N/A | -LEF1-F- | -LEF1-R- | L4 | 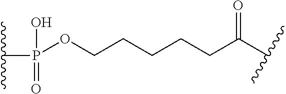 [SMILES: *C(=O)CCCCCOP(*)(=O)O] | 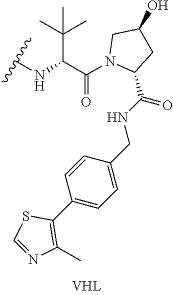 [SMILES: *N[C@@H](C(=O)N1C[C@@H](O)C[C@@H]1C(=O)NCc1ccc(-c2scnc2C)cc1)C(C)(C)C] |
| LEF1 OP-V2 | N/A | -LEF1-F- | -LEF1-R- | L5 | 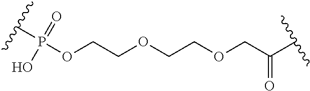 [SMILES: *C(=O)COCCOCCOP(*)(=O)O] | |
| LEF1 OP-V3 | N/A | -LEF1-F- | -LEF1-R- | L6 | 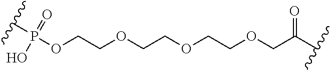 [SMILES: *C(=O)COCCOCCOCCOP(*)(=O)O] | |
| LEF1 | Biotin | -LEF1-F- | -LEF1-R- | L1-3 or L4-6 | | CRBN or VHL |
| Biotin- | | | | | | |
| OPs | | | | | | |
| LEF1 | FITC | -LEF1-F- | -LEF1-R- | L1-3 or L4-6 | | CRBN or VHL |
| FITC- | | | | | | |
| OPs | | | | | | |
| ERG OP-C1 | N/A | -ERG-F- | -ERG-R- | L1 | 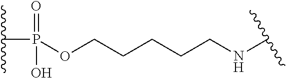 [SMILES: *NCCCCCOP(*)(=O)O] | 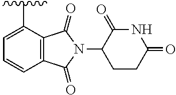 [SMILES: *c1cccc2c1C(=O)N(C1CCC(=O)NC1=O)C2=O] |
| ERG OP-C2 | N/A | -ERG-F- | -ERG-R- | L2 | 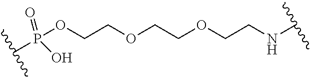 [SMILES: *NCCOCCOCCOP(*)(=O)O] | |
| ERG OP-C3 | N/A | -ERG-F- | -ERG-R- | L3 | 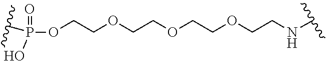 [SMILES: *NCCOCCOCCOCCOP(*)(=O)O] | |
| ERG OP-C- N1 | N/A | -ERG-F- | -ERG-R- | L7 | 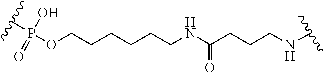 [SMILES: *NCCCC(=O)NCCCCCCOP(*)(=O)O] | |
| ERG OP-C- N2 | N/A | -ERG-F- | -ERG-R- | L8 | 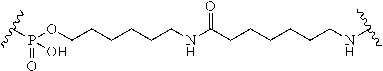 [SMILES: *NCCCCCCC(=O)NCCCCCCOP(*)(=O)O] | |
| ERG OP-C- A1 | N/A | -ERG-F- | -ERG-R- | L9 | 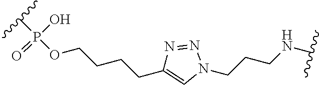 [SMILES: *NCCCn1cc(CCCCOP(*)(=O)O)nn1] | |
| ERG OP-C- A2 | N/A | -ERG-F- | -ERG-R- | L10 | 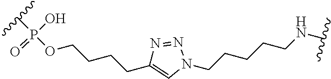 [SMILES: *NCCCCCn1cc(CCCCOP(*)(=O)O)nn1] | |
| ERG OP-V1 | N/A | -ERG-F- | -ERG-R- | L4 | 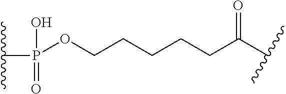 [SMILES: *C(=O)CCCCCOP(*)(=O)O] | 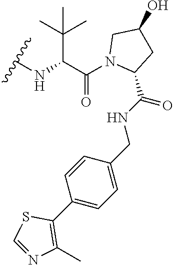 [SMILES: *N[C@@H](C(=O)N1C[C@@H](O)C[C@@H]1C(=O)NCc1ccc(-c2scnc2C)cc1)C(C)(C)C] |
| ERG OP-V2 | N/A | -ERG-F- | -ERG-R- | L5 | 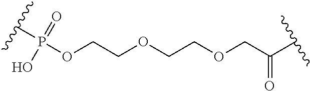 [SMILES: *C(=O)COCCOCCOP(*)(=O)O] | |
| ERG OP-V3 | N/A | -ERG-F- | -ERG-R- | L6 | 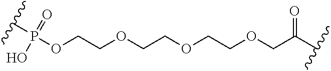 [SMILES: *C(=O)COCCOCCOCCOP(*)(=O)O] | |
| ERG | Biotin | -ERG-F- | -ERG-R- | L7-10 or L4-6 | | CRBN or VHL |
| Biotin- | | | | | | |
| OPs | | | | | | |
| ERG Control OP | N/A | -CTRL-F- | -CTRL-R- | L7 | 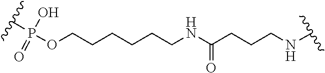 [SMILES: *NCCCC(=O)NCCCCCCOP(*)(=O)O] | CRBN |
| LEF1 Control OP | N/A | -CTRL-F- | -CTRL-R- | L4 | 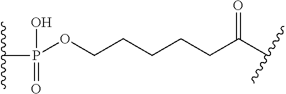 [SMILES: *C(=O)CCCCCOP(*)(=O)O] | CRBN |

### Cell Culture and Transfection

RWPE-1, C4-2, LNCaP, 22Rv1, VCaP, PC-3 and DU145 prostate cancer cell lines and 293T cell line were purchased from the American Type Culture Collection (ATCC). BPH1 cell line and LAPC4 cell line were obtained. 293T cells were maintained in DMVEM medium with 10% FBS. RWPE-1 cells were cultured in keratinocyte serum free medium supplemented with 0.05 mg/mL bovine pituitary extract, 5 ng/mL epidermal growth factor, and 100 U/mL penicillin-100 μg/mL streptomycin mixture. VCaP cells were cultured in RPMI medium with 15% FBS. LAPC4 cells were cultured in IMEM with 10% FBS. All other cell lines were maintained in RPMI medium with 10% FBS. Cells were transiently transfected with O′PROTAC using Lipofectamine 2000 or polyethylenimine (PEI) according to the manufacturer's instructions.

### Western Blot

Cell lysates were subjected to SDS-PAGE and proteins were transferred to nitrocellulose membranes (GE Healthcare Sciences). The membranes were blocked in Tris-buffered saline (TBS, pH 7.4) containing 5% non-fat milk and 0.1% Tween-20, washed twice in TBS containing 0.1% Tween-20, and incubated with primary antibody overnight at 4° C., followed by secondary antibody for 1 hour at room temperature. The proteins of interest were visualized using ECL chemiluminescence system (Thermo Fisher).

### Biotin Pulldown Assay

PC-3 cells were transfected with 100 nM of biotin-labelled LEF1 O′ PROTACs OP-V1 to V3 using PEI (Polysciences) for 36 hours. The cells were treated with MG132 for 12 hours before lysed in lysis buffer containing 50 mM Tris-HCl (pH7.5), 150 mM NaCl, 1% NP-40, 0.5% sodium deoxycholate and 1% proteinase inhibitor. Cell lysates were incubated with Streptavidin Sepharose High Performance beads (GE Healthcare) overnight at 4° C. The binding protein was eluted by elution buffer and subjected to western blot.

### RNA Extraction and RT-qPCR

RNA was extracted using TRIzol (Invitrogen) and reversely transcribed into cDNA with SuperScript III First-Strand Synthesis System (Promega). The quantitative PCR (qPCR) was performed in the iQ thermal cycler (Bio-Rad) using the iQ SYBR Green Supermix (Bio-Rad). Each sample was carried out in triplicate and three biological repeats were performed. The ΔCT was calculated by normalizing the threshold difference of a certain gene with glyceraldehyde-3-phosphate dehydrogenase (GAPDH). The primer sequences are listed as following:

| | | |
|---|---|---|
| CCND1 | (F)GATCAAGTGTGACCCGGACT | (R)CTTGGGGTCCATGTTCTGCT |
| | (SEQ ID NO: 116) | (SEQ ID NO: 117) |
| c-MYC | (F)TACAACACCCGAGCAAGGAC | (R)AGGCTGGTTTTCCACTACCC |
| | (SEQ ID NO: 118) | (SEQ ID NO: 119) |
| LEF1 | (F)AGCACGGAAAGAAAGACAGC | (R)TCTTGGACCTGTACCTGATGC |
| | (SEQ ID NO: 120) | (SEQ ID NO: 121) |
| GAPDH | (F)TCGGAGTCAACGGATTTGGT | (R)TTCCCGTTCTCAGCCTTGAC |
| | (SEQ ID NO: 122) | (SEQ ID NO: 123) |

### Immunofluorescent Cytochemistry Assay

PC-3 cells were seeded on the slides in 6-well plate overnight and reached to 60-70% of confluence and then transfected with LEF1 OP-V1 (0 nM or 100 nM). After 24 hours, cells were fixed by 4% paraformaldehyde and permeabilized with 0.05% Triton X-100. After a 1-hour block at room temperature, cells were subjected to immunoblot with LEF1 antibody (#2230S, Cell Signaling Technology) at 4° C. overnight. After washing, cells were incubated with anti-rabbit Alexa Fluor® 594 (A-11012, Thermo Fishers) for 1 hour at room temperature and mounted on the slides using the DAPI-containing counterstain solution (H-1200, Vector Laboratories) after washing. Images were taken by LSM 780 confocal microscope (Zeiss).

### Cell Growth Assay

Cell viability was measured using the MTS assay according to the manufacture's instruction (Promega). PC-3 and DU145 cells were transfected with LEF1 OP-V1 for 48 hours and 1,000 cells were seeded in each well of 96-well plates with 100 μL of medium. After cells adhered to the plate, at indicated time points, cell culture medium was replaced with 1×PBS and 10 μL of CellTiter 96R Aqueous One Solution Reagent (Promega) was added to each well. The plates were incubated for 2 hours at 37° C. in a cell incubator. Microplate reader was used to measure absorbance of 490 nm in each well.

### Nuclear Extraction and Electrophoretic Mobility Shift Assay (EMSA)

Nuclear protein was extracted using NE-PER™ Nuclear and Cytoplasmic Extraction Reagents (Cat #78833, Thermo Fisher Scientific). EMSA was performed according to the manufacturer's instruction by using the biotin-labeled LEF1 or ERG OPROTAC as probes. For supershift assay, ERG or LEF1 antibodies were added into the cell nuclear extract mixed with the biotin-labelled OPROTAC probes and the mixture were incubated with for 1 hour before loading into 6% of non-denatured polyacrylamide gel.

### Three-Dimensional (3D) Culture

Twenty-thousands of VCaP cells were resuspended in 250 μL plain medium and seeded on the top of a thin layer of Matrigel Matrigel matrix (BD Bioscience) in a 24-well plate. After 30 minutes, when the cells were settled down, they were covered with a layer of 10% Matrigel diluted with DMEM/F12 medium. Cells were transfected with ERG OP-C-N1 (200 nM), and the medium was changed with fresh and warm DMEM/F12 plus 10% FBS medium every 2-3 days.

### Mouse Xenograft and Drug Treatment

3×10⁶ PC-3 cells or DU145 cells mixed with Matrigel matrix (BD Bioscience) were injected subcutaneously into the left flank of six-week-old SCID male mice. When the tumor volume reached approximately 75 mm³, mice were randomly divided into three groups for treatment with 1×PBS, control OP, or LEF1 OP-V1 (10 mg/kg in PEI solution) via tail vein injection every other day. The volume of xenografts and mouse body weight were measured every three days. After 18-day (for PC-3 tumors) or 21-day (for DU145 tumors) treatment, mice were euthanized and xenografts were harvested for the measurement of weight. One part of tissues was formalin fixed and paraffin-embedded (FFPE) for IHC analysis and the rest of the tissues was used for RNA and protein extraction for RT-qPCR and Western blot analysis, respectively.

### Immunohistochemistry (IHC)

The FFPE xenograft tissues were cut consecutively at 4 micrometer for the IHC assay. The IHC staining was performed as previously reported (Hong et al., Mol. Cell, 79:1008 (2020)).

### Statistical Analysis

Statistical analysis was performed with one-sided or two-sided paired Student's t-test for single comparison. P value<0.05 was considered statistically significant. All values shown were expressed as means±SD.

### Example 6

### Synthesis of Dimethyl 3-((5-(((2-cyanoethoxy)(diisopropylamino) phosphaneyl)oxy)pentyl) amino)phthalate

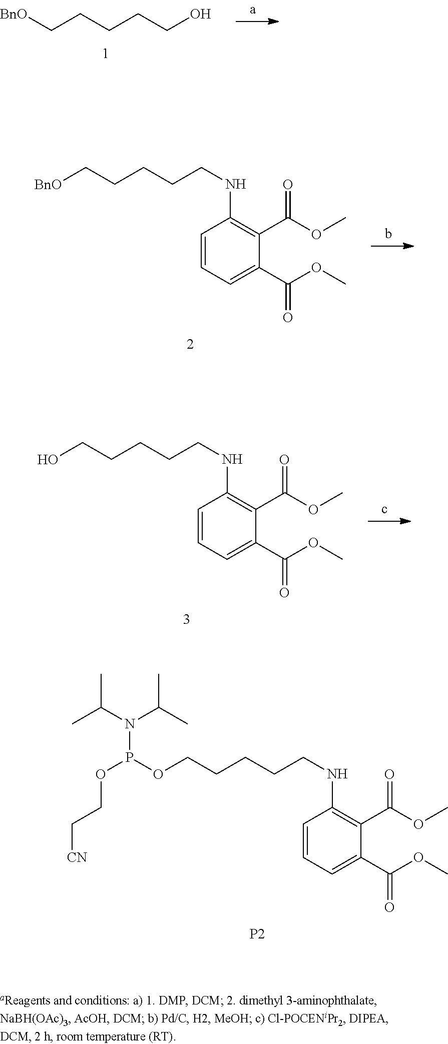

### Procedure:

Dimethyl 3-((5-(benzyloxy)pentyl)amino)phthalate (2): compound 1 (1.94 g, 10 mmol) was dissolved in DCM (30 mL), then DMP (5.5 g, 13 mmol) was added. The mixture was stirred at RT for 2 hours. The white solid was filtered off and washed with EA. The filtrate was concentrated. The residue was dissolved in Et₂O and washed with water. The organic phase was dried with Na₂SO₄, filtered and concentrated. The residue was dissolved in DCM (30 mL), then dimethyl 3-aminophthalate (836 mg, 4 mmol) and 3 drops of AcOH were added. The mixture was stirred at RT for 30 min, then NaBH (OAc)₃ (1.22, 6 mol) was added. The reaction was stirred at RT overnight. After completion, the reaction solution was diluted with DCM, and washed with water. The organic phase was dried with Na₂SO₄, filtered and concentrated. The residue was purified with flash chromatography (Hexane:EA=100:0 to 80:20), giving product as yellow oil (915 mg, 59.4%). ¹H NMR (400 MHz, CDCl₃) δ 7.35-7.30 (m, 6H), 6.80 (t, J=1.1 Hz, 1H), 6.79-6.77 (m, 1H), 4.50 (s, 2H), 3.86 (s, 3H), 3.82 (s, 3H), 3.49 (t, J=7.3, 2H), 3.16 (t, J=7.1 Hz, 2H), 1.71-1.63 (m, 4H), 1.53-1.47 (m, 2H).

Dimethyl 3-((5-hydroxypentyl)amino)phthalate (3): Compound 2 (900 mg, 2.33 mmol) was dissolved in MeOH (15 mL), then Pd/C (180 mg, 20% wt) was added. The mixture was stirred at RT under H2 atmosphere overnight. Pd/C was filtered off and washed with MeOH. The filtrate was concentrated and purified with flash chromatography (Hexane:EA=100:0 to 65:35), giving product as yellow oil (530 mg, 77%). ¹H NMR (400 MHz, CDCl₃) δ 7.33-7.27 (m, 1H), 6.77 (t, J=1.5 Hz, 1H), 6.75 (m, 1H), 3.85-3.82 (s, 3H), 3.81 (s, 3H), 3.65 (t, J=7.8, 2H), 3.16 (t, J=7.0 Hz, 2H), 1.67 (dd, J=14.6, 7.2 Hz, 2H), 1.63-1.56 (m, 2H), 1.51-1.42 (m, 2H).

Dimethyl 3-((5-(((2-cyanoethoxy)(diisopropylamino)phosphaneyl)oxy)pentyl) amino)phthalate (P2): Compound 3 (130 mg, 0.44 mmol) was dissolved in anhydrous DCM (5 mL), DIPEA (218 μL, 1.32 mmol) and Cl-POCEN′Pr₂ (147 μL, 0.66 mmol) was added. The mixture was stirred at RT for 1 hour. Solvent was removed, and the residue was purified with flash chromatography (Hexane:Actone (5% TEA)=100:0 to 75:25), giving product as colorless oil (135 mg, 62%). ¹H NMR (400 MHz, CDCl₃) δ 7.31 (t, J=8.0 Hz, 1H), 6.78 (s, 1H), 6.76 (t, J=2.8 Hz, 1H), 3.88-3.83 (m, 4H), 3.83-3.77 (m, 4H), 3.71-3.55 (m, 4H), 3.17 (dd, J=12.3, 6.9 Hz, 2H), 2.63 (t, J=6.5 Hz, 2H), 1.66 (m, 4H), 1.54-1.46 (m, 2H), 0.92-0.83 (m, 12H).

### Example 7: Exemplified Modifiers

The following compounds were prepared in accordance with the methods and procedures of Example 6, using appropriate commercially available starting materials.

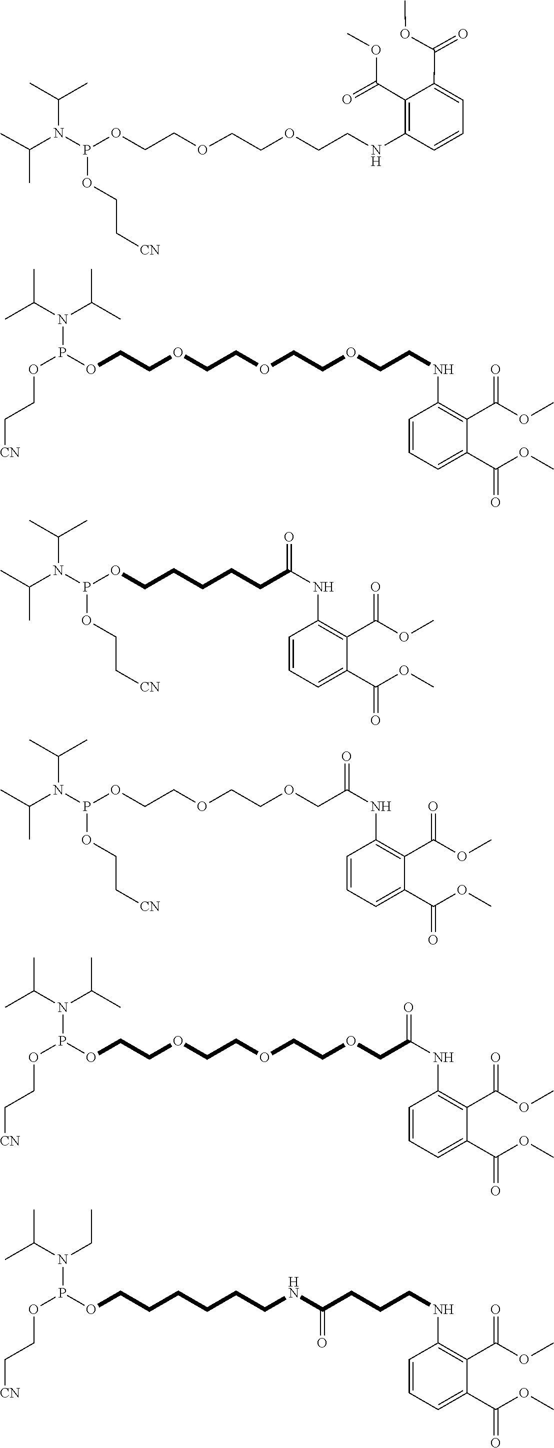

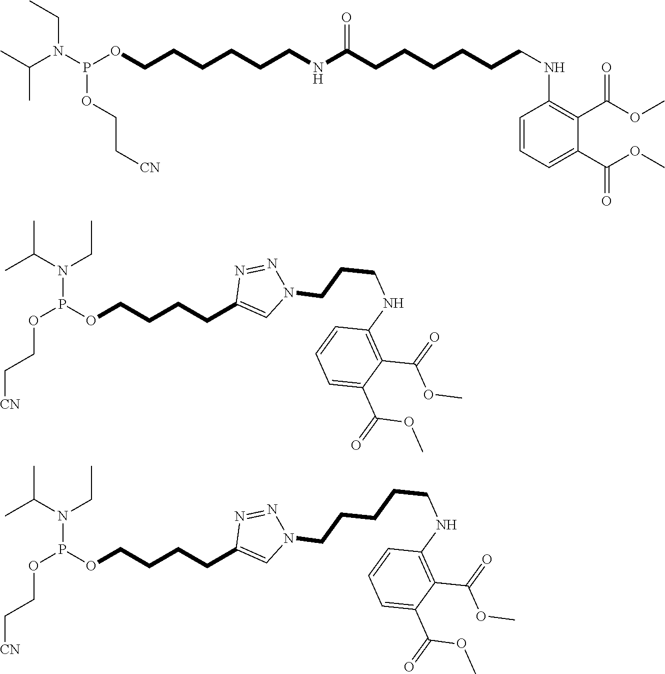

### Example 8: Development of phthalic acid-based O′PROTACs as degraders of ERG protein

Phosphoramidite chemistry was initially used to construct the pomalidomide- and VH032-based O′PROTACs (ERG OP-C1 to C3 and OP-V1 to V3) with different linker lengths to target ERG. Different from the mass spectrometry results of VH032-based ERG O′PROTACs, the mass spectrum of three pomalidomide-based ERG O′PROTACs showed that phthalic acid rather than phthalimide was the major product from the DNA synthesizer. These results suggest that pomalidomide was potentially susceptible to the deprotection condition during regular DNA synthesis (Scheme 2A). See Table 4 for design and composition of O′PROTACs.

Schemes 2A and 2B:

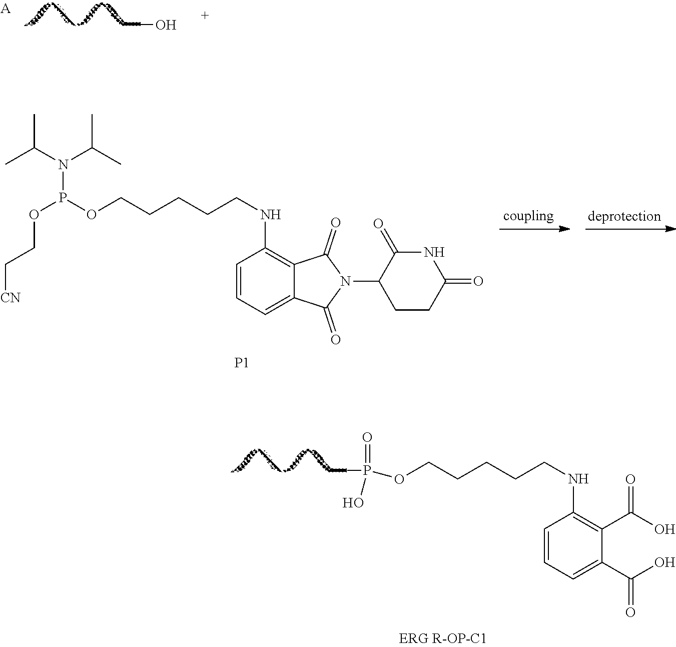

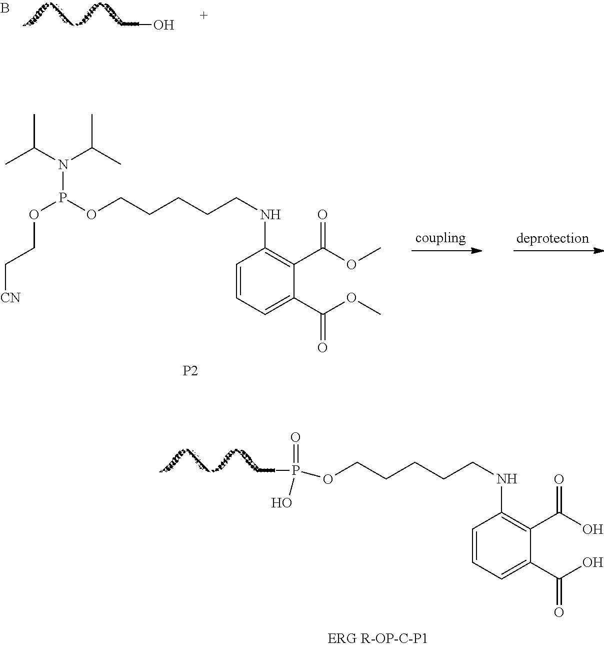

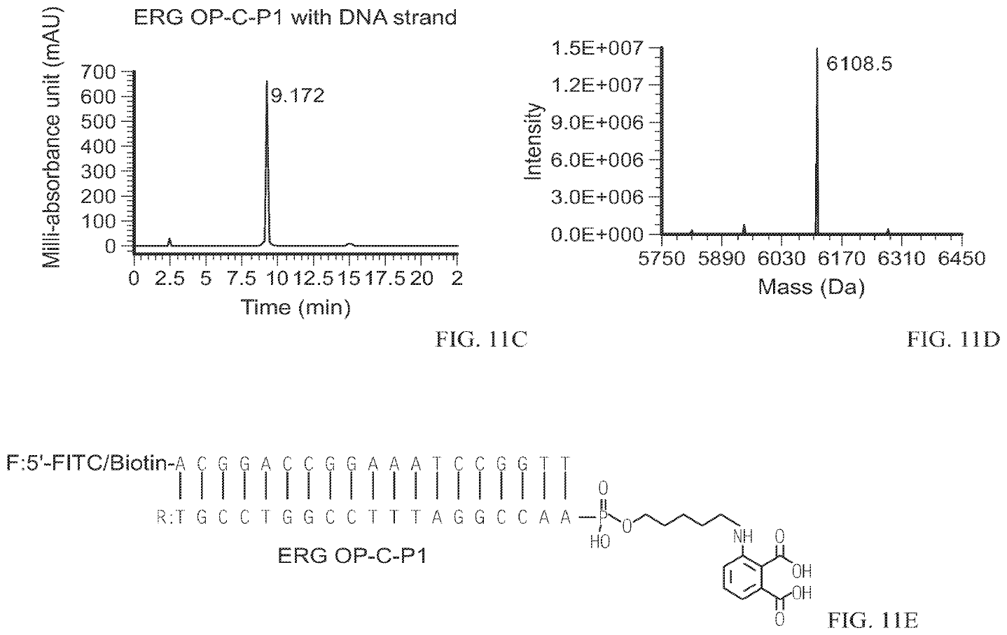

When 293T cells were transfected with ERG expression plasmid and treated with one of the three crude 3-N-substituted-aminophthalic acid-based O′PROTACs (OP-C1 to C3), two of them (C1 and C2) exhibited potent activity in ERG degradation (FIG. 11A). In contrast, VH032-based ERG O′PROTACs were inactive. These two ERG O′PROTACs (C1 and C2) also effectively decreased ERG protein in prostate cancer VCaP cells that expressed a high level of endogenous ERG protein due to the TRMPRSS2-ERG gene fusion (FIG. 11B).

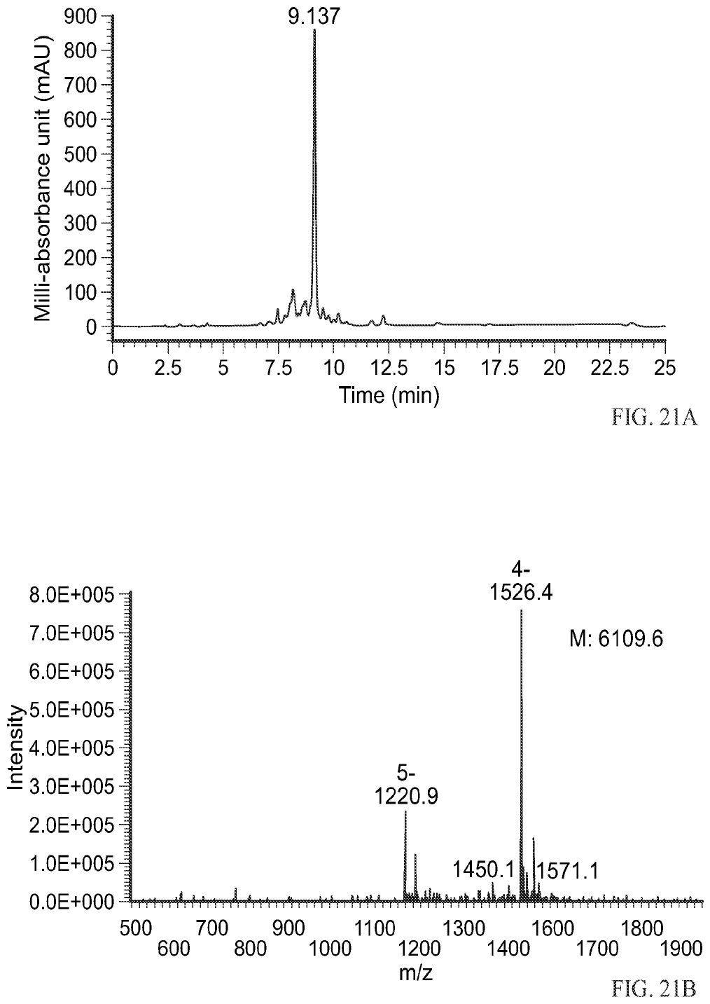

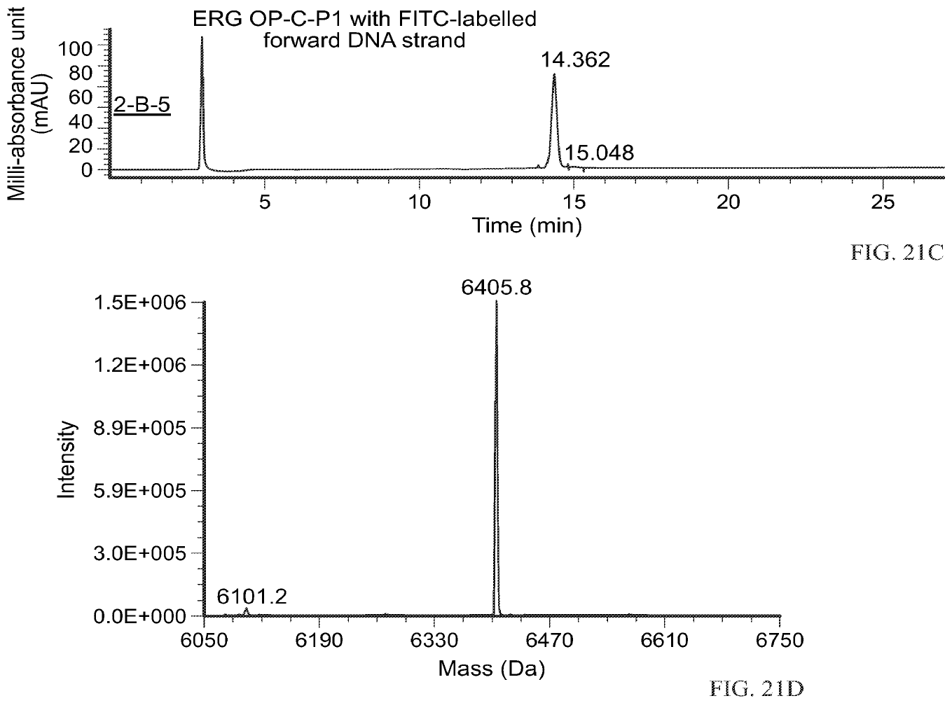

To test the hypothesis that phthalic acid was an E3 ligase recruiter of O′PROTACs that are effective in proteolytic degradation of a target protein, an ERG O′PROTAC (OP-C-P1) was synthesized by applying a synthetic route using phthalic acid dimethyl ester as the start material (Scheme 2B). The HPLC and mass spectrometry data indicated that ERG OP-C-P1 (containing a DNA oligo composed by phthalic acid-linked reverse strand and FITC-labeled forward strand) was successfully synthesized by phosphoramidite chemistry with high purity and expected molecular mass (FIGS. 11C, 11D, 21C, and 21D). This ERG OP-C-P1 (FIG. 11E) was used for further biochemical and functional studies.

TABLE 4
Design and Composition of O′PROTACs.

[symbol]

| | | | Reverse (3' to 5') + Linker + E3 ligase ligand | | | |
|---|---|---|---|---|---|---|
| O′PRO- | Forward (5′ to 3′) | | | Linker | | |
| TAC | Label | Sequence | Sequence | Name | Structure | E3 ligase ligand |
| ERG OP-C1 | N/A | -ERG-F- | -ERG-R- | L1 | 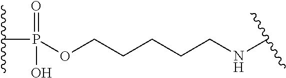 [SMILES: *NCCCCCOP(*)(=O)O] | 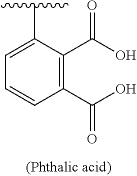 [SMILES: *c1cccc(C(=O)O)c1C(=O)O] |
| ERG OP-C2 | N/A | -ERG-F- | -ERG-R- | L2 | 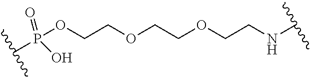 [SMILES: *NCCOCCOCCOP(*)(=O)O] | |
| ERG OP-C3 | N/A | -ERG-F- | -ERG-R- | L3 | 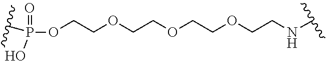 [SMILES: *NCCOCCOCCOCCOP(*)(=O)O] | |
| ERG OP-C-P1 | N/A | -ERG-F- | -ERG-R- | L1 | 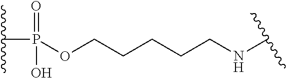 [SMILES: *NCCCCCOP(*)(=O)O] | |
| ERG OP-V1 | N/A | -ERG-F- | -ERG-R- | L4 | 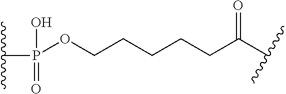 [SMILES: *C(=O)CCCCCOP(*)(=O)O] | 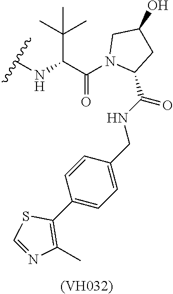 [SMILES: *N[C@@H](C(=O)N1C[C@@H](O)C[C@@H]1C(=O)NCc1ccc(-c2scnc2C)cc1)C(C)(C)C ; "(VH032)"] |
| ERG OP-V2 | N/A | -ERG-F- | -ERG-R- | L5 | 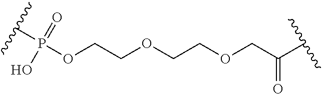 [SMILES: *C(=O)COCCOCCOP(*)(=O)O] | |
| ERG OP-V3 | N/A | -ERG-F- | -ERG-R- | L6 | 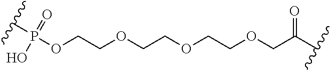 [SMILES: *C(=O)COCCOCCOCCOP(*)(=O)O] | |
| ERG | Biotin | -ERG-F- | -ERG-R- | L1-6 | | E3 ligase ligand |
| Biotin- | | | | | | |
| OPs | | | | | | |
| ERG | FITC | -ERG-F- | -ERG-R- | L1-6 | | E3 ligase ligand |
| FITC- | | | | | | |
| OPs | | | | | | |
| ERG Control OP | N/A | -CTRL-F- | -CTRL-R- | L7 | 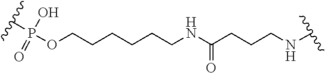 [SMILES: *NCCCC(=O)NCCCCCCOP(*)(=O)O] | 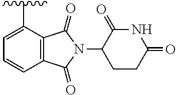 [SMILES: *c1cccc2c1C(=O)N(C1CCC(=O)NC1=O)C2=O] |

### Example 9: Phthalic Acid-Based ERG O′PROTAC Induces ERG Proteasome Degradation

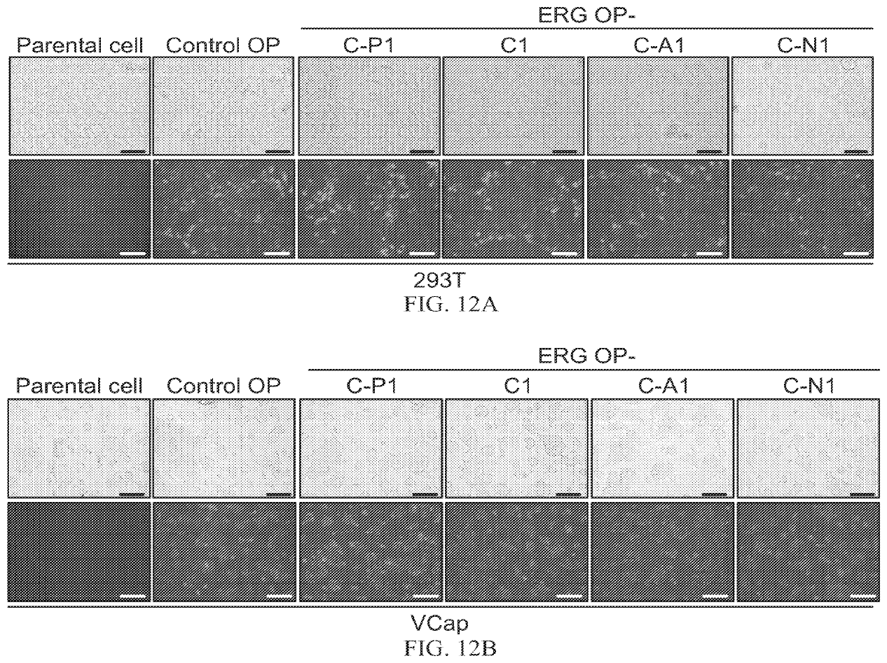

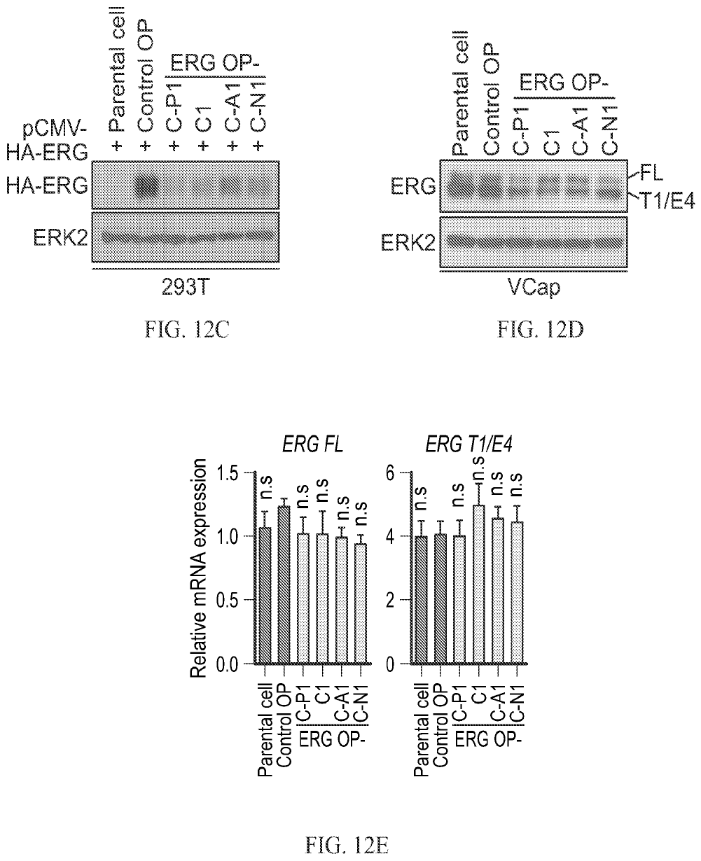

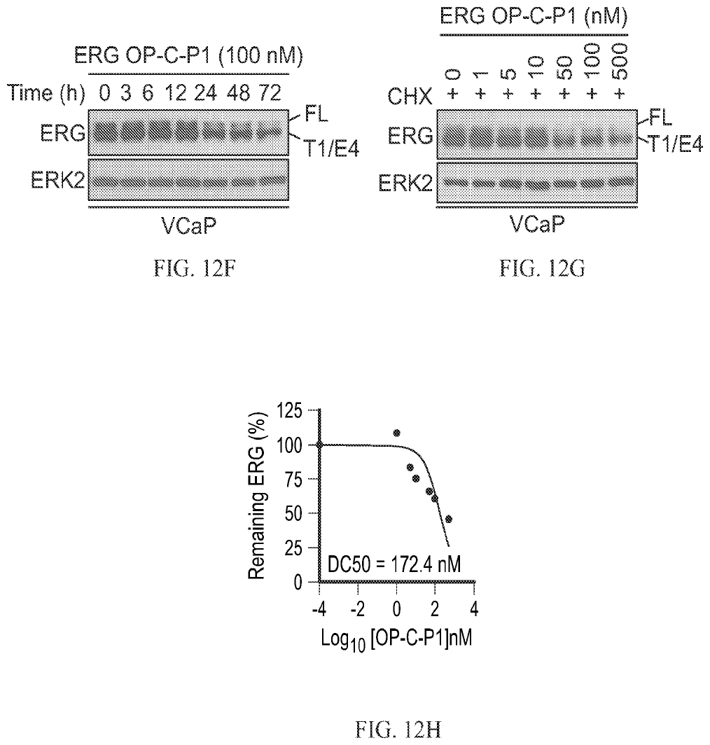

The efficacy of the phthalic acid-based ERG OPs (C-P1 with high purity and C1 with low purity) was compared with two pomalidomide-based ERG O′PROTACs synthesized via click reaction. FITC-labeled ERG O′PRORACs were used to assess the transfection efficiency of these O′PROTACs. Fluorescent microscopy analysis showed that phthalic acid-based ERG O′PROTACs were transfected as effectively as ERG O′PROTACs C-A1 and C-N1 in both 293T and VCaP cell lines (FIG. 12A, B).

Western blot analysis revealed that OP-C-P1 exhibited a slightly stronger inhibitory effect on downregulation of ectopically expressed full-length (FL) ERG protein than OP-C-A1 and OP-C-N1 in 293T cells (FIG. 12C), and similar results were obtained of the endogenous FL ERG in VCaP cells (FIG. 12D).

Further analysis revealed that these ERG OPs did not exerted an effect on mRNA levels of both FL and truncated ERG T1/E4 derived from TMPRSS2-ERG gene fusion (FIGS. 12D and 12E), suggesting that ERG OP-C-P1 inhibit ERG expression at the post-transcriptional level.

The kinetics of OP-C-P1 potency on protein degradation was evaluated. Time-course studies demonstrated that OP-C-P1 inhibited ERG protein expression starting from 24-hours post-transfection (FIG. 12F). Dose-course experiments further revealed that OP-C-P1 induced dramatic decrease in ERG protein level at a concentration as low as 50 nM (FIG. 12G). Little or no further increase in reduction of ERG protein level even much higher concentrations (100 or 500 nM) were used, implying that the amount of ERG OP-C-P1 in cells could be saturated or its up-take by cells could be limited due to transfection efficiency. The degradation concentration (DC) curve demonstrated that OP-C-P1 inhibited 50% of ERG protein at 172.4 nM (FIG. 12H).

### Example 10: Phthalic Acid-Based ERG OP Degrades ERG Via Proteasome Pathway

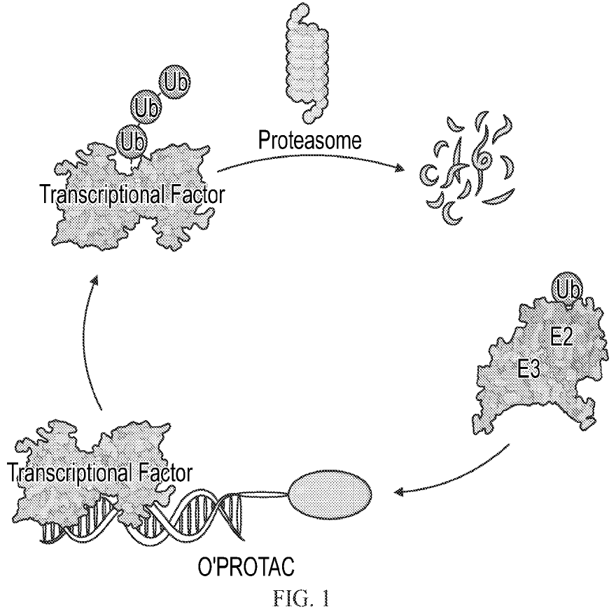

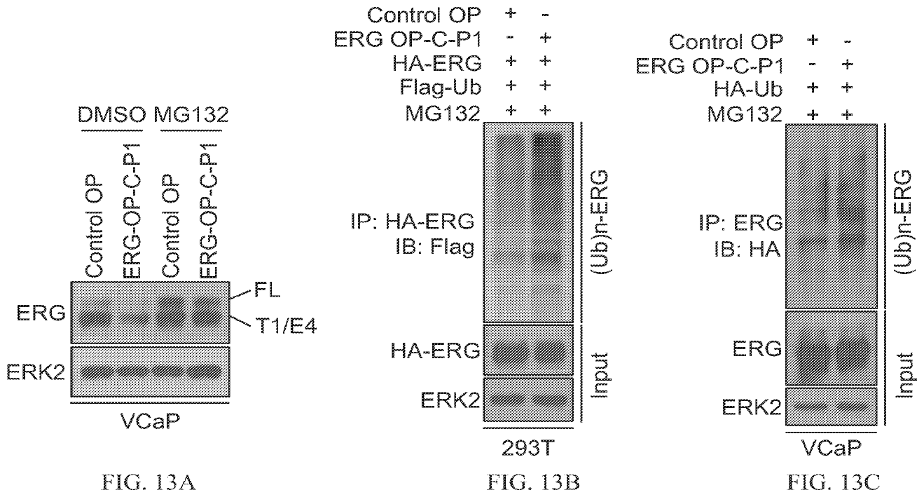

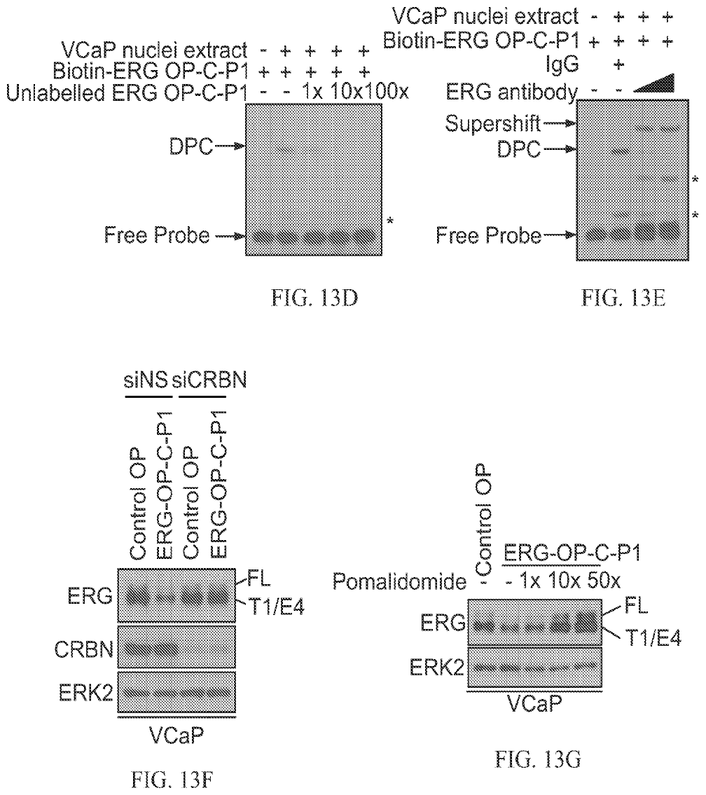

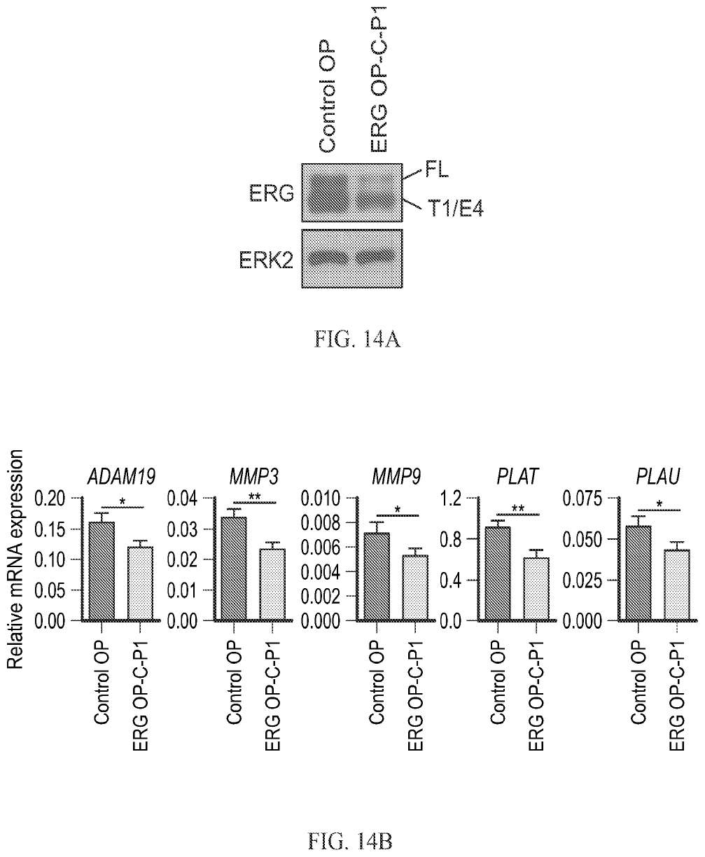

To determine whether phthalic acid-based ERG OP-C-P1-induced ERG protein downregulation is mediated through the ubiquitination and proteasome degradation pathway, VCaP cells were first transfected with OP-C-P1 and treated with the proteasome inhibitor MG132. MG132 treatment completely blocked the degradation of ERG protein (FIG. 13A), suggesting that ERG degradation is dependent on the proteasome pathway. Meanwhile, the ubiquitination assay showed that the treatment of OP-C-P1 enhanced the ubiquitination level of both exogenous and endogenous ERG in 293T and VCaP cells, respectively (FIGS. 13B and 1C).

To examine whether ERG OP-C-P1 can bind to ERG in vitro, an electrophoretic mobility shift assay (EMSA) was performed using nuclear extract of VCaP cells. Biotin-labeled ERG OP-C-P1 formed a DNA-protein complex (DPC) in the nuclear extract of VCaP cells. This binding was interrupted by the addition of competitive non-biotin-labeled ERG OP-C-P1 (FIG. 13D). Moreover, the addition of ERG antibody resulted in a super-shift of DPC (FIG. 13E), suggesting that the detected DPC contains ERG protein.

### Example 11: Phthalic Acid-Based ERG OP-Induced Degradation of ERG is Mediated by CRBN

Next, the following was performed to determine whether OP-C-P1-mediated degradation of ERG is dependent on cereblon (CRBN). CRBN was knocked down in VCaP cells, and the cells were treated with OP-C-P1. CRBN knockdown completely abolished OP-C-P1-induced degradation of ERG (FIG. 13F). The treatment of cereblon ligand pomalidomide also overcame the degradation of ERG protein induced by OP-C-P1, and this effect was dose dependent (FIG. 13G). These results demonstrate that OP-C-P1-induced degradation of ERG is mediated through CRBN E3 ligase.

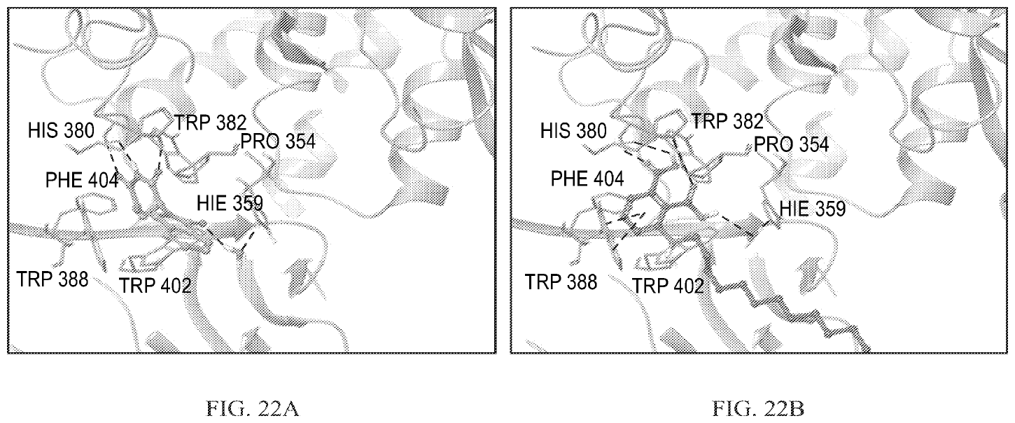

To understand the interaction between CRBN protein and 3-aminophthalic acid, docking was performed using 3-N-substituted phthalic acid and CRBN (PDB.4CI1). The interaction of phthalic acid was observed to be similar with thalidomide (FIG. 22). For example, the 1′-carboxylic acid group oriented toward the hydrophobic pocket and resulted in formation of two strong hydrogen bonds. The carbonyl oxygen and hydrogen of hydroxy groups interacted with the backbone of TRP382 and HIS380, respectively. These hydrogen bond interactions were resemblant with the glutarimide group of thalidomide, where interaction occurred between two carbonyl and amide to residues HIS380 and TRP382, respectively. Additionally, the other 2′-carboxylic acid group would be more solvent exposed. Due to the flexibility of C—C bond between benzene and carboxylic acid, the carbonyl oxygen could position itself facing to the hydrophobic pocket to retain hydrogen bond with imidazole side chain of HIS380; meanwhile, the hydroxy group formed weak water-mediated hydrogen bond with HIS359 side chain. Comparatively to thalidomide, the phthalimide was completely solvent exposed and accommodated with a water-mediated hydrogen bond with HIS359. There were also observed pi-pi interactions between indole of TRP388 and benzene ring of phthalic acid. The orientation of 3-amino group was completely solvent exposed similar to pomalidomide and lenalidomide, which contributed enormously for forming linkers with any potential warheads. This binding information provided an explanation for the observation that phthalic acid-based O′PROTAC showed comparable activity as pomalidomide-based O′PROTACs.

### Example 12: Phthalic Acid-Based ERG OP Impairs ERG Target Gene Expression and Cell Growth and Invasion

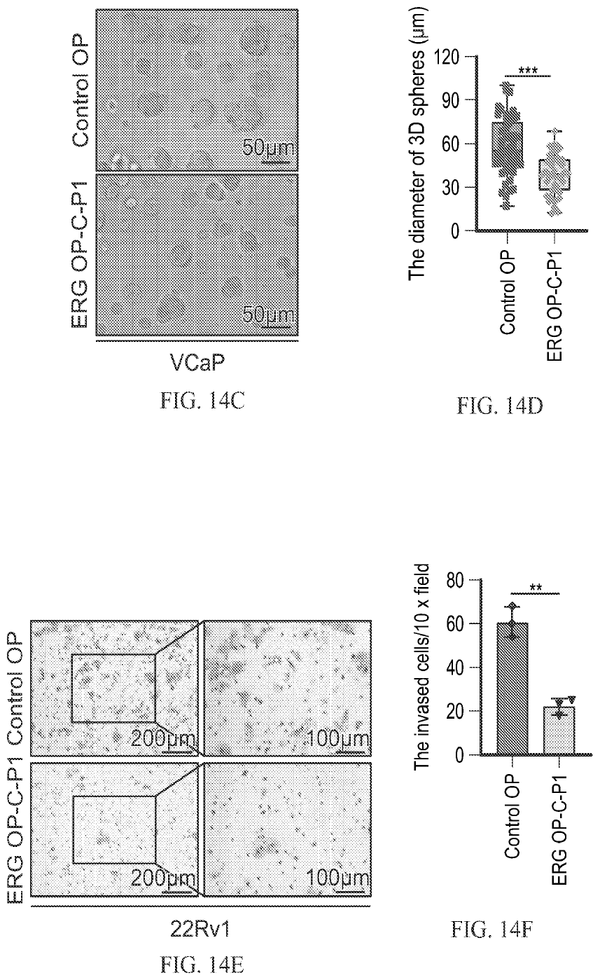

To determine whether ERG OP-C-P1 affects ERG signaling pathway, the transcriptional levels of ERG target genes were assessed. The downregulation of ERG by OP-C-P1 also significantly diminished mRNA expression of ERG target genes including ADAM19, MMP3, MMP9, PLAT and PLAU (FIGS. 14A and 14B). To examine the functional effects of OP-C-P1 on cell growth, a three-dimensional (3D) sphere formation assay was performed using VCaP cells. OP-C-P1 treatment largely decreased the diameters of the spheres of VCaP ccells, indicating that OP-C-P1 inhibited VCaP cell growth (FIGS. 14C and 1D). Considering the roles of ERG on cell invasion, a cell invasion assay was performed to detect whether this ERG OP can affect cell invasion. Treatment with OP-C-P1 decreased the invasion ability of VCaP cells (FIGS. 14E and 1F). Collectively, OP-C-P1-induced degradation of ERG effectively undermines the transcriptional activity of ERG and prostate cancer cell growth and invasion.

In summary, phthalic acid and 3-aminophthalic acid were identified as ligands of CRBN ligase. Phthalic acid-based ERG O′PROTAC significantly inhibited the protein level of ERG via ubiquitination-proteasome pathway and impaired ERG functions in cell growth and invasion. This ERG O′PROTAC provides clear evidence that phthalic acid functions actively as well as pomalidomide in O′PROTAC. These results demonstrate that this CRBN ligand can be employed to design O′PROTACs to degrade nucleic acid binders (e.g., transcription factors) or to design canonical PROTACs to degrade any appropriate POI including those that do not bind nucleic acid.

### Example 13

### DNA Binding Sequence for Gain of Function Mutants of p53

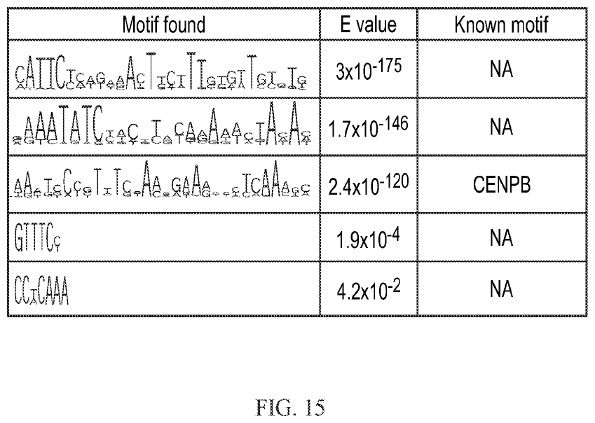

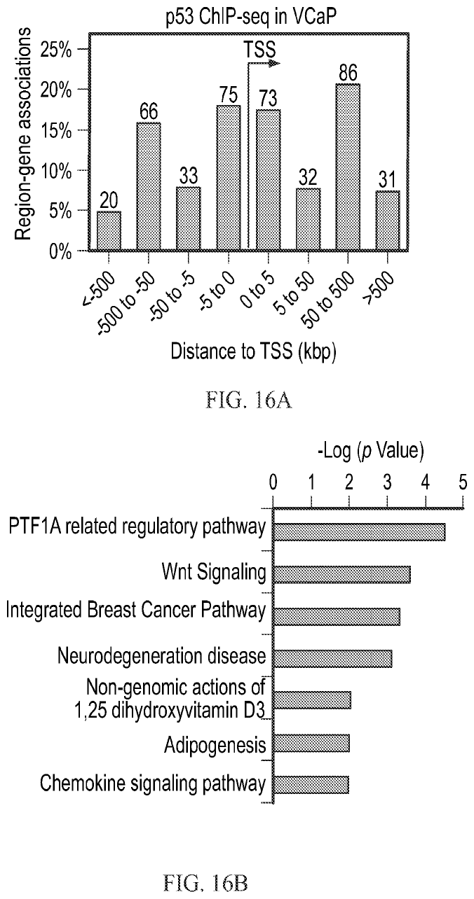

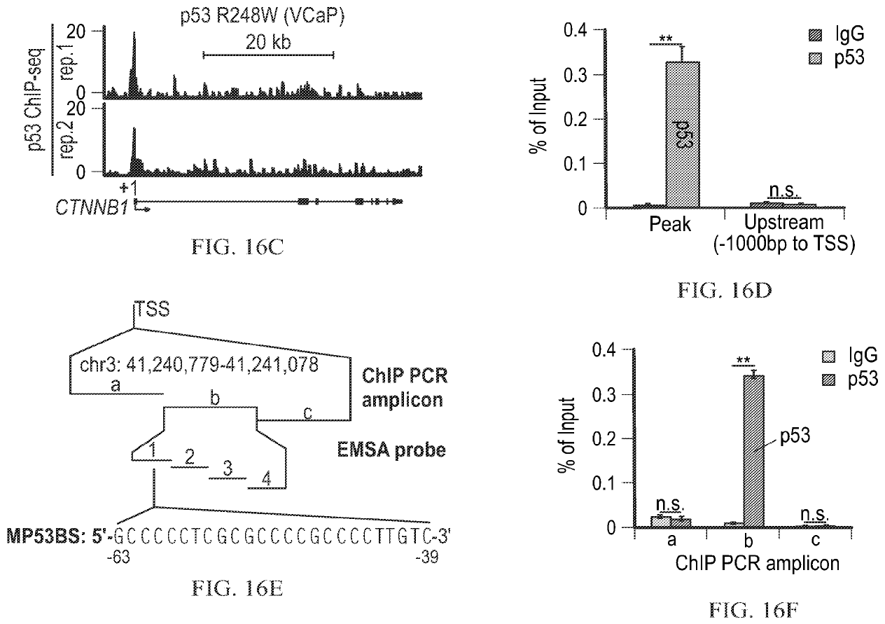

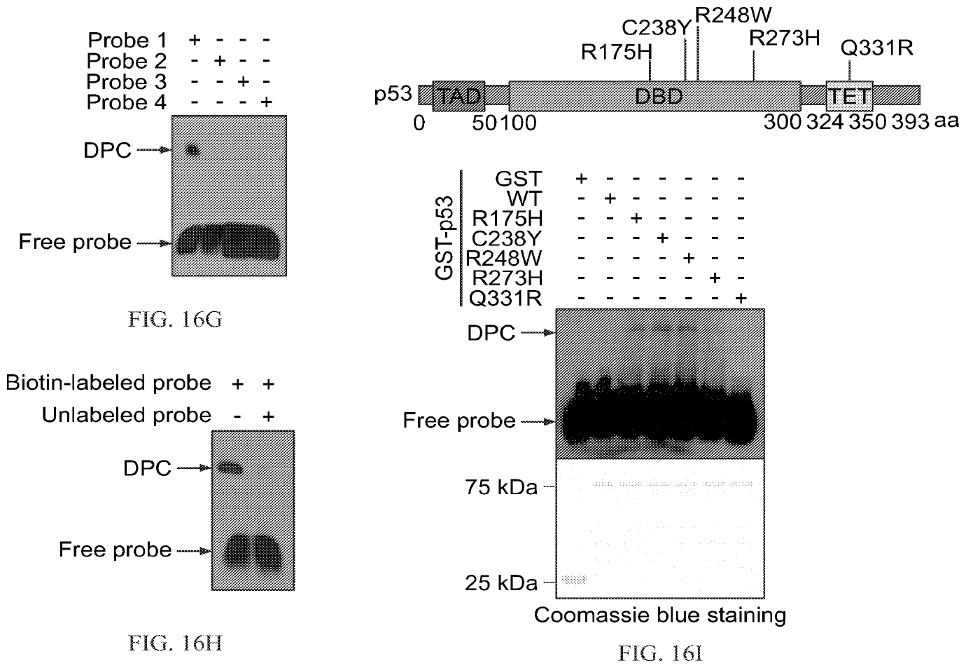

The following was performed to determine whether mutant p53 possessing gain of function (GOF) activity binds to the genomic loci of pyrimidine synthesis genes (PSGs). To this end, p53 ChIP-seq was performed in VCaP cells, and more than 400 (n=416) p53 R248W mutant-bound genomic loci in this cell line were identified (Table 5). DNA binding motif analysis showed that no specific transcription factor-binding motif was typically enriched (FIG. 15). The GOF p53-binding peaks were localized in both promoter and non-promoter regions, and none were present in the PSG loci in VCaP cells (FIG. 16A and Table 5), suggesting that p53 mutant may regulate PSG expression through indirect mechanism(s).

TABLE 5
p53 R248W mutant-bound genomic loci in VCaP PCa cell line.

| Peak location | Relative gene name |
|---|---|
| chr16: 2390693-2390894 (−58) | ABCA3 |
| chr2: 204193293-204193639 (+463) | ABI2 |
| chr17: 79367746-79367932 (+111968) | ACTG1 |
| chr5: 4855274-4855458 (−285077) | ADAMTS16 |
| chr8: 131632861-131633050 (+421716) | ADCY8 |
| chr2: 3522833-3523032 (+574) | ADI1 |
| chr15: 73075966-73076184 (+51) | ADPGK |
| chr9: 133982504-133982693 (+10736) | AIF1L |
| chr7: 91570304-91570499 (+221) | AKAP9 |
| chr13: 111567380-111567705 (−127) | ANKRD10 |
| chr7: 99698208-99698498 (−827) | AP4M1 |
| chr20: 3800723-3801014 (−334) | AP5S1 |
| chr12: 99995741-99996073 (+956988) | APAF1 |
| chr15: 29131409-29131681 (+425) | APBA2 |
| chr11: 120206767-120207253 (−777) | ARHGEF12 |
| chr13: 111805918-111806180 (+38425) | ARHGEF7 |
| chr8: 66528788-66529054 (+17521) | ARMC1 |
| chr8: 131632861-131633050 (−218739) | ASAP1 |
| chr17: 31899318-31899513 (−279410) | ASIC2 |
| chrX: 2171370-2171572 (+437577) | ASMT |
| chr11: 63438968-63439185 (+7) | ATL3 |
| chr2: 176566629-176566933 (−517446) | ATP5G3 |
| chrX: 40035178-40035365 (−404888) | ATP6AP2 |
| chr2: 27434590-27434978 (−111) | ATRAID |
| chr17: 42296408-42296605 (−21069) | ATXN7L3 |
| chr21: 30627637-30627821 (−44008) | BACH1 |
| chr7: 33168730-33168916 (−321) | BBS9 |
| chrX: 40035178-40035365 (−78616) | BCOR |
| chr1: 156186290-156186573 (−25321) | BGLAP |
| chr12: 26267030-26267216 (+10937) | BHLHE41 |
| chr9: 95527220-95527433 (−233) | BICD2 |
| chr14: 36417431-36417620 (+122002) | BRMS1L |
| chr14: 105705366-105705606 (−9425) | BTBD6 |
| chr11: 116643534-116643773 (+50), chr11: 116494708-116494909 (+148895) | BUD13 |
| chr11: 65686648-65686931 (−202) | C11orf68 |
| chr17: 6918011-6918206 (+295) | C17orf49 |
| chr20: 35243574-35243774 (+9449) | C20orf24 |
| chr2: 232526867-232527107 (+69412) | C2orf57 |
| chr6: 167846728-167846945 (+350702) | C6orf123 |
| chr7: 139025862-139026304 (+205) | C7orf55 |
| chr8: 50183556-50183759 (+198745) | C8orf22 |
| chr9: 95964241-95964471 (+144340) | C9orf129 |
| chr9: 26892529-26892881 (+97) | CAAP1 |
| chr3: 85205609-85205900 (−569877) | CADM2 |
| chr2: 47403526-47403930 (+12) | CALM2 |
| chr19: 47104151-47104482 (−176) | CALM3 |
| chr22: 21984313-21984531 (−2596) | CCDC116 |
| chr3: 123680103-123680374 (+325) | CCDC14 |
| chr17: 31899318-31899513 (−682888) | CCL2 |
| chr15: 73976313-73976631 (−82) | CD276 |
| chr20: 60192245-60192442 (+364862) | CDH4 |
| chr17: 31149515-31149817 (+336029) | CDK5R1 |
| chrY: 19561931-19562189 (+430040) | CDY2B |
| chr4: 68265579-68266489 (+145290) | CENPC |
| chr1: 215042851-215043097 (+266436) | CENPF |
| chr15: 93352448-93352706 (−90481) | CHD2 |
| chr13: 111133834-111134061 (+174334) | COL4A2 |
| chr12: 6833019-6833226 (+216) | COPS7A |
| chr1: 244998826-244999069 (+324) | COX20 |
| chr8: 145634703-145634920 (−59) | CPSF1 |
| chr17: 29035811-29036062 (+115857) | CRLF3 |
| chr17: 80231137-80231462 (+307) | CSNK1D |
| chr11: 10901550-10901737 (+473260) | CSNK2A3 |
| chr3: 41240868-41241089 (+49), chr3: 41855484-41855672 (+614648) | CTNNB1 |
| chr4: 49096612-49096882 (+108483), chr4: 49121143-49121360 (+132988), | CWH43 |
| chr4: 49132469-49132924 (+144433), chr4: 49146387-49146784 (+158322), | |
| chr4: 49151195-49151708 (+163188), chr4: 49155243-49155508 (+167112), | |
| chr4: 49289076-49289289 (+300919), chr4: 49316186-49316414 (+328036), | |
| chr4: 49318019-49318205 (+329848), chr4: 49324946-49325571 (+336995), | |
| chr4: 49637225-49637495 (+649096) | |
| chr4: 105537576-105537820 (−121647) | CXXC4 |
| chr11: 14913109-14913306 (+590) | CYP2R1 |
| chr8: 66528788-66529054 (−817603) | CYP7B1 |
| chr2: 15731847-15732189 (+716) | DDX1 |
| chr22: 18878053-18878574 (−15422), chr22: 18883687-18884010 (−9887) | DGCR6 |
| chr14: 24768937-24769124 (+8) | DHRS1 |
| chr17: 27229956-27230266 (−22) | DHRS13 |
| chr10: 12161018-12161226 (+50151) | DHTKD1 |
| chr12: 50898763-50898948 (−34) | DIP2B |
| chr17: 76572912-76573178 (+431) | DNAH17 |
| chr19: 4723813-4724015 (−39) | DPP9 |
| chr11: 65686648-65686931 (+62) | DRAP1 |
| chr18: 29078044-29078260 (+146) | DSG2 |
| chr12: 89366664-89366880 (+380276) | DUSP6 |
| chr20: 32287628-32287961 (−13585) | E2F1 |
| chr6: 20402468-20402658 (+165) | E2F3 |
| chr5: 83049568-83049768 (+630943) | EDIL3 |
| chrX: 68048726-68048980 (+13) | EFNB1 |
| chr15: 49170167-49170353 (+177) | EID1 |
| chr3: 186501000-186501199 (−236) | EIF4A2 |
| chr5: 49593788-49594173 (+143220) | EMB |
| chr6: 131949219-131949582 (−200) | ENPP3 |
| chr18: 108157-108471 (−58757), chr18: 10170-10492 (+39226) | ENSG00000173213 |
| chr16: 33963889-33964073 (−316285), chr16: 33963041-33963510 (−315580) | ENSG00000259680 |
| chr3: 90132408-90132651 (+975856) | EPHA3 |
| chr17: 19091397-19091591 (−49160) | EPN2 |
| chr12: 1179692-1180035 (+79460) | ERC1 |
| chr19: 45943118-45943356 (−16413) | ERCC1 |
| chr21: 40040505-40040719 (−6994) | ERG |
| chr16: 68269810-68270030 (+567) | ESRP2 |
| chr21: 40040505-40040719 (−136619) | ETS2 |
| chr9: 140317619-140317809 (0) | EXD3 |
| chr9: 133568849-133569047 (−160) | EXOSC2 |
| chr2: 420569-420792 (−132385) | FAM150B |
| chr15: 93352448-93352706 (−153389) | FAM174B |
| chr9: 67320532-67320819 (+465948), chr9: 66971117-66971403 (+815364), | FAM27E3 |
| chr9: 66819924-66820291 (+966516) | |
| chr12: 99995741-99996073 (−45624) | FAM71C |
| chr12: 1179692-1180035 (+523467) | FBXL14 |
| chr1: 224301695-224302017 (+67) | FBXO28 |
| chr1: 121485016-121485394 (−549268), chr1: 121484098-121484680 | FCGR1B |
| (−548452), chr1: 121478662-121478972 (−542880), chr1: 121354891-121355169 | |
| (−419093), chr1: 121353195-121353740 (−417531), chr1: 121351996- | |
| 121352288 (−416205) | |
| chr5: 44642366-44642562 (−253565) | FGF10 |
| chr6: 97087449-97087790 (+77160) | FHL5 |
| chr19: 45943118-45943356 (−28016) | FOSB |
| chr6: 41395657-41395863 (−118404) | FOXP4 |
| chr11: 65190233-65190527 (+36310) | FRMD8 |
| chr1: 230202442-230202674 (−398) | GALNT2 |
| chr8: 75532782-75533005 (+270277) | GDAP1 |
| chr19: 45582355-45582540 (−72) | GEMIN7 |
| chr14: 54955455-54955653 (+360) | GMFB |
| chr7: 100271217-100271468 (+189) | GNB2 |
| chr17: 29035811-29036062 (+231557) | GOSR1 |
| chr11: 64037325-64037921 (−14188) | GPR137 |
| chr7: 37802802-37803029 (+79441) | GPR141 |
| chr2: 133012312-133012914 (−161534), chr2: 133023177-133023551 | GPR39 |
| (−150783), chr2: 133025676-133025884 (−148367), chr2: 133033373-133033566 | |
| (−140677) | |
| chr6: 97087449-97087790 (+197719) | GPR63 |
| chr17: 19091397-19091591 (−140544) | GRAP |
| chrX: 108297584-108297772 (+427623) | GUCY2F |
| chr5: 83049568-83049768 (−32236) | HAPLN1 |
| chr19: 52207296-52207565 (+19790) | HAS1 |
| chr8: 123534074-123534351 (−880583) | HAS2 |
| chr1: 154244816-154245044 (−57) | HAX1 |
| chr5: 46345213-46345415 (−649061) | HCN1 |
| chr14: 31676971-31677156 (−375) | HECTD1 |
| chr3: 193852686-193852935 (−1123) | HES1 |
| chr8: 43092764-43093489 (+97529), chr8: 43094765-43095237 (+99403), | HGSNAT |
| chr8: 43095920-43096160 (+100442), chr8: 43096599-43097045 (+101224) | |
| chr6: 29894598-29894812 (+99949) | HLA-G |
| chr6: 44205665-44205858 (−9841) | HSP90AB1 |
| chr15: 90630376-90630595 (+15250) | IDH2 |
| chr13: 111364543-111364893 (−2611) | ING1 |
| chr12: 57824430-57824650 (−4003) | INHBC |
| chr13: 110438950-110439315 (−218), chr13: 110436939-110437154 (+1868) | IRS2 |
| chrX: 108297584-108297772 (−318027) | IRS4 |
| chr13: 48807203-48807417 (+16) | ITM2B |
| chr8: 41909521-41909718 (−115) | KAT6A |
| chr1: 215042851-215043097 (−213605) | KCNK2 |
| chr8: 140718091-140718298 (−2896) | KCNK9 |
| chr1: 215740559-215740855 (−28) | KCTD3 |
| chr4: 123073864-123074051 (+470) | KIAA1109 |
| chr18: 34409036-34409229 (+53) | KIAA1328 |
| chr12: 89366664-89366880 (−392534) | KITLG |
| chr13: 74149218-74149556 (+419799) | KLF12 |
| chr13: 74149218-74149556 (+516457) | KLF5 |
| chr7: 129710118-129710319 (−131) | KLHDC10 |
| chr12: 10507093-10507320 (+46790) | KLRD1 |
| chr12: 10507093-10507320 (+35410) | KLRK1 |
| chr11: 118306872-118307207 (−165) | KMT2A |
| chr12: 49454569-49454781 (−5568) | KMT2D |
| chr15: 40674993-40675207 (+178) | KNSTRN |
| chr17: 45727259-45727442 (+147) | KPNB1 |
| chr18: 7346609-7346801 (−228892) | LAMA1 |
| chr10: 103880192-103880391 (−82) | LDB1 |
| chr1: 212004012-212004220 (−2) | LPGAT1 |
| chr17: 62915466-62915663 (−662) | LRRC37A3 |
| chr3: 55361376-55361599 (−399387) | LRTM1 |
| chr11: 63933282-63934131 (−174) | MACROD1 |
| chr5: 108959504-108959876 (−65377) | MAN2A1 |
| chr21: 30627637-30627821 (+174856) | MAP3K7CL |
| chr14: 36417431-36417620 (+372356) | MBIP |
| chr1: 150552006-150552361 (−118) | MCL1 |
| chr22: 20861886-20862075 (+84) | MED15 |
| chr6: 131949219-131949582 (−32) | MED23 |
| chr3: 12598373-12598604 (−24) | MKRN2 |
| chr5: 44642366-44642562 (−166563) | MRPS30 |
| chr11: 1093135-1093332 (+18359) | MUC2 |
| chr11: 1093135-1093332 (−58346) | MUC5AC |
| chr19: 17530810-17531018 (+1024) | MVB12A |
| chr17: 34890655-34890876 (+49) | MYO19 |
| chr17: 31149515-31149817 (+54529) | MYO1D |
| chr2: 133033373-133033566 (−783475), chr2: 133025676-133025884 | MZT2A |
| (−775785), chr2: 133023177-133023551 | |
| (−773369), chr2: 133012312-133012914 (−762618) | |
| chr17: 59539182-59539462 (+129241) | NACA2 |
| chr1: 16840571-16840889 (+99252) | NBPF1 |
| chr6: 41395657-41395863 (+92233) | NCR2 |
| chr1: 16840571-16840889 (+73499) | NECAP2 |
| chr9: 140149613-140149812 (+88) | NELFB |
| chr7: 37802802-37803029 (−85283) | NME8 |
| chr14: 24768937-24769124 (−37) | NOP9 |
| chr9: 139430047-139430376 (+10102) | NOTCH1 |
| chr9: 140317619-140317809 (−88) | NOXA1 |
| chr20: 1447450-1447718 (−21) | NSFL1C |
| chr7: 151038891-151039139 (+218) | NUB1 |
| chr14: 105705366-105705606 (−57846) | NUDT14 |
| chr9: 133982504-133982693 (−18349) | NUP214 |
| chr7: 56440253-56440754 (−256411) | NUPR1L |
| chr6: 29242945-29243163 (−31349) | OR14J1 |
| chr6: 29242945-29243163 (+101743) | OR2J2 |
| chr5: 180586423-180586635 (+4586) | OR2V2 |
| chr11: 48894818-48895053 (+384667) | OR4A47 |
| chr11: 49985141-49985377 (+18812) | OR4C12 |
| chr11: 49985141-49985377 (+11316) | OR4C13 |
| chr11: 51580450-51580982 (+65434) | OR4C46 |
| chr11: 63753851-63754042 (+62) | OTUB1 |
| chr8: 81806026-81806246 (+218167) | PAG1 |
| chr21: 34144208-34144431 (−151) | PAXBP1 |
| chr7: 154794228-154794473 (+443) | PAXIP1 |
| chr4: 186717703-186717915 (−261147) | PDLIM3 |
| chr9: 123638626-123638828 (+879) | PHF19 |
| chr8: 75532782-75533005 (−203878) | PI15 |
| chr1: 27114609-27114805 (+21) | PIGV |
| chr17: 34890655-34890876 (−103) | PIGW |
| chr5: 108959504-108959876 (−213995) | PJA2 |
| chr20: 9049073-9049384 (+935957) | PLCB1 |
| chr11: 64037325-64037921 (+18604) | PLCB3 |
| chr20: 9049073-9049384 (−239218) | PLCB4 |
| chrX: 185033-187201 (−11742) | PLCXD1 |
| chr5: 10124-11780 (−129421) | PLEKHG4B |
| chr2: 87641562-87642062 (−392837) | PLGLB1 |
| chr2: 87641562-87642062 (−405794) | PLGLB2 |
| chr20: 56285036-56285231 (−176) | PMEPA1 |
| chr1: 156186290-156186573 (+3634) | PMF1 |
| chr19: 30067880-30068089 (−26939) | POP4 |
| chr22: 46544003-46544222 (−2386) | PPARA |
| chr1: 143283631-143283927 (+484102) | PPIAL4G |
| chr15: 64455271-64455570 (−17) | PPIB |
| chr1: 40204341-40204562 (−121) | PPIE |
| chr12: 111180559-111180989 (−30) | PPP1CC |
| chr2: 48782418-48782653 (+114600) | PPP1R21 |
| chr17: 48228319-48228524 (−545) | PPP1R9B |
| chrX: 459563-459810 (−112242) | PPP2R3B |
| chr18: 9502724-9502918 (+111735) | PPP4R1 |
| chr11: 64085477-64085814 (+86) | PRDX5 |
| chr20: 4667011-4667248 (+248) | PRNP |
| chr17: 1588111-1588419 (−134) | PRPF8 |
| chr17: 30771242-30771574 (+129) | PSMD11 |
| chr5: 40709833-40710038 (+30336) | PTGER4 |
| chr2: 232526867-232527107 (−46239) | PTMA |
| chr18: 12883502-12883753 (+606) | PTPN2 |
| chr18: 7346609-7346801 (−220075) | PTPRM |
| chr7: 158316688-158316900 (+63577) | PTPRN2 |
| chr20: 32287628-32287961 (+20311) | PXMP4 |
| chr12: 57824430-57824650 (+248) | R3HDM2 |
| chr8: 37761608-37761915 (−4790) | RAB11FIP1 |
| chr2: 73340286-73340568 (−281) | RAB11FIP5 |
| chr13: 111133834-111134061 (+80132) | RAB20 |
| chr11: 87300150-87300435 (+608342) | RAB38 |
| chr7: 6413962-6414208 (−85) | RAC1 |
| chr18: 9502724-9502918 (+27814) | RALBP1 |
| chr12: 26267030-26267216 (+155132) | RASSF8 |
| chr1: 192654633-192654845 (+49464) | RGS13 |
| chr1: 192654633-192654845 (−123432) | RGS2 |
| chr12: 49454569-49454781 (+9133) | RHEBL1 |
| chr10: 62817369-62817648 (−113504) | RHOBTB1 |
| chr22: 20689490-20689698 (−227808) | RIMBP3 |
| chr2: 86860434-86860746 (−86706) | RMND5A |
| chr2: 86860434-86860746 (−9612) | RNF103 |
| chr8: 101225206-101225507 (+90130) | RNF19A |
| chr18: 18519839-18520280 (+171752), chr18: 18518412-18519364 (+172924), | ROCK1 |
| chr18: 18517163-18517499 (+174481) | |
| chr2: 89874877-89875624 (+884089), chr2: 89879119-89879337 (+888066) | RPIA |
| chr1: 16563618-16563813 (−59) | RSG1 |
| chr3: 133969077-133969350 (+280) | RYK |
| chr11: 121028608-121028808 (−134454) | SC5D |
| chr11: 65190233-65190527 (−102168) | SCYL1 |
| chr9: 139430047-139430376 (−58071) | SEC16A |
| chr10: 12161018-12161226 (−10576) | SEC61A2 |
| chr18: 12948132-12948489 (+291) | SEH1L |
| chr14: 82698903-82699196 (−698845) | SEL1L |
| chr4: 77819223-77819410 (−51539) | 11-Sep |
| chrX: 459563-459810 (−125392) | SHOX |
| chr4: 77819223-77819410 (+463064) | SHROOM3 |
| chr19: 52207296-52207565 (−57377) | SIGLEC14 |
| chr20: 35243574-35243774 (+30945) | SLA2 |
| chr13: 99229367-99229634 (+175407) | SLC15A1 |
| chr6: 44205665-44205858 (+14399) | SLC29A1 |
| chr4: 41970877-41971061 (−21520) | SLC30A9 |
| chr2: 27434590-27434978 (+370) | SLC5A6 |
| chr17: 61920045-61920252 (+151) | SMARCD2 |
| chr6: 168899484-168899707 (+57765), chr6: 169055288-169055540 | SMOC2 |
| (+213583) | |
| chrX: 21959320-21959610 (+774) | SMS |
| chr8: 50183556-50183759 (−640575) | SNTG1 |
| chr4: 186717703-186717915 (+159997) | SORBS2 |
| chr8: 101225206-101225507 (+54701) | SPAG1 |
| chrX: 61745458-61745780 (+825599) | SPIN4 |
| chr7: 87849233-87849596 (−21) | SRI |
| chr22: 41253000-41253202 (−75) | ST13 |
| chr2: 191878709-191879075 (+84) | STAT1 |
| chr11: 63952478-63952971 (−862) | STIP1 |
| chr13: 99229367-99229634 (−55249) | STK24 |
| chr2: 48782418-48782653 (−13623) | STON1-GTF2A1L |
| chr1: 166845406-166845678 (+22) | TADA1 |
| chr20: 60192245-60192442 (+448522) | TAF4 |
| chr10: 105127605-105127795 (−24) | TAF5 |
| chr15: 102264480-102264717 (+208) | TARSL2 |
| chr17: 59539182-59539462 (+5515) | TBX4 |
| chr6: 167846728-167846945 (−48883) | TCP10 |
| chr11: 121028608-121028808 (+56826) | TECTA |
| chr4: 105537576-105537820 (−529752) | TET2 |
| chr17: 80317798-80318019 (+786), chr17: 80318319-80318796 (+1435) | TEX19 |
| chr13: 111805918-111806180 (−166966) | TEX29 |
| chr6: 169055288-169055540 (+598725), chr6: 168899484-168899707 | THBS2 |
| (+754543) | |
| chr17: 79367746-79367932 (−63365) | TMEM105 |
| chr14: 106056709-106056986 (+63908) | TMEM121 |
| chr11: 87300150-87300435 (+551244) | TMEM135 |
| chr2: 420569-420792 (+256758) | TMEM18 |
| chr2: 220406539-220406746 (−1742) | TMEM198 |
| chr19: 17530810-17531018 (+28462) | TMEM221 |
| chr11: 118401773-118402018 (−28) | TMEM25 |
| chr10: 62817369-62817648 (+395699) | TMEM26 |
| chr4: 41970877-41971061 (+33832) | TMEM33 |
| chr11: 129685636-129686050 (+129) | TMEM45B |
| chr21: 19970355-19970553 (−194481) | TMPRSS15 |
| chr21: 42879786-42880173 (+12) | TMPRSS2 |
| chr22: 41844406-41844636 (−1494), chr22: 41842826-41843024 (+102) | TOB2 |
| chr16: 33240538-33240873 (−20809) | TP53TG3B |
| chr16: 33240538-33240873 (+35726) | TP53TG3C |
| chr18: 34409036-34409229 (+25) | TPGS2 |
| chr21: 11186605-11186803 (−195822), chr21: 11143668-11143947 (−152926), | TPTE |
| chr21: 11011908-11012158 (−21151) | |
| chr11: 48894818-48895053 (−155568) | TRIM49B |
| chr5: 180586423-180586635 (+45643) | TRIM7 |
| chr11: 64085477-64085814 (−90) | TRMT112 |
| chr5: 40709833-40710038 (+46141) | TTC33 |
| chr2: 181845332-181845535 (+101) | UBE2E3 |
| chr17: 42296408-42296605 (+2487) | UBTF |
| chr3: 41855484-41855672 (+148344) | ULK4 |
| chr5: 176237352-176237680 (+38) | UNC5A |
| chr18: 108157-108471 (−50294) | USP14 |
| chr22: 18878053-18878574 (+245648), chr22: 18883687-18884010 (+251183) | USP18 |
| chr2: 61718794-61718987 (−20987) | USP34 |
| chr22: 20689490-20689698 (+55454) | USP41 |
| chr17: 80318319-80318796 (−13595) | UTS2R |
| chr11: 64863504-64863726 (−16) | VPS51 |
| chr19: 30067880-30068089 (+50579) | VSTM2B |
| chr2: 160142849-160143107 (+125) | WDSUB1 |
| chr9: 95964241-95964471 (+17144) | WNK2 |
| chr3: 55361376-55361599 (+162485) | WNT5A |
| chr22: 41253000-41253202 (−1) | XPNPEP3 |
| chr2: 61718794-61718987 (+46527) | XPO1 |
| chr22: 21984313-21984531 (−69) | YDJC |
| chrX: 2171370-2171572 (+247537) | ZBED1 |
| chr11: 10901550-10901737 (−22024) | ZBED5 |
| chr14: 64971586-64971856 (+429) | ZBTB1 |
| chr14: 64971586-64971856 (−1158) | ZBTB25 |
| chr9: 129622684-129622965 (−18802) | ZBTB34 |
| chr9: 129622684-129622965 (+55531) | ZBTB43 |
| chr1: 154973256-154973732 (−1801) | ZBTB7B |
| chr2: 113032973-113033242 (−63), chr2: 113033614-113033888 (+580) | ZC3H6 |
| chr2: 43454239-43454427 (−585) | ZFP36L2 |
| chr8: 123534074-123534351 (−259420) | ZHX2 |
| chr20: 45986392-45986656 (−1110) | ZMYND8 |
| chr11: 9482686-9483030 (+346) | ZNF143 |
| chr18: 74482695-74482904 (−53316) | ZNF236 |
| chr19: 45582355-45582540 (−2602) | ZNF296 |
| chr20: 26304186-26304530 (−636765), chr20: 26189806-26190159 (−522390), | ZNF337 |
| chr20: 26188743-26189163 (−521360) | |
| chr10: 42544497-42544802 (+589342), chr10: 42541440-42541749 (+592397), | ZNF33B |
| chr10: 42540015-42540294 (+593837), chr10: 42534957-42535220 (+598903), | |
| chr10: 42533454-42533854 (+600338), chr10: 42529451-42530254 (+604139), | |
| chr10: 42527582-42527898 (+606252), chr10: 42400227-42400626 (+733565), | |
| chr10: 42398482-42398897 (+735302), chr10: 42394181-42394923 (+739440), | |
| chr10: 42393324-42393647 (+740506), chr10: 42379899-42380287 (+753899), | |
| chr10: 42356259-42356510 (+777607) | |
| chr20: 32319851-32320136 (+531) | ZNF341 |
| chr7: 148844238-148844551 (−173) | ZNF398 |
| chr7: 56440253-56440754 (+767067) | ZNF479 |
| chr18: 74482695-74482904 (−275654) | ZNF516 |
| chr1: 249240245-249240510 (−87319) | ZNF692 |
| chr8: 81806026-81806246 (−19120) | ZNF704 |
| chr15: 90630376-90630595 (+85862) | ZNF710 |
| chr7: 148983045-148983319 (+175032) | ZNF777 |
| chr7: 148983045-148983319 (+23920) | ZNF783 |
| chr19: 58892363-58892553 (−69) | ZNF837 |
| chr6: 29894598-29894812 (−134326) | ZNRD1 |
| chrX: 58561257-58561614 (−624369) | ZXDA |

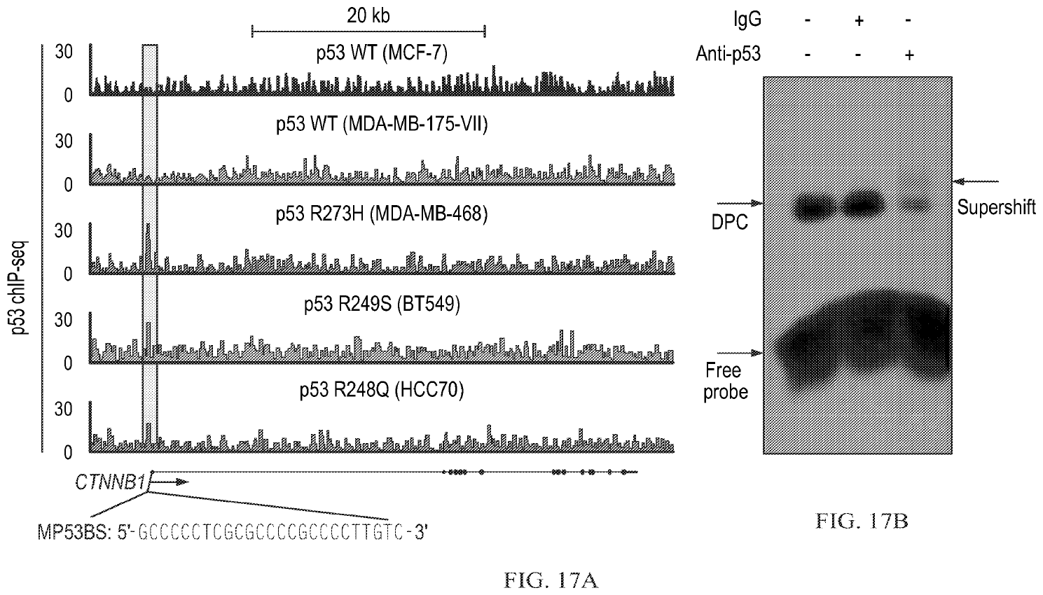

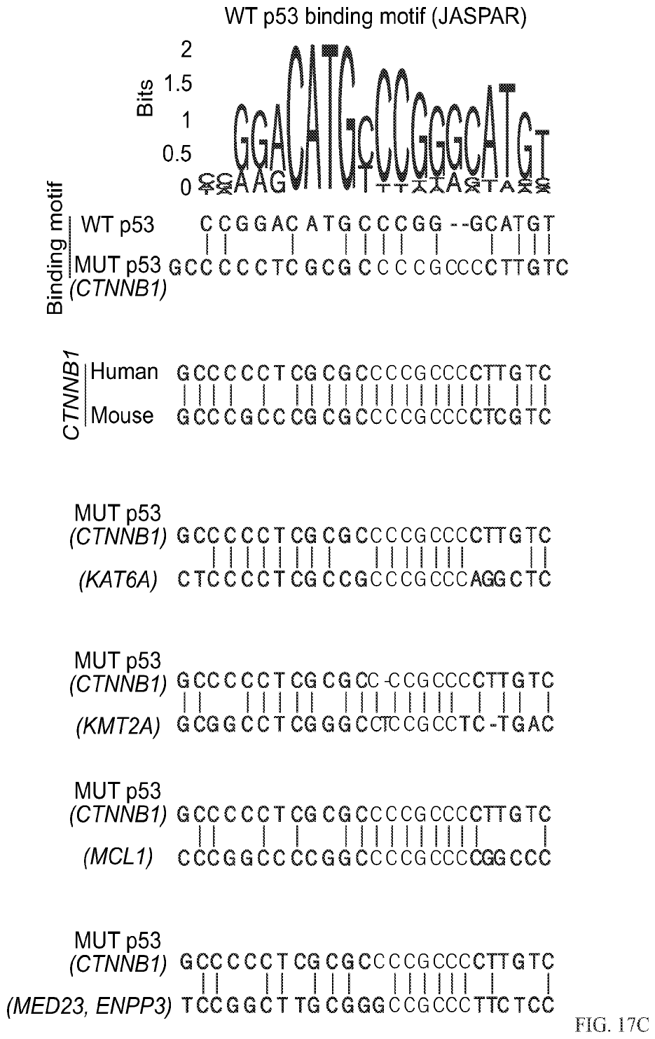

To define the potential downstream effector(s) underlying p53 mutant-mediated PSG expression, pathway enrichment analysis was conducted, and Wnt signaling was found to be one of the pathways enriched among the R248W-bound targets (FIG. 16B and Table 5). Specifically, a p53 mutant (R248W)-bound peak was detected in the promoter of CTNNB1 gene which encodes P3—Catenin, a core component of the Wnt signaling pathway (Clevers et al., Cell, 127:469-480 (2006)) (FIG. 16C). Specific occupancy of p53 R248W at the promoter of the CTNNB1 gene, but not in a non-promoter region, was verified by quantitative ChIP-PCR (ChIP-qPCR) in VCaP cells (FIG. 16D). Meta-analysis of p53 ChTP-seq data generated in different breast cancer cell lines expressing WT or GOF mutated p53 (Zhu et al. Nature, 525:206-211 (2015)) showed that p53 R273H, R249S and R248Q mutants, but not WT p53, invariably bound the CTNNB1 promoter (FIG. 17A).

To define the DNA sequence bound by GOF p53 mutant in the CTNNB1 promoter, p53 R248W ChIP-qPCR analysis was performed using a sequential set of primers (FIG. 16E). p53 R248W specifically occupied in the center (#2 amplicon) of the p53 mutant ChIP-seq peak in VCaP cells (FIG. 16F). By performing EMSA using VCaP cell lysate, this was further narrowed down to a 25-bp p53 mutant-bound DNA sequence (MP53BS) in the CTNNB1 gene promoter (FIGS. 16E and 16G). This motif shared approximately 50% of homology with the WT p53 binding consensus sequence, and was almost identical to mouse Ctnnb1 promoter (FIG. 17C). Notably, there was a CCCGCCC core motif that is also present in the promoters of many other GOF p53-bound cancer-related genes including those reportedly previously such as KAT6A and KMT2A (Zhu et al., Nature 525(7568): 206-211 (2015)) (FIG. 17C and Table 6). The EMSA signal of MP53BS was largely diminished by adding unlabeled probe or anti-p53 antibody in the assays (FIGS. 15H and 17B), indicating that the detected binding signal is p53 mutant (R248W) specific. Besides using cell nuclear extract, EMSA also was performed using glutathione-S transferase (GST)-p53 recombinant proteins purified from bacteria containing various mutations within the DNA binding domain (DBD) of p53 WT, including R175H p53, C238Y p53, R248W p53, R273H p53, and Q331R p53, and WT p53 (negative control). Except for WT and Q331R, all the DBD mutants of p53 bound to the DNA probe (FIG. 16I), suggesting that the DBD mutants of p53 directly bind to the MP53BS in the CTNNB1 gene promoter.

TABLE 6
MP53 binding sequence comparison among the genes with 10 base pair
unmatched compared to the MP53BS (25-bp) in the CTNNB1 gene promoter.

| Gene name | Location in chromatin and sequence (5′-3′) | SEQ ID NO |
|---|---|---|
| CR936796 | chr1:143283735:143283760: | 124 |
| | +:TCCCACAAGCTCCCATTTCCCTGTC | |
| MCL1 | chr1:150552238:150552263: | 125 |
| | −:CCCGGCCCCGGCCCCGCCCCGGCCC | |
| ZBTB7B | chr1:154973520:154973545: | 126 |
| | +:GCCCTCAGGGGCGCCTCACACTTTC | |
| PMF1 | chr1:156186429:156186454: | 127 |
| | −:GCCGCCTTCCCCGCCGCCCCCGGGT | |
| RSG1 | chr1:16563630:16563655: | 128 |
| | +:GCCCACTTCCGCCCCGCCTGCGTCC | |
| TADA1 | chr1:166845570:166845595: | 129 |
| | +:GCCGCCGTCAGTCCCGCCCCCAATC | |
| CROCCP3 | chr1:16840845:16840870: | 130 |
| | +:CCCCGCGCACTCCCGAGCCCTTTCC | |
| LPGAT1 | chr1:212004106:212004131: | 131 |
| | −:GCTTCCTCCCGCCGCCCCTCCCCGC | |
| KCTD3 | chr1:215740756:215740781: | 132 |
| | −:GCCGCCGGGCCGCCCGGGCCTCTTC | |
| FBX028 | chr1:224301863:224301888: | 133 |
| | −:GTCGCCTTGGCCGCCGCCTCCTTCC | |
| GALNT2 | chr1:230202602:230202627: | 134 |
| | −:CCCGCCGGCCGCCCCCTCCCCCGTG | |
| COX20 | chr1:244998937:244998962: | 135 |
| | −:AGCCCGGCTGGCCGCGCCCCTCCCC | |
| PIGV | chr1:27114719:27114744: | 136 |
| | +:GCTCCCTCGGGAGTGGTCCTTGGGC | |
| PPIE | chr1:40204460:40204485: | 137 |
| | −:GACCCCGCCCACCCTCGCGTTGGTC | |
| LDB1 | chr10:103880217:103880242: | 138 |
| | +:GCTCCCTCGCGCCGGCGCCGGCTCC | |
| TAF5 | chr10:105127710:105127735: | 139 |
| | −:GCCACCTCGCGCCGTCAAGCGTGAT | |
| KMT2A | chr11:118307020:118307045: | 140 |
| | −:GCGGCCTCGGGCCTCCGCCTCTGAC | |
| TMEM25 | chr11:118401857:118401882: | 141 |
| | +:GCCCCGCCTGGCGCAGCCCCCGCCC | |
| ARHGEF12 | chr11:120206969:120206994: | 142 |
| | −:GAGCTCGCGCTTCCCGTCCCCCGCC | |
| TMEM45B | chr11:129685829:129685854: | 143 |
| | −:GCCCCCTCTCCCTTCGAGCCCTCCT | |
| ALT3 | chr11:63439025:63439050: | 144 |
| | −:GCGCTTTCCCGCCCCGCCTCGCCTA | |
| MACROD1 | chr11:63933725:63933750: | 145 |
| | +:CCCCGCCTGAGCCCTTCGACTTCTC | |
| BAD | chr11:64037424:64037449: | 146 |
| | −:CCGCCCTCAGGCTCCGGGCTTTCCC | |
| PRDX5 | chr11:64085651:64085676: | 147 |
| | −:GGCCACTTCCACTCCGCCTCCTGGC | |
| VPS51 | chr11:64863665:64863690: | 148 |
| | +:GCCTCCTCCCCGTCCCCTTCCTTTC | |
| NEAT1 | chr11:65190373:65190398: | 149 |
| | −:CACCCCCAGCCCGCCCCCTCGTCTC | |
| DRAP1 | chr11:65686716:65686741: | 150 |
| | −:CGCCGCTCCCGCCCGCCCCCAAGGC | |
| TMEM135 | chr11:87300232:87300257: | 151 |
| | +:TGCCTCTTCAGCCCCGACCCTGACC | |
| ZNF143 | chr11:9482915:9482940: | 152 |
| | −:CCCTCTTTCACTCCCTCCCCTTGGC | |
| PPP1CC | chr12:111180785:111180810: | 153 |
| | −:GCCCCCTCCCGCGCCCCCTATGGCC | |
| ERC1 | chr12:1179999:1180024: | 154 |
| | +:GTTCCCTTGCATCTCTCCCCAGTTC | |
| BHLHE41 | chr12:26267059:26267084: | 155 |
| | +:GCCCGGCCGCGGCCCGCTCTAGACC | |
| RHEBLI | chr12:49454706:49454731: | 156 |
| | +:GCCCCCCTCCTCTCCGCCGCGGCCC | |
| DIP2B | chr12:50898871:50898896: | 157 |
| | +:CCCTCCTTCGGCCCCCTCTCTTGTC | |
| R3HDM2 | chr12:57824537:57824562: | 158 |
| | +:TCCGCCCGCCGCCCGGGCCCACGGC | |
| COPS7A | chr12:6833121:6833146: | 159 |
| | −:CCACCCCCGCGTTCCGACCGCTGAA | |
| IRS2 | chr13:110437054:110437079: | 160 |
| | −:CCGCCACCAGCCCCGGCTCCCTGTC | |
| IRS2 | chr13:110439115:110439140: | 161 |
| | +:CCCCTTTCTCCCCGGGCCGCGTTTC | |
| CARS2 | chr13:111364742:111364767 | 162 |
| | :−:GCCGCCGCGGGTCCCCTCCGGGGTG | |
| ANKRD10 | chr13:111567535:111567560: | 163 |
| | −:TCCGGCTCAAGCCCCGCCCCCTGCC | |
| ITM2B | chr13:48807256:48807281: | 164 |
| | +:CCCCGCCCCCGCCCCGCCTCCCAGG | |
| KLF12 | chr13:74149365:74149390: | 165 |
| | +:CCCCTCTGCCCAGCCGCCCCTACTG | |
| STK24 | chr13:99229480:99229505: | 166 |
| | −:GCTCCCGCGCGGCCACCGGCCTCTC | |
| abParts, ICHat, | chr14:106056711:106056736: | 167 |
| DKFZp686O16217 | +:GCCCAGTCCAGCCCAGCCCAATCTA | |
| HECTD1 | chr14:31677039:31677064: | 168 |
| | −:CGGCCGTCCCGCAGCGCCCCAGGTG | |
| GMFB | chr14:54955538:54955563: | 169 |
| | +:GCCCTCCTGGGCGCTGCCCGCCGCC | |
| ZBTB25 | chr14:64971703:64971728: | 170 |
| | −:GGCCCGTCGCCCTCCGGCCTGGGCC | |
| TARSL2 | chr15:102264682:102264707: | 171 |
| | −:GCCCCATCGCCCCGCCCCTCCCGGG | |
| APBA2 | chr15:29131500:29131525: | 172 |
| | −:ACCTCCGCGCCGCCCCCCACGCGTC | |
| KNSTRN | chr15:40675041:40675066: | 173 |
| | +:GGCTCCCGAAGCCCCGCCCCTGGAC | |
| PPIB | chr15:64455327:64455352: | 174 |
| | +:CCCCCCTCCGGCTCGGCGCCGGATA | |
| ADPGK | chr15:73076047:73076072: | 175 |
| | +:GCACCTGCGCGAACCAACTCCTTTC | |
| CD276 | chr15:73976517:73976542: | 176 |
| | −:GCCCCGCCCCGCCCCCAGGCCTGAC | |
| LOC100507217 | chr15:93352584:93352609: | 177 |
| | −:TGTCCCTCCGGGCCGGCCACGTGGC | |
| ABCA17P | chr16:2390834:2390859: | 178 |
| | +:GCTCCGGCCAGCCCAACCCCGCGCC | |
| LINC00273 | chr16:33963241:33963266: | 179 |
| | +:GCCTCTTGGCGCCCCCTCGATGCTC | |
| ESRP2 | chr16:68269906:68269931: | 180 |
| | −:GCCGCCCCCTCCCCCGGGCCCTGAC | |
| PRPF8 | chr17:1588270:1588295: | 181 |
| | −:ACCCCCTTGCGTTCAAAGCCTTTCC | |
| DHRS13 | chr17:27230101:27230126: | 182 |
| | −:ACCCGCACGCGCACACGCCCCTGCC | |
| SUZ12P1 | chr17:29035846:29035871: | 183 |
| | −:GCCTCCTCTCGCCTCCTCCCTGGAA | |
| PSMD11 | chr17:30771369:30771394: | 184 |
| | +:GGTCCCGCTCTCCCCACCCCTCAGC | |
| MYO1D | chr17:31149656:31149681: | 185 |
| | −:GCCCCACCGGGGCCCGCGCGGCCAC | |
| MYO19 | chr17:34890765:34890790: | 186 |
| | −:GCCGGCCGACCCCCGGCCACTTAGC | |
| UBTF | chr17:42296502:42296527: | 187 |
| | −:GCGCCCCCGCCTCCCGGCCGCCGTC | |
| KPNB1 | chr17:45727367:45727392: | 188 |
| | +:GCCCCCAGGGTCCCTCCCCCGCCGC | |
| PPP1R9B | chr17:48228395:48228420: | 189 |
| | +:TCCCCCCCGTGCCCCGAGCCTCGGT | |
| LRRC37A3 | chr17:62915621:62915646: | 190 |
| | −:GCGCCACCTCGCCCCGTCCCAGGCT | |
| RNASEK-C19orf49 | chr17:6918158:6918183: | 191 |
| | −:ACGTCATGGCCCCGCGCGCCTCCTC | |
| CSNKID | chr17:80231276:80231301: | 192 |
| | +:GCTCCTGCCCTCCCGGCCGCTTCCT | |
| PTPN2 | chr18:12883638:12883663: | 193 |
| | −:TCTTGCTCGCGGACAGCTCTTTCTC | |
| SEH1L | chr18:12948277:12948302: | 194 |
| | −:TTCCCCGCGCCGCCCACCCACTCCC | |
| DSG2 | chr18:29078132:29078157: | 195 |
| | −:GCGCGCACTCGGCTCCTCCCTGGCC | |
| TPGS2 | chr18:34409064:34409089: | 196 |
| | +:GCCCAGGCGCGACGCCCCGAGTGGC | |
| MVB12A | chr19:17530818:17530843: | 197 |
| | +:TCTCCGCCCCCCCCCCCCGCATGGC | |
| GEMIN7 | chr19:45582427:45582452: | 198 |
| | −:TTCCGCCCGCGCCGCCGCCACTGCC | |
| ERCC1 | chr19:45943266:45943291: | 199 |
| | −:CCCCCCGCGCGCTCCCCCTTCCGCT | |
| PPP5D1 | chr19:47104305:47104330: | 200 |
| | −:GCGCCCCGCCTCCCGGGCCCACTTC | |
| LINC00085 | chr19:52207406:52207431: | 201 |
| | +:GCCCCCTGGCCCGGCTCTACTTCTT | |
| ZC3H6 | chr2:113033105:113033130: | 202 |
| | −:TCCTCCGCGAGCCGCGGCCCTTGCC | |
| ZC3H6 | chr2:113033777:113033802: | 203 |
| | +:GCCCGGCCGCGCCCGCCTCCCCGAG | |
| ANKRD30BL | chr2:133025835:133025860: | 204 |
| | −:CTCTCCCCTTCCCCCACCCCTTGCC | |
| ANKRD30BL | chr2:133033446:133033471: | 205 |
| | −:GGCCTCCTTCCCTCTCCCCCTTGTC | |
| DDX1 | chr2:15732008:15732033: | 206 |
| | −:GCCTGCTCGCTCCCTCCCTCCCGAC | |
| WDSUB1 | chr2:160143003:160143028: | 207 |
| | −:GCCTGCTCGCGCGGGGCGTCTGAGC | |
| UBE2E3 | chr2:181845403:181845428: | 208 |
| | +:TCTCCCGCGTCCCCTCCGCCTCGCC | |
| STAT1 | chr2:191878916:191878941: | 209 |
| | +:CAGCGCTGGCGCCCGGCCCCCTTCC | |
| ABI2 | chr2:204193402:204193427: | 210 |
| | −:GGCCCCACGCCCCGAGGCGCGCGGC | |
| CHPF | chr2:220406671:220406696: | 211 |
| | −:TGCACCTGGCGCTGCGCCACCTGCT | |
| PTMA | chr2:232526907:232526932: | 212 |
| | +:GCCGTGTCGCGCCCCACCCTGCGCC | |
| SLC5A6 | chr2:27434809:27434834: | 213 |
| | −:GCCCCCGCGGGCTCCCGGCCAGGGC | |
| LOC100129726 | chr2:43454316:43454341: | 214 |
| | −:GCCACCCCCACCCCCACCGCGACTC | |
| C2orf61, CALM2 | chr2:47403656:47403681: | 215 |
| | +:CCTCCTCCGCCCCCAGCGCCTCATA | |
| STON1 | chr2:48782590:48782615: | 216 |
| | +:GACCCCCCCCACCTCCCTCCCGGAC | |
| RAB11FIP5 | chr2:73340504:73340529: | 217 |
| | +:GGCCGCGCTCTCCCCTCCTCCAGTC | |
| RNF103 | chr2:86860510:86860535: | 218 |
| | +:GACCCTGGATGCCCAGCCTCAAGTC | |
| NSFIC | chr20:1447585:1447610: | 219 |
| | +:GCCCGCCCGCAGCCCCGCCCCACGC | |
| LOC284801 | chr20:26188869:26188894: | 220 |
| | −:GGCGCCGCGGGACCGCCCTCGTGTC | |
| LOC284801 | chr20:26190010:26190035: | 22 |
| | +:GCTCCCTCAGGACCCACGCGCGGAC | |
| PXMP4 | chr20:32287726:32287751: | 222 |
| | +:CCCCCGCCTGGCCAGCCGCCTCGTC | |
| ZNF341 | chr20:32319981:32320006: | 223 |
| | −:GCCCGCGCCCCTCCCGCCCCGCGGC | |
| AP5S1 | chr20:3800888:3800913: | 224 |
| | +:GCTCCGAGCCGCGGCGCCCCCTCGC | |
| PRNP | chr20:4667120:4667145: | 225 |
| | +:GGCGCCGCGAGCTTCTCCTCTCCTC | |
| PMEPA1 | chr20:56285110:56285135: | 226 |
| | −:CGCAGCGCGCGCCCCCTCCCCGGCC | |
| PLCB4 | chr20:9049216:9049241: | 227 |
| | +:ACCCAGTCGGCGCCCACTCCTCGCC | |
| BAGE | chr21:11143693:11143718: | 228 |
| | +:GTCTCAGAGCCGCCCCCTCCTTGTC | |
| TMPRSS2 | chr21:42879944:42879969: | 229 |
| | +:GCGCCCAGGTTCCCCTCCCCAGCCC | |
| MED15 | chr22:20861934:20861959: | 230 |
| | −:TCCCCAGCTCCCGCCGCCCGTATCC | |
| YDJC | chr22:21984370:21984395: | 231 |
| | +:CGCTGCCCGGGCCCCGCCCCGACGC | |
| TOB2 | chr22:41842921:41842946: | 232 |
| | +:TTCCCAGCCAGCCCCGCGCCTCAGC | |
| TOB2 | chr22:41844477:41844502: | 233 |
| | +:CCTTTCCCCCTCCCCTTCCCTTCTC | |
| PPARA | chr22:46544035:46544060: | 234 |
| | +:TCCGTCCTGCGCCCTGCCGCTGGCC | |
| CCDC14 | chr3:123680230:123680255: | 235 |
| | +:ACCCCTCCAAGCTTCCCGGCTTGTC | |
| MKRN2 | chr3:12598514:12598539: | 236 |
| | −:GCCGGCGCACGCCGCCCCTCCGGTG | |
| RYK | chr3:133969140:133969165: | 237 |
| | −:CCCTCCCCGCCCCCCGCGCCCGGAT | |
| EIF4A2 | chr3:186501070:186501095: | 238 |
| | −:TTCCCCGCCCGCCCTGCTCCTACGG | |
| HES1 | chr3:193852795:193852820: | 239 |
| | +:GCCCCCCGCCTCCCTGCCCCCGGGG | |
| CTNNB1 | chr3:41241030:41241055: | 240 |
| | −:CTCCCCTCGCTCTCCGCTCCCGGGG | |
| SOWAHB | chr4:77819374:77819399: | 241 |
| | +:GCCTCCTCGTGTCCCTCCGCCCCCT | |
| MAN2A1 | chr5:108959516:108959541: | 242 |
| | −:ATGCCCTCTCTCACCCCTCCTATTC | |
| UNC5A | chr5:176237516:176237541: | 243 |
| | −:CGCCAGCCGCGCCGCGCACCGAGCC | |
| MED23, ENPP3 | chr6:131949453:131949478: | 244 |
| | −:TCCGGCTTGCGGGCCGCCCTTCTCC | |
| E2F3 | chr6:20402604:20402629: | 245 |
| | +:GCCGCCGCTGCCGCCGCCCCGGGCG | |
| DQ141194 | chr6:41395659:41395684: | 246 |
| | +:AGCCTCGGGGGCCCCGGGCCAGGTC | |
| HSP90AB1 | chr6:44205742:44205767: | 247 |
| | −:TCCCCGTCGCACCTCTCGCCGCCCC | |
| LUC7L2 | chr7:139025864:139025889: | 248 |
| | +:GCCCCTGTTTCCCCAAACCCTTGTT | |
| ZNF398 | chr7:148844412:148844437: | 249 |
| | −:CGCCCCTCCCCCCCAGCTCTTGGGC | |
| NUB1 | chr7:151039021:151039046: | 250 |
| | −:CCCCGCGGCCGCTCCACCCCTGGCC | |
| PAXIP1 | chr7154794351:154794376: | 251 |
| | +:CTTCCCTCCCGCACTCCCCCGGGCC | |
| BBS9 | chr7:33168827:33168852: | 252 |
| | +:AGCCAGTGACGCCCCGCCCCCACTA | |
| DQ584971 | chr7:56440560:56440585: | 253 |
| | +:GGCCTCCCGCGGCCAAGTCCCTGCC | |
| RAC1 | chr7:6414060:6414085: | 254 |
| | −:GGCCGCTCCCGCCCGCGCCGCTCCC | |
| SRI | chr7:87849397:87849422: | 255 |
| | +:GCCCCGCCCCGCCCTGCCGCTAGGG | |
| MCM7 | chr7:99698317:99698342: | 256 |
| | +:GCCCCCCCGGGCCGCAGCTCTCTCC | |
| SPAG1 | chr8:101225406:101225431: | 257 |
| | −:GCGCGCCCCTTGCCGGCCGCTTGCC | |
| CPSF1 | chr8:145634730:145634755: | 258 |
| | −:GAACCTTCCCGCCCAGCTTCTGGGC | |
| KAT6A | chr8:41909604:41909629: | 259 |
| | −:GCCTCCCCTCGCCGCCCGCCCAGGC | |
| ARMC1 | chr8:66528886:66528911: | 260 |
| | +:GCCTCTTCCCGGCCGCCATCACGTC | |
| PHF19 | chr9:123638714:123638739: | 261 |
| | +:GCCCCCGGGTGGCGCCCCCCCAGCC | |
| ZBTB34 | chr9:129622860:129622885: | 262 |
| | −:GCCCCCTGGACGCCCGCGCTTCCGG | |
| NOTCH1 | chr9:139430154:139430179: | 263 |
| | −:ACCCACTCCCACCCTGCACCCATTG | |
| NELFB | chr9:140149753:140149778: | 264 |
| | −:GCCCACTTCCGCCCCGCACCGCCCA | |
| EXD3, NOXA1 | chr9:140317715:140317740: | 265 |
| | −:TCCCATTCGGCCCCCGCCCCGGCCG | |
| CAAP1 | chr9:26892852:26892877: | 266 |
| | −:CCCCCCTGCCACCCCGGCCCATAAC | |
| BICD2 | chr9:95527279:95527304: | 267 |
| | +:GGGCCCTCGGGCGCAGCCACTGACG | |
| WNK2 | chr9:95964281:95964306: | 268 |
| | −:ACCCTCACCGGCCCAGCCTGGTTTC | |
| COL4A6 | chrX:108297664:108297689: | 269 |
| | +:GGCCCCCGGCGCCCCCCGGTGTCCC | |
| SMS | chrX:21959425:21959450: | 270 |
| | −:CCCCGCTCGGCGCACGCCCCCAGCC | |

### Chromatin Immunoprecipitation (ChIP) and ChIP-qPCR

VCaP cells were fixed and subjected to sonication by Bioruptor (Diagenode) as described elsewhere (Zhang et al., Nat Med 23(9): 1055-1062 (2017)). The supernatant was obtained and added by protein A/G beads and anti-p53 or anti-ERG antibodies. After incubation overnight, beads were washed, and the complex containing DNA was eluted at 65° C. The elution was further treated with RNAase and proteinase K. Enriched DNA was extracted for high throughput sequencing or quantitative PCR.

For the ChTP-seq assay, sequencing libraries were prepared as described elsewhere (Zhang et al., Nat Med. 23(9): 1055-1062 (2017)). The high-throughput sequencing was performed by Illumina HiSeq 4000 platform by Genome Analysis Core. The raw reads were subjected to the human reference genome (GRCh37/hg38) using bowtie2 (version 2.2.9). MACS2 (version 2.1.1) was run to perform the peak calling with a p value threshold of 1×10⁻⁵. BigWig files were generated for visualization using the UCSC Genome Browser. The assignment of peaks to potential target genes was performed by the Genomic Regions Enrichment of Annotations Tool (GREAT). ERG ChIP-seq data generated from the mouse prostate tissue was downloaded from NCBI Gene Expression Omnibus (GEO) with accession number GSE47119 (Chen et al., Nat Med. 19(8): 1023-1029 (2013)). β—Catenin ChIP-seq data was downloaded from GEO with accession number GSE53927 (Watanabe et al., PloS one 9, e92317 (2014)), p53 ChIP-seq data of breast cancer cell lines was downloaded from GEO with accession number GSE59176 (Zhu et al., Nature 525(7568): 206-211(2015)).

### GST Tagged Recombinant Protein Purification

GST-tagged p53 expression plasmids, including wild type (WT) and mutated p53, were transformed into E. coli BL21. The successful transformed BL21 were cultured in flasks in an incubator shaker and treated with 100 μM IPTG (Sigma) at 18° C. overnight. The induced BL21 were collected and resuspended in lysis buffer (50 mM Tris-HCl, pH 8.0) with protease inhibitor (Sigma) and sonicated. Glutathione Agarose (Thermo Fisher Scientific) were added to enrich the GST-p53 (WT/mutants) protein. The 10 mM reduced glutathione (Sigma) in 50 mM Tris-HCl, pH 8.0 was added and incubated with agarose for 1 hour at room temperature. The competed protein was collected by centrifuge and saved at −80° C. for further use.

### Nuclear Extraction and Electrophoretic Mobility Shift Assay (EMSA)

Double-stranded DNA oligonucleotides were labeled with biotin as probes by using the commercial kit (Thermo Fisher Scientific, Cat #89818) before use. The labeled probes were incubated with nuclear extraction prepared from VCaP cells using NE-PER™ Nuclear and Cytoplasmic Extraction Reagents (Thermo Fisher Scientific, Cat #78833) or purified GST-p53 protein according to the protocol provided by the manufacture (Thermo Fisher Scientific, Cat #20148). For supershift assay, anti-p53 antibodies were added into the cell nuclear extract mixed with the biotin-labeled probes and the mixture were incubated with for 1 hour at room temperature before loading into 6% of non-denatured polyacrylamide gel.

### Example 14: Therapeutic Targeting of the f-Catenin-LEF TCF Complex in ERG GOF p53 Mutant PCa

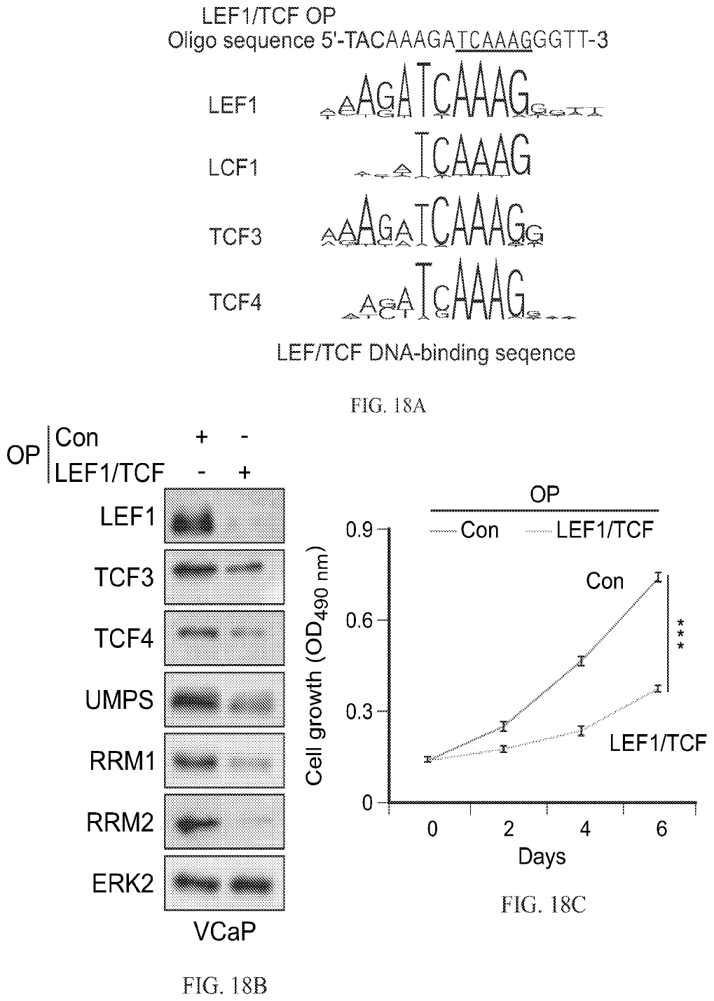

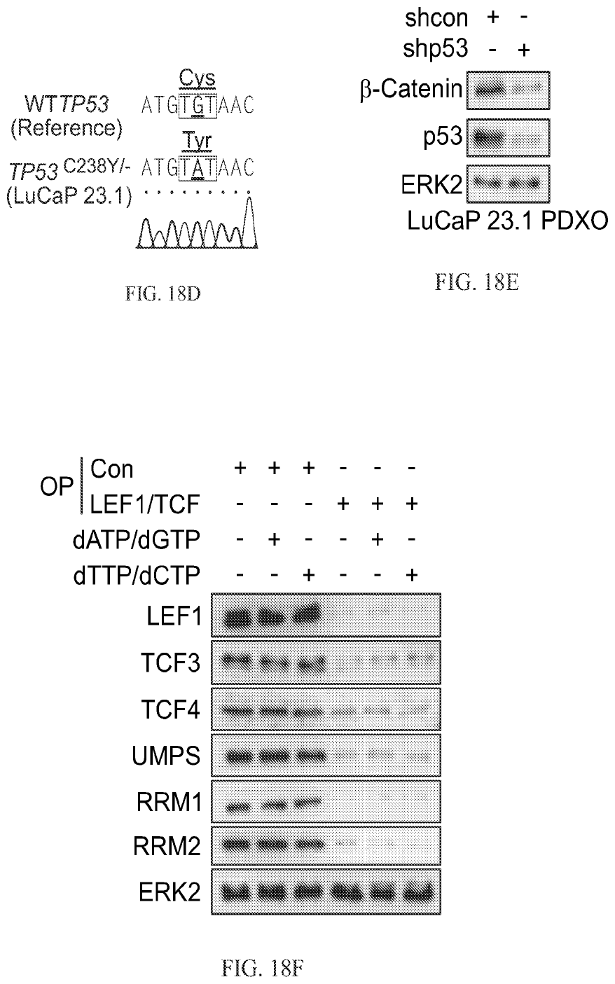

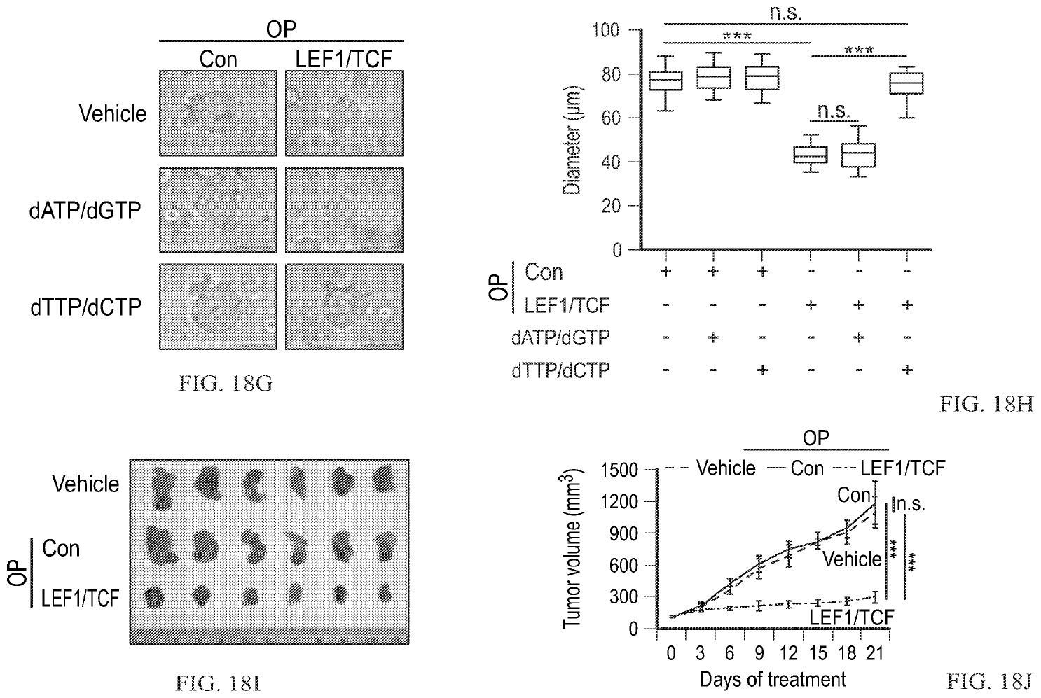

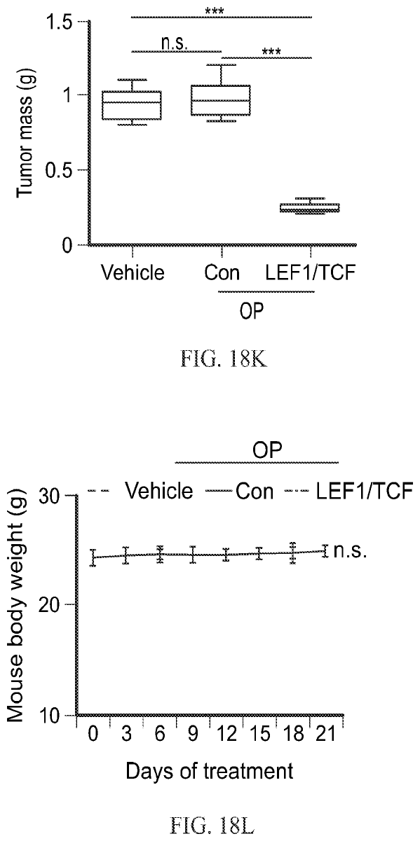

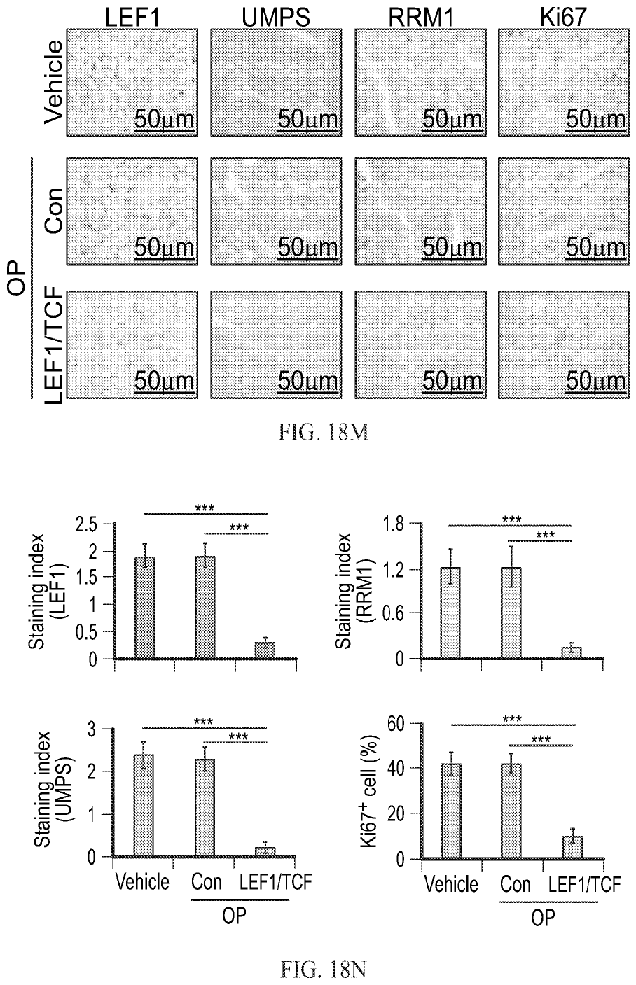

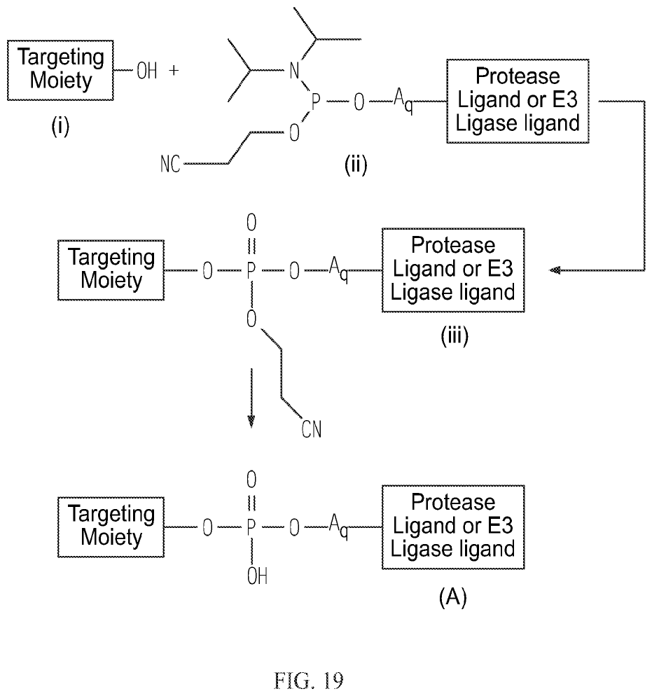

O′PROTACs were designed to target and destroy the LEF1 protein. β—Catenin transactivates its target genes by forming a protein complex with DNA binding partners LEF1 and other LEF/TCF family proteins including TCF1, TCF3 and TCF4 (Hrckulak et al., Cancers, 8:70 (2016)). Aberrant upregulation of β—Catenin in ERG/gain of function (GOF) p53 mutant PCa cells suggests that this cell type represents an ideal model to test the anti-cancer efficacy of LEF1 O′PROTAC. LEF1 OP-V1 ablated LEF1 protein in VCaP cells; and downregulated TCF3 and TCF4 protein to a certain degree, consistent with the observation that members of the LEF/TCF protein family bind the same core DNA sequence TCAAAG (FIGS. 18A and 18B). TCF1 was not examined because it was hardly detected in VCaP cells. The genotype-tissue expression (GTEx) RNA-seq data showed that TCF1 expression was undetectable in prostatic tissues (www.proteinatlas.org/). Importantly, this LEF1/TCF O′PROTAC also inhibited expression of pyrimidine synthesis enzyme proteins and growth of VCaP cells in culture (FIGS. 18B and 18C).

Next, the following was performed to determine the anti-cancer efficacy of LEF1/TCF O′PROTAC using ERG/GOF p53 mutant PCa organoids and PDXs. It has been reported that LuCaP 23.1 PDX and its androgen-independent (castration-resistant) subline LuCaP23.1AI are TMPRSS2-ERG positive and that one allele of TP53 is deleted (Kumar et al., PNAS, 108:17087 (2011)). The parental LuCaP 23.1 PDX tumors were found to harbor a C238Y mutation in p53 DBD (FIG. 18D). In agreement with the EMSA result that p53 C238Y mutant bound to MP53BS in the CTNNB1 promoter (FIG. 16I), p53 KD largely decreased β—Catenin protein expression in LuCaP23.1 PDX-derived organoids (PDXO) (FIG. 18E), highlighting that LuCaP23.1 is an ideal model system to test anti-cancer efficacy of inhibition of the β—Catenin-LEF/TCF pathway.

It was demonstrated that LEF1/TCF O′PROTAC treatment not only inhibited the expression of key pyrimidine synthesis enzyme proteins, but also effectively decreased the growth of LuCaP23.1 PDXO (FIGS. 18F-18H). Most importantly, this effect was almost completely reversed by supplementation of dTTP/dCTP, but not dATP/dGTP (FIGS. 18G and 18H), suggesting that the anti-cancer effect of LEF1/TCF O′PROTAC was largely mediated through the inhibition of pyrimidine synthesis. Compared to the effect of control OP or vehicle, treatment of LEF1/TCF O′PROTAC markedly blocked growth of LuCaP23.1 PDX tumors without causing any obvious reduction in body weight of mice (FIGS. 18I-18L). Immunohistochemistry (IHC) and Western blot analyses showed that LEF1/TCF O′PROTAC not only decreased expression of LEF1 and other LEF/TCF proteins and the pyrimidine synthesis enzymes examined such as UMPS and RRM1, but also largely reduced the number of Ki67-positive cells (FIGS. 18M and 18N). These results demonstrate that inhibition of 0—Catenin and PSG expression by targeting TCF/LEF proteins using O′PROTAC can effectively block the growth of PCa with TMPRSS2-ERG fusion and GOF p53 mutation.

### Cell and Organoid Culture

VCaP, DU145, LNCaP, and 293T cells were purchased from American Type Culture Collection (ATCC). DU145 and LNCaP cells were cultivated in RPMI 1640 media (Corning) with 10% fetal bovine serum (FBS) (Gbico). VCaP and 293T cells were grown in DMEM media (Corning) supplemented with 10% FBS (Millipore). All the cells were incubated at 37° C. supplied with 5% CO₂. Cells were treated with plasmocin (Invivogene) to eradicate mycoplasma in prior to the subsequent experiments.

Organoids were generated from LuCaP 23.1 patient-derived xenografts (PDXs) using the methods as described elsewhere (Drost et al., Nature Protocols, 11:347-358 (2016)). Briefly, organoids were cultured in 40 μL Matrigel (Sigma) mixed with FBS-free DMEMIF-12 medium supplemented with other factors.

### Transfection and Lentivirus Infection

Cells were transiently transfected with indicated plasmids using either Lipofectamine 2000 (Thermo Fisher Scientific) or polyethylenimine (PEI) (Polysciences, Catalog Number 23966) according to the manufactures' instructions. For lentivirus package, 293T cells were co-transfected with plasmids for psPAX2, pMDG.2 and shRNA using Lipofectamine 2000. Supernatant containing virus was harvested after 48 hours and added into cells after filtration by 0.45 m filter (Millipore). The indicated cells were added with the virus-containing supernatant in the presence of polybrene (5 μg/mL) (Millipore) and selected with 1 μg/mL puromycin (Selleck).

### Cell Growth Assay

VCaP cells were seeded at the density of 5,000 cells per well in 96-well plate overnight. At the indicated time points, optical density (OD) of cells was measured by microtiter reader (Biotek) at 490 nanometer after incubation with MTS (Promega) for 2 hours at 37° C. in a cell incubator. For the treatment with CP-2, ICG-001 or PRI-724, cells were seeded in 96-well plate overnight followed by adding indicated compounds. OD values were measured at the indicated time points.

### Hematoxylin and Eosin (H&E) Staining and Immunohistochemistry (IHC)

Four-μm sections were cut consecutively from formalin-fixed paraffin-embedded (FFPE) prostate tissues of indicated mice. Tissues were deparaffinized by xylene and subsequently rehydrated in turn through 100%, 95%, and 70% ethanal and water. After hematoxylin staining and Scott's Bluing solution (40.1 g MgSO4-7 H2O, 2 g sodium hydrogen carbonate, 1 L H2O) washing, tissues were counterstained with 1% eosin. After washing with 95% ethanol, tissues were dehydrated with 95% and 100% ethanol. Finally, the stained tissue was put in xylene and mounted with coverslips.

For IHC, tissues were rehydrated, destroyed endogenous peroxidase activity and antigen retrieval as described elsewhere (Blee et al., Clin. Cancer Res., 24:4551 (2018)). Antibodies for IHC as following: anti-AR (ab108341, Abcam), anti-ERG (ab92513, Abcam), anti-Ki67 (ab15580), anti-UMPS (NOVUS, #85896), anti-RRM1 (Cell signaling technology, #8637), anti-CBP (Santa Cruz Biotechnology, sc-583), and anti-LEF1 (Cell signaling technology, #2230S). For quantification, the staining score was determined by multiplying the percentage of positive cells and the intensity ranged from 1 (weak staining), 2 (median staining), and 3 (strong staining). For Ki67 quantification, cells with positive staining in the nucleus were included to calculate the percentage of Ki67 positive-staining cells.

### Example 15: Destruction of DNA-Binding Proteins by Programmable O′PROTAC: Oligonucleotide-Based PROTAC

### Abstract

DNA-binding proteins including transcription factors (TFs) play essential roles in gene transcription and DNA replication and repair during normal organ development and pathogenesis of diseases such as cancer, cardiovascular disease and obesity, deeming to be a large repertoire of attractive therapeutic targets. However, this group of proteins are generally considered undruggable as they lack enzymatic catalytic site or ligand binding pocket. PROteolysis-TArgeting Chimera (PROTAC) technology has been developed by engineering a bifunctional small molecule chimera to bring a protein of interest (POI) to the proximity of an E3 ubiquitin ligase for proteasome degradation, thus inducing ubiquitination of POI and further degradation through the proteasome pathway. Here we report the development of oligonucleotide-based PROTAC (O′PROTACs), a class of noncanonical PROTACs in which a TF-recognizing double-stranded oligonucleotide is incorporated as a binding moiety of POI. We demonstrate that O′PROTACs of ERG and LEF1, two highly cancer-related transcription factors selectively promote degradation of these proteins, inhibit their transcriptional activity, and inhibit cancer cell growth in vitro and in vivo. The programmable nature of O′PROTACs indicates that this approach is applicable to destruction of other TFs. O′PROTACs not only can serve as a research tool, but also can be harnessed as therapeutic arsenal to target disease-relevant TFs for effective treatment of diseases such as cancer.

### Introduction

A large group of DNA-binding proteins act as transcription factors (TFs) that transcriptionally activate or suppress gene expression by interacting with specific DNA sequence and transcription co-regulators. Approximately 2,000 TFs have been identified in eukaryotic cells and they are associated with numerous biological processes. Among them, approximately 300 TFs are associated with cancer development, which account for ˜19% of oncogenes¹. Therefore, targeting TFs associated with cancer development appear to be an appealing strategy for cancer treatment.

In the last decades, small molecule modulators have been developed to target nuclear receptors given that this class of TFs contain a clearly defined ligand-binding pocket². However, most of other TFs are difficult to target due to lack of ligand binding pocket. As the knowledge regarding the mechanisms of the assembly of transcription complexes has increased exponentially, different strategies to modulate the activity of TFs with small molecule compounds have emerged, including blocking protein/protein interactions, protein/DNA interactions, or chromatin remodeling/epigenetic reader proteins³. However, the development of traditional small molecules inhibiting non-ligand TFs remains very challenging, and a new targeting strategy to overcome the hurdle is very much needed.

PROTACs are heterobifunctional small molecules composed of a POI ligand as a warhead, a linker and an E3 ligase ligand, thus recruiting E3 ligase to POI and inducing prey protein to be degraded by the proteasome pathway. PROTAC technology has greatly advanced during the last decade. It has been proved that PROTACs are capable of degrading a variety of proteins, including enzymes and receptors⁴⁻⁸. Two PROTACs, ARV-110 and ARV-471 which are androgen receptor (AR) and estrogen receptor (ER) degraders, respectively have entered into phase I clinical trials⁹⁻¹¹. PROTACs offer several advantages over the other small molecule inhibitors including expanding target scope, improving selectivity, reducing toxicity and evading inhibitor resistance¹². This suggests that PROTAC technology is a new promising modality to tackle diseases, in particular for cancer. Most recently, PROTACs have been designed to degrade TFs. Wang's group developed a potent and signal transducers and activators of transcription 3 (STAT3)-specific degrader based on an STAT3 inhibitor SI-109 and demonstrated its targeting efficacy in vivo¹³. Crews' group reported the development of Transcription Factor Targeting Chimeras (TRAFTACs)¹⁴, which utilize haloPROTAC, dCas9-HT7 and dsDNA/CRISPR-RNA chimeras to degrade TFs. Nevertheless, this approach uses the artificially engineered dCas9-HT7 fusion protein as a mediator, which limits its potential use in clinic.

ETS-related gene (ERG) transcription factor belongs to the ETS family and is involved in bone development, hematopoiesis, angiogenesis, vasculogenesis, inflammation, migration and invasion¹⁵⁻¹⁶. Importantly, it is overexpressed in approximately 50% of all human prostate cancer cases including both primary and metastatic prostate cancer due to the fusion of ERG gene with the androgen-responsive TMPRSS2 gene promoter¹⁷⁻¹⁸. TMPRSS2-ERG gene fusion results in aberrant overexpression of truncated ERG, implying that increased expression of ERG is a key factor to drive prostate cancer progression¹⁹⁻²⁰. Therefore, therapeutic targeting ERG is urgently needed to effectively treat prostate cancer patients. Lymphoid enhancer-binding factor 1 (LEF1) is another highly cancer-related TF. It belongs to T cell factor (TCF)/LEF1 family. Complexed with 0-catenin, LEF1 promotes the transcription of Wnt target genes²¹. LEF1 also can facilitate epithelial-mesenchymal transition (EMT)²². Aberrant expression of LEF1 is implicated in several cancer types and related to cancer cell proliferation, migration, and invasion²³. Hence, LEF1 is another ideal target for cancer treatment.

In the present study we introduce a new strategy to target TFs using O′PROTACs, in which a double-stranded oligonucleotide is incorporated as POI binding moiety in PROTAC (FIG. 1). We demonstrate that ERG O′PROTAC promotes proteasomal degradation of ERG protein and inhibits ERG transcriptional activity. Akin to ERG degrader, LEF1 O′PROTAC induces the degradation of LEF1 and inhibits LEF1 transcriptional activity and prostate cancer cell growth in vitro and in mice. Consequently, its target gene expression and prostate cancer cell growth was also effectively inhibited.

### Results

### Design of O′PROTACs

ERG recognizes a highly conserved DNA binding consensus sequence including the 5′-GGAA/T-3′ core motif²⁴. We designed a 19-mer double-stranded oligonucleotide containing the sequence of

(SEQ ID NO: 3)
ACGGACCGGAAATCCGGTT

with the ERG binding moiety underscored. As for the E3 ligase-recruiting element, we selected the widely used pomalidomide and VH 032, which are capable of hijacking Cereblon and von Hippel-Lindau (VHL) respectively. PROTAC exerts its function based on the formation of ternary complex, in which a linker plays an important role. Therefore, we designed and synthesized six phosphoramidites with different linkers in different lengths and types, three of which are linked to pomalidomide and three with VH 032 (P1-6, Table 7). The phosphoramidite was attached to the 5′ terminal of one DNA strand through DNA synthesizer (Supporting Information). After annealing, we generated six O′PROTACs (OPs) for both ERG and LEF1, and three of them are designed to be bound by Cereblon (OP-C1-3 series) and three bound by VHL (OP-V1-3 series) (Table 8).
Chemical synthesis of P1-6

The synthesis of P1-6 was illustrated in Scheme 1. 4-Fluoro-thalidomide and VHL-032 were prepared according to literature procedures²⁵⁻²⁶. The straightforward nucleophilic aromatic substitution reaction of 4-fluoro-thalidomide with different amines provided key intermediates 8a-c. VH 032 was coupled with various carboxylic acids containing TBDPS protected hydroxyl group to deliver intermediates 8d-f. Subsequent acetylation of the hydroxyl groups in 8d-f and removal of the TBDPS protection produced intermediates 10a-c. Phosphitylation of 8a-c or 10a-c with Cl-POCEN′Pr₂ yielded P1-6 in the presence of DIPEA.

### ERG O′PROTACs Promote Proteasome Degradation of WT and TMPRSS2-ERG Proteins

The nucleic acid-based agents typically rely on lipid-mediated transfection to deliver them into cells. FITC-labelled ERG O′PROTAC was synthesized to determine the transfection efficiency under a fluorescent microscope. We transfected 293T cells with 100 or 1,000 nM of O′PROTAC with or without lipofectamine 2000. As expected, the presence of lipofectamine greatly enhanced the cellular uptake comparing with mock transfection (FIG. 2A). However, there was no difference in uptake efficacy between low (100 nM) and high concentration (1,000 nM) (FIG. 2A), probably owing to the saturation of the positively charged lipid with negatively charged oligonucleotide.

To assess the effects of ERG O′PROTACs on ERG proteins in cells, 293T cells were transfected with exogenously expressing HA-ERG plasmid and six ERG O′PROTACs at 100 nM for 48 hours and ERG protein level was measured by western blot. A significant decrease in ERG protein level was observed upon treatment with ERG OP-C1-3 attached with pomalidomide while the effects of ERG OP-V1-3 conjugated with VH 032 were much modest (FIG. 2B). To further demonstrate the cellular effect on endogenous ERG protein level, we tested ERG O′PROTACs in ERG-overexpressed human prostate cancer cell line VCaP which expresses both full-length ERG and TMPRSS2-ERG truncation. Similar to the effect on ectopically expressed ERG, ERG OP-C1-3 also effectively decreased endogenous ERG protein in VCaP cells (FIG. 2C). Intriguingly, a shorter linker such as five carbon atoms was favored for the more stable ternary complex. Although ERG OP-C1 significantly decreased ERG protein level, proteinase inhibitor MG132 blocked this degradation (FIG. 3), suggesting ERG O′PROTAC degrades ERG protein via proteasome pathway.

In vitro biotin pulldown assay showed that a significant amount of HA-ERG expressed in 293T cells was pulled down by biotin-labelled ERG OP-C1 and OP-C2 (FIG. 4), indicating that these two O′PROTACs strongly interact with ERG protein. This result also provides a plausible explanation for the better effect of these two O′PROTACS on ERG degradation.

Time-course studies showed that ERG O′PROTACs took effects starting from 12 hours until 48 hours examined (FIG. 5A). Consistent with the finding in 293T cells (FIG. 2A), the dose-course experiments revealed that 100 nM of ERG OP-C1 showed a significant inhibition of ERG protein level and this effect was not improved by higher concentrations such as 500 and 1,000 nM, indicating that ERG OP-C1 is probably saturated in a higher concentration (FIG. 5B). Additionally, treatment of VCaP cells with ERG OP-C1 inhibited mRNA expression of ERG target genes including ADAM19, MIP3, MAIP9, PLAT and PLAU (FIG. 5C), suggesting that ERG O′PROTAC inhibits ERG transcriptional activity in VCaP prostate cancer cells.

### Targeting Other TFs for Degradation by O′PROTACs

To extend the utility of O′PROTACs, we turned to another transcription factor LEF1. LEF1 acts as a DNA binding subunit in the 0-catenin/LEF1 complex and exerts transcriptional regulation via binding to the nucleotide sequence 5′-A/TA/TCAAAG-3′²⁷. We designed 18-mer double-stranded oligonucleotide containing the sequence of TACAAAGATCAAAGGGTT (SEQ ID NO:5) as the LEF1 binding moiety. Six LEF1 O′PROTACs (Table 8) were synthesized using the same protocol as for the ERG O′PROTACs.

We first evaluated the degradation capability of each LEF1 O′PROTACs in PC-3 prostate cancer cell line. Western blot assay was utilized to detect the expression of LEF1 protein. As shown in FIG. 6, LEF1 OP-V1 potently induced LEF1 degradation in PC-3 cells at a lower concentration (100 nM) while other LEF1 O′PROTACs were less or not active. This result is similar with ERG O′PROTACs, suggesting that both linker length and E3 ligase are important factors for degradation of a specific TF.

Next, we examined the effect of LEF1 O′PROTAC on the transcriptional activity of the β—Catenin/LEF1 complex. We found that treatment of PC-3 prostate cancer cells with LEF1 OP-V1 downregulated mRNA expression of CCND1 and c-MYC, two target genes of β—Catenin/LEF1 in a dose-dependent manner (FIGS. 7A and B). While LEF1 OP-V1 treatment did not affect mRNA expression of LEF1 gene, it markedly decreased expression of LEF1 and its target protein Cyclin D1 at the protein level in PC-3 (FIG. 7A). Importantly, LEF1 OP-V1 significantly inhibited PC-3 cell growth in a time- and dose-dependent fashion (FIGS. 7A and C). Similar results were obtained in another prostate cancer cell line DU145 (FIG. 7D-F). Collectively, LEF1 OP-V1 is a potent LEF1 degrader.

### Discussion

In this study we take a new strategy of degrading “undruggable” transcription factors by employing O′PROTACs. O′PROTAC exploits natural “ligand” of transcription factors, namely specific DNA sequence, attached to an E3 ligase ligand via a linker. The tactic has been successfully applied to degrade ERG and LEF1 TFs with potent efficacy in cultured cells.

Conventional PROTAC technology is rapidly evolving with some of them are in clinical trials; however, it inherits certain limitations. First, most of the reported PROTACs rely on the existing small molecules as targeting POI, which make it difficult to apply to “undruggable” targets like TFs. Additionally, due to their high molecular weight (600-1400 Da), PROTACs suffer from poor cell permeability, stability and solubility²⁹. In comparison with classic small molecule drugs, PROTACs are significantly less druggable. O′PROTACs hold enormous potentials to transcend the limitations of conventional PROTACs. Because of their modalities, degraders can be rationally programmed according to the DNA binding sequence of a given TF, thus theoretically making it possible to target any TF of interest. Our data suggest that the efficacy of O′PROTACs can be further optimized by the choice of the lengths and types of a linker and the E3 ligase ligand. Moreover, the synthesis of O′PROTAC is highly simple and efficient, which facilitates the rapid development of a O′PROTAC library for high-throughput screening of the most potent TF degraders. O′PROTAC could be applied to any proteins bound to DNA/DNA, DNA/RNA or RNA/RNA duplexes.

Hall and colleagues recently report a RNA-PROTAC, which utilizes single-stranded RNA (ssRNA) to recruit RNA-binding protein (RBP). The binding of RBP with single-stranded RNA heavily rely on both sequence motif and secondary structure³⁰. Predicting the interaction between ssRNA and RBP is challenging due to the high flexibility of ssRNA³¹. Our data show that the single-stranded O′PROTAC did not degrade either ERG or LEF1. However, double-stranded oligonucleotides bear a well-defined three-dimensional duplex structure; therefore, the protein binding region is accessible and predictable. Hence, O′PROTAC is programmable by changing the nucleotide sequence that binds protein. Additionally, compared with double-stranded oligonucleotide, ssRNA is susceptible to deleterious chemical or enzymatic attacks³¹. Taken together, O′PROTAC is desirable due to readily predictability and superior stability.

Oligonucleotide drug development has become a main stream for new drug hunting in the last decade³². The catalytic advantage of PROTACs³³ incorporated into oligonucleotide drugs could further fuel the field. Moreover, the delivery of oligonucleotide drugs has been advanced significantly in the recent years, notably for mRNA COVID-19 vaccine³⁴⁻³⁵. Therefore, O′PROTACs can be a complementary drug discovery and development platform to conventional PROTACs to derive clinical candidates and accelerate drug discovery.

### Experimental Section

### Synthesis of Phosphoramidites 1-6

Synthesis of phosphoramidites 1-6 was performed as described in Example 1.

### Synthesis of Oligonucleotides

All oligonucleotides used in this work were synthesized and reverse phase-HPLC purified by ExonanoRNA (Columbus, OH). Mass and purity (>95%) were confirmed by LC-MS from Novatia, LLC with Xcalibur system.

### Annealing Reaction

Single-stranded and reverse oligonucleotides were mixed in an assembly buffer (10 mM Tris-HCl [pH7.5], 100 mM NaCl, 1 mM EDTA), and heated to 90° C. for 5 min, then slowly cool down to 37° C. within 1 hour. Double-stranded O′PROTACs were mixed well, aliquoted and stored at −20° C. for the future use.

### Cell Culture and Transfection

VCaP, PC-3 and DU145 prostate cancer cell line and 293T cell line were obtained from the American Type Culture Collection (ATCC). 293T cells were maintained in DMEM medium with 10% FBS, PC-3 and DU145 cells were maintained in RPMI medium with 10% FBS. VCaP cells were cultured in RPMI medium with 15% FBS. Cells were transiently transfected using Lipofectamine 2000 (Thermo Fisher) for O′PROTAC according to the manufacturer's instructions.

### Western Blot

Cell lysate was subjected to SDS-PAGE and proteins were transferred to nitrocellulose membranes (GE Healthcare Sciences). The membranes were blocked in Tris-buffered saline (TBS, pH 7.4) containing 5% non-fat milk and 0.1% Tween-20, washed twice in TBS containing 0.1% Tween-20, and incubated with primary antibody overnight at 4° C., followed by secondary antibody for 1 hour at room temperature. The proteins of interest were visualized using ECL chemiluminescence system (Thermo Fisher).

### Biotin Pull-Down Assay

The 293T cells were transfected with 100 nM of biotin-labelled ERG O′ PROTACs and 1 μg of HA-ERG plasmid in 10-cm dishes using Lipofectamine 2000 (Thermo Fisher) for 36 h. The cells were treated with MG132 for 12 hours before lysed in lysis buffer containing 50 mM Tris-HCl (pH7.5), 150 mM NaCl, 1% NP-40, 0.5% sodium deoxycholate and 1% proteinase inhibitor. The cell lysate was incubated with Streptavidin Sepharose High Performance beads (GE Healthcare) overnight at 4° C. The binding protein was eluted by elution buffer and subjected to western blot.

### RNA Extraction and RT-qPCR

RNA was extracted using TRIzol (Invitrogen) and reversely transcribed into cDNA with SuperScript III First-Strand Synthesis System (Promega). The quantitative PCR (qPCR) was performed in the iQ thermal cycler (Bio-Rad) using the iQ SYBR Green Supermix (Bio-Rad). Each sample was carried out in triplicate and three biological repeats were performed. The ΔCT was calculated by normalizing the threshold difference of a certain gene with glyceraldehyde-3-phosphate dehydrogenase (GAPDH). The primer sequences are listed in Table 9.

### Cell Growth Assay

PC-3 and DU145 cells were transfected with LEF1 OP-V1 for 48 hours and seeded in 96-well plate at the density of 1,000 per well. After cells adhered to the plate, at indicated time points, the CellTiter 96 Aqueous One solution Cell Proliferation Assay (MTS) (Promega) was added to each well to measure cell viability. MTS was diluted at a ratio of 1:10 in PBS and added into the wells and incubated for 2 hours at 37° C. in a cell incubator. Microplate reader was used to measure absorbance of 490 nm in each well.

TABLE 8
The sequences of O′PROTACs

| O′PROTAC | Sequence | SEQ ID NO |
|---|---|---|
| ERG OP-C1 | Forward: 5′-ACGGACCGGAAATCCGGTT-3′ | 3 |
| | Reverse: 5′-P1-AACCGGATTTCCGGTCCGT-3′ | 109 |
| ERG OP-C2 | Forward: 5′-ACGGACCGGAAATCCGGTT-3′ | 3 |
| | Reverse: 5′-P2-AACCGGATTTCCGGTCCGT-3′ | 109 |
| ERG OP-C3 | Forward: 5′-ACGGACCGGAAATCCGGTT-3′ | 3 |
| | Reverse: 5′-P3-AACCGGATTTCCGGTCCGT-3′ | 109 |
| ERG OP-V1 | Forward: 5′-ACGGACCGGAAATCCGGTT-3′ | 3 |
| | Reverse: 5′-P4-AACCGGATTTCCGGTCCGT-3′ | 109 |
| ERG OP-V2 | Forward: 5′-ACGGACCGGAAATCCGGTT-3′ | 3 |
| | Reverse: 5′-P5-AACCGGATTTCCGGTCCGT-3′ | 109 |
| ERG OP-V3 | Forward: 5′-ACGGACCGGAAATCCGGTT-3′ | 3 |
| | Reverse: 5′-P6-AACCGGATTTCCGGTCCGT-3′ | 109 |
| FITC-ERG OP-C1 | Forward: 5′-FITC-ACGGACCGGAAATCCGGTT-3′ | 3 |
| | Reverse: 5′-P1-AACCGGATTTCCGGTCCGT-3′ | 109 |
| Biotin-ERG OP-C1 | Forward: 5′-Biotin-ACGGACCGGAAATCCGGTT-3′ | 3 |
| | Reverse: 5′-P1-AACCGGATTTCCGGTCCGT-3′ | 109 |
| LEF1 OP-C1 | Forward: 5′-TACAAAGATCAAAGGGTT-3′ | 5 |
| | Reverse: 5′-P1-AACCCTTTGATCTTTGTA-3′ | 115 |
| LEF1 OP-C2 | Forward: 5′-TACAAAGATCAAAGGGTT-3′ | 5 |
| | Reverse: 5′-P2-AACCCTTTGATCTTTGTA-3′ | 115 |
| LEF1 OP-C3 | Forward: 5′-TACAAAGATCAAAGGGTT-3′ | 5 |
| | Reverse: 5′-P3-AACCCTTTGATCTTTGTA-3′ | 115 |
| LEF1 OP-V1 | Forward: 5′-TACAAAGATCAAAGGGTT-3′ | 5 |
| | Reverse: 5′-P4-AACCCTTTGATCTTTGTA-3′ | 115 |
| LEF1 OP-V2 | Forward: 5′-TACAAAGATCAAAGGGTT-3′ | 5 |
| | Reverse: 5′-P5-AACCCTTTGATCTTTGTA-3′ | 115 |
| LEF1 OP-V3 | Forward: 5′-TACAAAGATCAAAGGGTT-3′ | 5 |
| | Reverse: 5′-P6-AACCCTTTGATCTTTGTA-3′ | 115 |
| LEF1 | Forward: 5′-TACAAAGATCAAAGGGTT-3′ | 5 |
| | Reverse: 3′-ATGTTTCTAGTTTCCCAA-5′ | 418 |
| LEF1 -F-Biotin | Forward: 5′-Biotin-TACAAAGATCAAAGGGTT-3′ | 5 |
| | Reverse: 3′-ATGTTTCTAGTTTCCCAA-5′ | 418 |
| LEF1-F-FITC | Forward: 5′-FITC-TACAAAGATCAAAGGGTT-3′ | 5 |
| | Reverse: 3′-ATGTTTCTAGTTTCCCAA-5′ | 418 |
| LEF1-V1 | Forward: 5′-TACAAAGATCAAAGGGTT-3′ | 5 |
| | Reverse: 3′-ATGTTTCTAGTTTCCCAA-L4-VHL-5′ | 418 |
| LEF1-V2 | Forward: 5′-TACAAAGATCAAAGGGTT-3′ | 5 |
| | Reverse: 3′-ATGTTTCTAGTTTCCCAA-L5-VHL-5′ | 418 |
| LEF1-V3 | Forward: 5′-TACAAAGATCAAAGGGTT-3′ | 5 |
| | Reverse: 3′-ATGTTTCTAGTTTCCCAA-L6-VHL-5′ | 418 |
| ERG | Forward: 5′-ACGGACCGGAAATCCGGTT-3′ | 3 |
| | Reverse: 3′-TGCCTGGCCTTTAGGCCAA-5′ | 419 |
| ERG-F-Biotin | Forward: 5′-Biotin-ACGGACCGGAAATCCGGTT-3′ | 3 |
| | Reverse: 3′-TGCCTGGCCTTTAGGCCAA-5′ | 419 |
| ERG-F-FITC | Forward: 5′-FITC-ACGGACCGGAAATCCGGTT-3′ | 3 |
| | Reverse: 3′-TGCCTGGCCTTTAGGCCAA-5′ | 419 |
| ERG-C-N1 | Forward: 5′-ACGGACCGGAAATCCGGTT-3′ | 3 |
| | Reverse: 3′-TGCCTGGCCTTTAGGCCAA-5′-L7-CRBN-5′ | 419 |
| ERG-C-N2 | Forward: 5′-ACGGACCGGAAATCCGGTT-3′ | 3 |
| | Reverse: 3′-TGCCTGGCCTTTAGGCCAA-5′-L8-CRBN-5′ | 419 |
| ERG-C-A1 | Forward: 5′-ACGGACCGGAAATCCGGTT-3′ | 3 |
| | Reverse: 3′-TGCCTGGCCTTTAGGCCAA-5′-L9-CRBN-5′ | 419 |
| ERG-C-A2 | Forward: 5′-ACGGACCGGAAATCCGGTT-3′ | 3 |
| | Reverse: 3′-TGCCTGGCCTTTAGGCCAA-5′-L10-CRBN-5′ | 419 |
| ERG-V1 | Forward: 5′-ACGGACCGGAAATCCGGTT-3′ | 3 |
| | Reverse: 3′-TGCCTGGCCTTTAGGCCAA-5′-L4-VHL-5′ | 419 |
| ERG-V2 | Forward: 5′-ACGGACCGGAAATCCGGTT-3′ | 3 |
| | Reverse: 3′-TGCCTGGCCTTTAGGCCAA-5′-L5-VHL-5′ | 419 |
| ERG-V3 | Forward: 5′-ACGGACCGGAAATCCGGTT-3′ | 3 |
| | Reverse: 3′-TGCCTGGCCTTTAGGCCAA-5′-L6-VHL-5′ | 419 |
| ERG-C1 | Forward: 5′-ACGGACCGGAAATCCGGTT-3′ | 3 |
| | Reverse: 3′-TGCCTGGCCTTTAGGCCAA-L1-phthalic acid-5′ | 419 |
| ERG-C2 | Forward: 5′-ACGGACCGGAAATCCGGTT-3′ | 3 |
| | Reverse: 3′-TGCCTGGCCTTTAGGCCAA-L2-phthalic acid-5′ | 419 |
| ERG-C3 | Forward: 5′-ACGGACCGGAAATCCGGTT-3′ | 3 |
| | Reverse: 3′-TGCCTGGCCTTTAGGCCAA-L3-phthalic acid-5′ | 419 |
| ERG-C-P1 | Forward: 5′-ACGGACCGGAAATCCGGTT-3′ | 3 |
| | Reverse: 3′-TGCCTGGCCTTTAGGCCAA-L1-phthalic acid-5′ | 419 |
| CTRL | Forward: 5′-TGTGCTAGCTGATGTGCTA-3′ | 420 |
| | Reverse: 3′-ACACGATCGACTACACGAT-5′ | 421 |
| CTRL-C-N1 | Forward: 5′-TGTGCTAGCTGATGTGCTA-3′ | 420 |
| | Reverse: 3′-ACACGATCGACTACACGAT-L7-pomalidomide-5′ | 421 |
| CTRL-V1 | Forward: 5′-TGTGCTAGCTGATGTGCTA-3′ | 420 |
| | Reverse: 3′-ACACGATCGACTACACGAT-L4-VHL-5′ | 421 |

TABLE 9
Primer sequence for RT-qPCR

| | | | SEQ | | SEQ |
|---|---|---|---|---|---|
| Gene | Usage | Forward (5′-3′) | ID NO | Reverse (5′-3′) | ID NO |
| ADAM19 | RT-qPCR | GCCTATGCCCCCTGAGAGTG | 422 | GCTTGAGTTGGCCTAGTTTGTTGTTC | 423 |
| GAPDH | RT-qPCR | GAAGGTGAAGGTCGGAGTC | 424 | GAAGATGGTGATGGGATTTC | 425 |
| MMP3 | RT-qPCR | TTCATTTTGGCCATCTCTTCCTTCAG | 426 | TATCCAGCTCGTACCTCATTTCCTCT | 427 |
| MMP9 | RT-qPCR | TGCCCGGACCAAGGATACAGT | 428 | AGCGCGTGGCCGAACTCAT | 429 |
| PLAT | RT-qPCR | CACTGGGCCTGGGCAAACATA | 430 | CACGTCAGCCTGCGGTTCTTC | 431 |
| PLAU | RT-qPCR | TACGGCTCTGAAGTCACCACCAAAAT | 432 | CCCCAGCTCACAATTCCAGTCAA | 433 |
| ERF FL | RT-qPCR | GAGAGTGTGCAAGAGATC | 434 | GCTTTTGGTCAACACGG | 435 |
| ERG T1/E4 | RT-qPCR | CGCCCGGAGGTGAAAGCG | 436 | GCTTTTGGTCAACACGG | 435 |

TABLE 9B
The sequences of O′PROTAC.

| O′PROTAC | Sequence | SEQ ID NO |
|---|---|---|
| ERG OP-C1 | Forward: 5′-ACGGACCGGAAATCCGGTT-3′ | 3 |
| | Reverse: 5′-P1-AACCGGATTTCCGGTCCGT-3′ | 109 |
| FITC-ERG OP-C1 | Forward: 5′-FITC-ACGGACCGGAAATCCGGTT-3′ | 3 |
| | Reverse: 5′-P1-AACCGGATTTCCGGTCCGT-3′ | 109 |
| Biotin-ERG OP-C1 | Forward: 5′-Biotin-ACGGACCGGAAATCCGGTT-3′ | 3 |
| | Reverse: 5′-P1-AACCGGATTTCCGGTCCGT-3′ | 109 |
| LEF1 OP-C1 | Forward: 5′-TACAAAGATCAAAGGGTT-3′ | 5 |
| | Reverse: 5′-P1-AACCCTTTGATCTTTGTA-3′ | 115 |

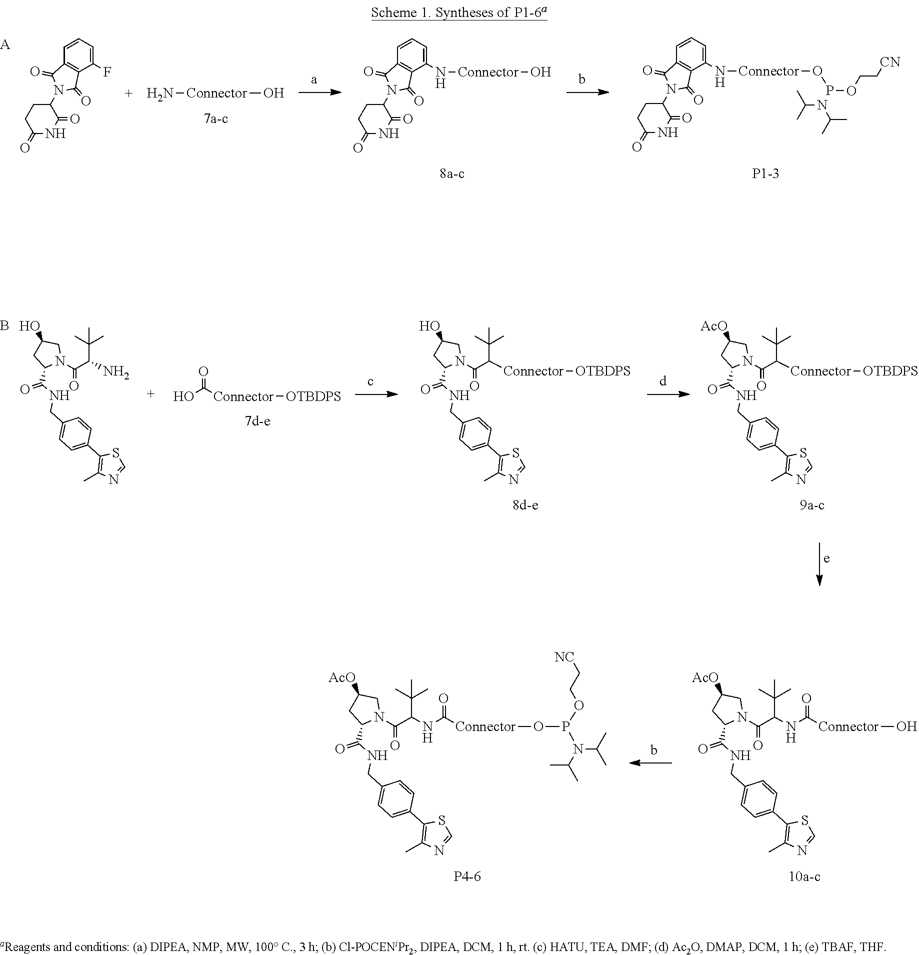

### REFERENCES

1. Lambert, M.; Jambon, S.; Depauw, S.; David-Cordonnier, M. H., Targeting Transcription Factors for Cancer Treatment. Molecules 2018, 23 (6).
2. Zhao, L.; Zhou, S.; Gustafsson, J.-A., Nuclear Receptors: Recent Drug Discovery for Cancer Therapies. Endocrine Reviews 2019, 40 (5), 1207-1249.
3. Hagenbuchner, J.; Ausserlechner, M. J., Targeting transcription factors by small compounds-Current strategies and future implications. Biochemical Pharmacology 2016, 107, 1-13.
4. Burslem, G. M.; Song, J.; Chen, X.; Hines, J.; Crews, C. M., Enhancing Antiproliferative Activity and Selectivity of a FLT-3 Inhibitor by Proteolysis Targeting Chimera Conversion. Journal of the American Chemical Society 2018, 140 (48), 16428-16432.
5. Cromm, P. M.; Samarasinghe, K. T. G.; Hines, J.; Crews, C. M., Addressing Kinase-Independent Functions of Fak via PROTAC-Mediated Degradation. Journal of the American Chemical Society 2018, 140 (49), 17019-17026.
6. Li, Z.; Lin, Y; Song, H.; Qin, X.; Yu, Z.; Zhang, Z.; Dong, G.; Li, X.; Shi, X.; Du, L.; Zhao, W.; Li, M., First small-molecule PROTACs for G protein-coupled receptors: Inducing alpha1A-adrenergic receptor degradation. Acta Pharmaceutica Sinica B 2020.
7. Khan, S.; Zhang, X.; Lv, D.; Zhang, Q.; He, Y; Zhang, P.; Liu, X.; Thummuri, D.; Yuan, Y; Wiegand, J. S.; Pei, J.; Zhang, W.; Sharma, A.; McCurdy, C. R.; Kuruvilla, V. M.; Baran, N.; Ferrando, A. A.; Kim, Y-M.; Rogojina, A.; Houghton, P. J.; Huang, G.; Hromas, R.; Konopleva, M.; Zheng, G.; Zhou, D., A selective BCL-X(L) PROTAC degrader achieves safe and potent antitumor activity. Nat Med 2019, 25 (12), 1938-1947.
8. Paiva, S.-L.; Crews, C. M., Targeted protein degradation: elements of PROTAC design, Curr Opin. Chem. Biol. 2019, 50, 111.
9.a) Mullard., A, Targeted protein degraders crowd in the clinic, Nat. Rev. Drug. Discov. 2021, 20, 247; b) Burslem, G. M.; Crews, C. M., Proteolysis-targeting chimeras as therapeutics and tools for biological discovery, Cell 2020, 181, 102.
10. Neklesa, T.; Snyder, L. B.; Willard, R. R.; Vitale, N.; Pizzano, J.; Gordon, D. A.; Bookbinder, M.; Macaluso, J.; Dong, H.; Ferraro, C.; Wang, G.; Wang, J.; Crews, C. M.; Houston, J.; Crew, A. P.; Taylor, I., ARV-110: An oral androgen receptor PROTAC degrader for prostate cancer. Journal of Clinical Oncology 2019, 37 (7_suppl), 259-259.
11. Gao, H.; Sun, X.; Rao, Y, PROTAC Technology: Opportunities and Challenges. ACS Med Chem Lett 2020, 11 (3), 237-240.
12. Pettersson, M.; Crews, C. M., PROteolysis TArgeting Chimeras (PROTACs)—Past, present and future. Drug Discov Today Technol 2019, 31, 15-27; b) Smith, B. E.; Wang, S. L.; Jaime-Figueroa, S.; Harbin, A.; Wang, J.; Hamman, B. D.; Crews C. M., Differential PROTAC substrate specificity dictated by orientation of recruited E3 ligase, Nat. Commun. 2019, 10, 131; c) Khan, S.; Zhang, X.; Lv, D.; Zhang, Q.; He, Y; Zhang, P.; Liu, X.; Thummuri, D.; Yuan, Y; Wiegand, J. S.; Pei, J., Zhang, W.; Sharma, A.; McCurdy, C. R.; Kuruvilla, V. M.; Baran, N.; Ferrando, A. A.; Kim, Y-M.; Rogojina, A.; Houghton, P. J.; Huang, G.; Hromas, R.; Konopleva, M.; Zheng, G.; Zhou, D., A selective BCL-XL PROTAC degrader achieves safe and potent antitumor activity, Nat. Med. 2019, 25, 1938; d) Buhimschi, A. D.; Armstrong, H. A.; Toure, M.; Jaime-Figueroa, S.; Chen, T. L.; Lehman, A. M.; Woyach, J. A.; Johnson, A. J.; Byrd, J. C.; Crews, C. M., Targeting the C481S Ibrutinib-resistance mutation in Bruton's tyrosine kinase using PROTAC-mediated degradation, Biochemistry 2018, 57, 3564-3575; e) Burslem, G. M.; Smith, B. E.; Lai, A. C.; Jaime-Figueroa, S.; McQuaid, D. C.; Bondeson, D. P.; Toure, M.; Dong, H.; Qian, Y; Wang, J.; Crew, A. P.; Hines, J.; Crews, C. M., The advantages of targeted protein degradation over inhibition: an RTK case study, Cell Chem. Biol. 2018, 25, 67.
13. Bai, L.; Zhou, H.; Xu, R.; Zhao, Y; Chinnaswamy, K.; McEachern, D.; Chen, J.; Yang, C. Y; Liu, Z.; Wang, M.; Liu, L.; Jiang, H.; Wen, B.; Kumar, P.; Meagher, J. L.; Sun, D.; Stuckey, J. A.; Wang, S., A Potent and Selective Small-Molecule Degrader of STAT3 Achieves Complete Tumor Regression In Vivo. Cancer Cell 2019, 36 (5), 498-511 e17.
14. Samarasinghe, K. T. G.; Jaime-Figueroa, S.; Dai, K.; Hu, Z.; Crews, C. M., Targeted Degradation of Transcription Factors by TRAFTACs. 2020.
15. Adamo, P.; Ladomery, M. R., The oncogene ERG: a key factor in prostate cancer. Oncogene 2016, 35 (4), 403-14.
16. Lambert, S. A., The Human Transcription Factors, Cell 2018, 172, 650.
17. The Molecular Taxonomy of Primary Prostate Cancer. Cell 2015, 163 (4), 1011-25.
18. Tomlins, S. A., Recurrent fusion of TMPRSS2 and ETS transcription factor genes in prostate cancer, Science 2005, 310, 644.
19. Perner, S.; Mosquera, J. M.; Demichelis, F.; Hofer, M. D.; Paris, P. L.; Simko, J.; Collins, C.; Bismar, T. A.; Chinnaiyan, A. M.; De Marzo, A. M.; Rubin, M. A., TMPRSS2-ERG fusion prostate cancer: an early molecular event associated with invasion. The American journal of surgical pathology 2007, 31 (6), 882-8.
20. Wang, X., Development of peptidomimetic inhibitors of the ERG gene fusion product in prostate cancer, Cancer Cell 2017, 31, 532.
21. Novak, A.; Dedhar, S., Signaling through beta-catenin and Lef/Tcf. Cellular and molecular life sciences: CMLS 1999, 56 (5-6), 523-37.
22. Kobayashi, W.; Ozawa, M., The transcription factor LEF-1 induces an epithelial-mesenchymal transition in MDCK cells independent of β-catenin. Biochemical and biophysical research communications 2013, 442 (1-2), 133-8.
23. Santiago, L.; Daniels, G.; Wang, D.; Deng, F.-M.; Lee, P., Wnt signaling pathway protein LEF1 in cancer, as a biomarker for prognosis and a target for treatment. American journal of cancer research 2017, 7 (6), 1389-1406.
24. Wei, G. H.; Badis, G.; Berger, M. F.; Kivioja, T.; Palin, K.; Enge, M.; Bonke, M.; Jolma, A.; Varjosalo, M.; Gehrke, A. R.; Yan, J.; Talukder, S.; Turunen, M.; Taipale, M.; Stunnenberg, H. G.; Ukkonen, E.; Hughes, T. R.; Bulyk, M. L.; Taipale, J., Genome-wide analysis of ETS-family DNA-binding in vitro and in vivo. EMBO J 2010, 29 (13), 2147-60.
25. Cheng, J.; Li, Y; Wang, X.; Dong, G.; Sheng, C., Discovery of Novel PDES Degraders for the Treatment of KRAS Mutant Colorectal Cancer. Journal of Medicinal Chemistry 2020, 63 (14), 7892-7905.
26. Crew, A. P.; Raina, K.; Dong, H.; Qian, Y; Wang, J.; Vigil, D.; Serebrenik, Y V.; Hamman, B. D.; Morgan, A.; Ferraro, C.; Siu, K.; Neklesa, T. K.; Winkler, J. D.; Coleman, K. G.; Crews, C. M., Identification and Characterization of Von Hippel-Lindau-Recruiting Proteolysis Targeting Chimeras (PROTACs) of TANK-Binding Kinase 1. Journal of Medicinal Chemistry 2018, 61 (2), 583-598.
27. Liang, J.; Li, X.; Li, Y; Wei, J.; Daniels, G.; Zhong, X.; Wang, J.; Sfanos, K.; Melamed, J.; Zhao, J.; Lee, P., LEF1 targeting EMT in prostate cancer invasion is mediated by miR-181a. American journal of cancer research 2015, 5 (3), 1124-32.
28. Shtutman, M.; Zhurinsky, J.; Simcha, I.; Albanese, C.; D'Amico, M.; Pestell, R.; Ben-Ze'ev, A., The cyclin D1 gene is a target of the beta-catenin/LEF-1 pathway. Proc Natl Acad Sci USA 1999, 96 (10), 5522-7.
29. Edmondson, S. D.; Yang, B.; Fallan, C., Proteolysis targeting chimeras (PROTACs) in ‘beyond rule-of-five’ chemical space: Recent progress and future challenges. Bioorganic & Medicinal Chemistry Letters 2019, 29 (13), 1555-1564.
30. Pan, X.; Rijnbeek, P.; Yan, J.; Shen, H. B., Prediction of RNA-protein sequence and structure binding preferences using deep convolutional and recurrent neural networks. BMC Genomics 2018, 19 (1), 511.
31. Pal, A.; Levy, Y, Structure, stability and specificity of the binding of ssDNA and ssRNA with proteins. PLoS Comput Biol 2019, 15 (4), e1006768.
32. Sridharan, K.; Gogtay, N. J., Therapeutic nucleic acids: current clinical status. Br J Clin Pharmacol 2016, 82 (3), 659-672.
33. Lai, A. C.; Crews, C. M., Induced protein degradation: an emerging drug discovery paradigm. Nat Rev Drug Discov 2017, 16 (2), 101-114.
34. Roberts, T. C.; Langer, R.; Wood, M. J. A., Advances in oligonucleotide drug delivery. Nat Rev Drug Discov 2020, 19 (10), 673-694.
35. Chung, J. Y; Thone, M. N.; Kwon, Y J., COVID-19 vaccines: The status and perspectives in delivery points of view. Adv Drug Deliv Rev 2020, 170, 1-25.

### Example 16: Discovery of 3-Aminophthalic Acid as a New Ligand of Cereblonfor Targeted Protein Degradation by O′PROTAC

### Abstract

Conventional proteolysis targeting chimera (PROTACs) and oligonucleotide-based PROTAC (O′PROTAC) tactics have been developed for the degradation of protein of interest (POI). In this current study, we reported the discovery of 3-aminophthalic acid as a new ligand of cereblon (CRBN) E3 ubiquitin ligase and the development of a phthalic acid-based O′PROTAC for targeted degradation of ERG transcription factor. Phthalic acid-O′PROTAC induced ERG protein degradation in a CRBN-dependent manner. We further showed that ERG phthalic acid-O′PROTAC not only suppressed the transcriptional activity of ERG, but also inhibited prostate cancer cell growth and invasion. Our findings suggest a new venue for development of PROTACs, especially O′PROTAC.

### Introduction

Proteolysis targeting chimeras (PROTACs) are heterobifunctional molecules composed of two active domains: a protein of interest (POI) ligand as a warhead and an E3 ligase ligand and a linker, which induce the proximity of POI and E3 ligase with consequent ubiquitination and degradation of POI. PROTAC utilizes event-driven pharmacology as the mode of action (MOA), thus it has potential advantages over traditional inhibitor, which is occupancy-driven MOA, with respect to reducing off-target effect, drug resistance and modulating ‘undruggable’ targets,¹ representing a promising approach to treat human disease.

An element of designing a potent PROTAC molecule is the E3 ligase ligand. The first PROTAC molecule was reported by Deshaies, and it utilized a peptide ligand for E3 ligase (3-TRCP². Peptide moieties caused poor cell permeability and biological instability, which hampered the development of PROTACs³. In the past decade, several small-molecule ligands have been identified to recruit E3 ligase, including von Hippel-Lindau (VHL)⁴, Mdm2⁵, CRBN⁶, IAPs⁷, DCAF15⁸, RNF4⁹, RNF114¹⁰, and DCAF16¹¹. However, only the CRBN and VHL ligands are frequently used E3 ligands for PROTAC design³.

CRBN is a subunit of the E3 ubiquitin ligase CUL4-RBX1-DDB1—CRBN, which ubiquitinates a number of target proteins. Thalidomide derivatives, referred to as immunomodulatory drugs (IMiDs), were demonstrated to bind to CRBN and mediate its function in the treatment of multiple myeloma and other B cell malignancies¹²⁻¹³. Thalidomide was originally marketed in 1957 for the treatment of insomnia and morning sickness. However, it was finally withdrawn from the market due to the strong teratogenicity¹⁴. Hiroshi's group demonstrated that the mechanism leading to teratogenic effects is that thalidomide binds to CRBN and inhibits its ubiquitin ligase activity¹⁵. Later, thalidomide analogs, pomalidomide and lenalidomide, were reported to induce the degradation of IKZF1 and IKZF3 through the involvement of CRBN¹²⁻¹³. The crystal structure of thalidomide with CRNB and IKZF was resolved in 2014.

In 2015, PROTAC molecules composed of CRBN ligand were designed to degrade BET and FKBP12⁶. Subsequently, the field of CRBN-recruiting PROTAC has expanded dramatically, with several PROTACs applying in clinic trials¹⁶.

Despite continuous progress in the development of potent CRBN-recruiting PROTACs, considerable challenges remain. IMiDs-based PROTACs have been described to remain the activity of IMiDs on Ikaros transcription factor, leading to the off-target effect¹⁷. Furthermore, thalidomide showed poor stability under physiological pH 7.4 due to the hydrolysis of phthalimide and glutarimide moiety¹⁸⁻¹⁹.

In this current study, we identified phthalic acid as a ligand of CRBN ligase. Phthalic acid-based ERG O′PROTAC (ERG OP-C-P1) showed a comparable or better efficacy in degrading ERG protein than pomalidomide O′PROTACs. ERG OP-C-P1 significantly reduced the transcriptional activity of ERG, suppressed its target gene expressions, and inhibited growth and invasion of ERG-positive prostate cancer cells.

### Results

### Development of Phthalic Acid-Based O′PROTACs as Degraders of ERG Protein

We initially used phosphoramidite chemistry to construct the pomalidomide- and VH032-based O′PROTACs (ERG OP-C1 to C3 and OP-V1 to V3) with different linker lengths to target ERG. Different from the mass spectrometry results of VH032-based ERG O′PROTACs, the mass spectrum of three pomalidomide-based ERG O′PROTACs showed that phthalic acid rather than phthalimide is the major product from DNA synthesizer (FIGS. 21A and 21). These results suggest that pomalidomide is susceptible to deprotection conditions during regular DNA synthesis (Scheme 2A).

When 293T cells were transfected with ERG expression plasmid and treated with one of the three crude 3-N-substituted-aminophthalic acid-based O′PROTACs (OP-C1 to C3), we found that two of them (C1 and C2) exhibited potent activity in ERG degradation (FIG. 11A). In contrast, VH032-based ERG O′PROTACs were inactive. These two ERG O′PROTACs (C1 and C2) also effectively decreased ERG protein in prostate cancer VCaP cells that expressed a high level of endogenous ERG protein due to the TRMPRSS2-ERG gene fusion (FIG. 11i).

To test the hypothesis that phthalic acid was a E3 ligase recruiter of O′PROTACs that are effective in proteolytic degradation of a target protein, we synthesized an ERG O′PROTAC (OP-C-P1) by applying a synthetic route using phthalic acid dimethyl ester as the start material (Scheme 2B). The HPLC and mass spectrometry data indicated that ERG OP-C-P1 (containing a DNA oligo composed by phthalic acid-linked reverse strand and FITC-labeled forward strand) was successfully synthesized by phosphoramidite chemistry with high purity and expected molecular mass (FIGS. 11C, 11D, 21C, and 21D). We, therefore, employed this ERG OP-C-P1 (FIG. 11E) for further biochemical and functional studies.

### Phthalic Acid-Based ERG O′PROTAC Induces ERG Proteasome Degradation

We firstly compared the efficacy of the phthalic acid-based ERG OPs (C-P1 with high purity and C1 with low purity) with two pomalidomide-based ERG O′PROTACs synthesized via click reaction. FITC-labeled ERG O′PRORACs were used to assess the transfection efficiency of these O′PROTACs. Fluorescent microscopy analysis showed that phthalic acid-based ERG O′PROTACs were transfected as effectively as ERG O′PROTACs C-A1 and C-N1 in both 293T and VCaP cell lines (FIG. 12A, B). Western blot analysis revealed that OP-C-P1 exhibited a slightly stronger inhibitory effect on downregulation of ectopically expressed full-length (FL) ERG protein than OP-C-A1 and OP-C-N1 in 293T cells (FIG. 12C), and similar results were obtained of the endogenous FL ERG in VCaP cells (FIG. 12D).

Further analysis revealed that these ERG OPs did not exerted an effect on mRNA levels of both FL and truncated ERG T1/E4 derived from TMPRSS2-ERG gene fusion (FIGS. 12D and 12E), suggesting that ERG OP-C-P1 inhibit ERG expression at the post-transcriptional level.

We then analyzed the kinetics of OP-C-P1 potency on protein degradation. Time-course studies demonstrated that OP-C-P1 inhibited ERG protein expression starting from 24-hours post-transfection (FIG. 12F). Dose-course experiments further revealed that OP-C-P1 induced dramatic decrease in ERG protein level at a concentration as low as 50 nM (FIG. 12G). Little or no further increase in reduction of ERG protein level even much higher concentrations (100 or 500 nM) were used, implying that the amount of ERG OP-C-P1 in cells could be saturated or its up-take by cells could be limited due to transfection efficiency. The degradation concentration (DC) curve demonstrated that OP-C-P1 inhibited 50% of ERG protein at 172.4 nM (FIG. 12H).

### Phthalic Acid-Based ERG OP Degrades ERG Via Proteasome Pathway

To determine whether phthalic acid-based ERG OP-C-P1-induced ERG protein downregulation is mediated through the ubiquitination and proteasome degradation pathway, VCaP cells were first transfected with OP-C-P1 and treated with the proteasome inhibitor MG132. MG132 treatment completely blocked the degradation of ERG protein (FIG. 13A), suggesting that ERG degradation is dependent on the proteasome pathway. Meanwhile, the ubiquitination assay showed that the treatment of OP-C-P1 enhanced the ubiquitination level of both exogenous and endogenous ERG in 293T and VCaP cells, respectively (FIGS. 13B and 1C).

To examine whether ERG OP-C-P1 can bind to ERG in vitro, we performed electrophoretic mobility shift assay (EMSA) using nuclear extract of VCaP cells. We demonstrated that biotin-labeled ERG OP-C-P1 formed a DNA-protein complex (DPC) in the nuclear extract of VCaP cells. This binding was interrupted by the addition of competitive non-biotin-labeled ERG OP-C-P1 (FIG. 13D). Moreover, the addition of ERG antibody resulted in a super-shift of DPC (FIG. 13E), suggesting that the detected DPC contains ERG protein.

### Phthalic Acid-Based ERG OP-Induced Degradation of ERG is Mediated by CRBN

Next, we investigated whether OP-C-P1-mediated degradation of ERG is dependent on cereblon (CRBN). We knocked down CRBN in VCaP cells and treated the cells with OP-C-P1. We found that CRBN knockdown completely abolished OP-C-P1-induced degradation of ERG (FIG. 13F). The treatment of cereblon ligand pomalidomide also overcame the degradation of ERG protein induced by OP-C-P1 and this effect was dose dependent (FIG. 13G). These data indicate that OP-C-P1-induced degradation of ERG is mediated through CRBN E3 ligase.

To understand the interaction between CRBN protein and 3-aminophthalic acid, we performed the docking using 3-N-substituted phthalic acid and CRBN (PDB: 4CI1). The interaction of phthalic acid was observed to be similar with thalidomide (FIG. 22). For example, the 1′-carboxylic acid group oriented toward the hydrophobic pocket and resulted in formation of two strong hydrogen bonds. The carbonyl oxygen and hydrogen of hydroxy groups interacted with the backbone of TRP382 and HIS380, respectively. These hydrogen bond interactions were resemblant with the glutarimide group of thalidomide, where interaction occurred between two carbonyl and amide to residues HIS380 and TRP382, respectively. Additionally, the other 2′-carboxylic acid group would be more solvent exposed. Due to the flexibility of C—C bond between benzene and carboxylic acid, the carbonyl oxygen could position itself facing to the hydrophobic pocket to retain hydrogen bond with imidazole side chain of HIS380; meanwhile, the hydroxy group formed weak water-mediated hydrogen bond with HIS359 side chain. Comparatively to thalidomide, the phthalimide was completely solvent exposed and accommodated with a water-mediated hydrogen bond with HIS359. There were also observed pi-pi interactions between indole of TRP388 and benzene ring of phthalic acid. The orientation of 3-amino group was completely solvent exposed similar to pomalidomide and lenalidomide, which contributed enormously for forming linkers with any potential warheads. This binding information provide an explanation for the observation that phthalic acid-based O′PROTAC showed comparable activity as pomalidomide-based O′PROTACs.

### Phthalic Acid-Based ERG OP Impairs ERG Target Gene Expression and Cell Growth and Invasion

To determine whether ERG OP-C-P1 affects ERG signaling pathway, we detected the transcriptional levels of ERG target genes. We demonstrated that the downregulation of ERG by OP-C-P1 also significantly diminished mRNA expression of ERG target genes including ADAM19, MMP3, MAIP9, PLAT and PLAU (FIGS. 14A and 14B). To examine the functional effects of OP-C-P1 on cell growth, we performed three-dimensional (3D) sphere formation assay using VCaP cells. We showed that OP-C-P1 treatment largely decreased the diameters of the spheres of VCaP ccells, indicating that OP-C-P1 inhibited VCaP cell growth (FIGS. 14C and 1D). Considering the roles of ERG on cell invasion²¹, cell invasion assay was performed to detect whether this ERG OP can affect cell invasion. We found that the treatment of OP-C-P1 decreased the invasion ability of VCaP cells (FIGS. 14E and 1F). Collectively, OP-C—P1-induced degradation of ERG effectively undermines the transcriptional activity of ERG and prostate cancer cell growth and invasion.

In summary, we identified phthalic acid as a ligand of CRBN ligase. Phthalic acid-based ERG O′PROTAC significantly inhibited the protein level of ERG via ubiquitination-proteasome pathway and impaired ERG functions in cell growth and invasion. This ERG O′PROTAC provides clear evidence that phthalic acid functions actively as well as pomalidomide in O′PROTAC. Our data suggest that this CRBN ligand can be employed to design O′PROTACs or canonical PROTACs to degrade other transcription factors or POIs.

### Experimental Section

### Synthesis of Dimethyl 3-((5-(((2-cyanoethoxy)(diisopropylamino) phosphaneyl)oxy)pentyl) amino)phthalate

Synthesis of Dimethyl 3-((5-(((2-cyanoethoxy)(diisopropylamino) phosphaneyl)oxy)pentyl) amino)phthalate was performed as described in Example 6.

### Synthesis of Oligonucleotides and Annealing Reaction

All oligonucleotides used in this study were synthesized by ExonanoRNA (Columbus, OH). For oligo annealing reaction, single-stranded forward and reverse oligonucleotides were mixed in an assembly buffer (10 mM Tris-HCl [pH7.5], 100 mM NaCl, 1 mM EDTA), and heated to 90° C. for 5 min, then slowly cooled down to 37° C. within 1 h. Double-stranded O′PROTACs were mixed well, aliquoted and stored at −20° C. for the future use.

### Plasmids and Reagents

The siRNA constructs (siNS and siCRBN) were purchased from GE Dharmacon. The mammalian expression vector for HA-Ub was purchased from Addgene while pMCV-HA-ERG was constructed using cDNA of VCaP cells as a template. Cycloheximide (CHX), MG132 were purchased from Sigma Aldrich. The antibodies used were: HA (Cat #MMS-101R) from Covance; Flag (M2) (Cat #F-3165) from Sigma; ERK2 (sc-1647) from Santa Cruz; CRBN (Cat #71810S) from Cell Signaling Technology; ERG from Biocare Medical (Cat #901-421-101520). For western blots, all the antibodies were diluted 1:1,000 with 5% BSA in TBST.

### Cell Lines, Cell Culture and Transfection

The immortalized human embryonic kidney cell line 293T and two PCa cell lines (VCaP and 22Rv1) were purchased from ATCC (Manassas, VA). The 293T and VCaP cells were cultured in Dulbecco's modified Eagle's medium (DMEM) supplemented with 10% of FBS (Thermo Fisher Scientific). The 22Rv1 cells were cultured in RPMI 1640 medium supplemented with 10% of FBS. The cells were maintained in a 37° C. humidified incubator supplied with 5% CO₂.

Transient transfection was performed by Lipofectamine 2000 (Cat #11668500, Thermo Fisher Scientific) according to the manufacturer's instruction. The siRNA sequences and information are listed in Table 10.

### Protein Extraction and Western Blot

The cells were washed with PBS once before being lysed into lysis buffer containing 25 mM Tris-HCl pH 7.4, 150 mM NaCl, 1 mM EDTA, 1% NP-40, and 5% glycerol for 30 minutes on ice. The lysate was centrifuged at 13,000 rpm for 15 minutes, and the supernatant containing 50 μg of total protein was applied to SDS-PAGE gel. The protein gel was transferred to the nitrocellulose membrane, which was blocked by 5% slim milk for 1 hour, followed by incubation with primary antibody at 4° C. overnight and secondary antibody at RT for 1 hour. The protein signal was developed with Pierce™ ECL Western Blotting Substrate (Cat #32106, Thermo Fisher Scientific).

### RNA Extraction and RT-qPCR

Total RNA was extracted and reversely transcribed into cDNA as previously described²², followed by quantitative PCR using iQ SYBR Green Supermix (Cat #1708880, Bio-Rad). The ΔCT was calculated by normalizing the threshold difference of a certain gene with glyceraldehyde-3-phosphate dehydrogenase (GAPDH). Primers used for RT-qPCR are listed in Table 11.

### Nuclear Extraction and Electrophoretic Mobility Shift Assay (EMSA)

The VCaP cell nuclear protein was extracted using NE-PER™ Nuclear and Cytoplasmic Extraction Reagents (Cat #78833, Thermo Fisher Scientific). EMSA was performed with LightShift™ Chemiluminescent EMSA Kit (Cat #20148, Thermo Fisher Scientific) according to the manufacturer's instruction. Briefly, ERG OP-C-P1 containing the potential ERG binding motif was incubated with VCaP nuclear protein for 30 minutes at RT, followed by separation with 6% acrylamide DNA gel. The biotin-labeled probe was incubated with 0.5 or 1 μg of ERG antibody for 1 hour before loading into 6% of Polyacrylamide DNA gel.

### Three-Dimension (3D) Sphere

˜120 μL of matrigel matrix (Cat #354234, BD Bioscience) was pre-coated onto the bottom of the wells of 24-well plate at 37° C. for 30 minutes. Approximately 20,000 VCaP cells transfected with ERG OP-C-P1 (200 nM) were resuspended in 250 μL of DMEM/F12 medium containing 10% FBS and seeded on the top of matrigel pre-coated wells. After 30 minutes, when the cells were settled down, they were covered with another layer of 10% matrigel diluted with DMEM/F12 medium. The medium was changed every 2-3 days.

### Cell Invasion

The 22Rv1 cells were transfected with 100 nM of OP-C-P1 and 0.5 μg of pCMV-HA-ERG. Approximately 50,000 transfected 22Rv1 cells were re-suspended with 200 μL of serum-free RPMI-1640 medium and seeded onto matrigel invasion chamber (Cat #354480, Corning). The chambers were then placed into the wells filled with 800 μL of RPMI-1640 medium supplemented with 10% FBS.

The O′PROTAC conjugate containing the phthalic acid E3 binding ligand (ERG O′PROTAC (OP-C-P1)) used in the protein degradation experiments (biochemical and functional studies) was obtained at the time of coupling of the targeting moiety to the intermediate P2 at the phosphate deprotection step. See Schemes 2A and 2B.

### Schemes 2A and 2B

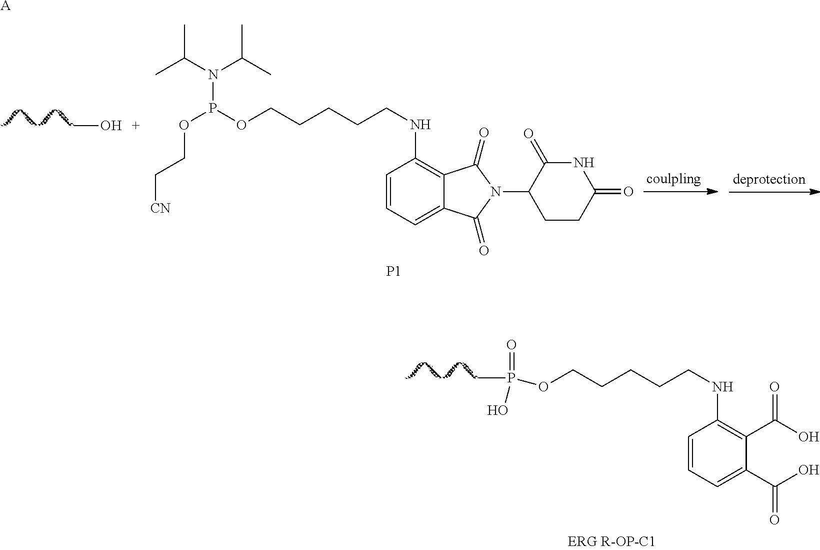

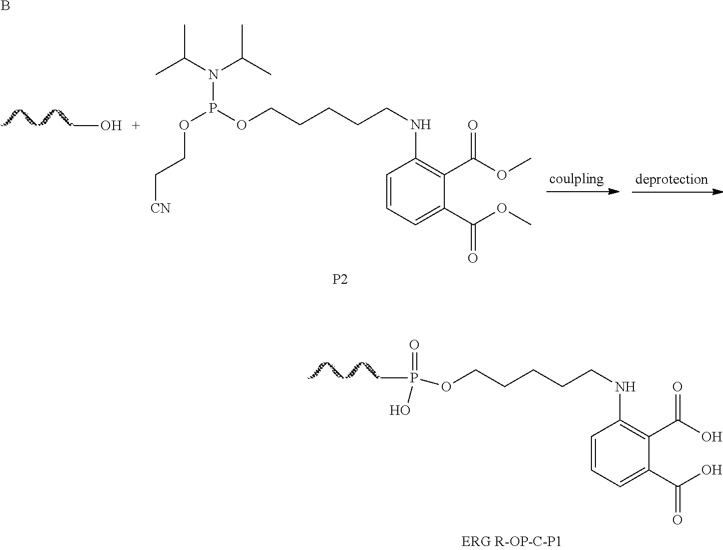

TABLE 10
SiRNA oligonucleotide sequences

| Name | Species | Catalog No. | Sequence (5′-3′) | SEQ ID NO |
|---|---|---|---|---|
| non-targeting | Human | M-021086-01-0005 | UAGCGACUAAACACAUCAAUU | 437 |
| siRNA (siNS) | | | | |
| siCRBN Pool | Human | M-021086-01-0005 | | |
| siCRBN-1 | Human | D-021086-06 | GAAUAAAUGUACUUCCCUU | 438 |
| siCRBN-2 | Human | D-021086-07 | GUGCUGAUAUGGAAGAAUU | 439 |
| siCRBN-3 | Human | D-021086-08 | CCAGCAAGCUAAAGUGCAA | 440 |
| siCRBN-4 | Human | D-021086-09 | AGACAAAGGUUCAAAGUCC | 441 |

TABLE 12
Sequences of control and ERG-bound DNA oligos in O′PROTACs

| Name^{a)} | DNA sequence | SEQ ID NO | Mass calc. | Mass obs. |
|---|---|---|---|---|
| ERG-F | 5′-ACGGACCGGAAATCCGGTT-3′ | 3 | 5837.8 | 5838.0 |
| ERG-F-FITC | 5′-FITC-ACGGACCGGAAATCCGGTT-3′ | 3 | 6404.3 | 6405.8 |
| ERG-F-Biotin | 5′-Biotin-ACGGACCGGAAATCCGGTT-3′ | 3 | 6273.3 | 6274.0 |
| ERG-R-C1^{b}) | 3′-TGCCTGGCCTTTAGGCCAA-L1-phthalic acid-5′ | 419 | 6108.9 | 6109.6 |
| ERG-R-C2^{b)} | 3′-TGCCTGGCCTTTAGGCCAA-L2-phthalic acid-5′ | 419 | 6154.9 | ND^{c)} |
| ERG-R-C3^{b}) | 3′-TGCCTGGCCTTTAGGCCAA-L3-phthalic acid-5′ | 419 | 6198.9 | ND^{c)} |
| ERG-R-C-P1 | 3′-TGCCTGGCCTTTAGGCCAA-L1-phthalic acid-5′ | 419 | 6108.9 | 6108.6 |
| ERG-R-V1 | 3′-TGCCTGGCCTTTAGGCCAA-L4-VHL-5′ | 419 | 6386.0 | 6386.2 |
| ERG-R-V2 | 3′-TGCCTGGCCTTTAGGCCAA-L5-VHL-5′ | 419 | 6418.0 | 6418.4 |
| ERG-R-V3 | 3′-TGCCTGGCCTTTAGGCCAA-L6-VHL-5′ | 419 | 6462.0 | 6462.5 |
| CTRL-F | 5′-TGTGCTAGCTGATGTGCTA-3′ | 420 | 5849.9 | 5850.3 |
| CTRL-R-C-N1 | 3′-ACACGATCGACTACACGAT-L7-pomalidomide-5′ | 421 | 6286.0 | 6286.6 |

^{a)}F, forward; R, reverse; ^{b)}not purified. ^{c)}ND, not determined.

### REFERENCES

1. Dale, B.; Cheng, M.; Park, K. S.; Kaniskan, H. U.; Xiong, Y; Jin, J., Advancing targeted protein degradation for cancer therapy. Nat Rev Cancer 2021.
2. Sakamoto, K. M.; Kim, K. B.; Kumagai, A.; Mercurio, F.; Crews, C. M.; Deshaies, R. J., Protacs: Chimeric molecules that target proteins to the Skp1—Cullin-F box complex for ubiquitination and degradation. Proceedings of the National Academy of Sciences 2001, 98 (15), 8554.
3. Ishida, T.; Ciulli, A., E3 Ligase Ligands for PROTACs: How They Were Found and How to Discover New Ones. SLAS discovery: advancing life sciences R & D 2021, 26 (4), 484-502.
4. Zengerle, M.; Chan, K.-H.; Ciulli, A., Selective Small Molecule Induced Degradation of the BET Bromodomain Protein BRD4. ACS Chemical Biology 2015, 10 (8), 1770-1777.
5. Schneekloth, A. R.; Pucheault, M.; Tae, H. S.; Crews, C. M., Targeted intracellular protein degradation induced by a small molecule: En route to chemical proteomics. Bioorganic & Medicinal Chemistry Letters 2008, 18 (22), 5904-5908.
6. Winter, G. E.; Buckley, D. L.; Paulk, J.; Roberts, J. M.; Souza, A.; Dhe-Paganon, S.; Bradner, J. E., DRUG DEVELOPMENT. Phthalimide conjugation as a strategy for in vivo target protein degradation. Science 2015, 348 (6241), 1376-1381.
7. Itoh, Y; Ishikawa, M.; Naito, M.; Hashimoto, Y, Protein Knockdown Using Methyl Bestatin-Ligand Hybrid Molecules: Design and Synthesis of Inducers of Ubiquitination-Mediated Degradation of Cellular Retinoic Acid-Binding Proteins. Journal of the American Chemical Society 2010, 132 (16), 5820-5826.
8. Li, L.; Mi, D.; Pei, H.; Duan, Q.; Wang, X.; Zhou, W.; Jin, J.; Li, D.; Liu, M.; Chen, Y, In vivo target protein degradation induced by PROTACs based on E3 ligase DCAF15. Signal Transduct Target Ther 2020, 5 (1), 129.
9. Ward, C. C.; Kleinman, J. I.; Brittain, S. M.; Lee, P. S.; Chung, C. Y S.; Kim, K.; Petri, Y; Thomas, J. R.; Tallarico, J. A.; McKenna, J. M.; Schirle, M.; Nomura, D. K., Covalent Ligand Screening Uncovers a RNF4 E3 Ligase Recruiter for Targeted Protein Degradation Applications. ACS Chem Biol 2019, 14 (11), 2430-2440.
10. Luo, M.; Spradlin, J. N.; Boike, L.; Tong, B.; Brittain, S. M.; McKenna, J. M.; Tallarico, J. A.; Schirle, M.; Maimone, T. J.; Nomura, D. K., Chemoproteomics-enabled discovery of covalent RNF114-based degraders that mimic natural product function. Cell Chem Biol 2021, 28 (4), 559-566 e15.
11. Zhang, X.; Crowley, V. M.; Wucherpfennig, T. G.; Dix, M. M.; Cravatt, B. F., Electrophilic PROTACs that degrade nuclear proteins by engaging DCAF16. Nat Chem Biol 2019, 15 (7), 737-746.
12. Kronke, J.; Udeshi, N. D.; Narla, A.; Grauman, P.; Hurst, S. N.; McConkey, M.; Svinkina, T.; Heckl, D.; Comer, E.; Li, X.; Ciarlo, C.; Hartman, E.; Munshi, N.; Schenone, M.; Schreiber, S. L.; Carr, S. A.; Ebert, B. L., Lenalidomide causes selective degradation of IKZF1 and IKZF3 in multiple myeloma cells. Science 2014, 343 (6168), 301-5.
13. Lu, G.; Middleton, R. E.; Sun, H.; Naniong, M.; Ott, C. J.; Mitsiades, C. S.; Wong, K. K.; Bradner, J. E.; Kaelin, W. G., Jr., The myeloma drug lenalidomide promotes the cereblon-dependent destruction of Ikaros proteins. Science 2014, 343 (6168), 305-9.
14. Ito, T.; Handa, H., Molecular mechanisms of thalidomide and its derivatives. Proc Jpn Acad Ser B Phys Biol Sci 2020, 96 (6), 189-203.
15. Ito, T.; Ando, H.; Handa, H., Teratogenic effects of thalidomide: molecular mechanisms. Cellular and molecular life sciences: CMLS 2011, 68 (9), 1569-79.
16. Mullard, A., Targeted protein degraders crowd into the clinic. Nat Rev Drug Discov 2021, 20 (4), 247-250.
17. Schapira, M.; Calabrese, M. F.; Bullock, A. N.; Crews, C. M., Targeted protein degradation: expanding the toolbox. Nat Rev Drug Discov 2019, 18 (12), 949-963.
18. Goracci, L.; Desantis, J.; Valeri, A.; Castellani, B.; Eleuteri, M.; Cruciani, G., Understanding the Metabolism of Proteolysis Targeting Chimeras (PROTACs): The Next Step toward Pharmaceutical Applications. J Med Chem 2020, 63 (20), 11615-11638.
19. Steinebach, C.; Ng, Y L. D.; Sosic, I.; Lee, C.—S.; Chen, S.; Lindner, S.; Vu, L. P.; Bricelj, A.; Haschemi, R.; Monschke, M.; Steinwarz, E.; Wagner, K. G.; Bendas, G.; Luo, J.; Gutschow, M.; Kronke, J., Systematic exploration of different E3 ubiquitin ligases: an approach towards potent and selective CDK6 degraders. Chemical Science 2020, 11 (13), 3474-3486.
20. Shao, J.; Yan, Y; Ding, D.; Wang, D.; He, Y; Pan, Y; Yan, W.; Kharbanda, A.; Li, H.-y.; Huang, H., Destruction of DNA-Binding Proteins by Programmable Oligonucleotide PROTAC (O′PROTAC): Effective Targeting of LEF1 and ERG. Advanced Science 2021, n a (n/a), 2102555.
21. Yang, Y; Blee, A. M.; Wang, D.; An, J.; Pan, Y; Yan, Y; Ma, T.; He, Y; Dugdale, J.; Hou, X.; Zhang, J.; Weroha, S. J.; Zhu, W. G.; Wang, Y A.; DePinho, R. A.; Xu, W.; Huang, H., Loss of FOXO1 Cooperates with TMPRSS2-ERG Overexpression to Promote Prostate Tumorigenesis and Cell Invasion. Cancer Res 2017, 77 (23), 6524-6537.
22. Yan, Y; An, J.; Yang, Y; Wu, D.; Bai, Y; Cao, W; Ma, L.; Chen, J.; Yu, Z.; He, Y; Jin, X.; Pan, Y; Ma, T.; Wang, S.; Hou, X.; Weroha, S. J.; Karnes, R. J.; Zhang, J.; Westendorf, J. J.; Wang, L.; Chen, Y; Xu, W; Zhu, R.; Wang, D.; Huang, H., Dual inhibition of AKT-mTOR and AR signaling by targeting HDAC3 in PTEN- or SPOP-mutated prostate cancer. EMBO Mol Med 2018, 10 (4).
23. Shao, J.; Yan, Y; Ding, D.; Wang, D.; He, Y; Pan, Y; Yan, W.; Kharbanda, A.; Li, H. Y; Huang, H., Destruction of DNA-Binding Proteins by Programmable Oligonucleotide PROTAC (O′PROTAC): Effective Targeting of LEF1 and ERG. Adv Sci (Weinh) 2021, e2102555.

### Example 17: Transcriptional Activity of GOF p53 Mutants Co-Opts TMPRSS2-ERG to Promote Pyrimidine Synthesis and Cancer Fitness

This Example describes a GOF role of p53 mutants in direct binding of a unique sequence in the CTNNB1 gene promoter and upregulation of β—Catenin gene expression. This Example also identifies β—Catenin and pyrimidine synthesis as therapeutic targets of ERG/GOF p53-positive PCa.

### Results

### TMPRSS2-ERG Fusion and TP53 Alteration are Co-Occurred in Human PCa

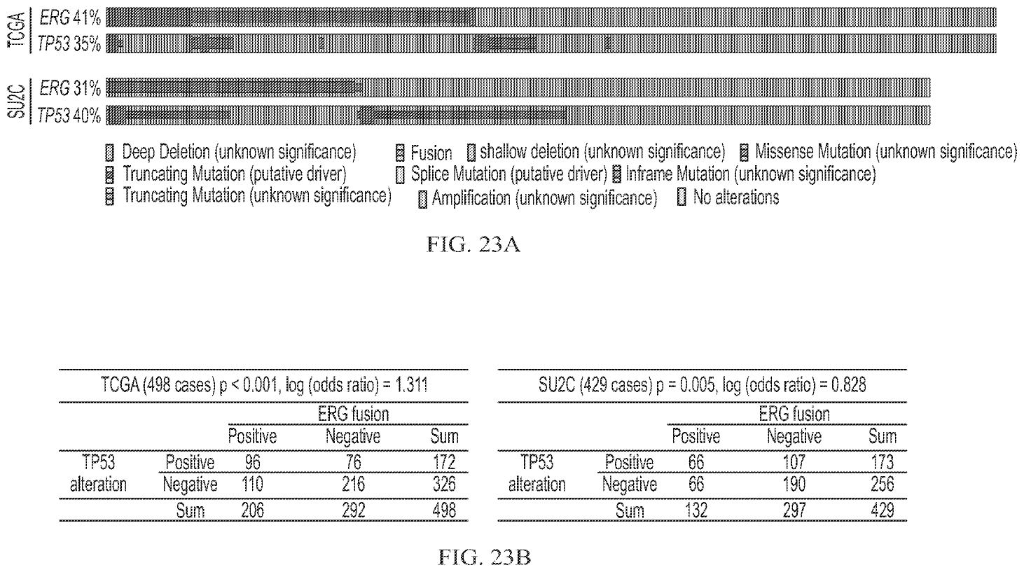

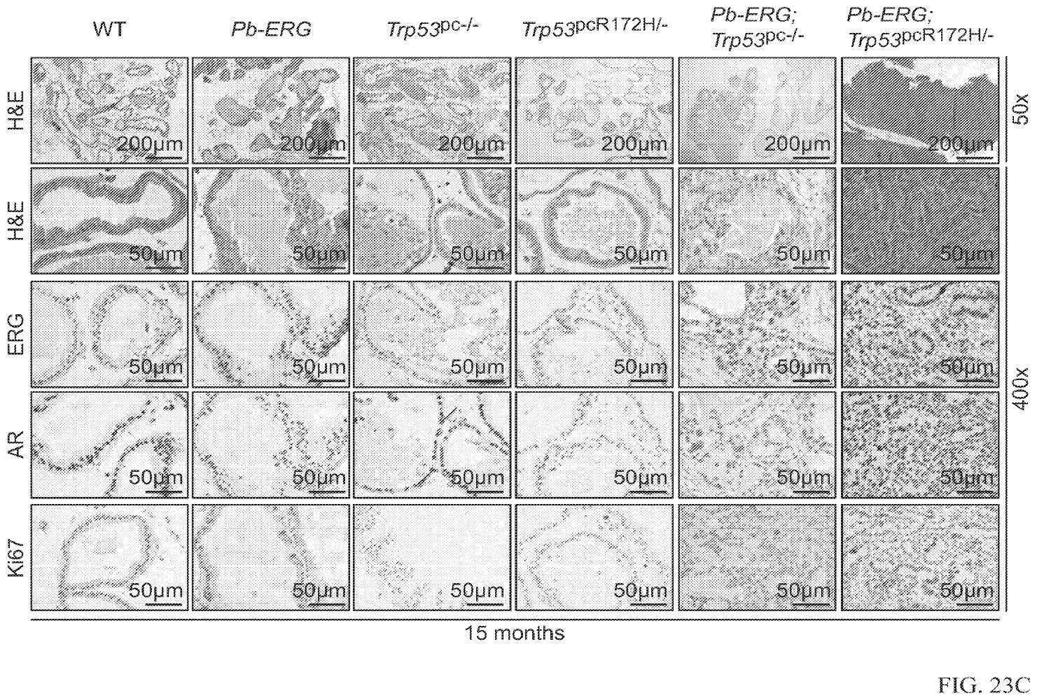

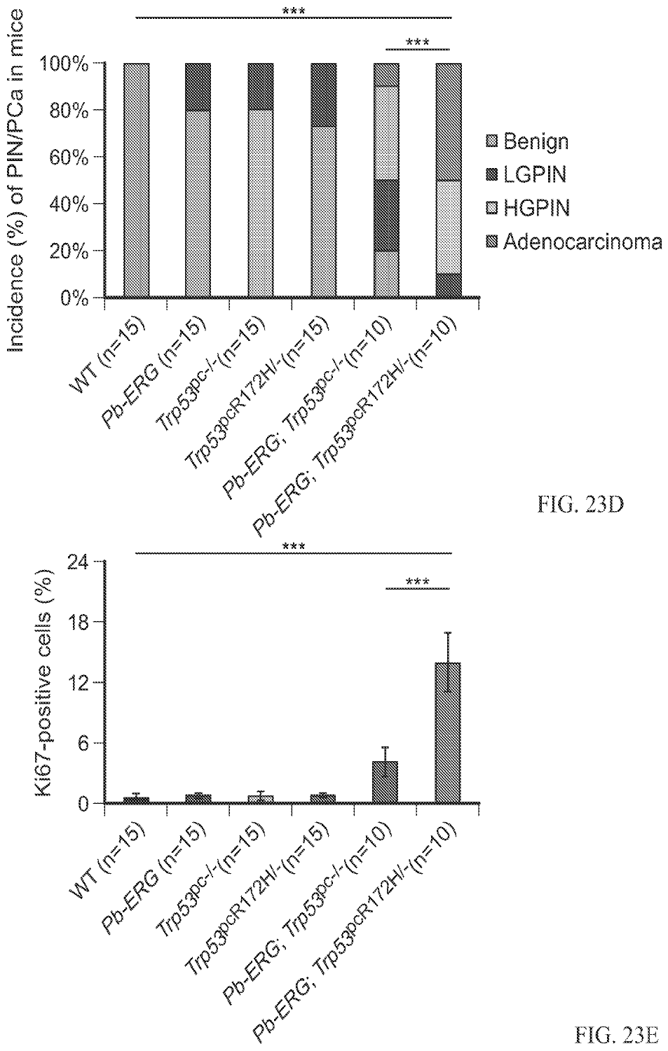

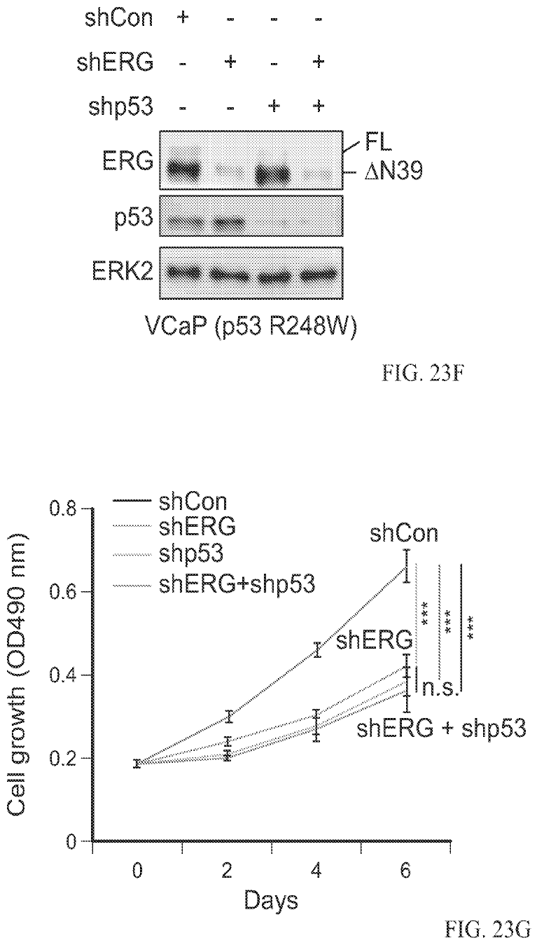

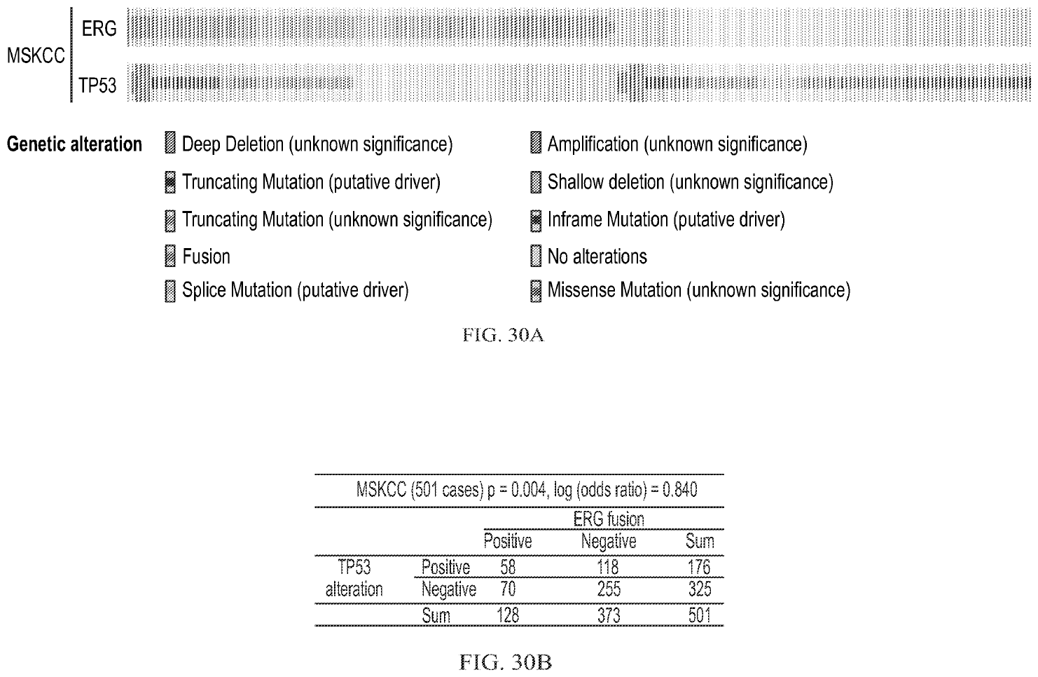

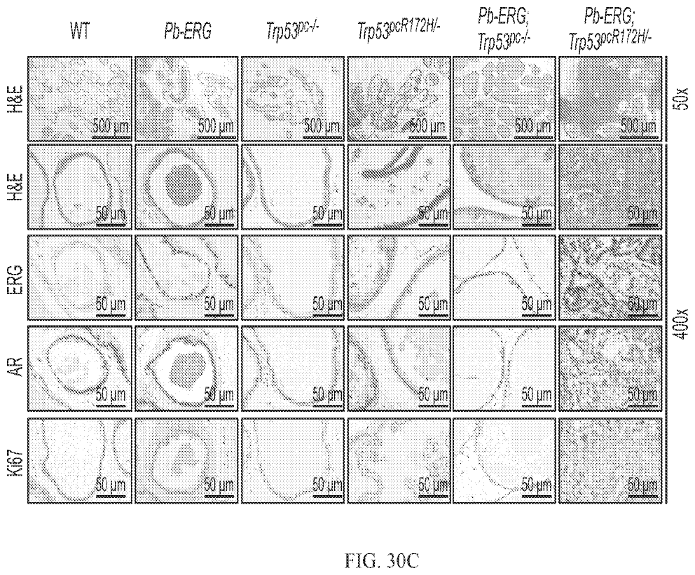

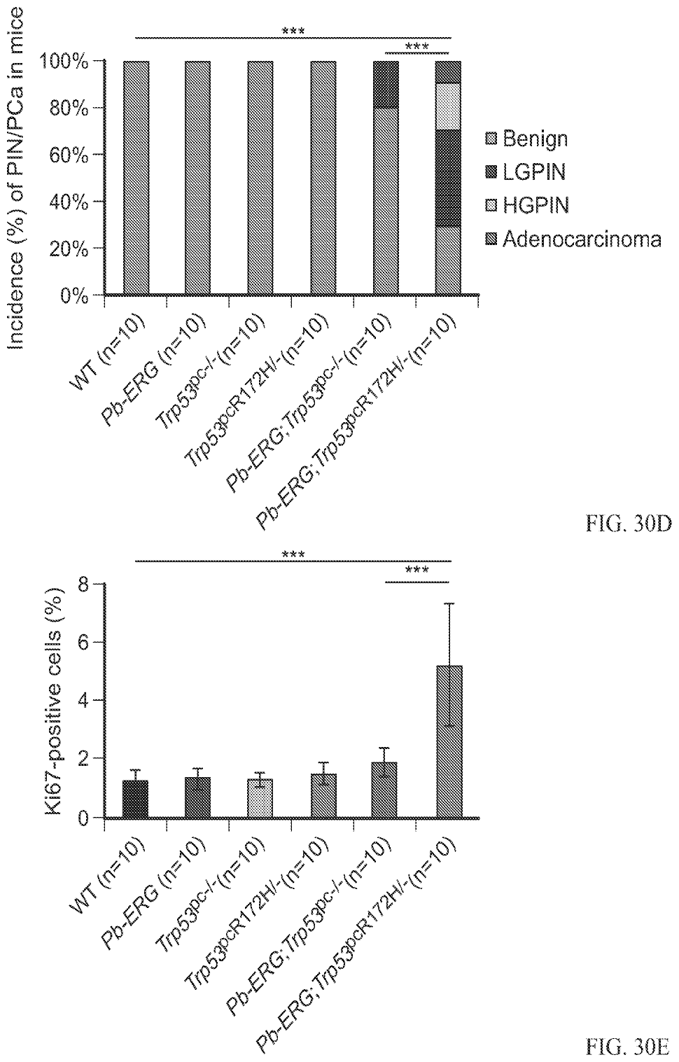

Whether TMPRSS2-ERG fusion and TP53 gene alteration (including both deletion and mutation) co-occur in patient specimens was examined. It was found that these two lesions significantly overlapped in approximately 1,500 cases of patient samples analyzed, which include primary PCa from the TCGA cohort, primary and advanced PCa in the MSKCC cohort and advanced PCa from the SU2C cohort (FIGS. 23A, 23B, 30A, and 30B). These results stress the importance of TMPRSS2-ERG fusion and TP53 alteration co-occurrence in PCa pathogenesis and progression in patients.

### A GOF Role of p53 Mutant in Early Onset of PCa in Mice

To determine whether co-occurrence of TMPRSS2-ERG fusion and TP53 alteration plays a causal role in prostate tumorigenesis, six genotypic GEM groups either with or without TMPRSS2-ERG overexpression, Trp53 gene knockout (KO) and/or GOF mutant knockin (KI) were generated (FIG. 23C): 1) “wild-type” (Cre-negative “WT” littermates); 2) ERG transgenic alone, with overexpression of PCa-associated ERGAN32, a truncated ERG lacking the first 32 amino acids at the N-terminus due to TMPRSS2-ERG gene fusion, driven by the AR-dependent Probasin (Pb) promoter (Pb-ERG); 3) prostate-specific Trp53 KO (Trp53^{pc-/-}); 4) prostate-specific Trp53 KO and KI of R172H (equivalent to R175H in human p53, a hotspot GOF mutation (Müller and Vousden, 2014)) (Trp53^{pcR172H/-}); 5) prostate-specific Pb-ERG;Trp53^{pc-/-}; and 6) prostate-specific Pb-ERG;Trp53^{pcR172H/-}. These groups of mice were generated by using Pb-driven Cre recombinase transgenic mice (Pb-Cre4), Pb-ERG transgenic mice, and Trp53^{loxp-stop-loxp-R172H/loxp} mice as original breeders.

Histological analyses showed that at 10 months of age approximately 10% of ERG/GOF p53 R172H KI (Pb-ERG;Trp53^{pcR172H/-}) mice developed focal adenocarcinoma and 60% of them had low grade prostatic intraepithelial neoplasia (LGPIN) and high grade PIN (HGPIN); however, no ERG/p53 KO (Pb-ERG;Trp53^{pc-/-}) mice exhibited focal adenocarcinoma, and only 20% of these mice had LGPIN and the rest of them displayed no neoplastic phenotype (FIGS. 30C and 30D). By 15 months of age, approximately 60% of Pb-ERG;Trp53^{pcR172H/-} mice developed focal or widespread adenocarcinoma and the rest of them exhibited LGPIN and/or HGPIN. In contrast, only 10% of Pb-ERG;Trp53^{pc-/-} mice developed focal adenocarcinoma (FIGS. 23C and 23D). There was no PIN formation in Pb-ERG mice by 10 months (FIGS. 30C and 30D). However, by 15 months of age approximately 20% of Pb-ERG mice displayed focal LGPIN lesions (FIG. 23D). The age-dependent disease progression further supports the notion that ERG overexpression requires secondary and/or tertiary mutations to drive prostate oncogenesis. Immunohistochemistry (IHC) analysis showed that all the lesions in both ERG/GOF p53 R172H KI and ERG/p53 KO mice were androgen receptor (AR) positive (FIGS. 23C and 30C). In agreement with the histological results, the percentage of Ki67-positive cells was much higher in the prostate tissues from ERG/GOF p53 R172H KI mice at both 10 and 15 months of age compared to ERG/p53 KO and other genotypic mice (FIGS. 23E and 30E). Thus, relative to Trp53 loss, p53 mutant (e.g. R172H) cooperates with TMPRSS2-ERG to induce early onset of PCa in mice, highlighting an in vivo GOF role of p53 mutant in prostate oncogenesis.

The importance of GOF p53 for human PCa cell growth was examined. One allele of TP53 is deleted and the other is mutated (R248W) in TMPRSS2-ERG fusion-positive human PCa cell line VCaP. Endogenous ERG (both full-length and ERGAN39, a truncated ERG lacking the first 39 amino acids at the N-terminus due to TMPRSS2-ERG fusion) and p53 R248W mutant were knocked down individually or together using small hairpin RNAs (shRNAs). It was demonstrated that knockdown of either ERG or p53 R248W markedly inhibited cell growth (FIGS. 23F and 23G). The results from both GEM models and human VCaP cells invariably support the notion that ERG cooperates with GOF p53 mutant to promote PCa oncogenesis and progression.

### Co-Regulation of PSGs by ERG and GOF p53 Mutant

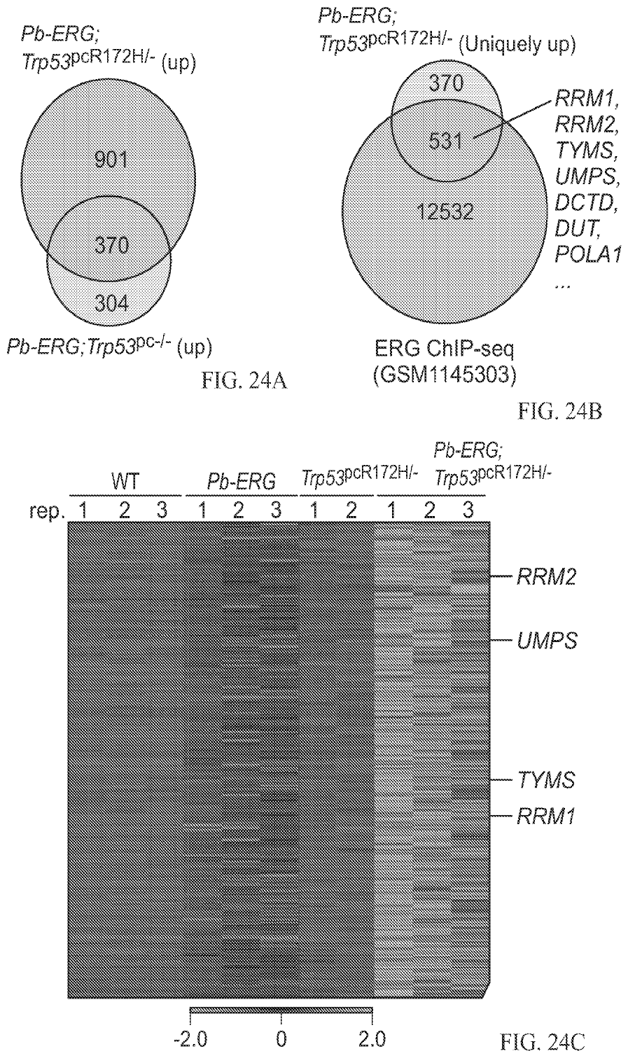

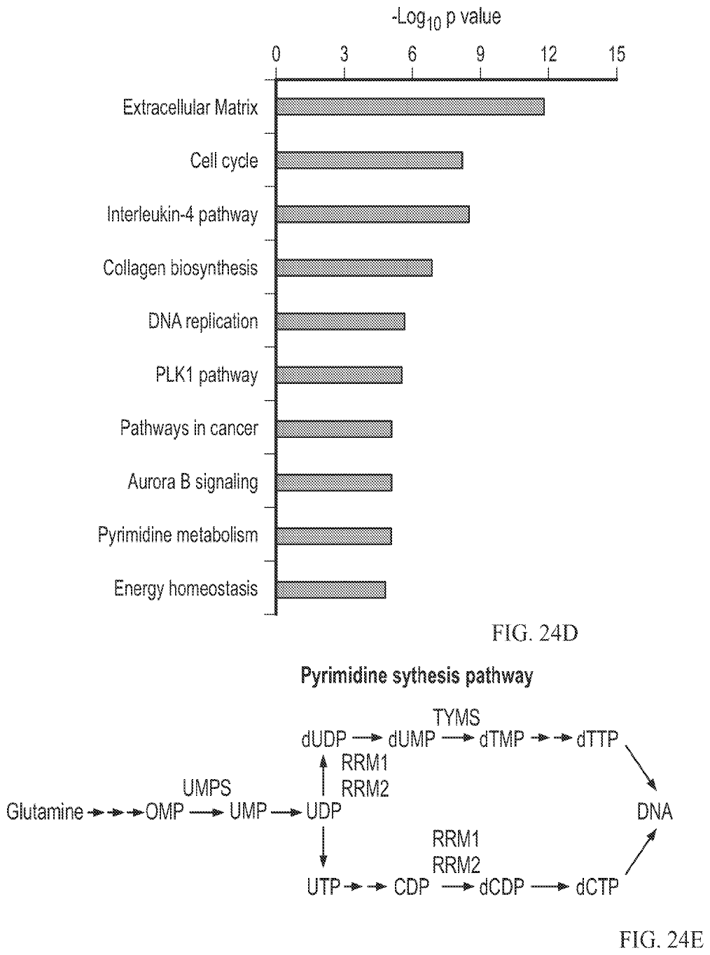

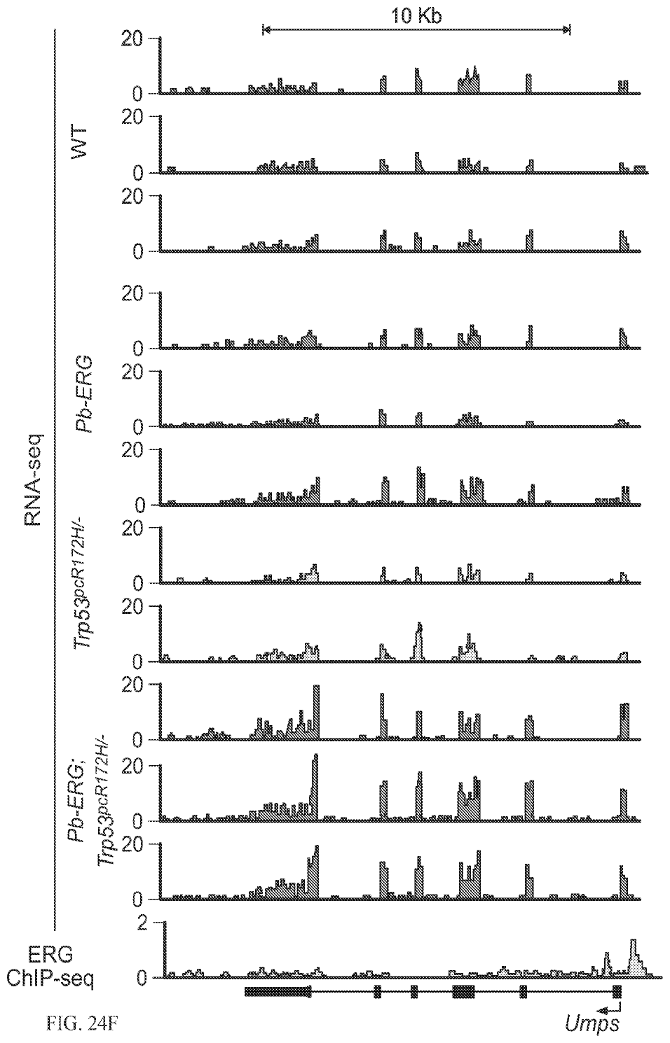

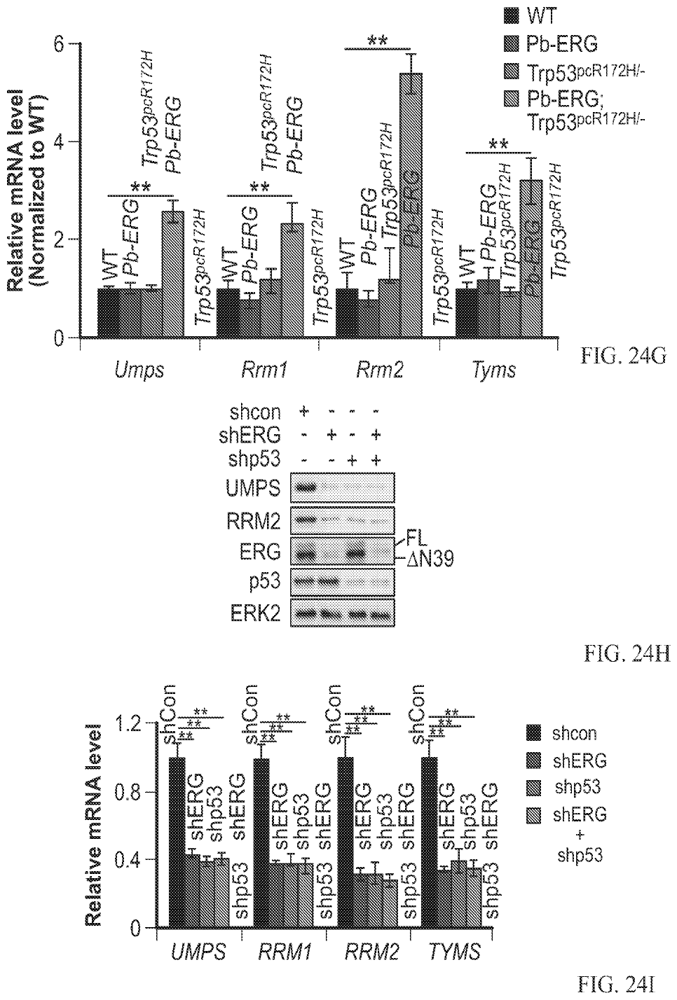

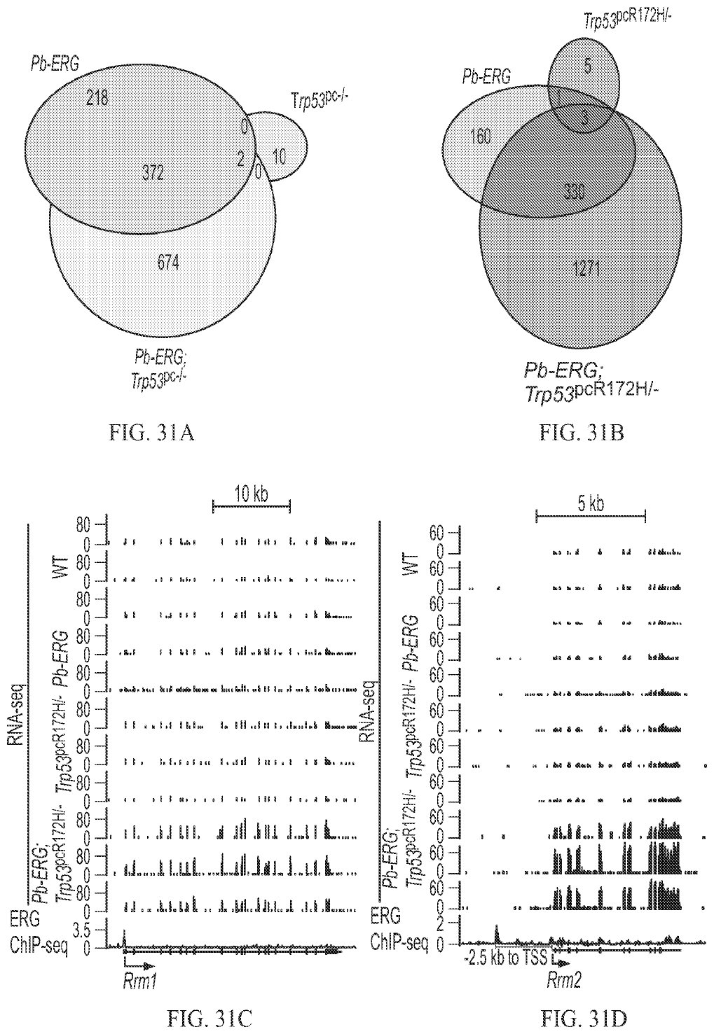

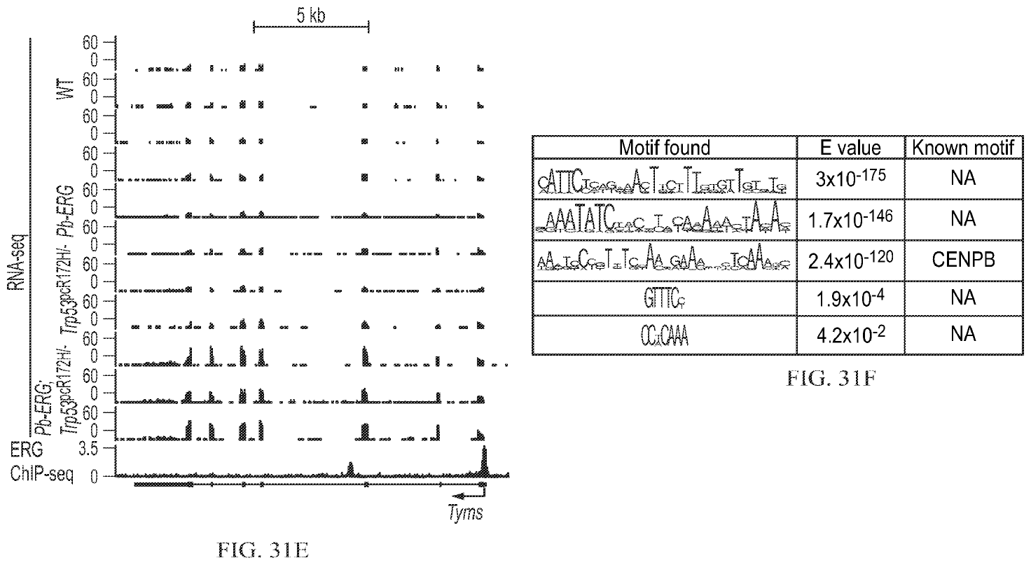

To understand the molecular mechanism underlying the accelerated prostate tumorigenesis induced by ERG overexpression and GOF p53 mutant (e.g. R172H) in mice, the downstream effectors uniquely altered in ERG/GOF p53 (Pb-ERG;Trp53^{pcR172H/-}) but not ERG/p53 KO (Pb-ERG;Trp53^{pc-/-}) mice were determined. RNA-seq analysis was performed in the prostate tissues of the six groups of mice shown in FIG. 23C. Clustering analysis of the RNA-seq data revealed that 901 genes were uniquely upregulated in tumors from ERG/GOF p53 mice compared to the ERG/p53 KO counterparts (FIGS. 24A, 31A, and 31B, and Table 13). Integration analysis of these upregulated genes and the ERG ChIP-seq data from murine prostate tumors revealed that 531 ERG target genes were highly upregulated in ERG/GOF p53 tumors (FIGS. 24B, 24C, and Table 14). IPA analysis showed that some of these genes are related to extracellular matrix, DNA replication, cell cycle and other cancer-relevant pathways (FIG. 24D). A group of PSGs, including the essential pyrimidine synthesis genes such as Upms, Rrm1, Rrm2 and Tyms, were highly upregulated in ERG/GOF p53 tumors compared to prostate tissues from ERG or GOF p53 alone mice (FIGS. 24C, 24E, 24F, and 31C-31E). Co-regulation of these essential PSGs by ERG and GOF p53 was further validated by RT-qPCR in ERG/GOF p53 tumors (FIG. 24G) and in VCaP human PCa cell line (FIGS. 24H and 24I).

TABLE 13
The list of 901 genes uniquely upregulated in tumors from ERG/p53
KIR172H mice compared to the ERG/p53 KO counterparts

| | |
|---|---|
| chr10: 102512221:102546560:RASSF9 | chr10: 110920176:110939599:CSRP2 |
| chr10: 109682659:110000219:NAV3 | chr10: 115817283:115849893:TSPAN8 |
| chr10: 121780990:122047315:SRGAP1 | chr11: 106654216:106750628:PECAM1 |
| chr10: 127078906:127093169:AGAP2 | chr11: 115381915:115396132:CDR2L |
| chr10: 127329888:127341589:GLI1 | chr11: 117199660:117362325:SEPT9 |
| chr10: 127538160:127621148:LRP1 | chr11: 118332359:118342500:BC100451 |
| chr10: 127724477:127731767:TAC2 | chr11: 118428498:118449963:C1QTNF1 |
| chr10: 128790952:128800824:MMP19 | chr11: 118444199:118454995:GM11747 |
| chr10: 128800035:128804370:TMEM198B | chr11: 22600334:22610879:GM26829 |
| chr10: 128882294:128891718:GDF11 | chr11: 29373657:29510808:CCDC88A |
| chr10: 128908918:128912816:CD63 | chr11: 43229562:43232264:GM12144 |
| chr10: 12939982:12964259:STX11 | chr11: 46143782:46147116:GM16033 |
| chr10: 23785346:23785475:SNORA33 | chr11: 46143896:46145014:GM16034 |
| chr10: 24223516:24302790:MOXD1 | chr11: 48887421:48902152:GM5431 |
| chr10: 28668359:28883815:THEMIS | chr11: 49057193:49064204:TGTP2 |
| chr10: 3740363:3967303:PLEKHG1 | chr11: 49671502:49712723:CNOT6 |
| chr10: 39369763:39565381:FYN | chr11: 5058127:5060385:RASL10A |
| chr10: 56377299:56390419:GJA1 | chr11: 5106264:5152257:EMID1 |
| chr10: 5799159:5805600:FBXO5 | chr11: 54303797:54364756:ACSL6 |
| chr10: 68723745:68782654:TMEM26 | chr11: 54340368:54353479:GM12224 |
| chr10: 70922831:71159700:BICC1 | chr11: 58379042:58390728:LYPD8 |
| chr10: 7444872:7473477:ULBP1 | chr11: 5861946:5872088:AEBP1 |
| chr10: 75923221:75932502:MMP11 | chr11: 58948919:58949533:HIST3H2BA |
| chr10: 79617939:79637918:SHC2 | chr11: 59306927:59333552:WNT9A |
| chr10: 79704490:79711969:BSG | chr11: 6658520:6677475:RAMP3 |
| chr10: 80057415:80102698:SBNO2 | chr11: 67455436:67688990:GAS7 |
| chr10: 80755205:80795461:DOT1L | chr11: 69045646:69051664:AURKB |
| chr10: 81084323:81098874:CREB3L3 | chr11: 69073426:69073561:SNORD118 |
| chr10: 82985497:83195900:CHST11 | chr11: 69667833:69667976:GM24029 |
| chr10: 87858264:87937042:IGF1 | chr11: 69823121:69837784:NLGN2 |
| chr10: 88322803:88357075:DRAM1 | chr11: 70224127:70229739:BCL6B |
| chr10: 89408822:89443967:GAS2L3 | chr11: 70459432:70466202:ZMYND15 |
| chr10: 91082939:91102607:IKBIP | chr11: 70790931:70812586:SCIMP |
| chr10: 91118290:91118536:GM24119 | chr11: 7206085:7213923:IGFBP3 |
| chr10: 92081745:92164748:RMST | chr11: 75513539:75526582:SCARF1 |
| chr10: 93247413:93311135:ELK3 | chr11: 76202014:76209416:FAM57A |
| chr10: 9627258:9675208:SAMD5 | chr11: 76210570:76217664:GEMIN4 |
| chr11: 100415696:100424824:FKBP10 | chr11: 78159399:78165589:TRAF4 |
| chr11: 101096321:101119893:FAM134C | chr11: 79239371:79254671:WSB1 |
| chr11: 101604849:101605040:GM26316 | chr11: 82035570:82037453:CCL2 |
| chr11: 102604395:102608058:FZD2 | chr11: 82979628:82991830:SLFN9 |
| chr11: 83002157:83020810:SLFN8 | chr12: 86678699:86692091:VASH1 |
| chr11: 83116848:83122670:SLFN1 | chr12: 8771322:8793715:SDC1 |
| chr11: 83175185:83190221:SLFN4 | chr13: 104287872:104494763:ADAMTS6 |
| chr11: 83191329:83215154:SLFN3 | chr13: 108316331:108389585:DEPDC1B |
| chr11: 83695274:83696185:GM11430 | chr13: 112800893:112867881:PPAP2A |
| chr11: 83703990:83706268:WFDC17 | chr13: 113209658:113218098:ESM1 |
| chr11: 86058137:86201193:BRIP1 | chr13: 16011850:16027211:INHBA |
| chr11: 87089152:87108708:PRR11 | chr13: 21715762:21716143:HIST1H2BL |
| chr11: 87443236:87443452:RNU3B1 | chr13: 21716421:21716814:HIST1H2AI |
| chr11: 9191941:9684259:ABCA13 | chr13: 21717658:21718069:HIST1H3H |
| chr11: 95261528:95269265:TAC4 | chr13: 21722097:21722478:HIST1H2BM |
| chr11: 95837215:95845734:GNGT2 | chr13: 21750193:21750505:HIST1H4K |
| chr11: 98036622:98053462:STAC2 | chr13: 21753434:21753827:HIST1H2AK |
| chr11: 98992942:99024189:TOP2A | chr13: 21779882:21780625:HIST1H1B |
| chr11: 99041243:99054392:IGFBP4 | chr13: 21786825:21787218:HIST1H2AN |
| chr12: 100549777:100725028:RPS6KA5 | chr13: 21787460:21789213:HIST1H2BP |
| chr12: 103763593:103773592:SERPINA1D | chr13: 22035163:22035568:HIST1H2AH |
| chr12: 103853588:103863555:SERPINA1A | chr13: 22035869:22036345:HIST1H2BK |
| chr12: 103946930:103958975:SERPINAIE | chr13: 22040635:22041362:HIST1H4I |
| chr12: 105563171:105593071:BDKRB2 | chr13: 22042459:22042944:HIST1H2AG |
| chr12: 108306269:108328300:HHIPL1 | chr13: 22043213:22043676:HIST1H2BJ |
| chr12: 108554719:108688513:EVL | chr13: 23533905:23534304:HIST1H2AF |
| chr12: 112106682:112127573:ASPG | chr13: 23535433:23535860:HIST1H3G |
| chr12: 112588783:112615556:INF2 | chr13: 23542969:23543357:HIST1H2BH |
| chr12: 112760654:112768986:PLD4 | chr13: 23544464:23545312:HIST1H3F |
| chr12: 113258767:113260236:IGHA | chr13: 23551257:23551648:HIST1H4F |
| chr12: 116405401:116463531:NCAPG2 | chr13: 23555086:23555830:HIST1H1D |
| chr12: 117516478:117756978:RAPGEF5 | chr13: 23570661:23571121:HIST1H2AE |
| chr12: 24708240:24714146:RRM2 | chr13: 23571407:23571884:HIST1H2BG |
| chr12: 24831598:24960301:MBOAT2 | chr13: 23573735:23574196:HIST1H2BF |
| chr12: 26306796:26415256:RNF144A | chr13: 23574469:23574932:HIST1H2AD |
| chr12: 27334263:27342574:SOX11 | chr13: 23575762:23576322:HIST1H3D |
| chr12: 29938035:30017658:PXDN | chr13: 23683448:23683924:HIST1H2AC |
| chr12: 58264719:58269258:CLEC14A | chr13: 23746789:23747241:HIST1H2BB |
| chr12: 65132733:65172580:MIS18BP1 | chr13: 23751125:23751598:HIST1H2AB |
| chr12: 81026807:81186414:SMOC1 | chr13: 23756202:23757620:4930558J22RIK |
| chr12: 81631368:81664941:TTC9 | chr13: 23757012:23757409:HIST1H4B |
| chr12: 84783211:84876532:LTBP2 | chr13: 23760691:23761230:HIST1H4A |
| chr12: 85686668:85709087:BATF | chr13: 23761852:23762386:HIST1H3A |
| chr13: 23763716:23764358:HIST1H1A | chr14: 51986388:51988829:GM16617 |
| chr13: 24582188:24733816:FAM65B | chr14: 54631991:54641364:CDH24 |
| chr13: 27345682:27354216:PRL8A2 | chr14: 55769057:55784042:ADCY4 |
| chr13: 28460777:28885620:2610307P16RIK | chr14: 55784995:55788857:RIPK3 |
| chr13: 32965208:32979067:SERPINB6B | chr14: 56129555:56132608:GZMD |
| chr13: 33003249:33017957:SERPINB9 | chr14: 59647530:60197179:ATP8A2 |
| chr13: 33879815:33905708:SERPINB6C | chr14: 61309752:61311936:ARL11 |
| chr13: 3882564:3918220:NET1 | chr14: 63943673:63950732:SOX7 |
| chr13: 51431040:51567084:SHC3 | chr14: 65400672:65425472:PNOC |
| chr13: 55445333:55460925:GRK6 | chr14: 65805836:65817822:PBK |
| chr13: 55473428:55488111:DBN1 | chr14: 67676330:67715841:CDCA2 |
| chr13: 56288646:56296551:CXCL14 | chr14: 69609067:69695834:LOXL2 |
| chr13: 56609602:56639339:TGFBI | chr14: 69767471:69784403:TNFRSF10B |
| chr13: 60842620:60864416:4930486L24RIK | chr14: 78569608:78725089:DGKH |
| chr13: 62836883:62858400:FBP2 | chr14: 79288755:79301645:RGCC |
| chr13: 64192544:64274973:CDC14B | chr14: 79766771:79771312:PCDH8 |
| chr13: 67810245:67811200:GM9625 | chr14: 79836711:79958726:GM6999 |
| chr13: 73467196:73516422:LPCAT1 | chr14: 84443562:84537060:PCDH17 |
| chr13: 73818533:73847631:NKD2 | chr14: 93015511:93888732:PCDH9 |
| chr13: 75089825:75132498:PCSK1 | chr15: 100641076:100669553:BIN2 |
| chr13: 89655311:89742509:VCAN | chr15: 100691812:100729376:GALNT6 |
| chr13: 92354782:92389053:DHFR | chr15: 100870682:101045929:SCN8A |
| chr13: 95601803:95618459:F2R | chr15: 101224206:101232755:GRASP |
| chr13: 96924688:96950912:GCNT4 | chr15: 101293231:101297426:6030408B16RIK |
| chr14: 102976580:102982637:KCTD12 | chr15: 102296292:102324356:ESPL1 |
| chr14: 102978219:102982528:GM26778 | chr15: 103344288:103366763:ITGA5 |
| chr14: 116925296:117979529:GPC6 | chr15: 10568978:10714631:RAI14 |
| chr14: 120478460:120507194:RAP2A | chr15: 11064789:11346867:ADAMTS12 |
| chr14: 16430841:16575472:RARB | chr15: 31568904:31590119:CMBL |
| chr14: 19751256:19811787:NID2 | chr15: 32240568:32244662:SNHG18 |
| chr14: 21733393:21748626:DUSP13 | chr15: 3270766:3280508:SEPP1 |
| chr14: 25459223:25666743:ZMIZ1 | chr15: 38294412:38300707:KLF10 |
| chr14: 25548389:25554369:GM26772 | chr15: 39076931:39087119:CTHRC1 |
| chr14: 31139012:31168641:STAB1 | chr15: 42424726:42676977:ANGPT1 |
| chr14: 32191853:32192050:GM23946 | chr15: 42676259:42704616:GM17473 |
| chr14: 32322018:32347820:OGDHL | chr15: 5233398:5244187:PTGER4 |
| chr14: 32785962:32817968:1810011H11RIK | chr15: 54250618:54278484:TNFRSF11B |
| chr14: 44851234:44859375:PTGDR | chr15: 58510047:58662933:FER1L6 |
| chr14: 51255265:51256112:RNASE2A | chr16: 30269301:30283256:LRRC15 |
| chr15: 66891319:66923201:WISP1 | chr16: 30599722:30602797:FAM43A |
| chr15: 67102874:67113992:ST3GAL1 | chr16: 32735885:32782391:MUC4 |
| chr15: 74721203:74724639:THEM6 | chr16: 33954781:33967038:UMPS |
| chr15: 74724317:74728034:SLURP1 | chr16: 36934982:36963212:HCLS1 |
| chr15: 74732246:74734329:LYPD2 | chr16: 37011785:37095417:POLQ |
| chr15: 74747851:74753046:LYNX1 | chr16: 37776872:37836514:FSTL1 |
| chr15: 74762055:74763620:LY6D | chr16: 38396118:38433145:PLA1A |
| chr15: 74834124:74841643:CYP11B1 | chr16: 4710058:4719356:NMRAL1 |
| chr15: 75155239:75159126:LY6G | chr16: 48994184:49019705:C330027C09RIK |
| chr15: 75596627:75599481:GPIHBP1 | chr16: 52031548:52208047:CBLB |
| chr15: 76703552:76710559:RECQL4 | chr16: 5211827:5222299:AU021092 |
| chr15: 77729120:77736381:APOL9B | chr16: 62814675:62824346:STX19 |
| chr15: 78480552:78495066:IL2RB | chr16: 65815632:65863057:VGLL3 |
| chr15: 78523345:78529625:C1QTNF6 | chr16: 85421532:85550417:CYYR1 |
| chr15: 78926724:78930465:LGALS1 | chr16: 85793826:85803113:ADAMTS1 |
| chr15: 80091333:80119501:SYNGR1 | chr16: 90719311:90727404:MIS18A |
| chr15: 80173720:80215519:MGAT3 | chr16: 90936091:91011308:SYNJ1 |
| chr15: 83149643:83149794:RNU12 | chr16: 92498133:92541243:CLIC6 |
| chr15: 83602582:83725021:SCUBE1 | chr16: 92612823:92620032:GM26626 |
| chr15: 85859706:85876572:GTSE1 | chr16: 94328419:94336935:RIPPLY3 |
| chr15: 89499622:89560261:SHANK3 | chr17: 12919584:12919722:GM26130 |
| chr15: 9111984:9155424:SKP2 | chr17: 13108616:13131791:UNC93A |
| chr15: 93499113:93595891:PRICKLE1 | chr17: 14829330:14934653:WDR27 |
| chr15: 96248957:96254616:4833422M21RIK | chr17: 21966174:21968272:GM7809 |
| chr15: 97792663:97844502:HDAC7 | chr17: 21967500:21968242:RP24-113B3.2 |
| chr15: 99074972:99083407:TROAP | chr17: 24223231:24251409:CCNF |
| chr15: 99590848:99594829:AQP5 | chr17: 24657329:24658457:NPW |
| chr16: 10959274:10993121:LITAF | chr17: 25162460:25171913:CCDC154 |
| chr16: 13715056:13730983:PLA2G10 | chr17: 25718925:25727419:CHTF18 |
| chr16: 15623896:15637400:MCM4 | chr17: 25748613:25754327:MSLN |
| chr16: 17797281:17808287:SCARF2 | chr17: 28769306:28778698:MAPK13 |
| chr16: 18621810:18629938:SEPT5 | chr17: 29360941:29379553:FGD2 |
| chr16: 18780446:18811972:CDC45 | chr17: 31677932:31681722:CRYAA |
| chr16: 19946498:19983037:KLHL6 | chr17: 33524203:33553768:ADAMTS10 |
| chr16: 20702963:20716117:CLCN2 | chr17: 33810519:33822918:KANK3 |
| chr16: 23058249:23082068:KNG1 | chr17: 34039436:34066685:COL11A2 |
| chr16: 23110755:23110933:SNORA81 | chr17: 34197789:34201454:PSMB8 |
| chr16: 23111616:23111755:GM24616 | chr17: 34564267:34588503:NOTCH4 |
| chr16: 23889580:23890844:SST | chr18: 67289252:67319146:IMPA2 |
| chr17: 35860917:35866886:PPP1R18OS | chr18: 67933256:68255549:LDLRAD4 |
| chr17: 35861317:35865402:NRM | chr18: 74195298:74207818:SKA1 |
| chr17: 35865592:35875596:PPP1R18 | chr19: 10182887:10196870:FADS1 |
| chr17: 37001162:37010635:ZFP57 | chr19: 10857821:10869940:TMEM132A |
| chr17: 39848102:39848827:AY036118 | chr19: 11067470:11081102:MS4A8A |
| chr17: 46564450:46629504:PTK7 | chr19: 11469365:11482192:MS4A6C |
| chr17: 48454900:48468686:UNC5CL | chr19: 11586603:11604849:MS4A6D |
| chr17: 49992256:50190674:RFTN1 | chr19: 12466340:12501996:DTX4 |
| chr17: 53674785:53689333:SGOL1 | chr19: 16435666:16610818:GNA14 |
| chr17: 56123084:56140343:SEMA6B | chr19: 18631949:18652194:NMRK1 |
| chr17: 56303320:56323486:UHRF1 | chr19: 23302608:23448322:MAMDC2 |
| chr17: 57105384:57107757:TNFSF9 | chr19: 29321343:29325356:INSL6 |
| chr17: 57358685:57483529:EMR1 | chr19: 34473785:34475135:CH25H |
| chr17: 67697264:67822645:LAMA1 | chr19: 34492317:34527474:LIPA |
| chr17: 71496099:71526857:NDC80 | chr19: 34922357:34975731:KIF20B |
| chr17: 71781946:71858351:CLIP4 | chr19: 37376402:37421859:KIF11 |
| chr17: 75435904:75529043:RASGRP3 | chr19: 38097078:38114263:FFAR4 |
| chr17: 7738568:7804974:FNDC1 | chr19: 41766587:41802084:ARHGAP19 |
| chr17: 79706952:79715041:CYP1B1 | chr19: 42036037:42045110:ANKRD2 |
| chr17: 83215291:83225070:PKDCC | chr19: 42045791:42070953:HOGA1 |
| chr17: 8525371:8986648:PDE10A | chr19: 42197970:42202252:SFRP5 |
| chr18: 22345088:22530227:ASXL3 | chr19: 42788946:43388311:HPSE2 |
| chr18: 33437018:33464029:NREP | chr19: 4425458:4439432:RHOD |
| chr18: 3507956:3516404:BAMBI | chr19: 5344704:5349574:CST6 |
| chr18: 35209010:35215024:LRRTM2 | chr19: 5651184:5663707:SIPA1 |
| chr18: 35554491:35554632:MIR1949 | chr19: 5824707:5845478:NEAT1 |
| chr18: 37320380:37323913:PCDHB5 | chr19: 5844285:5844437:GM27533 |
| chr18: 37341701:37345207:PCDHB7 | chr19: 58795750:58860984:HSPA12A |
| chr18: 37477767:37483035:PCDHB16 | chr19: 59425103:59458635:EMX2OS |
| chr18: 37485020:37488289:PCDHB17 | chr1: 118834131:119053619:GLI2 |
| chr18: 37674334:37841873:PCDHGA12 | chr1: 119415464:119422248:INHBB |
| chr18: 37972623:37997574:ARAP3 | chr1: 12692276:12861192:SULF1 |
| chr18: 38267091:38284401:PCDH12 | chr1: 128588198:128592290:CXCR4 |
| chr18: 4634928:4682868:9430020K01RIK | chr1: 12866548:12992650:SLCO5A1 |
| chr18: 58008622:58209926:FBN2 | chr1: 132036804:132067945:GM26892 |
| chr18: 60293379:60303016:F830016B08RIK | chr1: 132298625:132307357:KLHDC8A |
| chr18: 60376028:60392627:IIGP1 | chr1: 135584772:135599948:GM4793 |
| chr18: 61696836:61707635:PCYOX1L | chr1: 135799401:135810989:TNNI1 |
| chr18: 65265528:65393887:ALPK2 | chr1: 88227019:88262289:MROH2A |
| chr1: 138483836:138620218:NEK7 | chr1: 92831644:92860779:GPC1 |
| chr1: 149829617:149961290:PLA2G4A | chr2: 104754794:104816696:QSER1 |
| chr1: 150562523:150993051:HMCN1 | chr2: 105126528:105173616:WT1 |
| chr1: 152516759:152766351:RGL1 | chr2: 109280737:109341747:KIF18A |
| chr1: 153503014:153550045:NPL | chr2: 118123450:118124047:RP23-75E19.2 |
| chr1: 155096360:155099636:IER5 | chr2: 118598210:118641591:BUB1B |
| chr1: 155212470:155244444:BC034090 | chr2: 118754157:118762661:A430105I19RIK |
| chr1: 155520172:155527111:GM5532 | chr2: 118829873:118834122:GM14091 |
| chr1: 162217622:162225550:DNM3OS | chr2: 119325783:119335962:DLL4 |
| chr1: 164115263:164150026:SELP | chr2: 119618297:119651244:NUSAP1 |
| chr1: 164143431:164146366:GM16587 | chr2: 122637849:122641191:AA467197 |
| chr1: 165592180:165634541:MPZL1 | chr2: 124089968:124667770:SEMA6D |
| chr1: 169655500:169695813:RGS5 | chr2: 125207528:125217512:GM14002 |
| chr1: 170644531:170682789:OLFML2B | chr2: 125247189:125258608:DUT |
| chr1: 171588623:171607410:LY9 | chr2: 125300593:125507993:FBN1 |
| chr1: 171839696:171890718:CD84 | chr2: 125505088:125507881:GM9913 |
| chr1: 178529124:178932855:KIF26B | chr2: 126627441:126676337:GABPB1 |
| chr1: 182467255:182517495:CAPN2 | chr2: 127633225:127656695:MAL |
| chr1: 182565006:182632352:CAPN8 | chr2: 129198763:129211616:SLC20A1 |
| chr1: 184727139:184732619:HLX | chr2: 129205957:129226438:9830144P21RIK |
| chr1: 189207930:189343832:KCNK2 | chr2: 129268209:129297212:CKAP2L |
| chr1: 191537364:191575534:DTL | chr2: 130295168:130370481:EBF4 |
| chr1: 191821443:191833050:NEK2 | chr2: 131127279:131146321:HSPA12B |
| chr1: 192512827:192771213:HHAT | chr2: 131186948:131198497:CDC25B |
| chr1: 193221629:193264054:HSD11B1 | chr2: 131909927:131938429:PRNP |
| chr1: 3214481:3671498:XKR4 | chr2: 131950860:131956130:PRND |
| chr1: 36548641:36558381:SEMA4C | chr2: 133552158:133562885:BMP2 |
| chr1: 39194211:39363234:NPAS2 | chr2: 148436639:148443563:CD93 |
| chr1: 39900912:40026310:MAP4K4 | chr2: 150181754:150193279:GM14139 |
| chr1: 40515361:40551705:IL18RAP | chr2: 150470990:150485091:ZFP345 |
| chr1: 43746965:43827800:UXS1 | chr2: 152337421:152344032:TRIB3 |
| chr1: 44106545:44118808:KDELC1 | chr2: 152353614:152354566:GM14165 |
| chr1: 46807543:46854046:SLC39A10 | chr2: 153066001:153081735:CCM2L |
| chr1: 71027497:71103146:BARD1 | chr2: 156840006:156855570:TGIF2 |
| chr1: 71652836:71662843:APOL7D | chr2: 157015798:157079254:SOGA1 |
| chr1: 72226239:72226430:GM25360 | chr2: 160363702:160367065:MAFB |
| chr1: 74153988:74161246:CXCR2 | chr2: 160731309:160775760:PLCG1 |
| chr1: 86099025:86111970:HTR2B | chr2: 163054686:163084688:MYBL2 |
| chr1: 86666290:86670571:NPPC | chr2: 85136224:85139923:APLNR |
| chr2: 163492317:163502612:R3HDML | chr2: 85160777:85193325:GM13713 |
| chr2: 164579518:164585447:WFDC6A | chr2: 85162333:85196699:LRRC55 |
| chr2: 164596457:164613626:WFDC8 | chr2: 85196931:85198675:GM13716 |
| chr2: 164613521:164618212:WFDC6B | chr2: 91982327:92024502:CREB3L1 |
| chr2: 165055626:165072948:CD40 | chr2: 93187547:93201759:TRP53I11 |
| chr2: 166073088:166155663:SULF2 | chr3: 103102603:103169769:DENND2C |
| chr2: 167688914:167690418:CEBPB | chr3: 103860279:103912247:PTPN22 |
| chr2: 168476409:168601657:NFATC2 | chr3: 105001914:105053146:CTTNBP2NL |
| chr2: 172356189:172370535:AURKA | chr3: 106482430:106503030:DENND2D |
| chr2: 179442430:179899373:CDH4 | chr3: 115710432:115715055:S1PR1 |
| chr2: 181387761:181459426:ZBTB46 | chr3: 123507936:123508066:SNORA24 |
| chr2: 181688421:181693977:RGS19 | chr3: 126363826:126440374:ARSJ |
| chr2: 21205723:21215009:THNSL1 | chr3: 130180890:130599877:COL25A1 |
| chr2: 23115605:23156024:MASTL | chr3: 131110470:131224356:LEF1 |
| chr2: 25180757:25183339:NRARP | chr3: 132085291:132180293:DKK2 |
| chr2: 25291180:25319187:GRIN1 | chr3: 133112277:133234949:ARHGEF38 |
| chr2: 25291219:25298925:GRIN1OS | chr3: 135212562:135273540:CENPE |
| chr2: 25352289:25356359:DPP7 | chr3: 141465563:141834924:UNC5C |
| chr2: 25372320:25378213:SAPCD2 | chr3: 142620601:142638008:GBP2 |
| chr2: 25705849:25709716:A230005M16RIK | chr3: 151437886:151545086:ELTD1 |
| chr2: 25777016:25780279:LCN11 | chr3: 159495432:159529955:DEPDC1A |
| chr2: 26586638:26635634:GM20532 | chr3: 27097221:27153878:ECT2 |
| chr2: 26637847:26637974:SNORA43 | chr3: 31902506:32200180:KCNMB2 |
| chr2: 28025416:28030437:GM13372 | chr3: 34650404:34652461:SOX2 |
| chr2: 30078583:30091019:PKN3 | chr3: 36564865:36572150:CCNA2 |
| chr2: 30266202:30282149:PHYHD1 | chr3: 53041527:53261679:LHFP |
| chr2: 30471536:30474219:IER5L | chr3: 57285610:57301919:TM4SF1 |
| chr2: 31950259:31973442:AIF1L | chr3: 59006977:59318410:MED12L |
| chr2: 33216068:33247717:ANGPTL2 | chr3: 65528409:65555145:TIPARP |
| chr2: 34983330:35061438:HC | chr3: 67457997:67463926:LXN |
| chr2: 35313985:35336976:STOM | chr3: 69004737:69034623:SMC4 |
| chr2: 49787687:49948849:LYPD6B | chr3: 69574185:69598861:B3GALNT1 |
| chr2: 54084092:54085552:RPRM | chr3: 81036415:81214040:PDGFC |
| chr2: 59612041:59846149:TANC1 | chr3: 81037407:81040437:GM16000 |
| chr2: 60251992:60284488:CD302 | chr3: 82892578:82903973:LRAT |
| chr2: 62500942:62574075:FAP | chr3: 83007858:83015053:FGG |
| chr2: 66082765:66124994:GALNT3 | chr3: 83026152:83033615:FGA |
| chr2: 72476158:72486893:CDCA7 | chr3: 83766320:83774316:SFRP2 |
| chr2: 76663043:76673070:FKBP7 | chr4: 150853918:150868892:ERRFI1 |
| chr3: 83773852:83789956:GM26771 | chr4: 150920154:150946102:TNFRSF9 |
| chr3: 86995833:86999441:CD1D1 | chr4: 152274231:152285337:GPR153 |
| chr3: 87078592:87174747:KIRREL | chr4: 152390741:152477871:KCNAB2 |
| chr3: 87749096:87824306:PEAR1 | chr4: 155650654:155653384:MMP23 |
| chr3: 87971092:87980451:NES | chr4: 156022007:156031643:GM16008 |
| chr3: 90511033:90514392:S100A1 | chr4: 156026163:156028895:TNFRSF18 |
| chr3: 90669070:90670034:S100A8 | chr4: 24496450:24602950:MMS22L |
| chr3: 93278140:93279073:RPLP0-PS1 | chr4: 32657118:32775217:MDN1 |
| chr3: 96220360:96220880:HIST2H2AC | chr4: 4133530:4138477:PENK |
| chr3: 96261681:96263311:HIST2H4 | chr4: 4138320:4188703:A830012C17RIK |
| chr3: 96267074:96270289:GM20628 | chr4: 41569774:41638158:DNAIC1 |
| chr3: 96268653:96269141:HIST2H3B | chr4: 44092631:44109311:GM12503 |
| chr3: 96269720:96279001:HIST2H2BB | chr4: 44300875:44364675:MELK |
| chr3: 96414436:96414859:TERC | chr4: 47091908:47123070:GALNT12 |
| chr3: 96705891:96708560:NUDT17 | chr4: 47208160:47313167:COL15A1 |
| chr3: 97032415:97053634:GJA5 | chr4: 52439242:52488260:SMC2 |
| chr4: 100095790:100444765:ROR1 | chr4: 53261355:53270232:AI427809 |
| chr4: 108181933:108217922:ZYG11A | chr4: 63350410:63353846:GM11212 |
| chr4: 108400389:108406961:GPX7 | chr4: 63558780:63586357:6330416G13RIK |
| chr4: 108579453:108614833:ORC1 | chr4: 82897919:83052339:FREM1 |
| chr4: 11191353:11204779:CCNE2 | chr4: 86656564:86670060:PLIN2 |
| chr4: 114406723:114615098:TRABD2B | chr4: 87769924:88033364:MLLT3 |
| chr4: 114909256:114921118:FOXD2OS | chr4: 89274470:89294653:CDKN2A |
| chr4: 117096074:117115383:PTCH2 | chr4 JH584295 random:65:1479:CD99 |
| chr4: 117128654:117133963:PLK3 | chr5: 105078393:105139539:GBP9 |
| chr4: 117159638:117182639:KIF2C | chr5: 105519387:105613018:LRRC8C |
| chr4: 118471190:118490061:TIE1 | chr5: 110286305:110337474:POLE |
| chr4: 132270055:132270213:RNU11 | chr5: 115845643:116008947:CIT |
| chr4: 132310239:132310369:SNORA61 | chr5: 117378102:117389047:RFC5 |
| chr4: 132838382:132838547:GM22767 | chr5: 124862690:124888630:ZFP664 |
| chr4: 134068451:134092504:AIM1L | chr5: 125017152:125179219:NCOR2 |
| chr4: 138250402:138261332:SH2D5 | chr5: 129020068:129024323:RAN |
| chr4: 140700540:140723220:RCC2 | chr5: 129895722:129903623:ZBED5 |
| chr4: 141576061:141606052:FBLIM1 | chr5: 131437787:131616605:AUTS2 |
| chr4: 143267430:143299564:PDPN | chr5: 134656038:134688590:LIMK1 |
| chr4: 14502429:14621805:SLC26A7 | chr5: 134703780:134747241:ELN |
| chr4: 147611936:147642513:2610305D13RIK | chr5: 135406522:135545122:HIP1 |
| chr4: 149649167:149701590:PIK3CD | chr5: 136218148:136244903:SH2B2 |
| chr4: 149896282:149955043:SPSB1 | chr5: 91074616:91093649:EREG |
| chr5: 136987018:136996648:PLOD3 | chr5: 92226973:92253159:PPEF2 |
| chr5: 137350108:137374532:EPHB4 | chr5: 92321330:92328079:CXCL9 |
| chr5: 138164582:138172422:MCM7 | chr5: 92346637:92348889:CXCL10 |
| chr5: 139271875:139325622:ADAP1 | chr5: 93093456:93174958:SEPT11 |
| chr5: 140207384:140210751:GM16120 | chr5: 96373954:96784728:FRAS1 |
| chr5: 144244436:144264574:BRI3 | chr5: 98931719:99037035:PRKG2 |
| chr5: 146845070:146847726:RASL11A | chr6: 112473683:112489808:OXTR |
| chr5: 149368475:149430682:GM20488 | chr6: 121343075:121365775:SLC6A12 |
| chr5: 149411748:149431723:MEDAG | chr6: 122727808:122742745:SLC2A3 |
| chr5: 150522629:150569746:BRCA2 | chr6: 123229842:123247021:CLEC4N |
| chr5: 20986644:21055911:PTPN12 | chr6: 125121723:125121859:GM23751 |
| chr5: 23915276:24030690:FAM126A | chr6: 125161852:125166467:GAPDH |
| chr5: 30013160:30019968:IL6 | chr6: 125168006:125191701:NCAPD2 |
| chr5: 30058826:30073617:TYMS | chr6: 125186358:125186683:SCARNA10 |
| chr5: 30913401:30921278:EMILIN1 | chr6: 125186360:125186641:RP23-436K10.5 |
| chr5: 31251705:31253202:KRTCAP3 | chr6: 125232621:125237010:CD27 |
| chr5: 33213517:33218238:SPON2 | chr6: 126923049:126939587:RAD51AP1 |
| chr5: 3343892:3523218:CDK6 | chr6: 127961395:128143563:TSPAN9 |
| chr5: 33658127:33678995:TACC3 | chr6: 129350243:129365303:CLEC12A |
| chr5: 34153920:34169445:HAUS3 | chr6: 129408861:129424764:CLEC9A |
| chr5: 36017179:36398139:SORCS2 | chr6: 129426683:129452000:CLEC1A |
| chr5: 36490603:36586265:TBC1D14 | chr6: 131299143:131316398:STYK1 |
| chr5: 37242079:37292132:CRMP1 | chr6: 131364857:131388450:YBX3 |
| chr5: 38668483:38684826:ZFP518B | chr6: 131380272:131380406:GM22362 |
| chr5: 39613934:39755475:HS3ST1 | chr6: 134929154:134951718:1190002F15RIK |
| chr5: 43515568:43618817:CIQTNF7 | chr6: 136872435:136875805:MGP |
| chr5: 43557284:43601730:GM15866 | chr6: 138140315:138156755:MGST1 |
| chr5: 43818892:43843247:BST1 | chr6: 147252100:147264167:PTHLH |
| chr5: 45669921:45700544:NCAPG | chr6: 15720660:15802169:MDFIC |
| chr5: 4753872:4758035:FZD1 | chr6: 17988939:18030585:WNT2 |
| chr5: 53998416:54121057:STIM2 | chr6: 29735693:29761365:SMO |
| chr5: 64924810:64932761:TLR1 | chr6: 30541581:30564476:CPA2 |
| chr5: 66298860:66618828:APBB2 | chr6: 30738049:30748455:MEST |
| chr5: 72603695:72642752:CNGA1 | chr6: 3962588:3968357:TFPI2 |
| chr5: 72647795:72671078:NIPAL1 | chr6: 4003942:4008445:GNG11 |
| chr5: 72695977:72752773:TXK | chr6: 48647233:48660875:GIMAP8 |
| chr5: 75152290:75198204:PDGFRA | chr6: 48684548:48692060:GIMAP4 |
| chr5: 88764995:88783281:DCK | chr6: 48701581:48708225:GIMAP6 |
| chr5: 89673840:89883334:ADAMTS3 | chr7: 27486909:27490316:SERTAD1 |
| chr6: 48895253:48909188:AOC1 | chr7: 28716803:28738144:FBXO17 |
| chr6: 5220851:5256286:PON3 | chr7: 31290518:31291821:SCGB1B2 |
| chr6: 52242105:52245810:HOXA11 | chr7: 31375591:31376916:SCGB1B3 |
| chr6: 66535389:66541033:MAD2L1 | chr7: 33364342:33366322:SCGB2B20 |
| chr6: 72544390:72562983:CAPG | chr7: 43797576:43803822:KLK8 |
| chr6: 72957346:72958748:TMSB10 | chr7: 43995876:43999875:KLK1B11 |
| chr6: 73017605:73221651:DNAH6 | chr7: 44225436:44229617:KLK1 |
| chr6: 82725024:82774454:HK2 | chr7: 45082912:45092221:RCN3 |
| chr6: 86404218:86433403:TIA1 | chr7: 4784594:4789656:TMEM238 |
| chr6: 87133853:87335775:ANTXR1 | chr7: 48959096:49610090:NAV2 |
| chr6: 87913934:87936629:HMCES | chr7: 66109514:66173789:CHSY1 |
| chr6: 92772698:92943492:ADAMTS9 | chr7: 68737044:68749241:ARRDC4 |
| chr6: 92869356:92884411:GM15737 | chr7: 79660195:79698134:TICRR |
| chr7: 100176669:100184869:KCNE3 | chr7: 80454992:80535119:BLM |
| chr7: 101289614:101302088:ATG16L2 | chr7: 81600480:81706925:HOMER2 |
| chr7: 102441694:102469771:RRM1 | chr7: 82867332:82871563:MEX3B |
| chr7: 105752988:105787550:DCHS1 | chr7: 83932856:84086502:CEMIP |
| chr7: 109521279:109521409:GM24888 | chr7: 87246648:87398710:NOX4 |
| chr7: 110046363:110046547:SNORA23 | chr7: 92734165:92741468:4632427E13RIK |
| chr7: 112225855:112355194:MICAL2 | chr7: 98051059:98119524:MYO7A |
| chr7: 114631479:114636357:CALCA | chr7: 98494221:98501831:LRRC32 |
| chr7: 118597296:118675086:TMC5 | chr7: 98835130:98855195:WNT11 |
| chr7: 121865110:121918515:SCNN1B | chr7: 99345374:99353230:SERPINH1 |
| chr7: 122159436:122169875:PLK1 | chr8: 102864778:102865853:GM8730 |
| chr7: 127027731:127042420:KIF22 | chr8: 104101624:104144502:CDH5 |
| chr7: 130936202:130985660:HTRA1 | chr8: 105605228:105622194:FAM65A |
| chr7: 133883198:134225097:ADAM12 | chr8: 106168874:106198704:SLC7A6 |
| chr7: 140954838:140955961:IFITM2 | chr8: 106510912:106556908:CDH3 |
| chr7: 141061273:141072119:B4GALNT4 | chr8: 11198422:11312826:COL4A1 |
| chr7: 141292005:141294999:DRD4 | chr8: 11312804:11449287:COL4A2 |
| chr7: 141475239:141493427:TSPAN4 | chr8: 11399185:11417892:GM15419 |
| chr7: 141839069:141873084:MUC5B | chr8: 115682941:115707794:MAF |
| chr7: 143212154:143296549:KCNQ1OT1 | chr8: 122568014:122573128:CDT1 |
| chr7: 143667613:143685872:TNFRSF23 | chr8: 122628738:122699109:CBFA2T3 |
| chr7: 14561359:14609289:NLRP5-PS | chr8: 123653928:123663884:RHOU |
| chr7: 24541698:24546071:PINLYP | chr8: 125418062:125492710:SIPA1L2 |
| chr7: 24978166:25005937:ATP1A3 | chr8: 128359072:128505462:NRP1 |
| chr7: 25400052:25412886:CXCL17 | chr8: 15011024:15033333:KBTBD11 |
| chr7: 27168429:27181086:GM21983 | chr9: 21755441:21760286:SPC24 |
| chr8: 22168151:22185819:CKAP2 | chr9: 21800183:21852635:DOCK6 |
| chr8: 23669657:24063105:ZMAT4 | chr9: 28994749:29963129:NTM |
| chr8: 25808473:25814723:STAR | chr9: 30899154:30922452:ADAMTS15 |
| chr8: 35375740:35388124:PPP1R3B | chr9: 32696021:32757820:ETS1 |
| chr8: 40926232:40990785:PDGFRL | chr9: 34486125:35036716:KIRREL3 |
| chr8: 48099091:48153233:DCTD | chr9: 35116727:35130922:4930581F22RIK |
| chr8: 53586866:53639065:NEIL3 | chr9: 36708481:36726658:CHEK1 |
| chr8: 57304264:57320735:RP24-459A4.1 | chr9: 37528077:37538319:ESAM |
| chr8: 57320982:57324517:HAND2 | chr9: 39587509:39603687:AW551984 |
| chr8: 57320986:57324233:RP24-459A4.3 | chr9: 41011097:41158062:UBASH3B |
| chr8: 57523827:57653031:GALNT7 | chr9: 43221277:43239816:OAF |
| chr8: 69880368:69887687:CILP2 | chr9: 44334693:44336077:H2AFX |
| chr8: 70493157:70504081:CRLF1 | chr9: 5298516:5307265:CASP1 |
| chr8: 70594480:70597288:ISYNA1 | chr9: 53771534:53818161:SLC35F2 |
| chr8: 71406009:71409904:ANKLE1 | chr9: 54286485:54341786:GLDN |
| chr8: 71611023:71624909:COLGALT1 | chr9: 54586510:54604661:IDH3A |
| chr8: 77659247:77768970:4933431K23RIK | chr9: 55541147:55546180:ISL2 |
| chr8: 83955506:83970197:ASF1B | chr9: 58287722:58313212:LOXL1 |
| chr8: 85636587:85690973:NETO2 | chr9: 58488602:58499742:6030419C18RIK |
| chr8: 92960078:93001667:SLC6A2 | chr9: 59707636:59718874:GRAMD2 |
| chr8: 94137203:94139031:MT4 | chr9: 59966930:60511035:THSD4 |
| chr8: 94214596:94315066:NUP93 | chr9: 62858103:62875918:CALML4 |
| chr8: 94902868:94918098:CCDC102A | chr9: 64137143:64173104:ZWILCH |
| chr8: 95017691:95045247:GPR97 | chr9: 64811339:64919667:DENND4A |
| chr9: 107569116:107572776:HYAL2 | chr9: 65554385:65580040:PLEKHO2 |
| chr9: 107950962:107972268:TRAIP | chr9: 65587159:65595967:PIF1 |
| chr9: 110865710:110880113:TMIE | chr9: 70031495:70038088:GCNT3 |
| chr9: 114375133:114390675:CRTAP | chr9: 70407688:70421554:CCNB2 |
| chr9: 116087697:116175363:TGFBR2 | chr9: 71215788:71296243:ALDH1A2 |
| chr9: 120128779:120128935:GM24044 | chr9: 71626508:71771602:CGNL1 |
| chr9: 121489824:121495689:CCK | chr9: 7445821:7455972:MMP3 |
| chr9: 123259057:123260789:TMEM158 | chr9: 75625731:75637773:LYSMD2 |
| chr9: 15314844:15314981:GM25791 | chr9: 78430525:78443237:MB21D1 |
| chr9: 15315188:15315321:GM22620 | chr9: 86743648:86758443:PRSS35 |
| chr9: 15315521:15315595:GM24357 | chr9: 88521051:88522890:SNHG5 |
| chr9: 15316488:15316588:GM23455 | chr9: 88723284:88731914:BCL2A1D |
| chr9: 15316675:15316808:GM22579 | chr9: 8899832:8968611:PGR |
| chr9: 20770049:20815067:COL5A3 | chr9: 8971790:8975773:GM16485 |
| chr9: 21165713:21213248:PDE4A | chrX: 160488548:160499870:GM15241 |
| chr9: 90054266:90076089:CTSH | chrX: 163909016:163933666:AP1S2 |
| chr9: 90163068:90208071:ADAMTS7 | chrX: 167346321:167382749:PRPS2 |
| chr9: 92275601:92297752:PLSCR2 | chrX: 21484543:21489164:AGTR2 |
| chr9: 92542222:92608428:PLOD2 | chrX: 36328352:36362341:LONRF3 |
| chr9: 95399291:95406722:CHST2 | chrX: 38189792:38197046:ZBTB33 |
| chr9: 98422960:98446575:RBP1 | chrX: 41401127:41678601:GRIA3 |
| chrX: 100729941:100738894:GDPD2 | chrX: 48025145:48034853:APLN |
| chrX: 102141715:102157091:ERCC6L | chrX: 53055206:53057160:C430049B03RIK |
| chrX: 104077433:104201185:C77370 | chrX: 53669176:53670408:CXX1B |
| chrX: 106143228:106160493:TLR13 | chrX: 53724825:53738441:4930502E18RIK |
| chrX: 106360455:106384071:GM6325 | chrX: 57231484:57338729:ARHGEF6 |
| chrX: 106920624:106933900:LPAR4 | chrX: 6779305:6948362:DGKK |
| chrX: 107397098:107403376:ITM2A | chrX: 71962624:71972722:PRRG3 |
| chrX: 134308083:134362639:CENPI | chrX: 71991848:72010218:CNGA2 |
| chrX: 143802230:143827414:CAPN6 | chrX: 74177258:74208500:TKTL1 |
| chrX: 153832292:153834243:SPIN2C | chrX: 8271150:8280179:SLC38A5 |
| chrX: 155323917:155338467:PRDX4 | chrX: 93304766:93632155:POLA1 |
| chrX: 159414571:159498757:MAP7D2 | chrX: 9435251:9469324:CYBB |
| chrX: 159627271:159978069:SH3KBP1 | chrX: 99136129:99148991:EFNB1 |
| chrX: 160390689:160498070:GPR64 | |

TABLE 14
The gene list of 501 ERG target genes highly
upregulated in ERG/p53 KIR172H tumors

| | | | | |
|---|---|---|---|---|
| 1810011H11RIK | AEBP1 | ARSJ | BDKRB2 | CAPN2 |
| 4930486L24RIK | ALDH1A2 | ASF1B | BICC1 | CAPN8 |
| A430105I19RIK | ALPK2 | ASPG | BLM | CBFA2T3 |
| AA467197 | ANGPT1 | ASXL3 | BMP2 | CBLB |
| ADAM12 | ANGPTL2 | ATG16L2 | BRI3 | CCDC102A |
| ADAMTS1 | ANKRD2 | ATP8A2 | BRIP1 | CCDC88A |
| ADAMTS10 | ANTXR1 | AURKA | BSG | CCK |
| ADAMTS12 | AP1S2 | AURKB | BST1 | CCM2L |
| ADAMTS15 | APBB2 | AUTS2 | BUB1B | CCNA2 |
| ADAMTS3 | APLNR | B3GALNT1 | C1QTNF1 | CCNB2 |
| ADAMTS6 | AQP5 | B4GALNT4 | C1QTNF6 | CCNE2 |
| ADAMTS7 | ARAP3 | BAMBI | C1QTNF7 | CCNF |
| ADAMTS9 | ARHGEF38 | BARD1 | C330027C09RIK | CD27 |
| ADAP1 | ARL11 | BC034090 | CALCA | CD302 |
| ADCY4 | ARRDC4 | BCL6B | CAPG | CD93 |
| CDC14B | CRTAP | ERRFI1 | GM9913 | IDH3A |
| CDC25B | CTSH | ESAM | GNA14 | IER5 |
| CDC45 | CTTNBP2NL | ESPL1 | GNG11 | IER5L |
| CDCA2 | CXCL17 | ETS1 | GNGT2 | IFITM2 |
| CDCA7 | CXCL9 | EVL | GPIHBP1 | IGF1 |
| CDH24 | CXCR4 | F2R | GRAMD2 | IGFBP3 |
| CDH3 | CYBB | FADS1 | GRASP | IGFBP4 |
| CDH4 | CYP1B1 | FAM126A | GRK6 | IIGP1 |
| CDH5 | CYYR1 | FAM43A | GTSE1 | IKBIP |
| CDK6 | DCHS1 | FBLIM1 | HAND2 | IL2RB |
| CDR2L | DCTD | FBN2 | HAUS3 | IL6 |
| CDT1 | DENND2C | FBP2 | HC | IMPA2 |
| CEBPB | DENND2D | FBXO5 | HDAC7 | INF2 |
| CEMIP | DENND4A | FER1L6 | HHAT | INHBA |
| CGNL1 | DEPDC1A | FGD2 | HHIPL1 | INHBB |
| CH25H | DEPDC1B | FKBP7 | HIP1 | INSL6 |
| CHST11 | DGKH | FRAS1 | HIST1H2AC | ISYNA1 |
| CHST2 | DGKK | FREM1 | HIST1H2AD | ITGA5 |
| CHSY1 | DHFR | FSTL1 | HIST1H2AE | KANK3 |
| CIT | DKK2 | FYN | HIST1H2AF | KBTBD11 |
| CLCN2 | DLL4 | FZD1 | HIST1H2AI | KCNE3 |
| CLEC14A | DNAH6 | FZD2 | HIST1H2BG | KCNK2 |
| CLEC1A | DNAIC1 | GABPB1 | HIST1H2BJ | KCNMB2 |
| CLEC9A | DOCK6 | GALNT3 | HIST1H4I | KDELC1 |
| CLIC6 | DOT1L | GALNT6 | HIST2H4 | KIF11 |
| CLIP4 | DPP7 | GALNT7 | HK2 | KIF18A |
| CMBL | DRAM1 | GAPDH | HLX | KIF20B |
| CNOT6 | DTL | GAS7 | HMCES | KIF22 |
| COL11A2 | DTX4 | GBP9 | HMCN1 | KIF26B |
| COL15A1 | DUT | GCNT3 | HOMER2 | KIF2C |
| COL25A1 | EBF4 | GEMIN4 | HOXA11 | KIRREL |
| COL4A1 | ECT2 | GIMAP4 | HPSE2 | KIRREL3 |
| COL4A2 | EFNB1 | GIMAP6 | HS3ST1 | KLF10 |
| COL5A3 | ELK3 | GJA1 | HSD11B1 | KLHDC8A |
| COLGALT1 | ELN | GJA5 | HSPA12A | KLHL6 |
| CPA2 | EMID1 | GLDN | HSPA12B | KRTCAP3 |
| CREB3L1 | EMILIN1 | GLI2 | HTR2B | LAMA1 |
| CRLF1 | EPHB4 | GM15737 | HTRA1 | LDLRAD4 |
| CRMP1 | ERCC6L | GM5431 | HYAL2 | LEF1 |
| LHFP | MMS22L | PCDH8 | QSER1 | SEMA4C |
| LIMK1 | MOXD1 | PCDH9 | RAI14 | SEMA6B |
| LITAF | MPZL1 | PCSK1 | RAN | SEMA6D |
| LONRF3 | MROH2A | PCYOX1L | RAP2A | 11-Sep |
| LOXL1 | MS4A8A | PDE4A | RAPGEF5 | 5-Sep |
| LOXL2 | MUC4 | PDGFC | RARB | 9-Sep |
| LPAR4 | NAV2 | PDGFRA | RASGRP3 | SERPINB9 |
| LRP1 | NAV3 | PDGFRL | RASL10A | SERPINH1 |
| LRRC32 | NCAPD2 | PECAM1 | RASL11A | SERTAD1 |
| LRRC55 | NCAPG | PENK | RASSF9 | SFRP2 |
| LRRC8C | NCAPG2 | PHYHD1 | RBP1 | SH2B2 |
| LRRTM2 | NCOR2 | PIK3CD | RCC2 | SH2D5 |
| LTBP2 | NDC80 | PINLYP | RCN3 | SH3KBP1 |
| LXN | NEK2 | PKDCC | RFC5 | SHANK3 |
| LYPD2 | NEK7 | PKN3 | RFTN1 | SHC3 |
| LYPD6B | NES | PLA1A | RGCC | SIPA1 |
| LYSMD2 | NET1 | PLA2G10 | RGL1 | SIPA1L2 |
| MAD2L1 | NETO2 | PLA2G4A | RGS19 | SKP2 |
| MAF | NFATC2 | PLCG1 | RHOU | SLC20A1 |
| MAFB | NID2 | PLEKHG1 | RIPK3 | SLC2A3 |
| MAL | NIPAL1 | PLEKHO2 | RNASE2A | SLC35F2 |
| MAP4K4 | NMRK1 | PLK3 | RNF144A | SLC39A10 |
| MAPK13 | NOTCH4 | PLOD2 | ROR1 | SLC6A2 |
| MASTL | NPAS2 | PLOD3 | RPRM | SLCO5A1 |
| MBOAT2 | NPL | PNOC | RPS6KA5 | SLFN1 |
| MCM4 | NPPC | POLA1 | RRM1 | SLFN3 |
| MDFIC | NRARP | POLQ | RRM2 | SMC2 |
| MDN1 | NREP | PON3 | S100A1 | SMC4 |
| MEDAG | NRP1 | PPEF2 | S1PR1 | SMO |
| MELK | NTM | PPP1R18 | SAMD5 | SOGA1 |
| MEX3B | NUDT17 | PPP1R3B | SBNO2 | SORCS2 |
| MGP | NUP93 | PRICKLE1 | SCARF1 | SOX11 |
| MGST1 | NUSAP1 | PRKG2 | SCGB1B2 | SOX2 |
| MICAL2 | OLFML2B | PRND | SCIMP | SOX7 |
| MIS18A | ORC1 | PTCH2 | SCN8A | SPC24 |
| MIS18BP1 | OXTR | PTGER4 | SCNN1B | SPIN2C |
| MLLT3 | PBK | PTK7 | SCUBE1 | SPON2 |
| MMP19 | PCDH12 | PTPN12 | SDC1 | SPSB1 |
| MMP3 | PCDH17 | PXDN | SELP | SRGAP1 |
| SST | TBC1D14 | TMEM198B | TSPAN8 | WSB1 |
| ST3GAL1 | TGFBI | TMEM238 | TSPAN9 | XKR4 |
| STAB1 | TGFBR2 | TMEM26 | TXK | YBX3 |
| STIM2 | TGIF2 | TMIE | TYMS | ZBTB33 |
| STOM | THEM6 | TMSB10 | UBASH3B | ZBTB46 |
| STX11 | THEMIS | TNFRSF18 | UHRF1 | ZFP518B |
| STX19 | THSD4 | TNFRSF9 | UMPS | ZFP664 |
| STYK1 | TIE1 | TNNI1 | UNC5C | ZMAT4 |
| SULF1 | TIPARP | TRABD2B | UXS1 | ZMIZ1 |
| SULF2 | TLR1 | TRAF4 | VCAN | ZWILCH |
| SYNJ1 | TLR13 | TRAIP | VGLL3 | |
| TAC4 | TM4SF1 | TRIB3 | WDR27 | |
| TACC3 | TMC5 | TRP53I11 | WISP1 | |
| TANC1 | TMEM158 | TSPAN4 | WNT11 | |

### Identification of CTNNB1 Gene as a Binding Target of Mutant p53

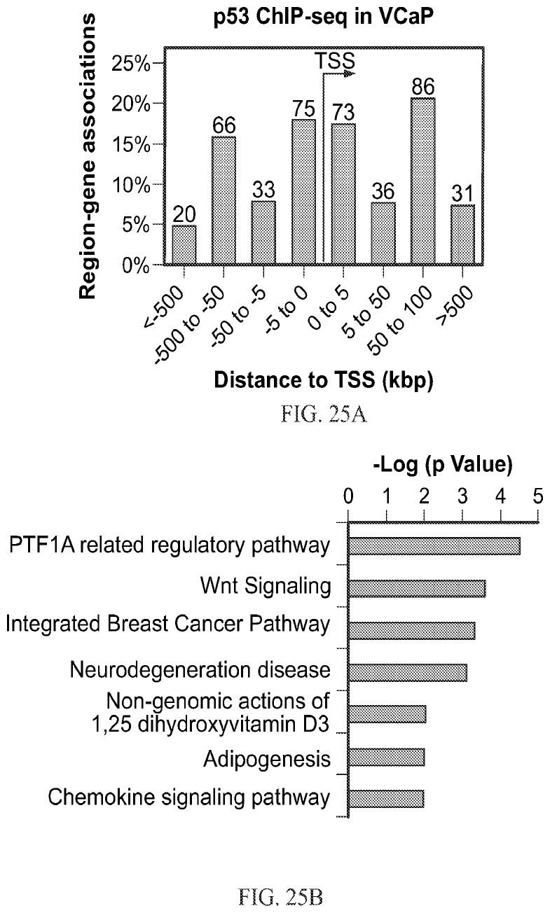

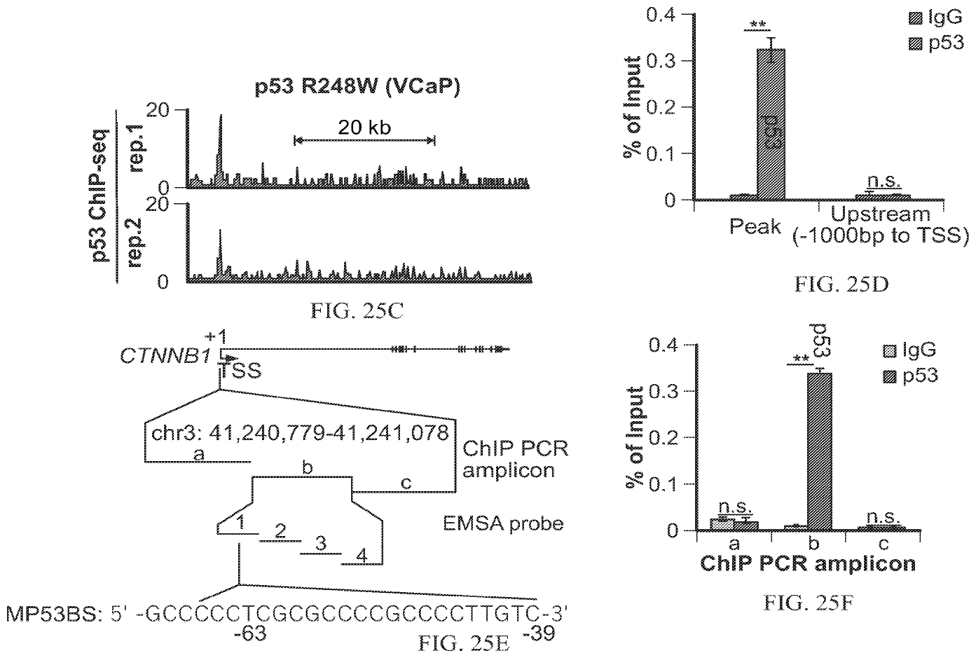

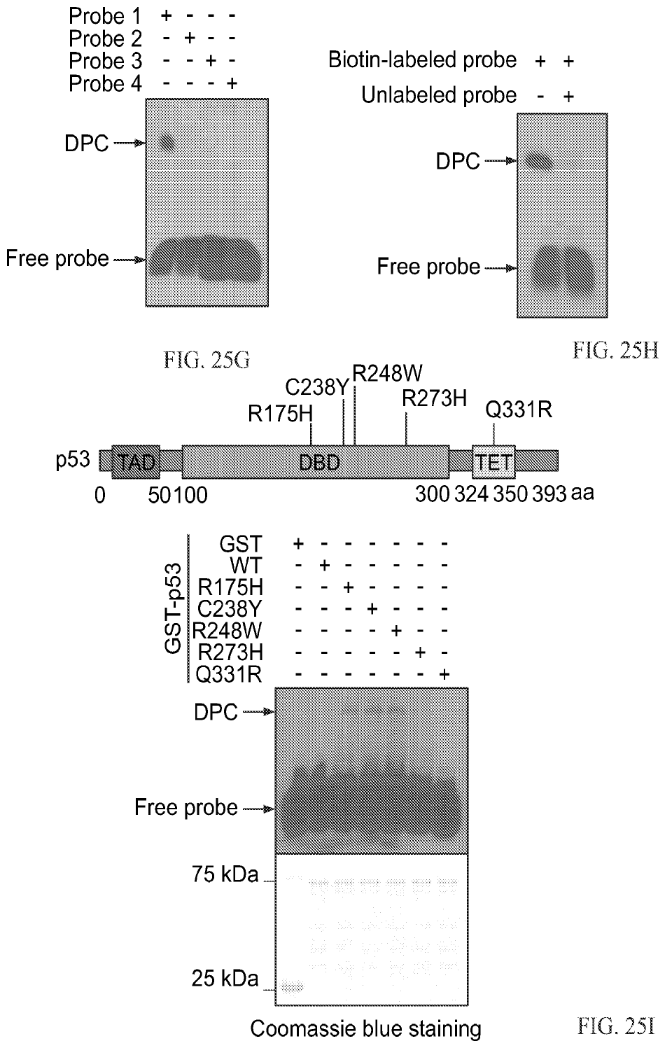

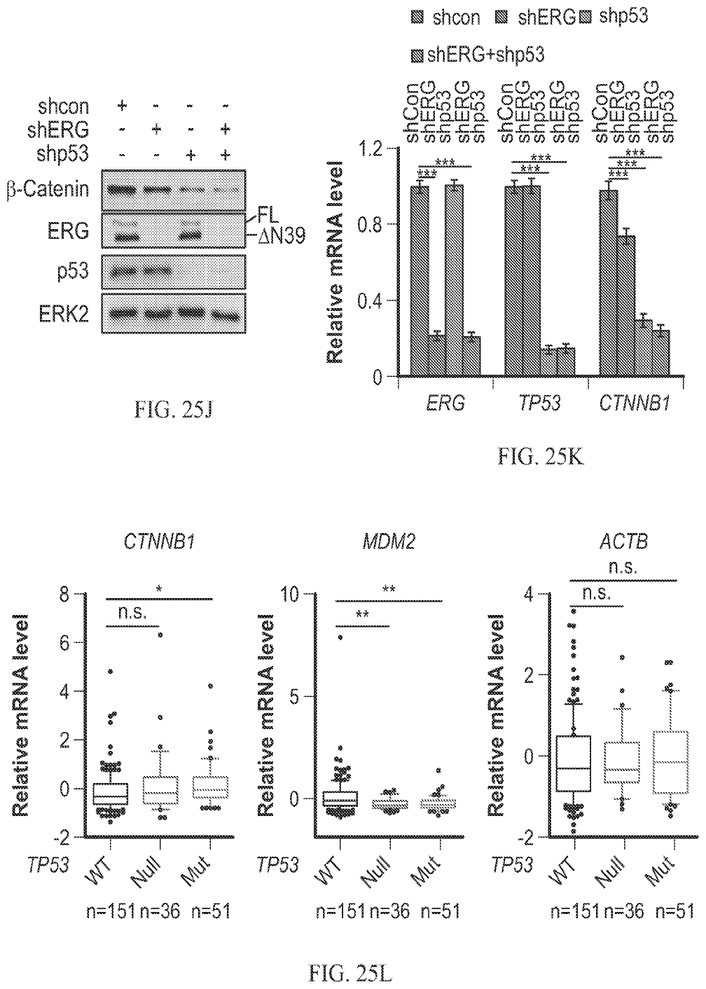

Given that several key PSGs are co-regulated by ERG and GOF p53 (R172H in GEM tumors and R248W in human VCaP cells) (FIGS. 24C-24H) and that ERG occupies in the promoters of these PSGs (FIGS. 24F and 31C-31E), we sought to determine whether mutant p53 also binds to the genomic loci of these PSGs. To this end, we performed p53 ChIP-seq in VCaP cells and identified more than 400 (n=416) p53 R248W mutant highly enriched genomic loci in this cell line (FIG. 25A and Table 5; note: 416 peaks identified are located in 359 gene loci). DNA binding motif analysis showed that except the DNA-binding protein centromere protein B (CENPN) binding element, no typical transcription factor binding motif was specifically enriched (FIG. 31F). The GOF p53-binding peaks were localized in both promoter and non-promoter regions, but to our surprise none of them are present in the PSG loci in VCaP cells (FIG. 25A and Table 5), suggesting that p53 mutant may regulate PSG expression through indirect mechanism(s).

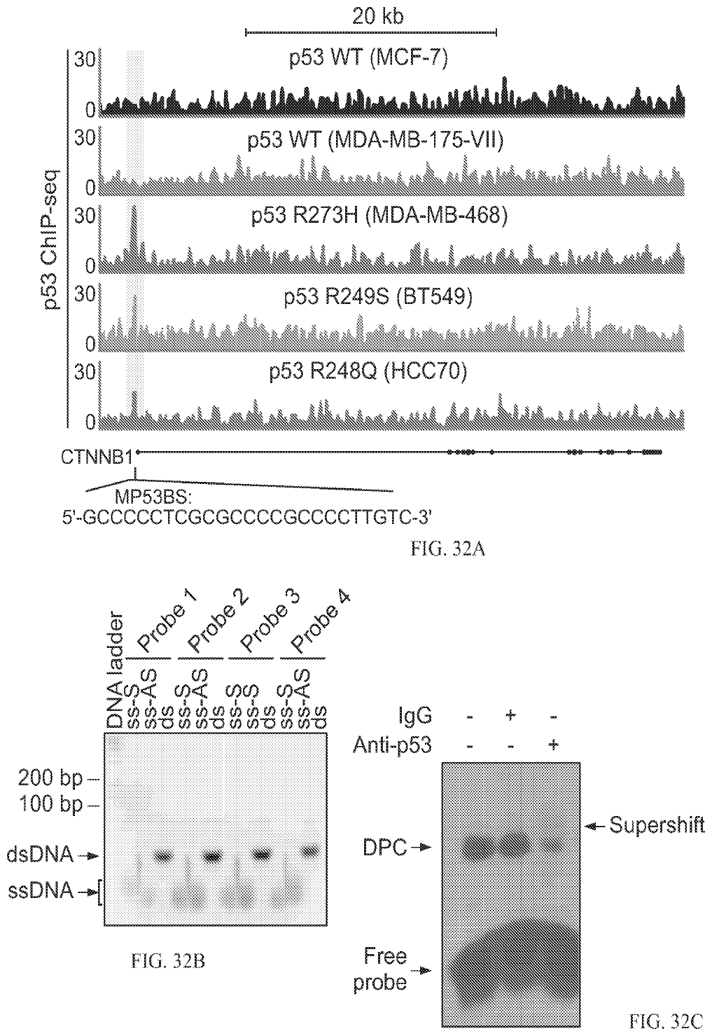

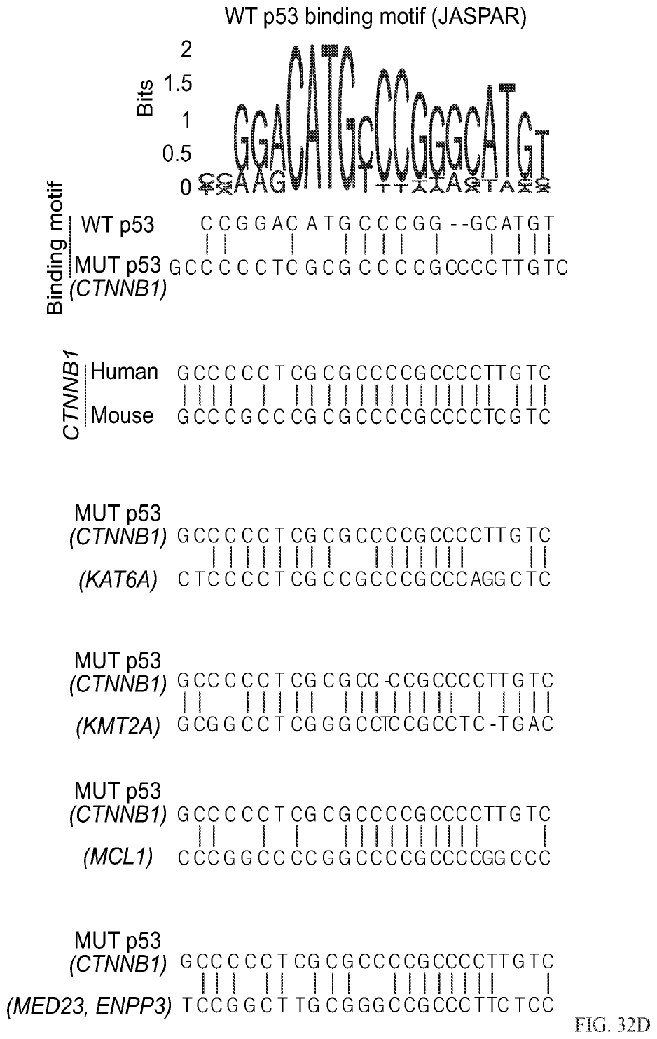

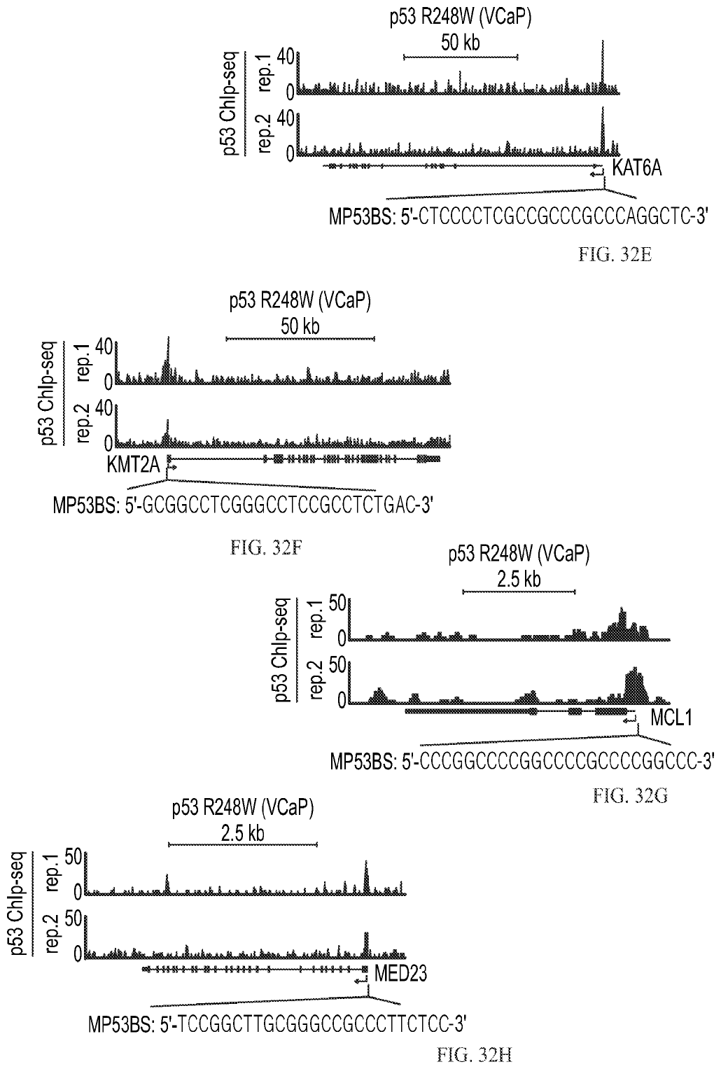

To define the potential downstream effector(s) underlying p53 mutant-mediated PSG expression, pathway enrichment analysis was conducted and it was found that Wnt signaling was one of the pathways enriched among the R248W-bound targets (FIG. 25B and Table 5). Specifically, a p53 mutant (R248W)-bound peak was detected in the promoter of CTNNB1 gene which encodes β—Catenin, a core component of the Wnt signaling pathway (FIG. 25C). Specific occupancy of p53 R248W at the promoter of the CTNNB1 gene, but not in a non-occupied region was verified by quantitative ChIP-PCR (ChIP-qPCR) in VCaP cells (FIG. 25D). Meta-analysis of p53 ChIP-seq data generated in different breast cancer cell lines expressing WT or GOF mutated p53 showed that p53 R273H, R249S and R248Q mutants, but no WT p53 invariably bound the CTNNB1 promoter (FIG. 32A).

To define the DNA sequence bound by GOF p53 mutant in the CTNNB1 promoter, p53 R248W ChIP-qPCR analysis was performed using a sequential set of primers (FIG. 25E). It was demonstrated that p53 R248W specifically occupied in the center (#b amplicon) of the p53 mutant ChIP-seq peak in VCaP cells (FIG. 25F). To explore the minimal mutant p53 binding sequence, electrophoresis mobility shift assay (EMSA) was performed using VCaP cell lysate and four biotin-labeled double-stranded probes covering the #b amplicon (FIGS. 25E and 32B). The binding sequence was narrowed down a 25-bp mutant p53-bound DNA sequence (MP53BS) in the CTNNB1 gene promoter (FIGS. 25E and 25G). The EMSA signal of MP53BS was largely diminished by adding unlabeled probe or anti-p53 antibody in the assays (FIGS. 25H and 32C), indicating that the detected binding signal is p53 mutant (R248W) specific. Besides using cell nuclear extract, EMSA was also performed using glutathione S-transferase (GST)-p53 recombinant proteins purified from bacteria, which include WT p53 and the mutants relevant to this study including R175H (equivalent to R172H used in GEM), C238Y (LuCaP 23.1 patient-derived xenograft (PDX)), R248W (VCaP cell line), R273H (MDA-MB-468 breast cancer cell line) and Q331R, a residue outside of DBD (22Rv1 cells). It was found that except WT and Q331R, all the DBD mutants of p53 bound to the DNA probe (FIG. 25I), suggesting that the DBD mutants of p53 can directly bind to the MP53BS in the CTNNB1 gene promoter. This motif shared approximately 50% (can be more or less) of homology with the WT p53 binding consensus sequence, but almost identical to the motif in the mouse Ctnnb1 promoter (FIG. 32D). A similar motif, especially a CCCGCCC core sequence can be found in the promoters of many other GOF p53-bound cancer-related genes such as KAT6A and KMT2A (FIGS. 32D, 32E, and Table 6).

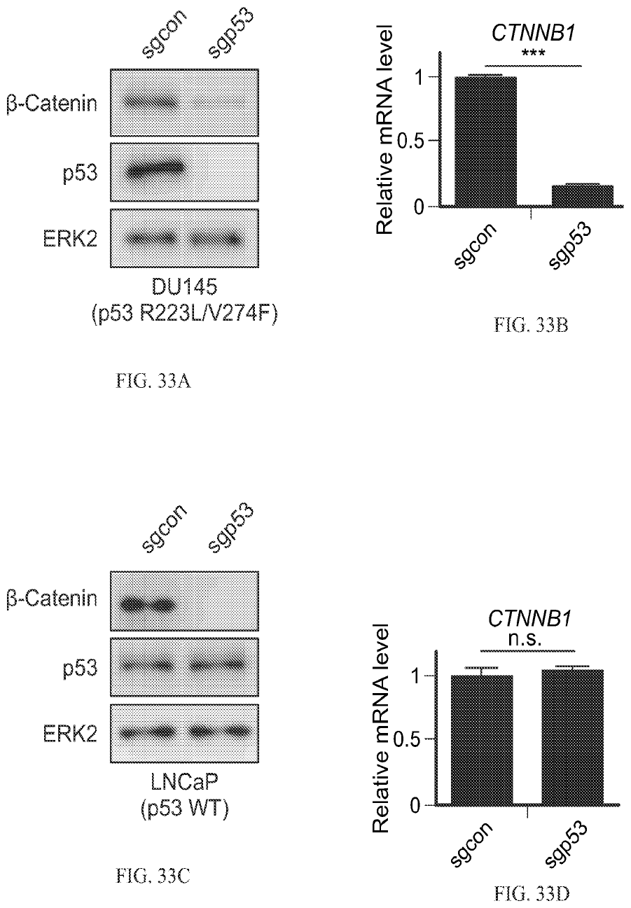

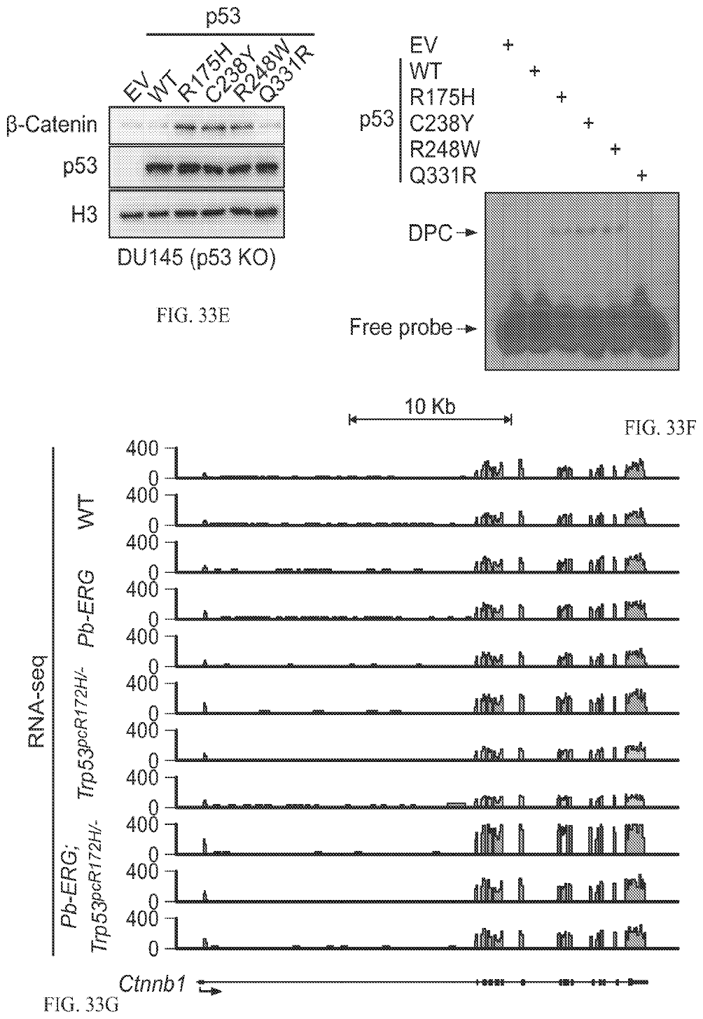

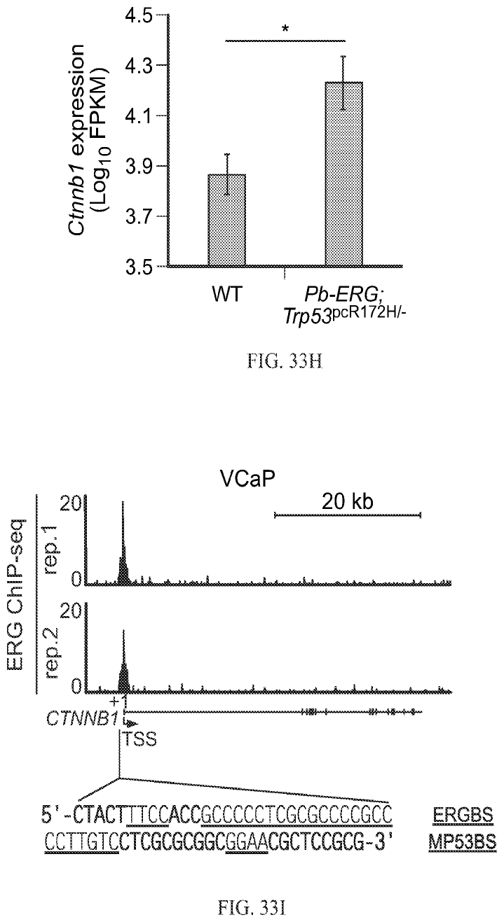

In agreement with the p53 mutant ChIP-seq and EMSA results, it was found that knockdown (KD) of endogenous p53 R248W inhibited β—Catenin expression at both mRNA and protein levels in VCaP cells (FIGS. 25J and 25K), indicating an important role of GOF p53 mutant in regulation of β—Catenin expression. Similar to VCaP cells, the TP53 gene is mutated (R223L/V274F) and ETS family proteins (e.g. ETV4) are expressed in DU145 PCa cell line. KO of GOF p53 mutants also decreased β—Catenin expression in DU145 cells (FIGS. 33A and 33B). In contrast, KO of endogenous WT p53 in LNCaP cells had no obvious effect on β—Catenin mRNA and protein expression (FIGS. 33C and 33D).

Expression of WT p53 or different mutants in p53-KO/ETV4-expressing DU145 cells was restored. Consistent with the EMSA results, rescued expression of the DBD mutants R175H, C248Y and R248W, but not WT p53 and Q331R induced β—Catenin expression (FIGS. 33E and 33F). These data suggest that GOF p53 mutants shared the ability to upregulate β—Catenin expression in PCa cells. In agreement with these observations, RNA-seq results showed that co-expression of ERG and p53 R172H increased Ctnnb1 mRNA expression in murine prostate tumors in GEM mice (FIGS. 33G and 33H). p53 R172H knockin alone was insufficient to upregulate Ctnnb1 gene expression in the mouse prostate (FIG. 33G), implying that ERG overexpression primes GOF p53 mutant regulation of CTNNB1 expression. This notion is supported by our ChIP-seq data that ERG also bound the CTNNB1 gene promoter and two core elements of the ERB binding sequence (ERGBS) are located in flanks of MP53BS in this locus (FIG. 33I). Furthermore, it was shown that ERG KD in VCaP cells also downregulated expression of CTNNB1 mRNA and β—Catenin protein, and the effects were enhanced by KD of both (FIGS. 25J and 25K). Finally, meta-analysis of SU2C data from patients with advanced PCa was performed. It was found that CTNNB1 mRNA level was significantly higher in tumors with mutations in the DNA binding domain (DBD) of p53 compared to the samples with p53 WT or homozygous deletion (FIG. 25L). Together, these data support the notion that GOF p53 mutants bind to the promoter and upregulate CTNNB1 gene.

### ERG and β—Catenin Co-Regulate PSG Expression in PCa

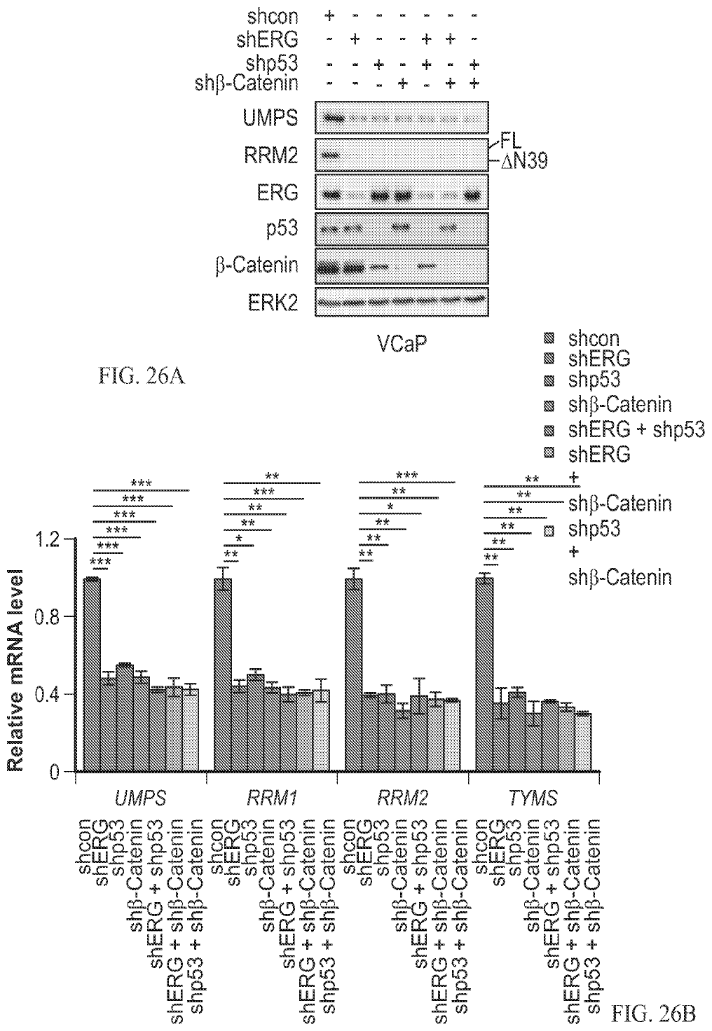

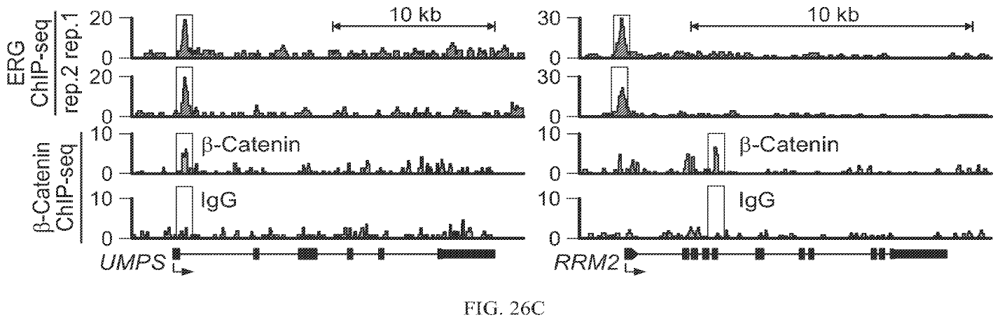

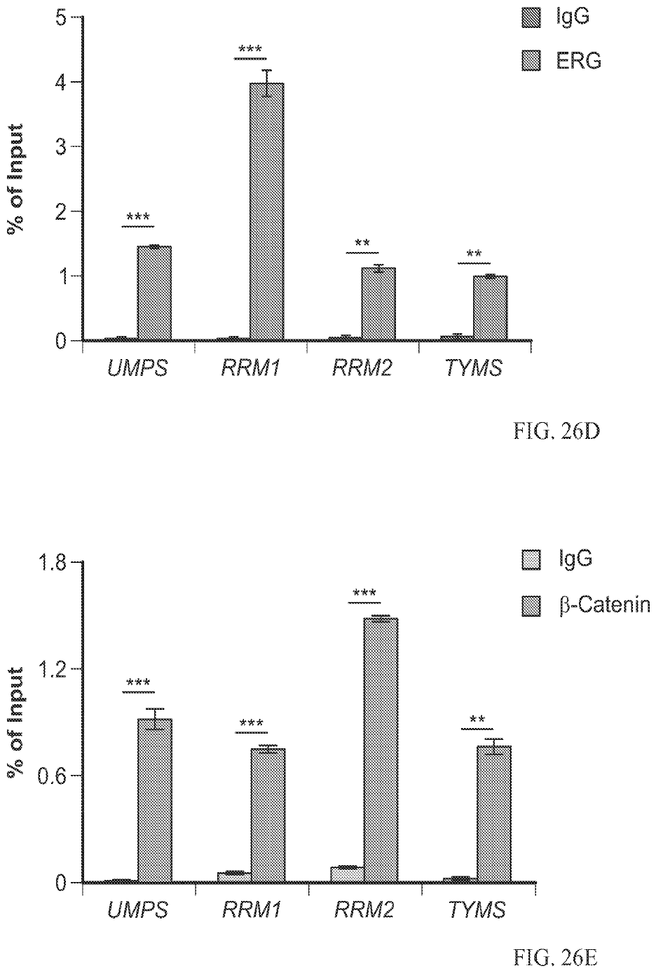

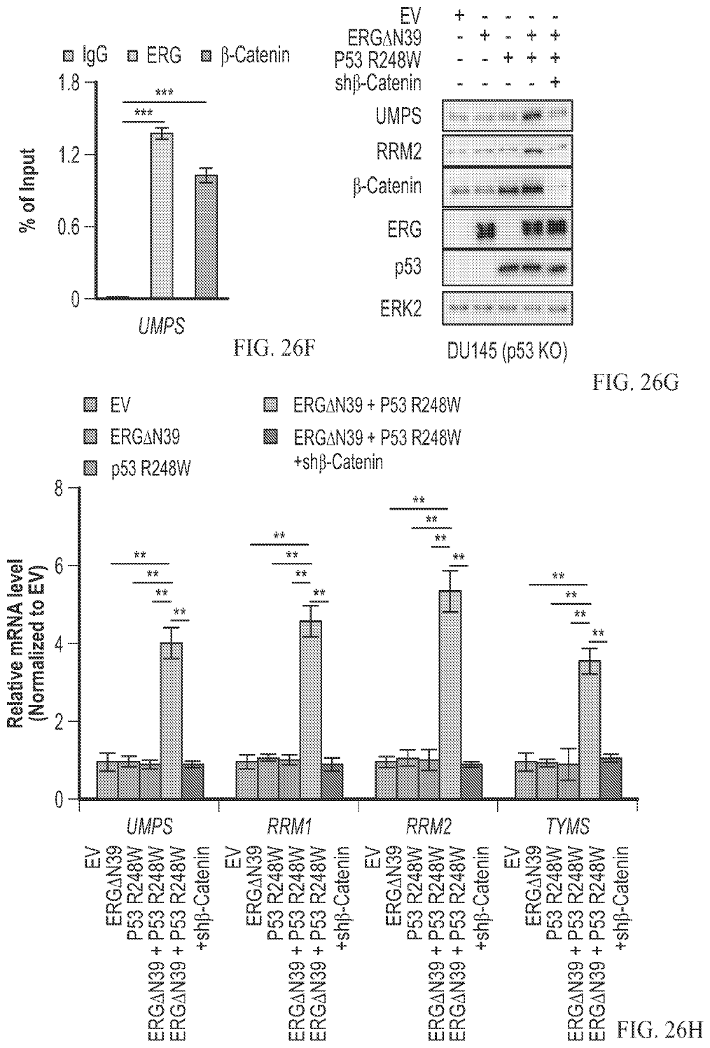

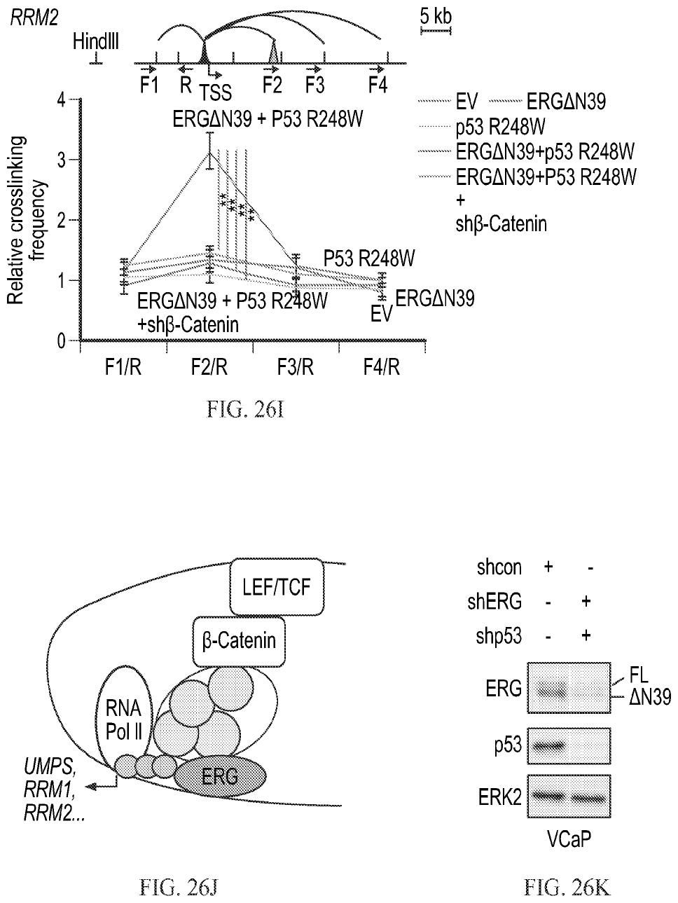

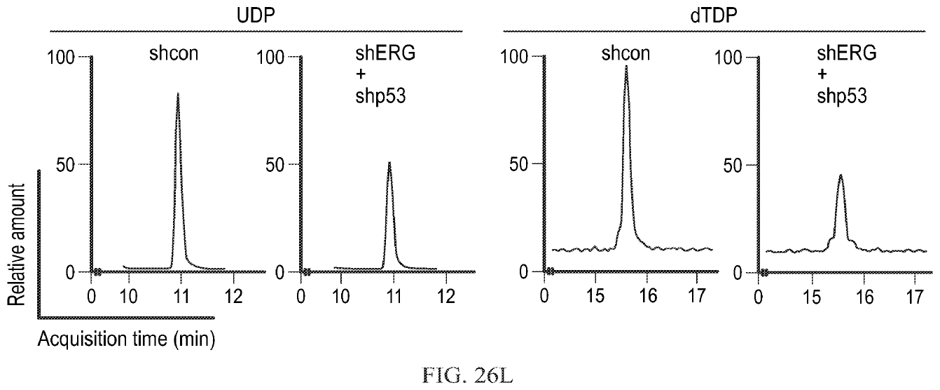

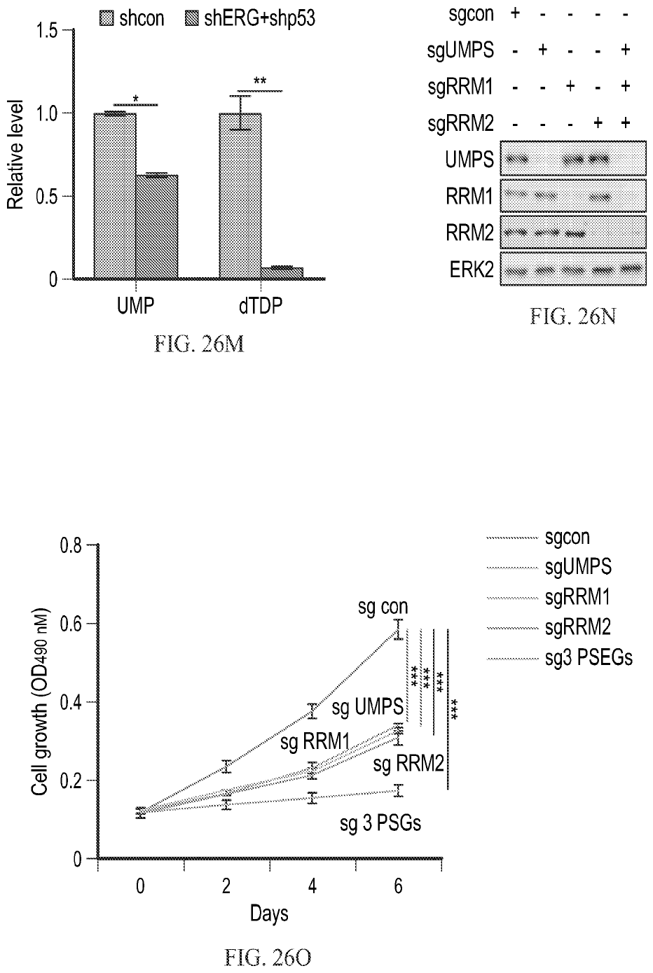

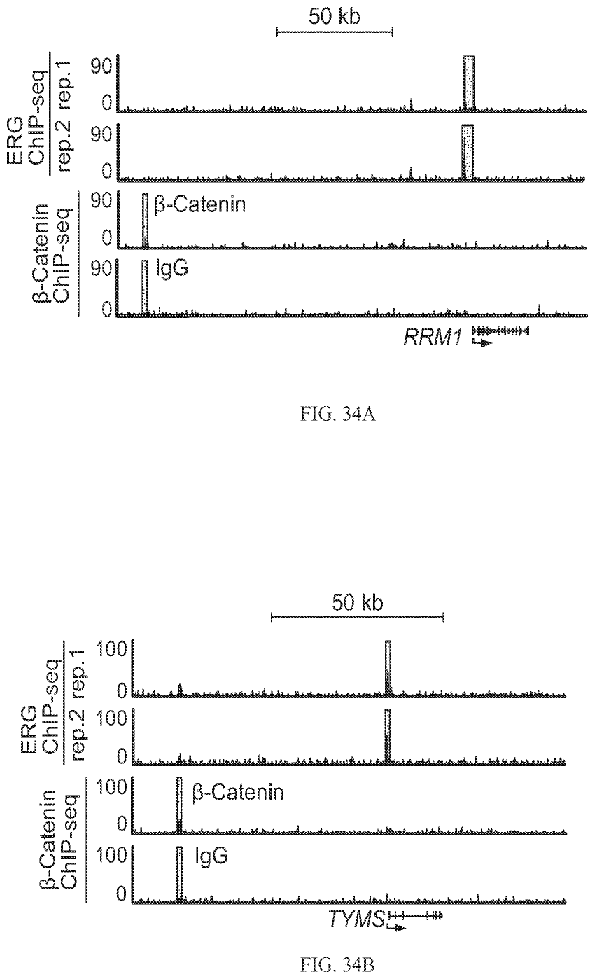

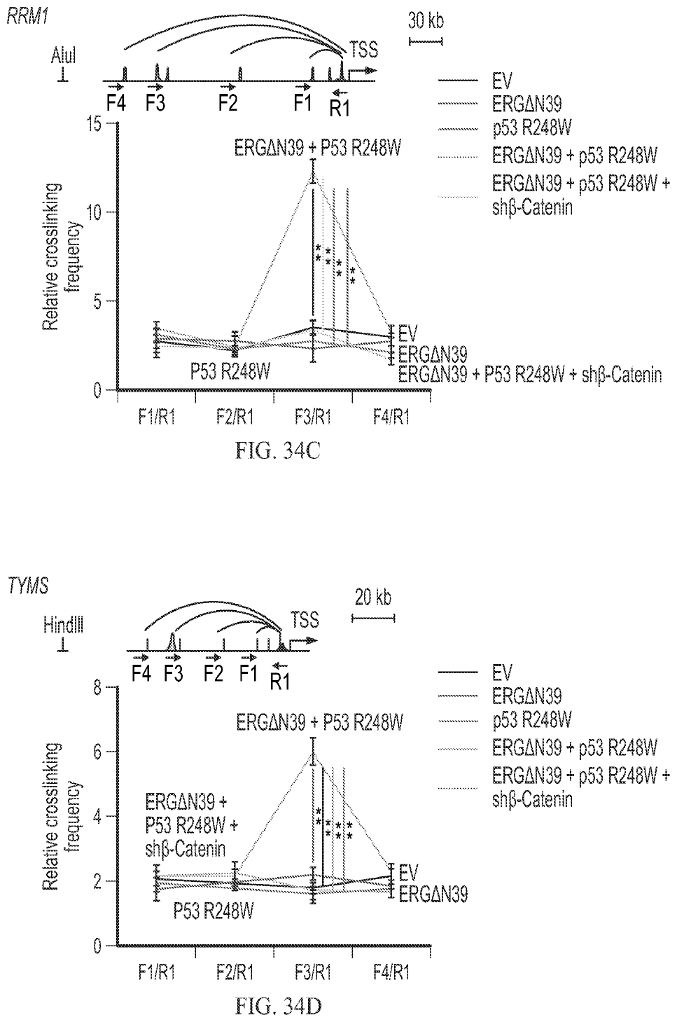

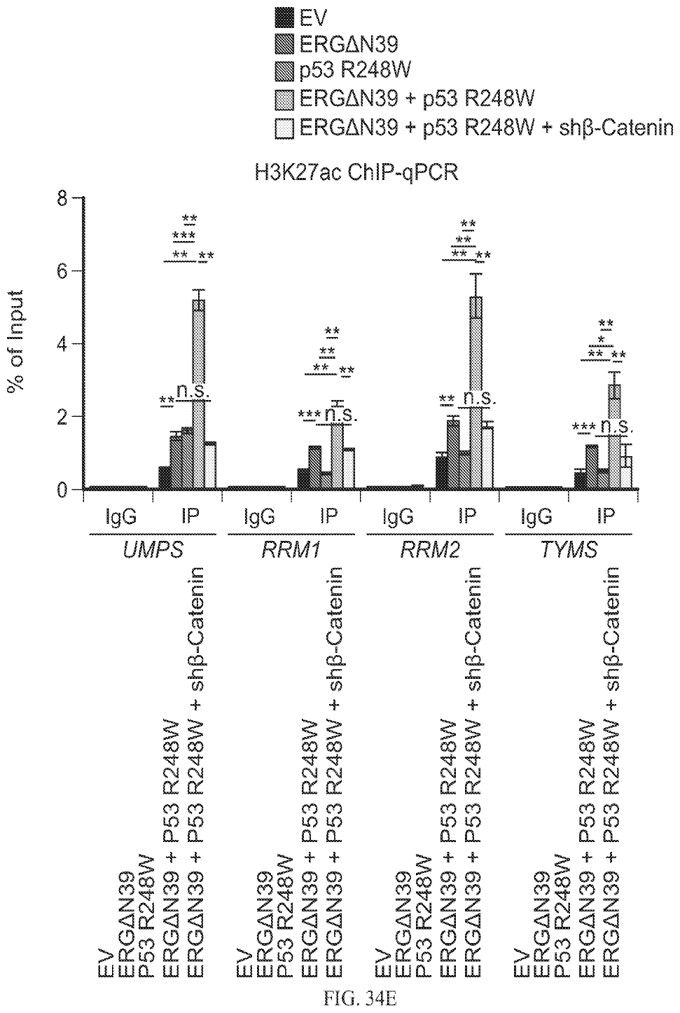

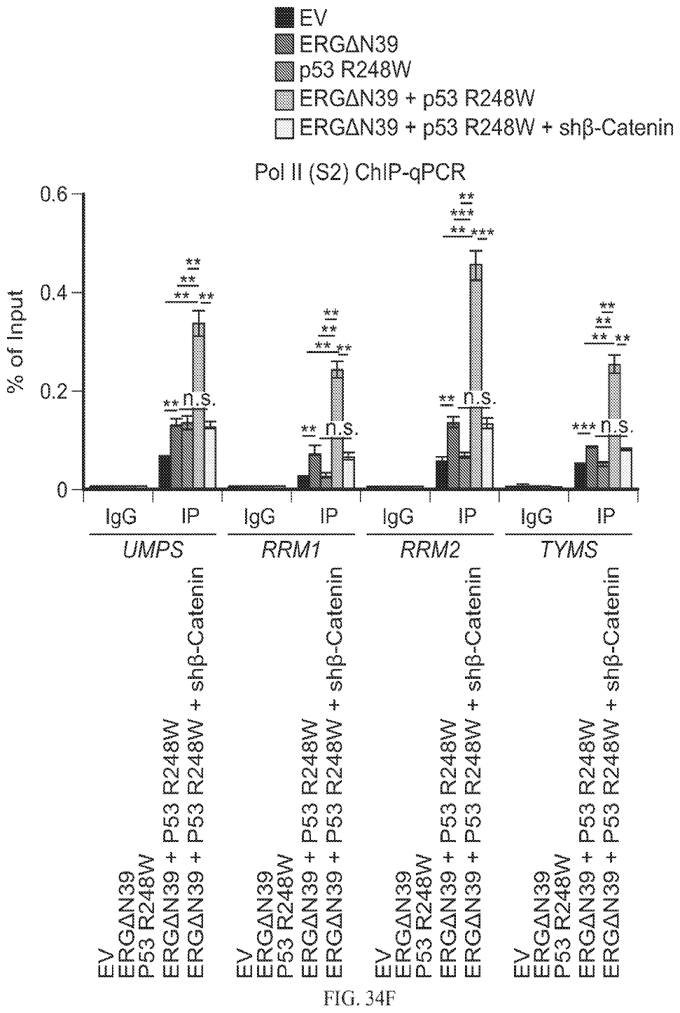

UMPS, RRM1, RRM2 and TYMS are key enzymes required for pyrimidine synthesis (FIG. 24E). Similar to the effect of ERG or p53 R248W KD, β—Catenin KD alone also inhibited expression of these PSGs at both protein and mRNA levels in VCaP cells (FIGS. 26A and 26B). ERG or p53 R248W KD failed to further decrease expression of these genes in β—Catenin deficient cells (FIGS. 26A and 26B), suggesting that β—Catenin is an essential downstream mediator of regulation of PSG expression by p53 mutant and ERG. In support of this hypothesis, ChIP-seq and ChIP-qPCR data analysis showed that both ERG and β—Catenin bound to the promoter and/or putative enhancer at these PSG loci (FIGS. 26C-26E, 34A, and 34B).

To determine the possible interaction between ERG binding in the promoter and 3—Catenin occupancy in the putative enhancer at RRM1, RRM2 and TYMS gene loci, chromatin conformation capture (3C) assay was performed. It was found that only co-expression of both ERGAN39 and p53 mutant (R248W), but not each alone substantially increased expression of these PSGs at mRNA level in p53-KO DU145 cells (FIGS. 26G and 26H) and induced spatial looping between the ERG- and β—Catenin-occupied sites in these PSG loci (FIGS. 261, 34C, and 34D). However, the effect of ERGAN39 and p53 R248W on chromatin looping and expression of these PSGs was completely reversed by β—Catenin KD (FIGS. 26G-26I, 34C, and 34D). The chromatin looping results were also consistent with enhanced enrichment of histone H3 lysine 27 acetylation (H3K27ac) and serine-2 phosphorylated RNA polymerase II (Pol II S2-p) in these loci (FIGS. 34E and 34F). These data support a hypothetical model wherein chromatin looping occurs between ERG- and β—Catenin-binding sites in the PSG loci, causing an increase in H3K27ac level, recruitment of Pol II and expression of these PSGs (FIG. 26J).

Next, the impact of ERG and p53 mutant expression on pyrimidine synthesis was determined. Endogenous ERGAN39 and p53 R248W were knocked down in VCaP cells and measured the level of UMP and dTDP, two key intermediates for pyrimidine synthesis (FIG. 24E). It was demonstrated that KD of both ERG and p53 R248W significantly decreased the level of UMP and dTDP in VCaP cells (FIGS. 26K-26M). Most importantly, depletion of UMPS, RRM1 and RRM2, three key enzymes for pyrimidine synthesis (FIG. 24E) individually or together largely inhibited VCaP cell growth (FIGS. 26N and 26O). These data indicate that increased expression of these PSGs is important for the growth of TMPRSS2-ERG/p53 mutant-positive PCa cells.

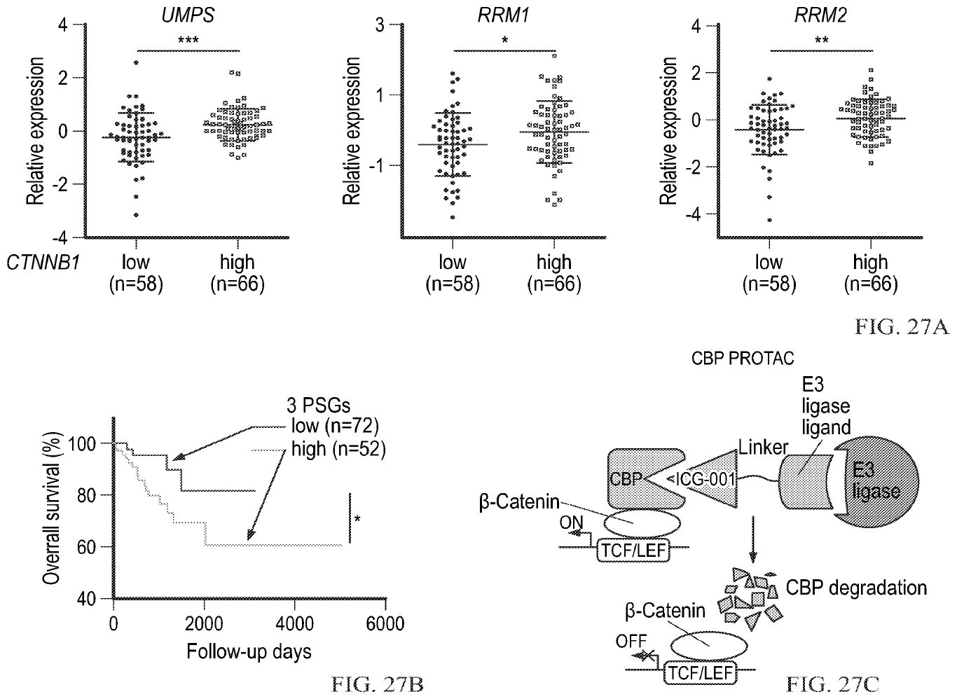

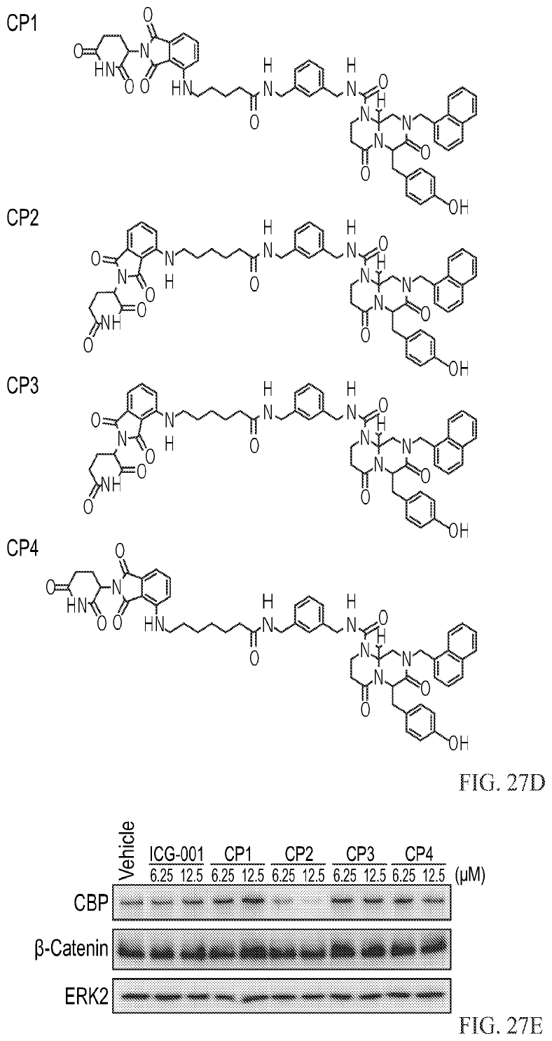

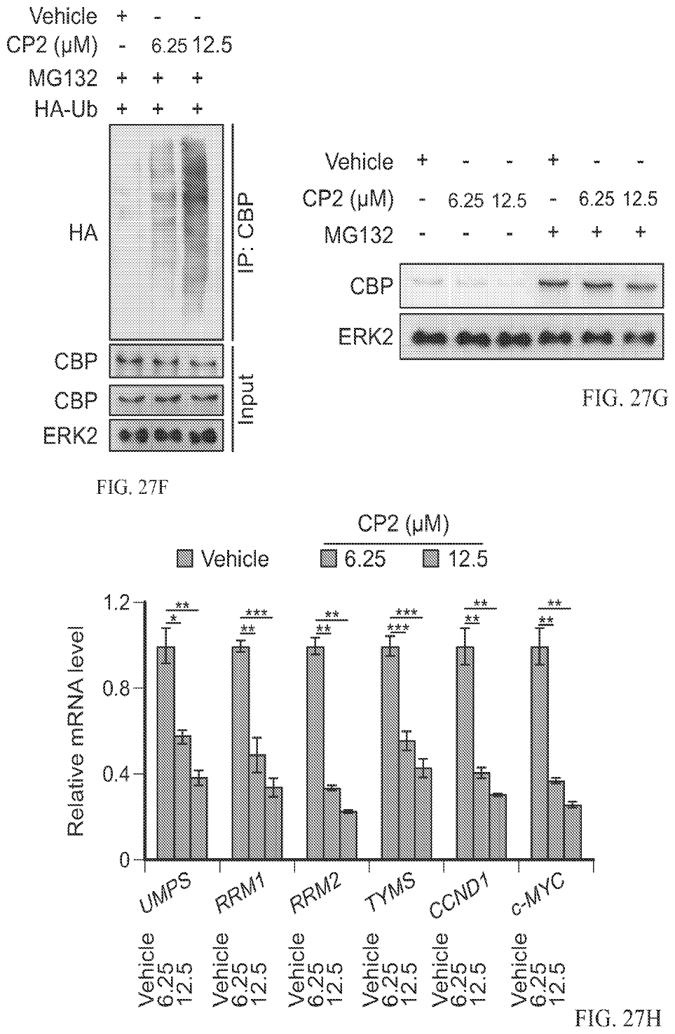

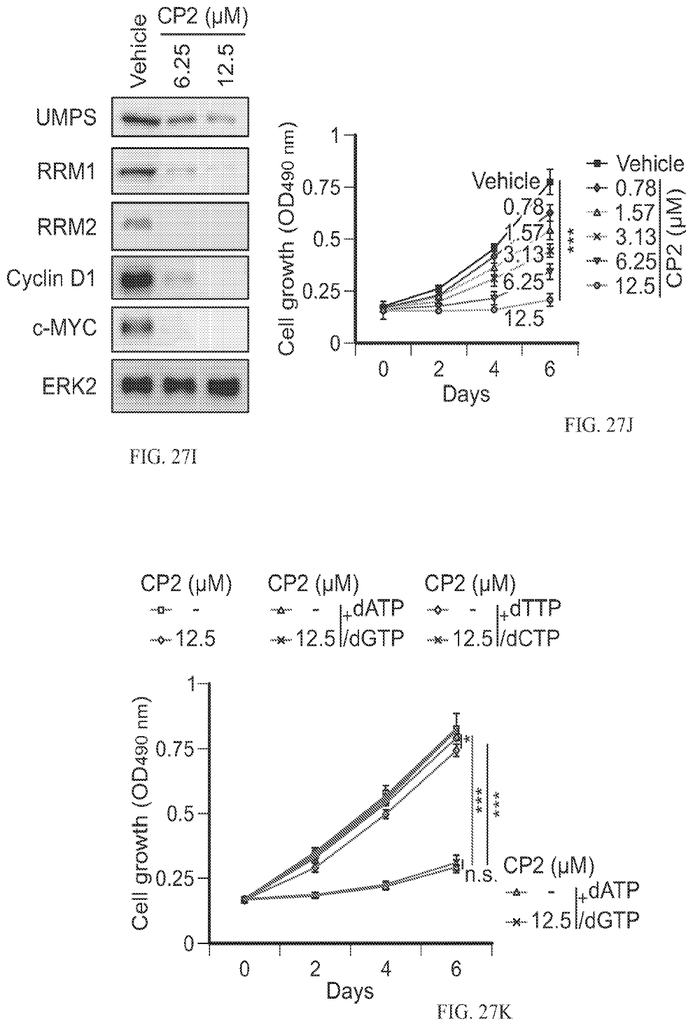

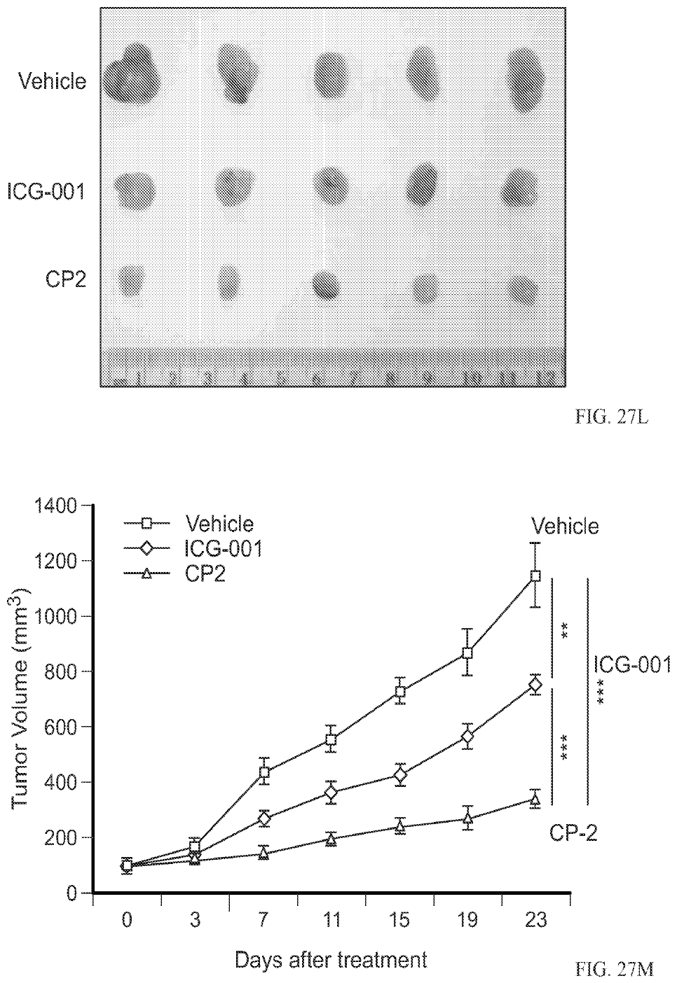

To determine the clinical relevance of co-regulation of PSGs by ERG and β—Catenin, meta-analysis of RNA-seq data was performed in the TCGA cohort of PCa. It was found that among the TMPRSS2-ERG positive patient samples CTNNB1 mRNA expression positively correlated with the levels of the key PSGs examined, including UMPS, RRM1 and RRM2 (FIG. 27A). Further analysis revealed that high level expression of these three PSGs significantly associated with poor overall survival of those patients (FIG. 27B). The data from culture cell lines and patient specimens suggest that both ERG and β—Catenin are important for the upregulation of PSGs in PCa cells.

### β—Catenin Inhibition by CBP PROTAC Suppresses PSG Expression and Tumor Growth

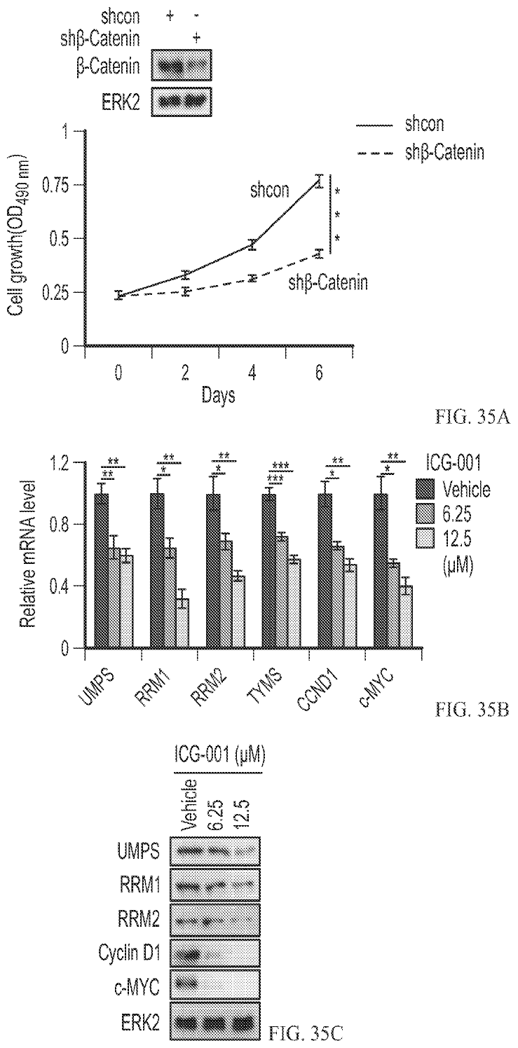

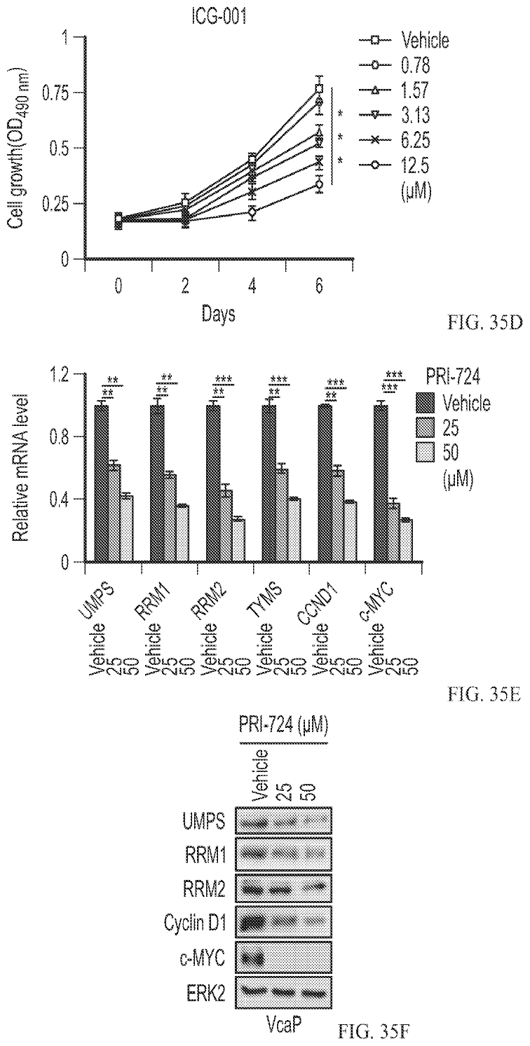

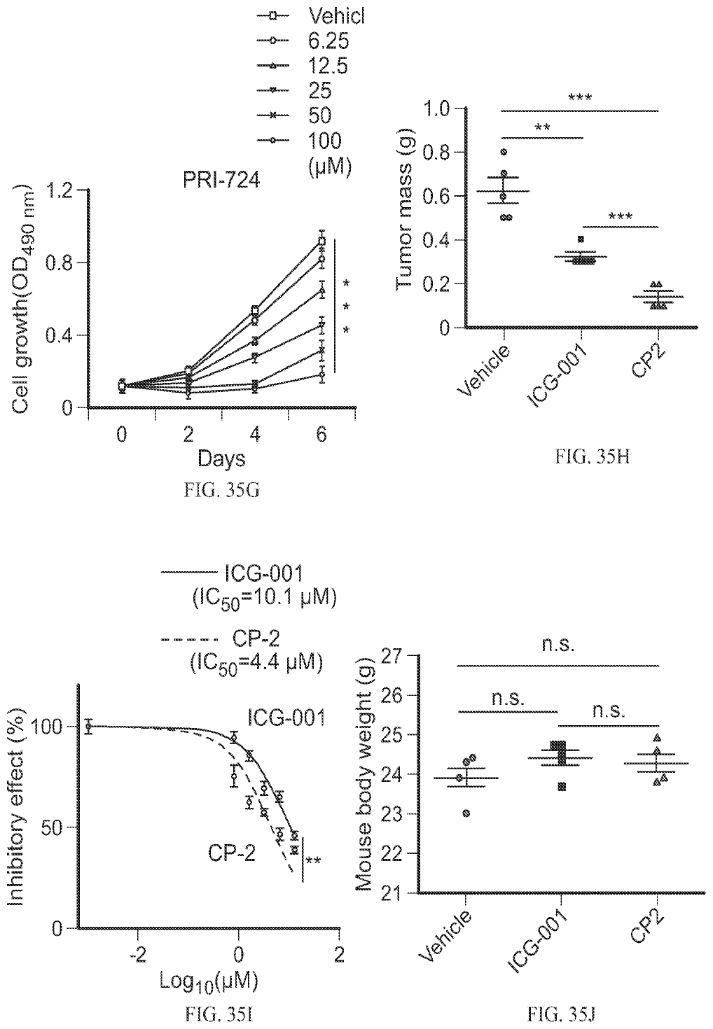

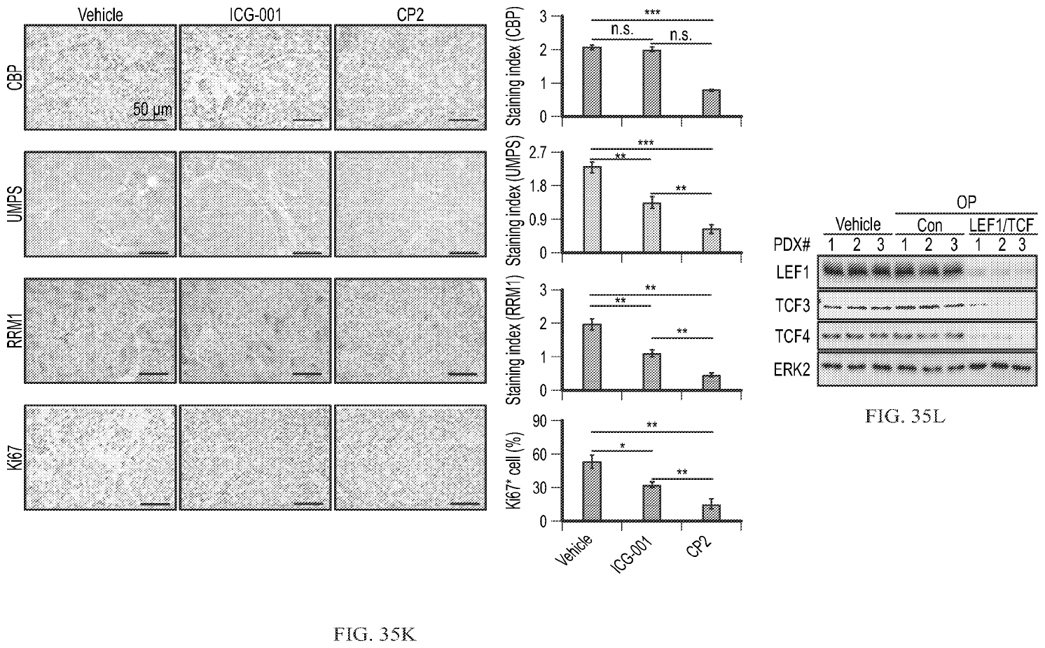

In agreement with the importance of β—Catenin in expression of PSGs in VCaP cells, it was demonstrated that β—Catenin is also required for VCaP cell growth (FIG. 35A). It was demonstrated that treatment of VCaP cells with ICG-001 decreased expression of PSGs and canonical β—Catenin target genes CCND1 and c-MYC at both mRNA and protein levels and inhibited cell growth in a dose-dependent manner (FIG. 35B-35D). PRI-724 is a pro-drug of C-82, a second-generation specific β—Catenin/CBP antagonist. Similar to the effect of ICG-001, PRI-724 treatment resulted in inhibition of expression of PSGs, CCND1 and c-MYC and growth of VCaP cells (FIGS. 35E-35G).

PROTAC technology has been developed by engineering a bifunctional small molecule chimera to induce ubiquitination and proteasomal degradation of a protein of interest (POI) by bring the POI to the proximity of an E3 ubiquitin ligase. A series of CBP PROTACs (CP1 to CP4) were synthesized by using ICG-001 as a CBP-binding ligand (FIGS. 27C and 27D). It was found that CP2 treatment effectively induced downregulation of CBP protein in VCaP cells (FIG. 27E). This effect was likely mediated by CP2-induced proteasomal degradation of CBP because CP2 treatment largely increased CBP poly-ubiquitination and the effect was blocked by the proteasome inhibitor MG132 (FIGS. 27F and 27G).

The effect of CP2 on β—Catenin target gene expression and growth of ERG/GOF p53-positive PCa cells was next examined. CP2 treatment largely decreased expression of PSGs, CCND1 and c-MYC at both mRNA and protein levels in VCaP cells (FIGS. 27H and 27I). CP2 also inhibited VCaP cell growth (FIG. 27J); however, this effect was reversed by supplement of dTTP/dCTP, but not dATP/dGTP in culture medium (FIG. 27K). These data imply that the inhibitory effect of CP2 on cell growth is largely mediated through the inhibition of pyrimidine synthesis pathway.

To evaluate the effect of CBP PROTAC on tumor growth, VCaP xenografts were generated and mice were treated with vehicle, O-Catenin/CBP small molecule inhibitor ICG-001 (positive control) or CP2. CP2 treatment inhibited growth of VCaP tumors in mice and the inhibitory effect was much greater than ICG-001 (FIGS. 27L, 27M, and 35H), consistent with the finding that IC50 of CP2 was lower than that of ICG-001 (FIG. 34I). On the contrary, treatment with CP2 or ICG-001 did not result in obvious deleterious effect on mouse body weight (FIG. 35J), indicating that the used dose of these two compounds did not induce any general toxicity in mice. In agreement with tumor growth, IHC analysis showed that CP2 treatment decreased the expression level of CBP, pyrimidine synthesis enzyme proteins such as UMPS and RRM1, and Ki67 (FIG. 35K). Taken together, these results indicate that inhibition of the pyrimidine synthesis pathway by targeting the signaling nodule β—Catenin/CBP represents a viable therapeutic option for TMPRSS2-ERG/GOF p53-positive PCa.

### Therapeutic Targeting of the β—Catenin-LEF TCF Complex in ERG GOF p53 Mutant PCa

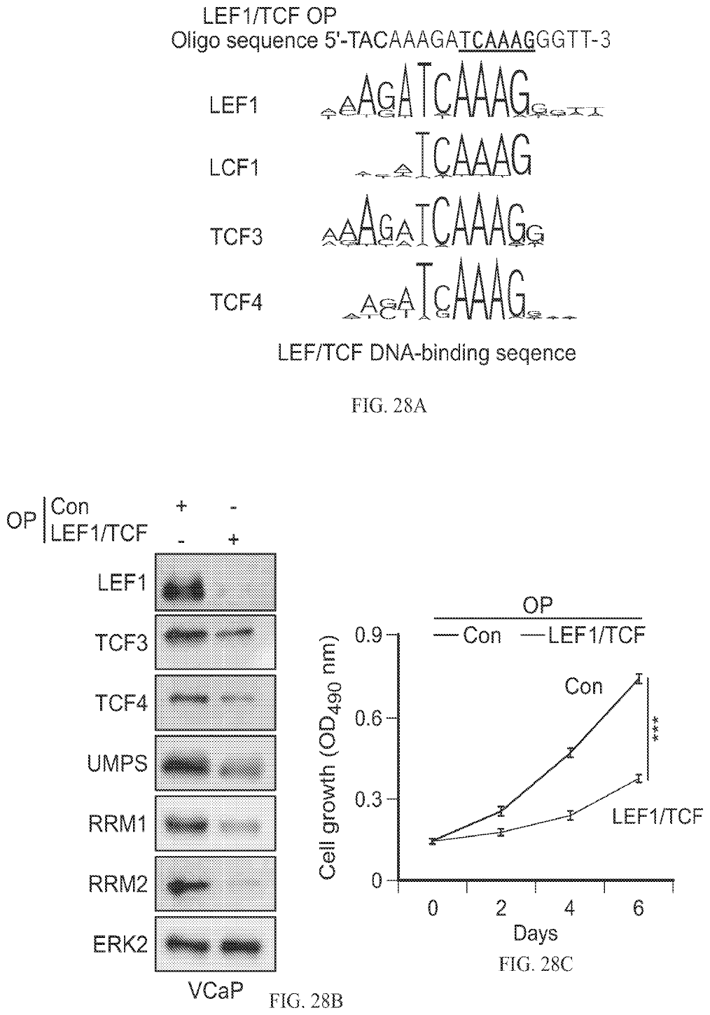

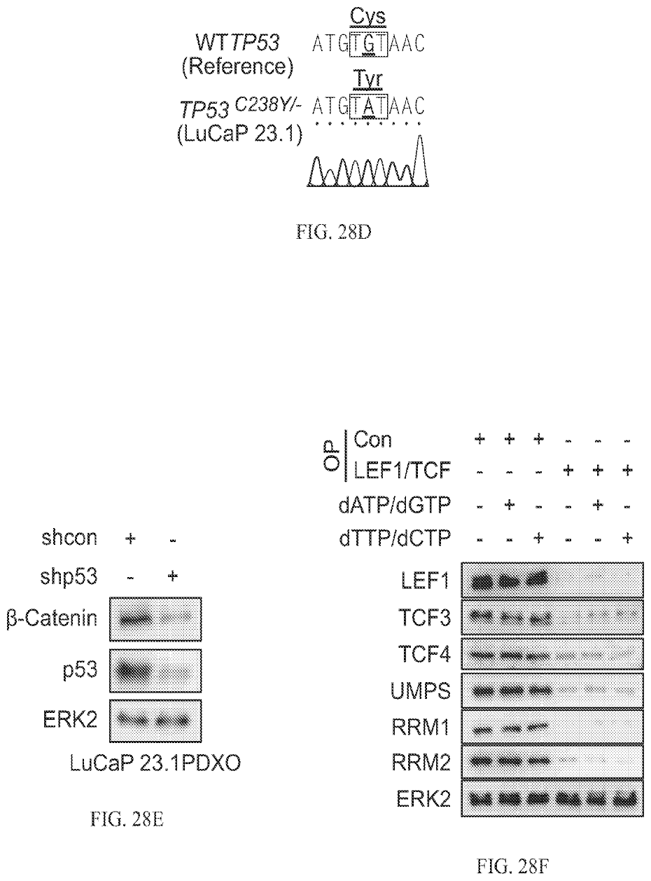

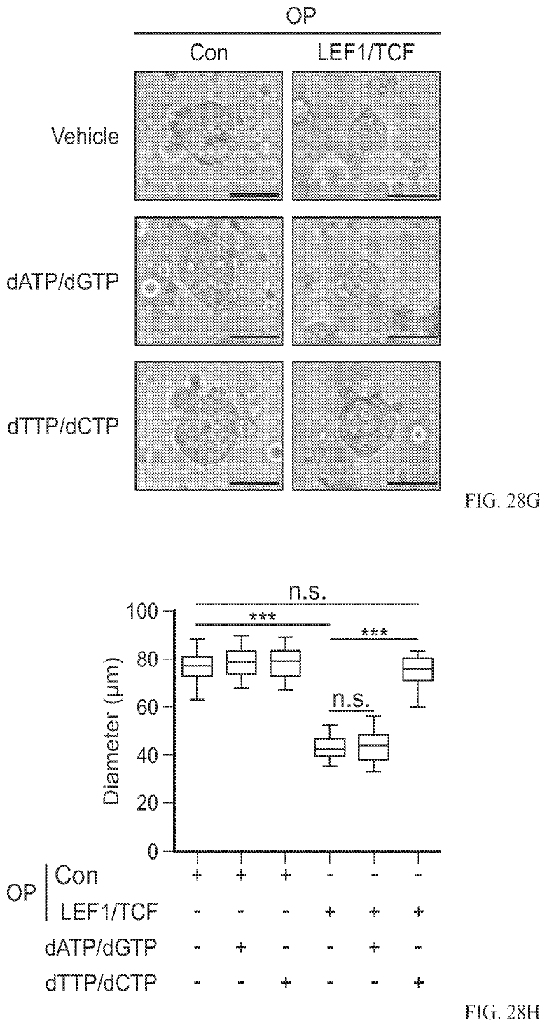

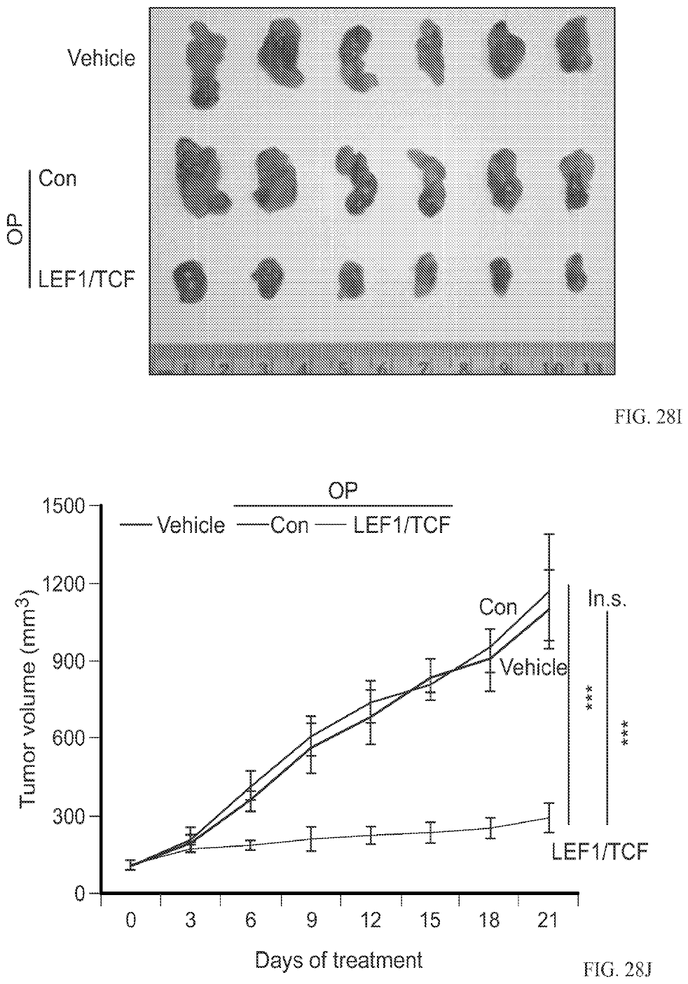

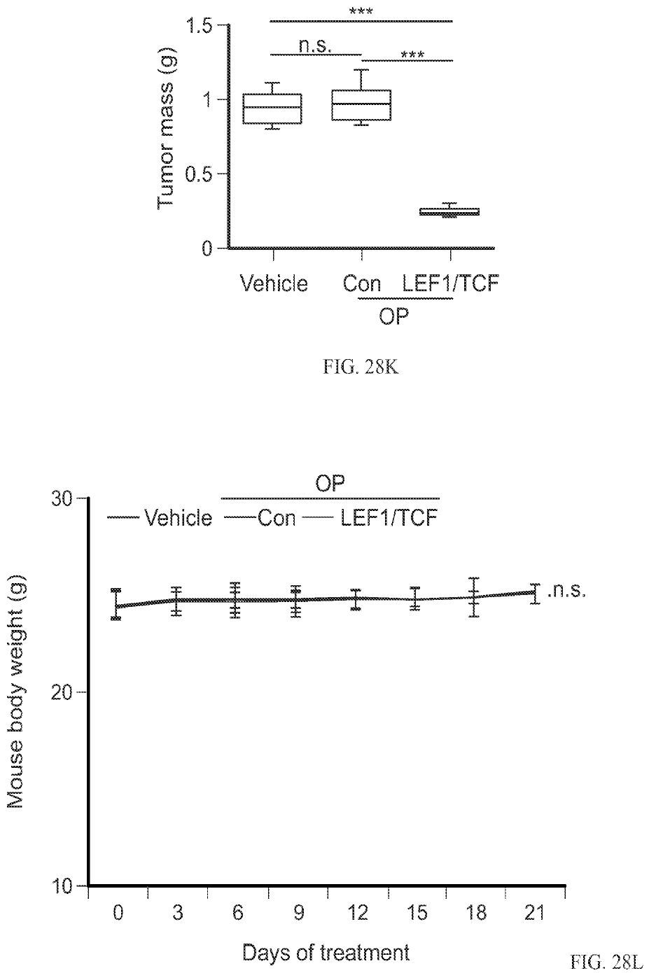

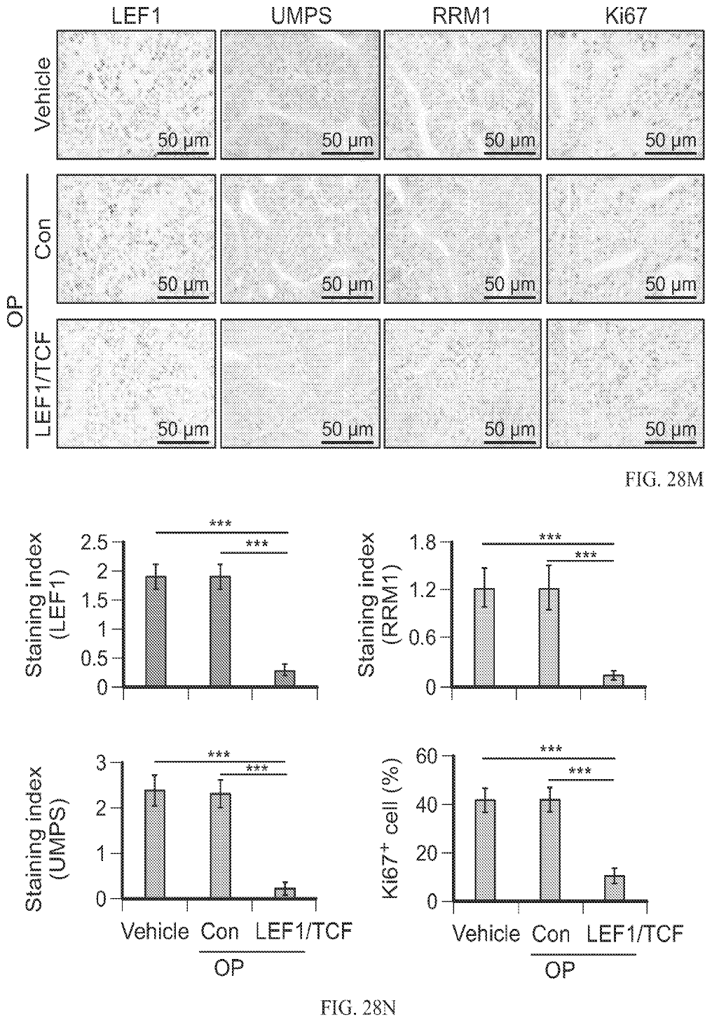

β—Catenin transactivates its target genes by forming a protein complex with DNA binding partners LEF1 and other LEF/TCF family proteins including TCF1, TCF3 and TCF4. Aberrant upregulation of β—Catenin in ERG/GOF p53 mutant PCa cells presages that this cell type represents an ideal model to test the anti-cancer efficacy of LEF1 O′PROTAC. An effective LEF1 O′PROTAC (OP-V1) almost completely ablated LEF1 protein in VCaP cells. This O′PROTAC also downregulated TCF3 and TCF4 protein to a certain degree, consistent with the observation that members of the LEF/TCF protein family bind core DNA sequences similar to TCAAAG (FIGS. 28A and 28B). TCF1 was not examined because it was hardly detected in VCaP cells, which is consistent with the genotype-tissue expression (GTEx) RNA-seq data showing that TCF1 expression is undetectable in prostatic tissues (www.proteinatlas.org/). Importantly, this LEF1/TCF OP also inhibited expression of pyrimidine synthesis enzyme proteins and growth of VCaP cells in culture (FIGS. 28B and 28C).

Next, it was sought to determine the anti-cancer efficacy of LEF1/TCF O′PROTAC using ERG/GOF p53 mutant PCa organoids and PDXs. LuCaP 23.1 PDX and its androgen-independent (castration-resistant) subline LuCaP 23.1AI are TMPRSS2-ERG positive and one allele of TP53 is deleted (Kumar et al., 2011). The parental LuCaP 23.1 PDX tumors also harbor a C238Y mutation in p53 DBD (FIG. 28D). In agreement with the EMSA result that p53 C238Y mutant bound to MP53BS in the CTNNB1 protomer (FIG. 25I), p53 KD largely decreased β—Catenin protein expression in LuCaP 23.1 PDX-derived organoids (PDXO) (FIG. 28E), highlighting that LuCaP 23.1 is an ideal model system to test anti-cancer efficacy of inhibition of the O-Catenin-LEF/TCF pathway.

It was demonstrated that LEF1/TCF OP treatment not only inhibited protein expression of key pyrimidine synthesis enzymes, but also effectively decreased growth of LuCaP 23.1 PDXO (FIGS. 28F-28H). Most importantly, this effect was almost completely reversed by supplementation of dTTP/dCTP, but not dATP/dGTP (FIGS. 28G and 28H), suggesting that the anti-cancer effect of LEF1/TCF OP is largely mediated through the inhibition of pyrimidine synthesis. Compared to the effect of control OP or vehicle, treatment of LEF1/TCF OP markedly blocked growth of LuCaP 23.1 PDX tumors without causing any obvious reduction in body weight of mice (FIGS. 28I-28L). Immunohistochemistry (IHC) and Western blot analyses showed that LEF1/TCF OP not only decreased expression of LEF1 and other LEF/TCF proteins and the pyrimidine synthesis enzymes such as UMPS and RRM1, but also largely reduced the number of Ki67-positive cells (FIGS. 28M, 28N, and 35L). These results indicate that inhibition of β—Catenin and PSG expression by targeting LEF/TCF proteins using O′PROTAC can effectively block growth of PCa with TMPRSS2-ERG fusion and GOF p53 mutation.

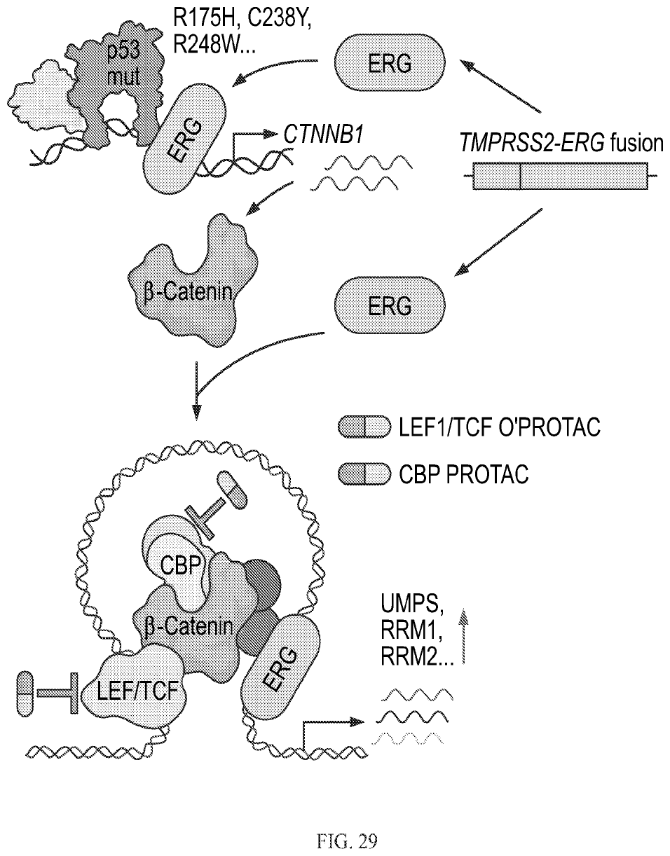

Together, these results demonstrate that β—Catenin may be a therapeutic target of ERG/GOFG p53 mutant PCa (FIG. 29). For example, inhibiting β—Catenin using CBP PROTAC and/or LEF1/TCF O′PROTAC can be effective to treat ERG/GOF p53-positive PCa, as well as other cancer types such as the hematologic malignancies and solid tumors expressing GOF p53 mutant protein.

### Experimental Model and Subject Details

### Cell and Organoid Culture

VCaP, DU145, LNCaP, and 293T cells were purchased from American Type Culture Collection (ATCC). DU145 and LNCaP cells were cultivated in RPMI 1640 media (Corning) with 10% fetal bovine serum (FBS) (Gbico). VCaP and 293T cells were grown in DMEM media (Corning) supplemented with 10% FBS (Millipore). All the cells were incubated at 37° C. supplied with 5% CO₂. Cells were treated with plasmocin (Invivogene) to eradicate mycoplasma in prior to the subsequent experiments.

Organoids were generated from LuCaP 23.1 patient-derived xenografts (PDXs) using the methods as described (Drost et al., 2016). Briefly, organoids were cultured in 40 μL Matrigel (Sigma) mixed with FBS-free DMEM/F-12 medium supplemented with other factors.

### Transfection and Lentivirus Infection

Cells were transiently transfected with indicated plasmids using either Lipofectamine 2000 (Thermo Fisher Scientific) or polyethylenimine (PEI) (Polysicences, 23966) according to the manufactures' instructions. For lentivirus package, 293T cells were co-transfected with plasmids for psPAX2, pMDG.2 and shRNA using Lipofectamine 2000. Supernatant containing virus was harvested after 48 hours and added into cells after filtration by 0.45 m filter (Millipore). The indicated cells were added with the virus-containing supernatant in the presence of polybrene (5 μg/mL) (Millipore) and selected with 1 μg/mL puromycin (Selleck).

### Cell Growth Assay

VCaP cells were seeded at the density of 5,000 cells per well in 96-well plate overnight. At the indicated time points, optical density (OD) of cells was measured by microtiter reader (Biotek) at 490 nanometer after incubation with MTS (Promega) for 2 hours at 37° C. in a cell incubator. For the treatment with CP-2, ICG-001 or PRI-724, cells were seeded in 96-well plate overnight followed by adding indicated compounds. OD values were measured at the indicated time points.

### Genetically Engineered Mouse Model and Genotyping

The indicated groups of target and control mice were generated by crossing the following mice: Probasin (Pb)-driven Cre4 recombinase transgenic mice, acquired from the National Cancer Institute (NCI) Mouse Repository; transgenic ERG mice purchased from the Jackson Laboratory (Cat #010929); Trp53 loxp/loxp conditional mice, acquired from the NCI Mouse Repository; and Trp53 loxp-STOP-loxp-R172H conditional mice, acquired from the NCI Mouse Repository. PCR genotyping primers are listed in Table 6.

### Hematoxylin and Eosin (H&E) Staining and Immunohistochemistry (IHC)

Four-μm sections were cut consecutively from formalin-fixed paraffin-embedded (FFPE) prostate tissues of indicated mice. Tissues were deparaffinized by xylene and subsequently rehydrated in turn through 100%, 95%, and 70% ethanal and water. After hematoxylin staining and Scott's Bluing solution (40.1 g MgSO₄-7 H₂O, 2 g sodium hydrogen carbonate, 1 L H₂O) washing, tissues were counterstained with 1% eosin. After washing with 95% ethanol, tissues were dehydrated with 95% and 100% ethanol. Finally, the stained tissue was put in xylene and mounted with coverslips.

For IHC, tissues were rehydrated, endogenous peroxidase activity was destroyed, and antigens were retrieval. Antibodies for IHC as following: anti-AR (ab108341, Abcam), anti-ERG (ab92513, Abcam), anti-Ki67 (ab15580), anti-UMPS (NOVUS, #85896), anti-RRM1 (Cell signaling technology, #8637), anti-CBP (Santa Cruz Biotechnology, sc-583), anti-LEF1 (Cell signaling technology, #2230S). For quantification, the staining score was determined by multiplying the percentage of positive cells and the intensity ranged from 1 (weak staining), 2 (median staining), and 3 (strong staining). For Ki67 quantification, cells with positive staining in the nucleus were included to calculate the percentage of Ki67 positive-staining cells.

### RNA Extraction and RT-qPCR

The total RNA was extracted from cultured cells or organoids using Trizol reagent (Thermo Fisher Scientific) according to the manufacturer's instructions. Complementary DNA was synthesized using reverse transcriptase (Promega). mRNA expression level was determined by real-time quantitative PCR (qPCR) using SYBR Green Mix (Thermo Fisher Scientific) with the realtime PCR system (Bio-Rad). Relative gene expression was normalized to the expression of house-keeping gene Actin Beta (ACTB). Primer sequences used for qPCR are listed in Table 15.

TABLE 15
Oligonucleotides involved in the study related to the STAR Method.
The Primers for the mouse genotyping.

| | Forward(F)/ | | |
|---|---|---|---|
| Gene name | Reverse(R) | Sequence (5′-3′) | SEQ ID NO |
| Cre | F | TTGCCTGCATTACCGGTCGAT | 271 |
| Cre | R | GATCCTGGCAATTTCGGCTAT | 272 |
| Tg-ERG | F-1# | AACGAGCGCAGAGTTATCGT | 273 |
| Tg-ERG | F-2# | CTAGGCCACAGAATTGAAAGATCT | 274 |
| Tg-ERG | R-1# | GTGAGCCTCTGGAAGTCGTC | 275 |
| Tg-ERG | R-2# | GTAGGTGGAAATTCTAGCATCATCC | 276 |
| Trp53 loxp | F | CACAAAAACAGGTTAAACCCAG | 277 |
| Trp53 loxp | R | AGCACATAGGAGGCAGAGAC | 278 |
| Trp53 loxp-STOP-loxp-R172H | F (WT) | GTAGTACTGTTCGTTCCATTCCG | 279 |
| Trp53 loxp-STOP-loxp-R172H | F (loxp) | AGCTAGCCACCATGGCTTGAGTAAGTCTGCA | 280 |
| Trp53 loxp-STOP-loxp-R172H | R | CTTGGAGACATAGCCACACTG | 281 |

The oligonucleotides sequence of shRNAs.

| | Sequence | |
|---|---|---|
| shRNA name | (5′-3′) | |
| shcontrol (shcon) | CCGGCAACAAGATGAAGAGCACCAACTCGAGTTGGT | 282 |
| | GCTCTTCATCTTGTTGTTTTT | 283 |
| shERG#1 | CCGGGCCCATCAACAGACGTTGATACT | 284 |
| | CGAGTATCAACGTCTGTTGATGGGCTTTTT | 285 |
| shERG#2 | CCGGGCTCATATCAAGGAAGCCTTACTCGAGTAA | 286 |
| | GGCTTCCTTGATATGAGCTTTTT | 287 |
| shp53#1 | CCGGCGGCGCACAGAGGAAGAGAATCTCGA | 288 |
| | GATTCTCTTCCTCTGTGCGCCGTTTTT | 289 |
| shp53#2 | CCGGGTCCAGATGAAGCTCCCAGAACTCGAGTTCTG | 290 |
| | GGAGCTTCATCTGGACTTTTT | 291 |
| shß-Catenin#1 | CCGGTCTAACCTCACTTGCAATAATCTCGAGATTATT | 292 |
| | GCAAGTGAGGTTAGATTTTTG | 293 |
| shß-Catenin#2 | CCGGTTGTTATCAGAGGACTAAATACTCGAGTATTTAGT | 294 |
| | CCTCTGATAACAATTTTTG | 295 |

RT-qPCR primers.

| | Forward(F)/ | | |
|---|---|---|---|
| Gene name | Reverse(R) | Sequence (5′-3′) | |
| mouse Umps | F | GTCTTCTCAGTCAGGTCGCA | 296 |
| mouse Umps | R | GAGCATGGGAATGTGATTGGC | 297 |
| mouse Rrm1 | F | GCCGAGAGAGGTGCTTTCAT | 298 |
| mouse Rrm1 | R | AAACCCTGCTTCCAACCGTA | 299 |
| mouse Rrm2 | F | GGATTCCAGCTGTTTTCGCC | 300 |
| mouse Rrm2 | R | GGGCGTGTTCTCCTTGTCAG | 301 |
| mouse Tyms | F | TTGGGATTTTCTGCCCGACA | 302 |
| mouse Tyms | R | CTCCTTGTCCCGAGTAATCTGA | 303 |
| mouse Actb | F | AGAAGCTGTGCTATGTTGCTCTA | 304 |
| mouse Actb | R | ACAGGATTCCATACCCAAGAAGGA | 305 |
| CTNNB1 | F | AGGTCTGAGGAGCAGCTTCA | 306 |
| CTNNB1 | R | CAAATACCCTCAGGGGAACAGG | 307 |
| ERG | F | AAGCGCTACGCCTACAAGTT | 308 |
| ERG | R | TTCATCTTCTGTGGGTGGGC | 309 |
| TP53 | F | TGCTCAAGACTGGCGCTAAA | 310 |
| TP53 | R | CAGTCTGGCTGCCAATCCA | 311 |
| UMPS | F | GAGCAGCGGTTAGAATGGC | 312 |
| UMPS | R | TCCTCCTGCTTCCAACTGAAC | 313 |
| RRM1 | F | TCCTGCTCAGATCACCATGAAA | 314 |
| RRM1 | R | GGCTGCCAGGATAGCATAGTC | 315 |
| RRM2 | F | CTGGAGTGAGGGGTCGC | 316 |
| RRM2 | R | GCGGCGTGTTCTCCTTGT | 317 |
| TYMS | F | GAGCTGTCTTCCAAGGGAGT | 318 |
| TYMS | R | CAACTCCCTGTCCTGAATAATCTGA | 319 |
| ACTB | F | AGCACAGAGCCTCGCCTTT | 320 |
| ACTB | R | ATCACGCCCTGGTGCCT | 321 |

ChIP-seq primers.

| | Forward(F)/ | | |
|---|---|---|---|
| Gene name/ChIP | Reverse(R) | Sequence (5′-3′) | |
| CTNNB1/p53 | F | GACTACTTTCCACCGCCCCC | 322 |
| CTNNB1/p53 | R | TAAAATGGCGCCGCACAAGG | 323 |
| Upstream(−3000 bp)/p53 | F | GTTGCAGCTTCGACAAACGTCA | 324 |
| Upstream(−3000 bp)/p53 | R | AGCTATCGATTAAGCAGCCTCCA | 325 |
| CTNNB1-a/p53 | F | CACCCCGGGGAGCGTC | 326 |
| CTNNB1-a/p53 | R | GGTGGAAAGTAGTCCCCGCG | 327 |
| CTNNB1-b/p53 | F | GCCCCCTCGCGCCCC | 328 |
| CTNNB1-b/p53 | R | GAGCTCTTATAAGTCGCGCAGAAGCCG | 329 |
| CTNNB1-c/p53 | F | CTTGTGCGGCGCCATTTTAA | 330 |
| CTNNB1-c/p53 | R | TCAGACCTTCCTCCGTCTCC | 331 |
| UMPS/ERG | F | CAAGCCGGGAAAGCTGCAG | 332 |
| UMPS/ERG | R | TGCTTCAAATTCCCAGGCGC | 333 |
| RRM1/ERG | F | CTGACCCAGCGGGCTCTAG | 334 |
| RRM1/ERG | R | ATATGGACATGCCCGGCGG | 335 |
| RRM2/ERG | F | AAGTCGCGCTAACCTTGGCC | 336 |
| RRM2/ERG | R | CTCCTCTGCATTCCCAGCCT | 337 |
| TYMS/ERG | F | CTCAGCTGTGGCCCTGGG | 338 |
| TYMS/ERG | R | TCTTCCTGCTCGGCGGG | 339 |
| UMPS/ß-Catenin | F | CCAGGAGAAGCACAAACTGGC | 340 |
| UMPS/ß-Catenin | R | GAAGTCCCGCCTCTTCCGC | 341 |
| RRMI/ß-Catenin | F | GCAAGAGGTAGAGAGGTGACCTG | 342 |
| RRMI/ß-Catenin | R | GCTGTGGTTGTGACGCCTTTTAG | 343 |
| RRM2/ß-Catenin | F | ATCGGAGGACCCCAGAAGAC | 344 |
| RRM2/ß-Catenin | R | GGCACCACTTACTATGCCCC | 345 |
| TYMS/ß-Catenin | F | GCCCACATTCCTTCCTGACG | 346 |
| TYMS/ß-Catenin | R | CGGGACCTGCAGGTGACG | 347 |
| UMPS/pol II Ser2 | F | AGACAGCCACAGTCCTGTCTG | 348 |
| UMPS/pol II Ser2 | R | CTGCACTCCATCCTGGGC | 349 |
| RRM1/pol II Ser2 | F | GGACAAGACCAGCGGCTAATC | 350 |
| RRM1/pol II Ser2 | R | GAGCACACCATGGCTGCTG | 351 |
| RRM2/pol II Ser2 | F | GGCGAGTATCAGAGGATGGGA | 352 |
| RRM2/pol II Ser2 | R | GGTGTGGCTAGTTGGTAACACTT | 353 |
| TYMS/pol II Ser2 | F | TTAGGGGTTGGGCTGGATGC | 354 |
| TYMS/pol II Ser2 | R | CATTTGCCAGTGGCAACATCC | 355 |

The probe sequence of EMSA.

| | Forward(F)/ | | |
|---|---|---|---|
| Gene name | Reverse(R) | Sequence (5′-3′) | |
| CTNNB1-probe1 | F | GCCCCCTCGCGCCCCGCCCCTTGTC | 106 |
| CTNNB1-probel | R | GACAAGGGGCGGGGCGCGAGGGGGC | 356 |
| CTNNB1-probe2 | F | CTCGCGCGGCGGAACGCTCCGCGCT | 357 |
| CTNNB1-probe2 | R | AGCGCGGAGCGTTCCGCCGCGCGAG | 358 |
| CTNNB1-probe3 | F | GCGCCGGTGGCGGCAGGATACAGCG | 359 |
| CTNNB1-probe3 | R | CGCTGTATCCTGCCGCCACCGGCGC | 360 |
| CTNNB1-probe4 | F | GCTTCTGCGCGACTTATAAGAGCTC | 361 |
| CTNNB1-probe4 | R | GAGCTCTTATAAGTCGCGCAGAAGC | 362 |

The oligonucleotides sequence of sgRNAs.

| | Forward(F)/ | | |
|---|---|---|---|
| Gene name | Reverse(R) | Sequence (5′-3′) | |
| sgUMPS-1 | F | CACCGCCGCAGATCGATGTAGATGG | 363 |
| sgUMPS-1 | R | AAACCCATCTACATCGATCTGCGGC | 364 |
| sgUMPS-2 | F | CACCGGCCCCGCAGATCGATGTAGA | 365 |
| sgUMPS-2 | R | AAACTCTACATCGATCTGCGGGGCC | 366 |
| sgUMPS-3 | F | CACCGCCCCGCAGATCGATGTAGAT | 367 |
| sgUMPS-3 | R | AAACATCTACATCGATCTGCGGGGC | 368 |
| sgRRM1-1 | F | CACCGGTAATCCAAGGCTTGTACAG | 369 |
| sgRRM1-1 | R | AAACCTGTACAAGCCTTGGATTACC | 370 |
| sgRRM1-2 | F | CACCGGTCAGGGTGCTTAGTAGTCA | 371 |
| sgRRM1-2 | R | AAACTGACTACTAAGCACCCTGACC | 372 |
| sgRRM1-3 | F | CACCGCAAGCCTTGGATTACTTTCA | 373 |
| sgRRM1-3 | R | AAACTGAAAGTAATCCAAGGCTTGC | 374 |
| sgRRM2-1 | F | CACCGGGGGCTCAGCTTGGTCGACA | 375 |
| sgRRM2-1 | R | AAACTGTCGACCAAGCTGAGCCCCC | 376 |
| sgRRM2-2 | F | CACCGCTTGGTCGACAAGGAGAACA | 377 |
| sgRRM2-2 | R | AAACTGTTCTCCTTGTCGACCAAGC | 378 |
| sgRRM2-3 | F | CACCGGCCGCTGAAGGGGCTCAGCT | 379 |
| sgRRM2-3 | R | AAACAGCTGAGCCCCTTCAGCGGCC | 380 |

The oligonucleotides sequence for TP53 cDNA generation from LuCaP 23.1

| | | | |
|---|---|---|---|
| TP53-PDX | F | ATGGAGGAGCCGCAGTCAGATCCT | 381 |
| TP53-PDX | R | TCAGTCTGAGTCAGGCCCTTCTGTCTT | 382 |

### Co-Immunoprecipitation (Co-IP) Assay

VCaP cells were collected after treated with CP2 at the indicated concentration for 24 hours and 20 μM MG132 (Millipore) for another 8 hours. After washing, cells were lysed in IP buffer (0.5% NP-40, 20 mM Tris-HCl, pH=8.0, 10 mM NaCl, 1 mM EDTA) with protease inhibitor (Sigma). Anti-CBP antibodies were added into cell lysate and incubated with Protein A/G beads (Millipore) overnight. Beads were washed and boiled with protein loading dye (Bio-Rad) for the further analysis by western blot.

### GST Tagged Recombinant Protein Purification

GST-tagged p53 expression plasmids, including wild type (WT) and mutated p53, were transformed into E. coli BL21. The successful transformed BL21 were cultured in flasks in an incubator shaker and treated with 100 μM IPTG (Sigma) at 18° C. overnight. The induced BL21 were collected and resuspended in lysis buffer (50 mM Tris-HCl, pH 8.0) with protease inhibitor (Sigma) and sonicated. Glutathione Agarose (Thermo Fisher Scientific) were added to enrich the GST-p53 (WT/mutants) protein. The 10 mM reduced glutathione (Sigma) in 50 mM Tris-HCl, pH 8.0 was added and incubated with agarose for 1 hour at room temperature. The competed protein was collected by centrifuge and saved at −80° C. for further use.

### Nuclear Extraction and Electrophoretic Mobility Shift Assay (EMSA)

Double-stranded DNA oligonucleotides were labeled with biotin as probes by using the commercial kit (Thermo Fisher Scientific, Cat #89818) before use. The labeled probes were incubated with nuclear extraction prepared from VCaP cells using NE-PER™ Nuclear and Cytoplasmic Extraction Reagents (Thermo Fisher Scientific, Cat #78833) or purified GST-p53 protein according to the protocol provided by the manufacture (Thermo Fisher Scientific, Cat #20148). For supershift assay, anti-p53 antibodies were added into the cell nuclear extract mixed with the biotin-labeled probes and the mixture were incubated with for 1 hour at room temperature before loading into 6% of non-denatured polyacrylamide gel.

### RNA-Seq Analysis

Prostate glands from mice were dissected and collected for RNA extraction by RNeasy Plus Mini Kit (Qiagen). The extracted RNA was subjected to the sequencing in Genome Analysis Core at Mayo Clinic. High quality total RNA with RNA integrity number >9.0 was used to generate the RNA-seq library by using the TruSeq RNA Sample Prep Kit v2 (Illumina). RNA samples from biological triplicates were sequenced by Illumina HiSeq 4000 following manufacture's protocol. Paired-end raw reads were subjected to the alignment of the mouse reference genome (GRCm38/mm10) using RNA-seq spliced read mapper STAR (v2.7.7a). Gene raw and normalized read counts were performed using RSeQC package (v2.3.6). Differential gene expression analysis was carried out by using DESeq2 (version 1.30.1). The false discovery rate (FDR) threshold 0.001 was applied to obtain the differentially genes.

### Chromatin Immunoprecipitation (ChIP) and ChIP-qPCR

VCaP cells were fixed and subjected to sonication by Bioruptor (Diagenode). The supernatant was obtained and added by protein A/G beads and anti-p53 or anti-ERG antibodies. After incubation overnight, beads were washed, and the complex containing DNA was eluted at 65° C. The elution was further treated with RNAase and proteinase K. Enriched DNA was extracted for high throughput sequencing or quantitative PCR.

For the ChIP-seq assay, sequencing libraries were prepared, and high-throughput sequencing was performed by Illumina HiSeq 4000 platform. The raw reads were subjected to the human reference genome (GRCh37/hg38) using bowtie2 (version 2.2.9). MACS2 (version 2.1.1) was run to perform the peak calling with a p value threshold of 1×10⁻⁵. BigWig files were generated for visualization using the UCSC Genome Browser. The assignment of peaks to potential target genes was performed by the Genomic Regions Enrichment of Annotations Tool (GREAT). ERG ChIP-seq data generated from the mouse prostate tissue was downloaded from NCBI Gene Expression Omnibus (GEO) with accession number GSE47119. β—Catenin ChIP-seq data was downloaded from GEO with accession number GSE53927, p53 ChIP-seq data of breast cancer cell lines was downloaded from GEO with accession number GSE59176.

### Chromosome Conformation Capture (3C) Assay

The 3C assay was carried out as described elsewhere (see, e.g., Hagege et al., Nature Protocols, 2:1722-1733 (2007)). Briefly, cells were crosslinked and lysed. Chromation was digested with the indicated restriction enzymes. After reverse and ligation, DNA was purified and subjected to the further analysis. GAPDH was used as an internal control.

### Generation and Treatment of PCa Xenografts in Mice

Six-week SCID male mice were used in the study. Mice were subcutaneously injected with VCaP cells (5×10⁶) mixed with Matrigel mixture (1×PBS: Matrigel (BD Biosciences)=1:1). After the xenografts reached a size of approximately 100 mm³, mice were treated intraperitoneally with vehicle (90% corn oil (Sigma-Aldrich)+10% DMSO), ICG-001 or CBP PROTAC CP2 at 25 mg/kg for 5 days per week. For LEF1/TCF O′PROTAC administration, mice were transplanted with LuCaP23.1 PDX tumors in the approximately same volume. The LEF1/TCF OP was administrated intravenously into mice when the PDX volume reached 100 mm³. Tumor length (L) and width (W) were measured every 3 days, and tumor volumes were calculated by the formula: (L×W²)/2. Mice were euthanized manner and tumor grafts were excised after treatment for indicated days. Tumor tissues were subjected to formalin fixation and paraffin embedding or lysed for protein extraction.

### Methods

### Design of ICG-001 Derived PROTACs

The small molecule ICG-001 was originally identified to bind CBP and inhibit 0—Catenin-LEF/TCF complex function. Given that a biotinylated derivative of ICG-001 was synthesized and used for successful pulldown of CBP, it was reasoned that the attachment of the biotin-linker to meta-position of the phenyl-methanamine group in ICG-001 did not influence the binding of this small molecule to CBP, suggesting that the linker of the PROTACs can also be attached to ICG-001 at the same position (Scheme 1).

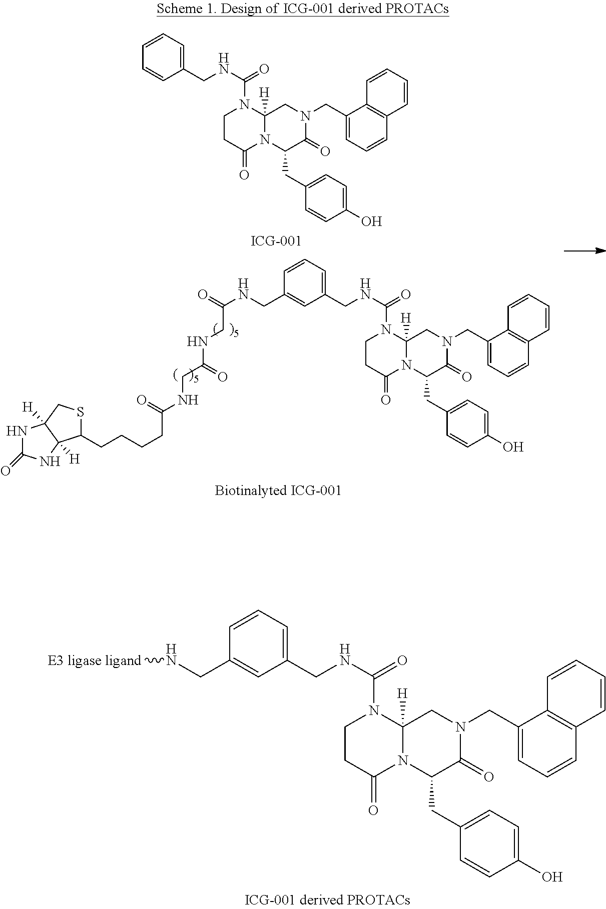

### Synthesis of ICG-001 Derived PROTACs

The synthesis of ICG-001 derived PROTACs was started with a partial protection on one amine group of 1,3-phenylenedimethanamine with Fmoc-protecting group, receiving compound 1. After that, the other amine group was subjected to an isocyanating reaction with Triphosgene followed by urea formation reaction with tert-butyl 3-aminopropanoate hydrochloride, receiving compound 2. Then, after de-protection from tert-butyl group by trifluoroacetic acid, the resulting molecule was subjected to an amide formation reaction with (S)-2-amino-3-(4-(tert-butoxy)phenyl)—N-(2,2-diethoxyethyl)—N-(naphthalen-1-ylmethyl)propanamide catalyzed by HATU. The received compound 3 was followed by a cyclization reaction with formic acid, receiving compound 4. After that, compound 5 was received by a de-protection reaction with diethylamine. The resulting compound was then subjected to a HATU catalyzed amide formation reaction with respective E3 ligase ligands conjugated with linkers of different lengths, receiving PROTAC derived compounds with linkers of different lengths respectively.

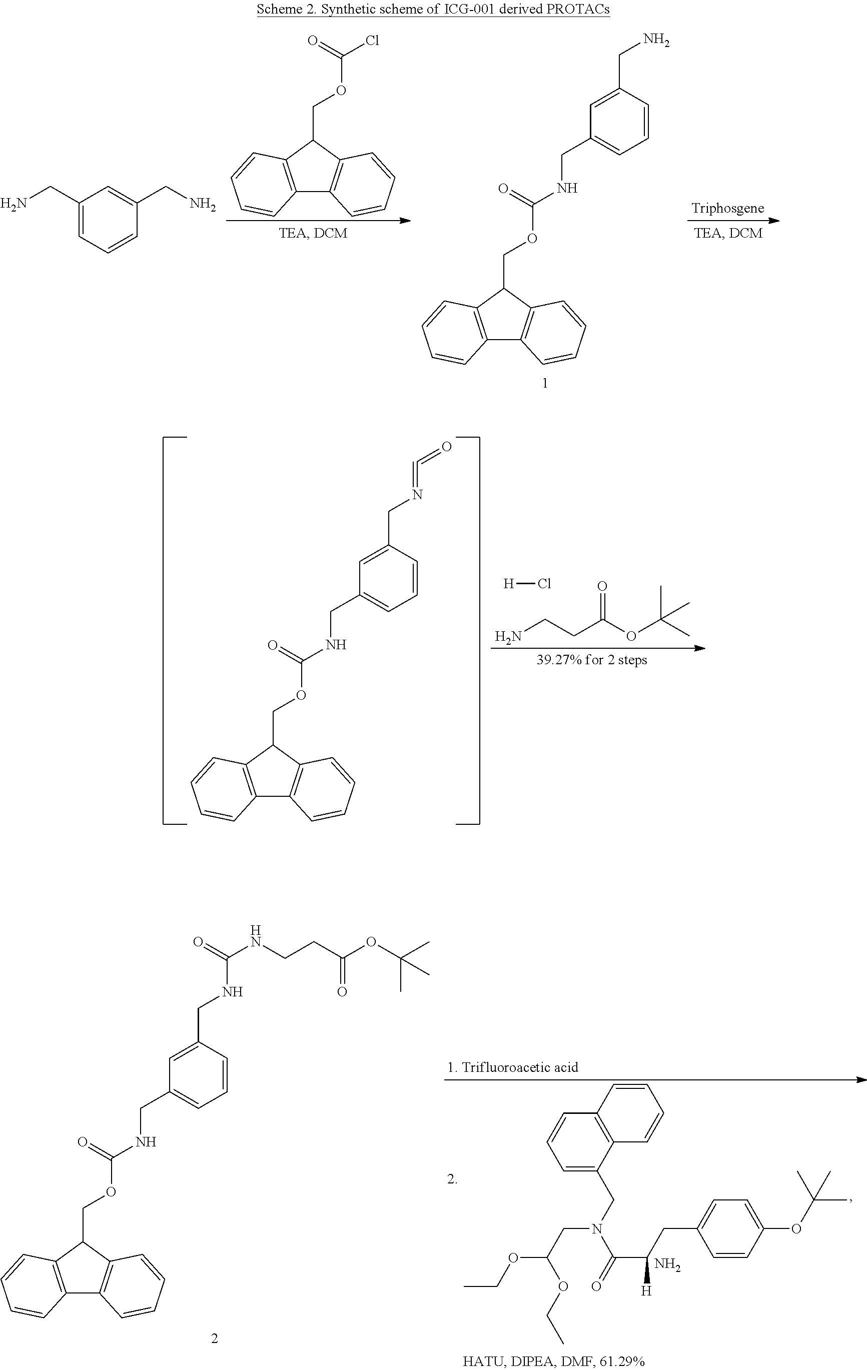

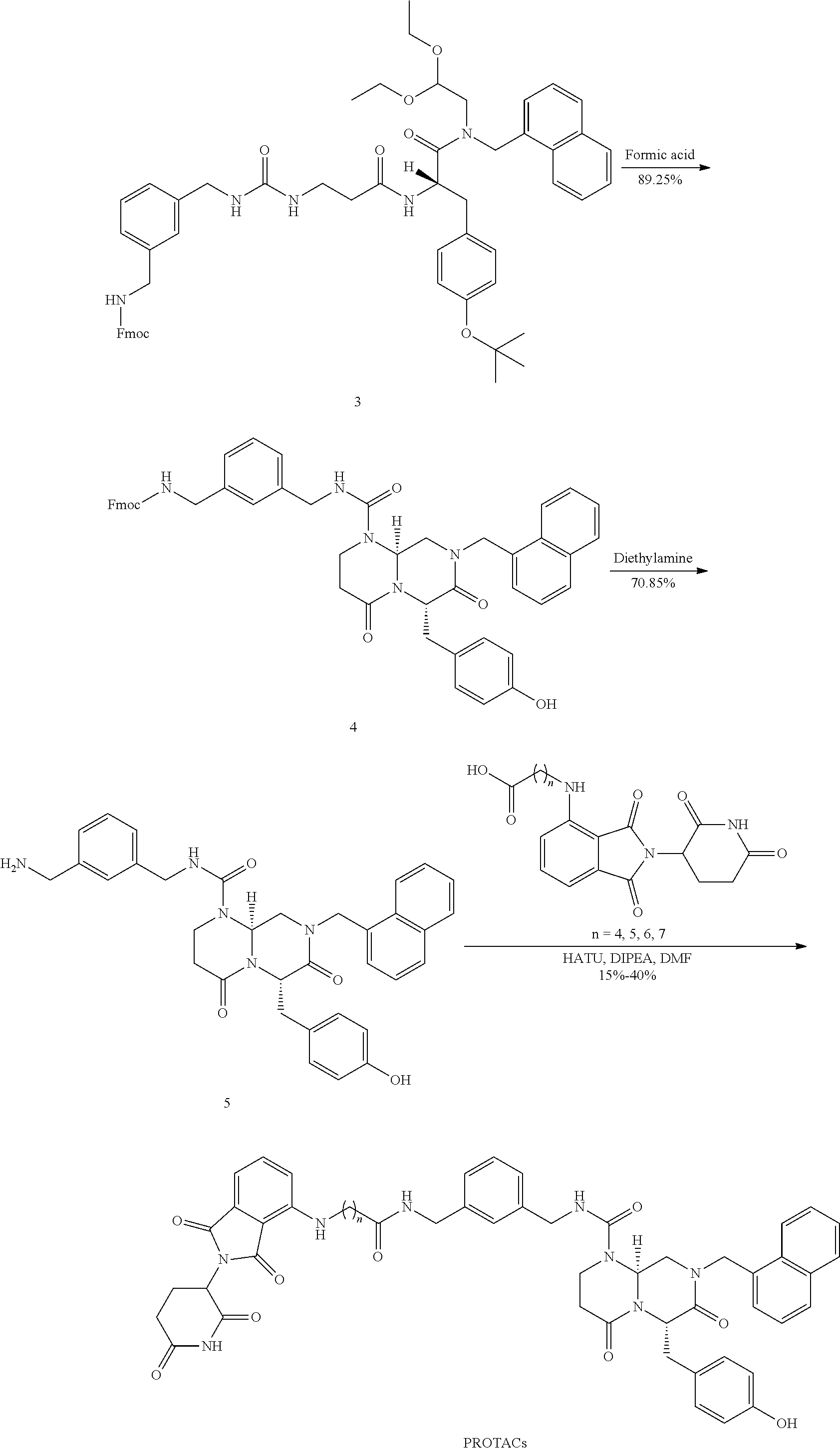

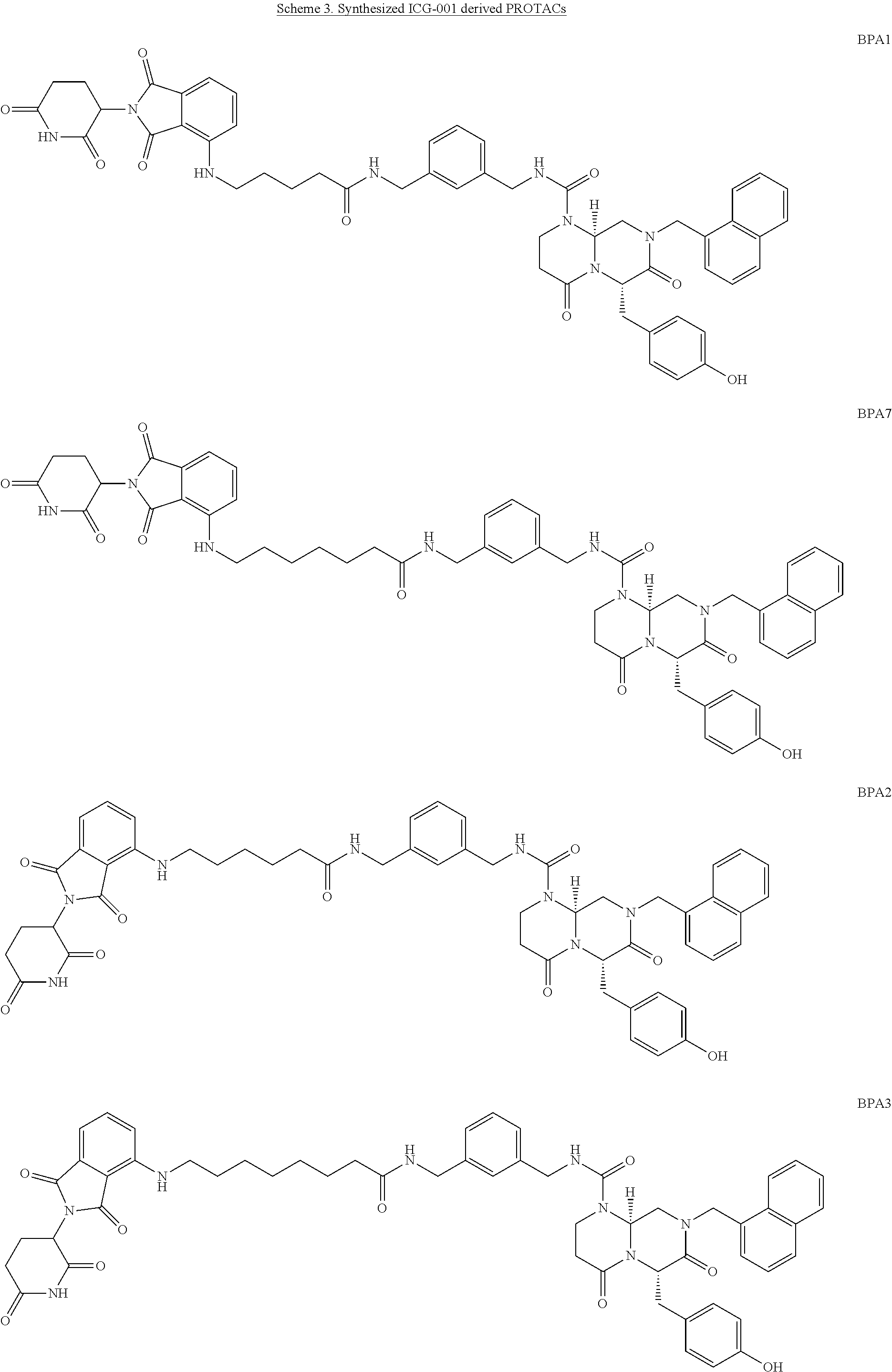

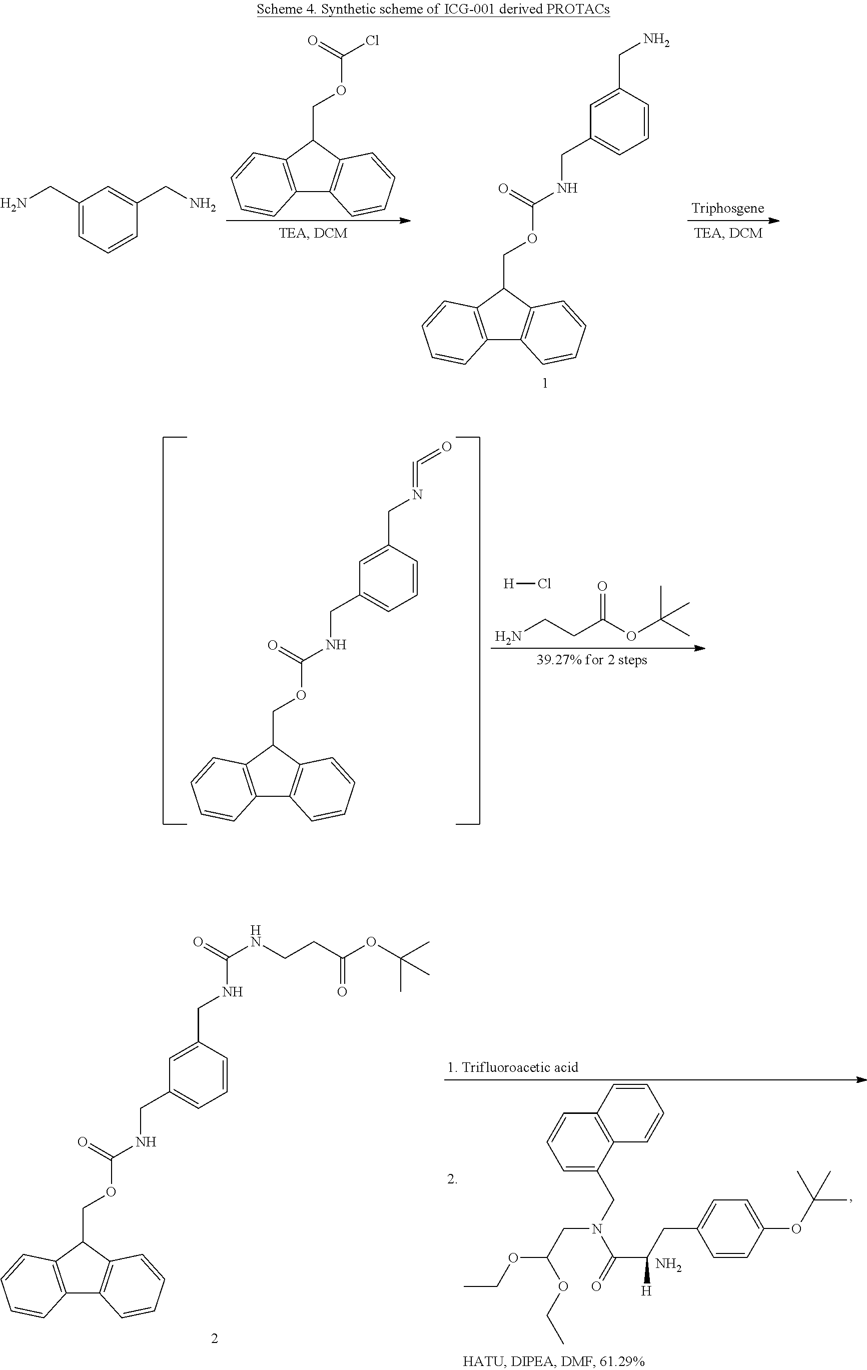

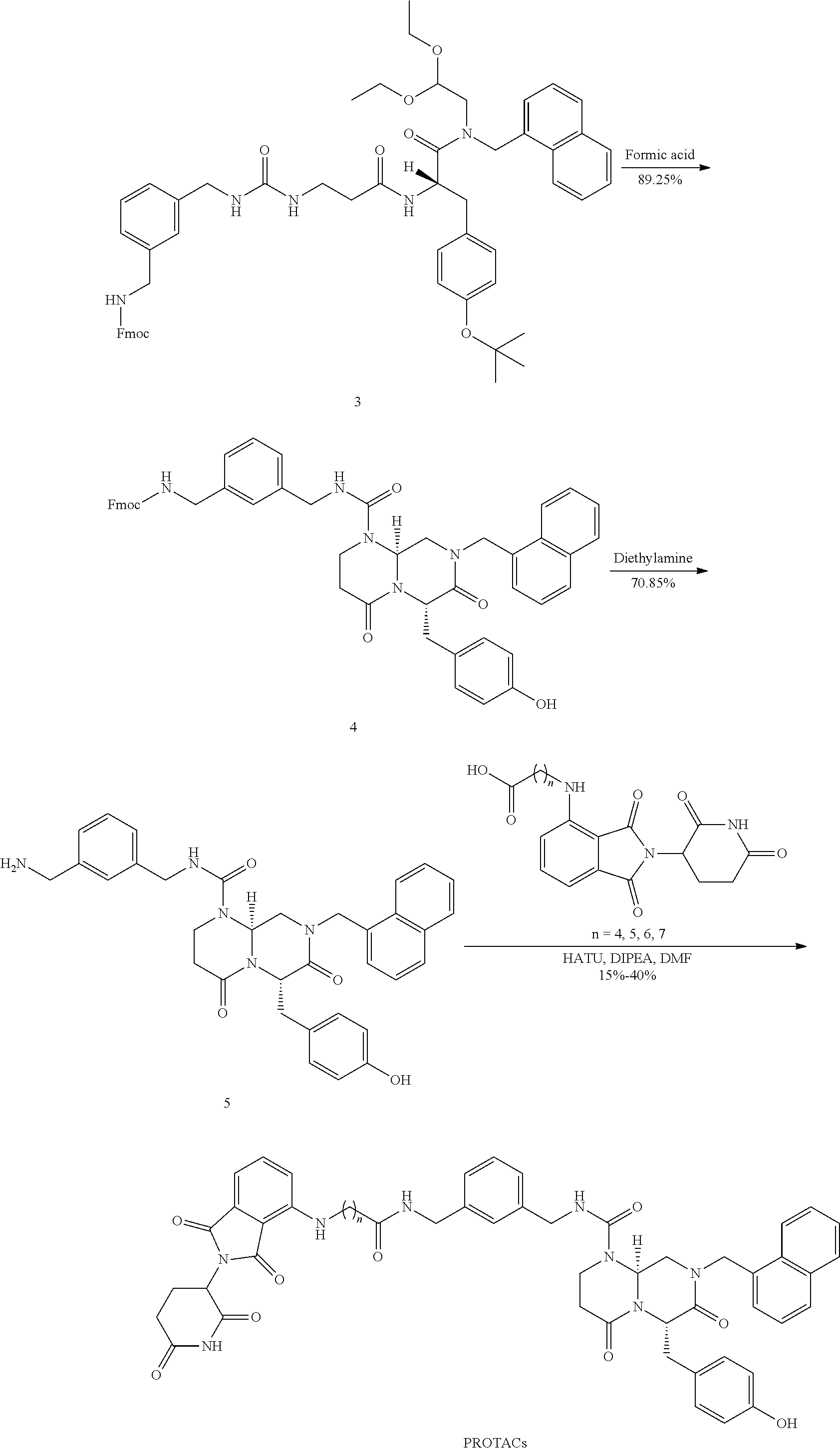

Synthesis of 1: ADCM solution (10 mL, anhydrous) containing Fmoc chloride (0.65 g, 2.5 mmol) was added to a DCM solution (10 mL, anhydrous) containing 1,3-phenylenedimethanamine (0.68 g, 5.0 mmol) and trimethylamine (1.4 mL, 10 mmol). The mixture was stirred on ice bath for 1 hour under N₂ atmosphere. After the termination of the reaction was verified by TLC, water (20 mL) and DCM (20 mL×3) were added, and the organic layers were collected, dried over anhydrous Na₂SO₄, and concentrated in vacuo. The resulting solid was used for next step directly. MS m/z [M+1] 358.9.

Synthesis of 2: Triphosgene (0.74 g, 2.5 mmol) was added to a DCM solution (20 mL, anhydrous) containing compound 1 (2.5 mmol) and trimethylamine (1.05 mL, 7.5 mmol). The mixture was stirred on ice bath for 1 hour under N₂ atmosphere. After the termination of the reaction was verified by TLC, H-Beta-Ala-OtBu HCl (0.45 g, 2.5 mmol) was added to the solution. The mixture was stirred for another 8 hours under N₂ atmosphere. Then, the resulting solution was concentrated in vacuo. Flash chromatography (EA/Hexane 0-80%) yielded A-SM2 as a white solid (0.52 g, 39.27%). MS m/z [M+1] 529.8. ¹H NMR (400 MHz, dmso) δ 7.88 (dd, J=10.4, 7.0 Hz, 3H), 7.70 (d, J=7.4 Hz, 2H), 7.42 (t, J=7.4 Hz, 2H), 7.33 (dd, J=10.9, 4.0 Hz, 2H), 7.25 (t, J=7.8 Hz, 1H), 7.09 (dd, J=10.2, 7.5 Hz, 3H), 6.55 (t, J=5.9 Hz, 1H), 6.07 (t, J=5.9 Hz, 1H), 4.33 (d, J=6.9 Hz, 2H), 4.23 (t, J=6.9 Hz, 1H), 4.17 (s, 2H), 4.16 (s, 2H), 3.22-3.15 (m, 2H), 2.32 (t, J=6.6 Hz, 2H), 1.39 (s, 9H).

Synthesis of 3: Compound 2 (2.50 g, 4.72 mmol) was added to a mixture solution (DCM:TFA=3:1, 40 mL). The mixture was stirred overnight. Then, the reaction liquid was concentrated in vacuo. After that, DMF (30 mL) was added to the resulting oil on ice bath, and A3 (2.48 g, 5.04 mmol), HATU (5.57 g, 6.75 mmol) and DIPEA (2.35 mL, 13.50 mmol) were added to the solution. The mixture was stirred overnight under N₂ atmosphere. Then, water (50 mL) and EA (50 mL×3) were added, and the organic layers were collected, washed with H₂O (50 mL×2) and brine (50 mL), dried over anhydrous Na₂SO₄, and concentrated in vacuo. Flash chromatography (EA) yielded B4 as a peach-colored solid (2.87 g, 67.26%). MS m/z [M+1]948.6. ¹H NMR (400 MHz, dmso) δ 8.05-7.99 (m, 1H), 7.98-7.92 (m, 1H), 7.87 (dd, J=12.7, 7.4 Hz, 4H), 7.70 (d, J=7.4 Hz, 2H), 7.59-7.51 (m, 2H), 7.47-7.37 (m, 3H), 7.32 (t, J=7.4 Hz, 2H), 7.24 (dd, J=9.2, 6.9 Hz, 2H), 7.09 (t, J=9.7 Hz, 5H), 6.86 (d, J=8.4 Hz, 1H), 6.78 (d, J=8.2 Hz, 2H), 6.49-6.42 (m, 1H), 5.94 (d, J=5.7 Hz, 1H), 5.15-4.99 (m, 2H), 4.33 (d, J=6.9 Hz, 2H), 4.29-4.20 (m, 2H), 4.19-4.08 (m, 4H), 4.05-3.95 (m, 1H), 3.63-3.38 (m, 4H), 3.30-3.18 (m, 2H), 3.19-3.09 (m, 2H), 2.95-2.86 (m, 2H), 2.23 (t, J=6.9 Hz, 2H), 1.20 (s, 9H), 0.99 (t, J=6.9 Hz, 6H).

Synthesis of 4: Compound 3 (2.75 g, 2.90 mmol) was dissolved in formic acid (40 mL) and the mixture was stirred at room temperature for 12 hours under N₂ atmosphere. Then, the solution was concentrated in vacuo. Flash chromatography (EA) yielded A7 as a white solid (82 mg, 80.12%). MS m/z [M+1] 800.0. ¹H NMR (400 MHz, dmso) δ 8.32 (d, J=7.6 Hz, 1H), 8.17-8.11 (m, 1H), 7.97 (dd, J=6.9, 2.5 Hz, 1H), 7.93-7.86 (m, 2H), 7.86-7.81 (m, 1H), 7.57 (ddt, J=9.6, 6.6, 3.5 Hz, 4H), 7.52-7.46 (m, 1H), 7.42 (dd, J=7.4, 1.1 Hz, 1H), 7.39 (t, J=4.2 Hz, 1H), 7.34 (td, J=7.4, 1.2 Hz, 1H), 7.24 (dd, J=15.7, 8.3 Hz, 2H), 7.18 (s, 1H), 7.05 (d, J=7.3 Hz, 1H), 6.98 (d, J=8.5 Hz, 1H), 6.91 (d, J=8.5 Hz, 2H), 6.65 (d, J=8.5 Hz, 1H), 6.53 (d, J=8.5 Hz, 2H), 6.28 (s, 1H), 5.75 (dd, J=10.7, 4.0 Hz, 1H), 5.18-5.07 (m, 2H), 4.92 (d, J=15.0 Hz, 1H), 4.30 (dd, J=15.2, 5.8 Hz, 1H), 4.26-4.05 (m, 3H), 4.01 (dt, J=7.2, 5.7 Hz, 1H), 3.91-3.81 (m, 1H), 3.68 (s, 2H), 3.56 (t, J=11.1 Hz, 1H), 3.50 (s, 1H), 3.18-3.13 (m, 1H), 3.06 (dd, J=12.4, 6.8 Hz, 2H), 2.07 (s, 2H).

Synthesis of 5: Compound 4 (2.30 g, 2.88 mmol) was dissolved in DCM (20 mL). Subsequently, diethylamine (DEA) (10 mL, excess) was added, and the mixture was stirred at room temperature for 3 hours. After the termination of the reaction was verified by TLC, DCM was distilled away under reduced pressure. Flash chromatography (MeOH/DCM 0-10%) yielded B6 as a yellow solid (1.11 g, 66.83%). MS m/z [M+1] 578.1. ¹H NMR (400 MHz, dmso) δ 8.47 (s, 2H), 8.14 (d, J=7.4 Hz, 1H), 7.99-7.94 (m, 1H), 7.90 (d, J=8.3 Hz, 1H), 7.63-7.52 (m, 3H), 7.52-7.46 (m, 1H), 7.38 (d, J=7.0 Hz, 1H), 7.25 (dd, J=15.5, 8.1 Hz, 2H), 7.19 (s, 1H), 7.07 (d, J=7.3 Hz, 1H), 6.91 (d, J=8.4 Hz, 2H), 6.53 (d, J=8.4 Hz, 2H), 5.78-5.71 (m, 1H), 5.18-5.07 (m, 2H), 4.91 (d, J=15.0 Hz, 1H), 4.30 (dd, J=15.3, 5.9 Hz, 1H), 4.16 (dd, J=15.3, 5.2 Hz, 1H), 3.91-3.80 (m, 1H), 3.71 (s, 2H), 3.60-3.52 (m, 1H), 3.50 (s, 1H), 3.13-3.09 (m, 1H), 3.09-2.98 (m, 2H), 2.14-2.04 (m, 2H).

Synthesis of ICG-001 derived PROTACs (general procedure): Compound 5 (44 mg, 76.17 umol), the respective E3 ligase ligand-linker acid (43 mg, 99-115 umol), HATU (43 mg, 114.25 umol) and TEA (40 uL, 228.50 umol) were dissolved into 3 mL DMF. The solution was stirred overnight under N₂ atmosphere. After the termination of the reaction was verified by TLC, DMF was distilled away under reduced pressure. Flash chromatography (MeOH/DCM 0-8%) followed by Preparation TLC yielded ICG-001 derived PROTACs as yellow solid (9-16 mg, 15%-40%).

Synthesis of CP1: CP1 was synthesized following the general procedure of ICG-001 derived PROTACs. MS m/z [M+1] 933.1. ¹H NMR (400 MHz, dmso) δ 11.10 (s, 1H), 9.18 (s, 1H), 8.31 (t, J=6.0 Hz, 1H), 8.14 (d, J=7.5 Hz, 1H), 7.99-7.94 (m, 1H), 7.89 (d, J=8.2 Hz, 1H), 7.60-7.52 (m, 3H), 7.51-7.44 (m, 1H), 7.38 (d, J=7.0 Hz, 1H), 7.24 (t, J=7.8 Hz, 1H), 7.13-7.04 (m, 4H), 7.01 (d, J=7.0 Hz, 1H), 6.92 (d, J=8.4 Hz, 2H), 6.55 (d, J=8.4 Hz, 3H), 5.75 (dd, J=10.3, 4.2 Hz, 1H), 5.14 (dd, J=8.7, 4.7 Hz, 1H), 5.09 (s, 1H), 5.04 (dd, J=12.9, 5.4 Hz, 1H), 4.92 (d, J=15.0 Hz, 1H), 4.27 (dd, J=15.5, 5.9 Hz, 1H), 4.22 (d, J=5.9 Hz, 2H), 4.16 (dd, J=15.3, 5.2 Hz, 1H), 4.03 (dd, J=14.3, 7.1 Hz, 1H), 3.91-3.80 (m, 1H), 3.56 (t, J=11.1 Hz, 1H), 3.31-3.25 (m, 2H), 3.14 (dd, J=11.5, 3.9 Hz, 1H), 3.05 (ddd, J=22.5, 13.8, 9.0 Hz, 2H), 2.93-2.82 (m, 1H), 2.60 (s, 1H), 2.56 (s, 1H), 2.16 (t, J=6.8 Hz, 2H), 2.08 (d, J=5.1 Hz, 2H), 2.05-1.97 (m, 2H), 1.56 (d, J=5.7 Hz, 4H). ¹³C NMR (101 MHz, dmso) δ 172.82, 171.90, 170.11, 168.91, 167.30, 165.89, 165.19, 156.02, 155.85, 146.36, 140.30, 139.62, 136.25, 133.45, 132.21, 131.60, 131.08, 130.23, 128.64, 128.23 (2C), 126.83 (2C), 126.48, 126.03, 125.97, 125.50, 125.42, 125.28, 123.52, 117.17, 114.95, 110.37, 109.02, 60.23, 59.77, 55.84, 48.53, 47.28, 43.56, 41.99, 41.52, 36.09, 35.47, 34.97, 31.37, 30.99, 28.36, 22.64, 22.17, 20.79, 14.11.

Synthesis of CP2: CP2 was synthesized following the general procedure of ICG-001 derived PROTACs. MS m/z [M+1] 947.2. ¹H NMR (400 MHz, dmso) δ 11.11 (s, 1H), 8.30 (t, J=5.9 Hz, 1H), 8.14 (d, J=7.9 Hz, 1H), 7.99-7.92 (m, 1H), 7.89 (d, J=8.2 Hz, 1H), 7.61-7.51 (m, 3H), 7.50-7.44 (m, 1H), 7.38 (d, J=6.8 Hz, 1H), 7.26 (t, J=7.8 Hz, 1H), 7.15-6.98 (m, 5H), 6.93 (d, J=8.5 Hz, 2H), 6.57 (d, J=8.5 Hz, 3H), 5.81-5.73 (m, 1H), 5.20-5.13 (m, 1H), 5.09 (d, J=8.2 Hz, 1H), 5.08-5.01 (m, 1H), 4.92 (d, J=15.0 Hz, 1H), 4.30 (dd, J=15.5, 5.8 Hz, 1H), 4.23 (d, J=5.9 Hz, 2H), 4.18 (dd, J=15.5, 5.2 Hz, 1H), 4.05 (s, 1H), 3.86 (dd, J=13.9, 3.8 Hz, 1H), 3.56 (t, J=11.1 Hz, 1H), 3.25 (t, J=7.0 Hz, 2H), 3.17-3.12 (m, 1H), 3.11-3.00 (m, 2H), 2.88 (ddd, J=17.5, 14.1, 5.3 Hz, 1H), 2.61 (d, J=2.7 Hz, 1H), 2.60-2.53 (m, 1H), 2.20-2.06 (m, 4H), 2.02 (ddd, J=10.3, 6.8, 4.6 Hz, 2H), 1.55 (dt, J=14.8, 7.5 Hz, 4H), 1.31 (dt, J=9.4, 7.6 Hz, 2H). ¹³C NMR (101 MHz, dmso) δ 172.89, 172.14, 170.18, 169.01, 167.37, 165.98, 165.28, 156.09, 155.93, 146.44, 140.35, 139.71, 136.31, 133.51, 132.23, 131.63, 131.14, 130.30, 128.69, 128.28 (2C), 126.89 (2C), 126.52, 126.07, 125.99, 125.56, 125.47, 125.34, 123.57, 117.19, 115.02, 110.45, 109.06, 69.85, 60.32, 55.92, 54.96, 48.67, 48.61, 47.35, 43.65, 42.04, 41.81, 36.15, 35.52, 35.33, 31.43, 31.05, 28.53, 26.06, 25.11, 22.23.

Synthesis of CP3: CP3 was synthesized following the general procedure of ICG-001 derived PROTACs. MS m/z [M+1] 975.2. ¹H NMR (400 MHz, dmso) δ 11.10 (s, 1H), 9.19 (s, 1H), 8.27 (t, J=5.8 Hz, 1H), 8.14 (d, J=8.3 Hz, 1H), 7.96 (d, J=7.8 Hz, 1H), 7.89 (d, J=8.2 Hz, 1H), 7.56 (t, J=6.8 Hz, 3H), 7.47 (t, J=7.6 Hz, 1H), 7.38 (d, J=6.9 Hz, 1H), 7.25 (t, J=7.5 Hz, 1H), 7.20-7.03 (m, 4H), 7.01 (d, J=6.9 Hz, 1H), 6.92 (d, J=7.8 Hz, 2H), 6.60-6.46 (m, 3H), 5.75 (d, J=7.1 Hz, 1H), 5.14 (d, J=9.8 Hz, 1H), 5.09 (s, 1H), 5.07-4.99 (m, 1H), 4.92 (d, J=14.8 Hz, 1H), 4.28 (dd, J=15.6, 5.6 Hz, 1H), 4.21 (d, J=5.6 Hz, 2H), 4.17 (d, J=10.5 Hz, 1H), 4.03 (dd, J=14.3, 7.3 Hz, 1H), 3.85 (d, J=12.7 Hz, 1H), 3.56 (t, J=11.0 Hz, 1H), 3.26 (dd, J=13.0, 7.1 Hz, 2H), 3.14 (d, J=7.7 Hz, 1H), 3.04 (dd, J=20.8, 12.0 Hz, 2H), 2.94-2.81 (m, 1H), 2.60 (s, 1H), 2.56 (s, 1H), 2.24-2.04 (m, 4H), 2.01 (d, J=17.6 Hz, 2H), 1.51 (dd, J=16.1, 8.3 Hz, 4H), 1.37-1.18 (m, 6H). ¹³C NMR (101 MHz, dmso) δ 172.83, 172.10, 170.13, 168.95, 167.31, 165.89, 165.19, 156.02, 155.86, 146.41, 140.28, 139.70, 136.28, 133.46, 132.20, 131.60, 131.08, 130.24, 128.65, 128.21 (2C), 126.83 (2C), 126.47, 126.03, 125.93, 125.47, 125.42, 125.25, 123.53, 117.17, 114.94, 110.37, 108.99, 60.23, 59.78, 55.84, 48.54, 47.29, 43.58, 41.94, 41.81, 36.09, 35.46, 35.31, 31.37, 30.99, 28.67, 28.51, 26.26, 25.26, 22.15.

Synthesis of CP4: CP4 was synthesized following the general procedure of ICG-001 derived PROTACs. MS m/z [M+1] 961.2. ¹H NMR (400 MHz, dmso) δ 11.10 (s, 1H), 9.18 (s, 1H), 8.28 (t, J=6.0 Hz, 1H), 8.14 (d, J=7.7 Hz, 1H), 7.96 (dd, J=7.1, 2.4 Hz, 1H), 7.89 (d, J=8.2 Hz, 1H), 7.60-7.52 (m, 3H), 7.51-7.44 (m, 1H), 7.38 (d, J=6.8 Hz, 1H), 7.29-7.22 (m, 1H), 7.13-7.04 (m, 4H), 7.01 (d, J=7.0 Hz, 1H), 6.92 (d, J=8.5 Hz, 2H), 6.60-6.48 (m, 3H), 5.75 (dd, J=10.6, 3.9 Hz, 1H), 5.14 (dd, J=8.7, 4.8 Hz, 1H), 5.09 (s, 1H), 5.05 (dd, J=12.9, 5.4 Hz, 1H), 4.92 (d, J=15.0 Hz, 1H), 4.28 (dd, J=15.5, 6.0 Hz, 1H), 4.22 (d, J=5.9 Hz, 2H), 4.17 (dd, J=15.6, 5.5 Hz, 1H), 4.03 (q, J=7.1 Hz, 1H), 3.89-3.80 (m, 1H), 3.56 (t, J=11.1 Hz, 1H), 3.29-3.23 (m, 2H), 3.18-3.10 (m, 1H), 3.09-2.98 (m, 2H), 2.87 (ddd, J=17.6, 14.1, 5.4 Hz, 1H), 2.60 (d, J=2.8 Hz, 1H), 2.56 (s, 1H), 2.10 (dd, J=14.0, 6.5 Hz, 4H), 2.06-1.98 (m, 2H), 1.60-1.45 (m, 4H), 1.36-1.25 (m, 4H). ¹³C NMR (101 MHz, dmso) δ 172.83, 172.07, 170.13, 168.95, 167.31, 165.89, 165.19, 156.03, 155.86, 146.40, 140.29, 139.70, 136.28, 133.46, 132.20, 131.60, 131.08, 130.24, 128.65, 128.21 (2C), 126.83 (2C), 126.48, 126.03, 125.93, 125.48, 125.43, 125.26, 123.53, 117.16, 114.95, 110.38, 109.00, 60.24, 59.78, 55.85, 48.54, 47.29, 43.58, 41.95, 41.81, 36.09, 35.47, 35.30, 31.37, 30.99, 28.59, 28.44, 26.11, 25.25, 22.16, 20.79, 14.11.

### Quantification and Statistical Analysis

### Meta-Analysis of Patient Data

The status of TP53 gene mutation or deletion in the SU2C cohort was obtained through ciBioPortal (www.cbioportal.org/): (1) wild type (WT), (2) homozygous deletion (null) and (3) GOF p53 mutation (Mut) in the DNA binding domain of p53. The Z-score (FPKM) of CTNNB1 reflecting mRNA level was downloaded and subjected to the comparison based on the status of TP53 gene alterations. Mann-Whitney U test was carried out to generate p value for the comparison.

For the correlation of UMPS, (2015a)RRM1, RRM2 mRNA expression with CTNNB1 level, The relative expression was represented as Z-scores by using formula: Z=(x−μ)/σ, while the x means raw log 2 (FPKM), is the average value and a is the standard deviation for all samples of a gene. ERG fusion-positive patients from TCGA database were divided into two groups with either low (<average) or high (>average) CTNNB1 expression level. Mann-Whitney U test was carried out to generate p value for the comparison. Log-rank (Mantel-Cox) test was performed to determine the statistical differences between stratified groups used for Kaplan-Meier Survival curve analyses.

### Statistical Analysis

P values were determined by a two-tailed Student's t test, two-way ANOVA test, log-rank test, Fisher exact test or χ² test. All data are shown as mean values±S.D. for experiments representing three independent experiments. P values<0.05 were considered statistically significant.

### Example 18: Design of Mutant p53 O′PROTACs

4 O′PROTACs were designed for each sequence, and were attached to an E3 ligand at the 5′-forward strand as shown below.

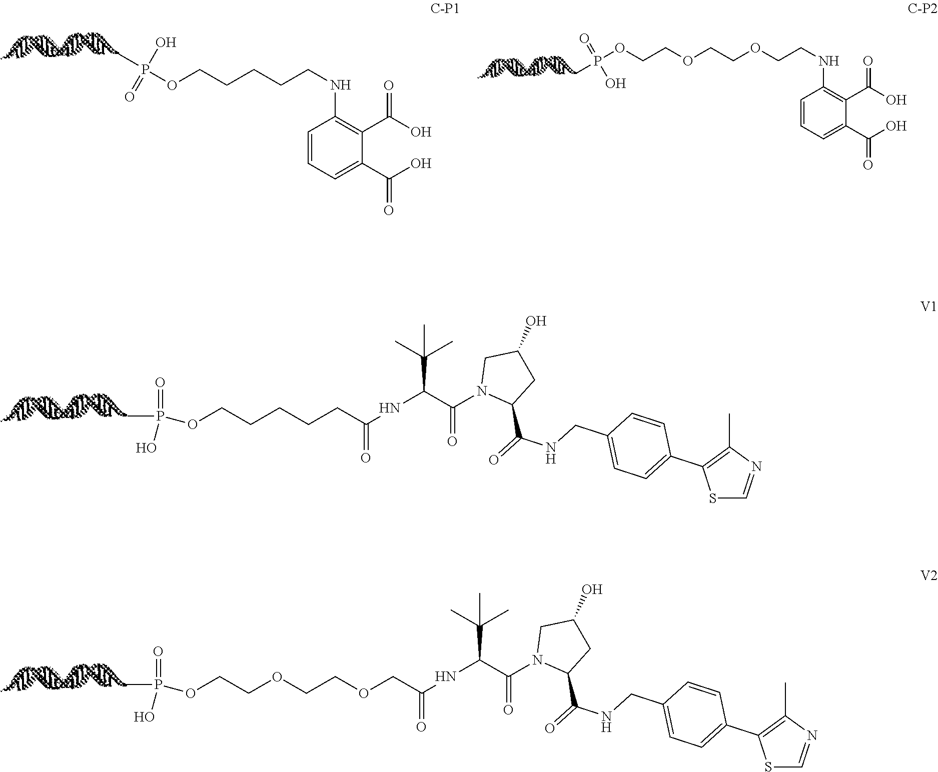 [SMILES: CP(=O)(O)OCCCCCNc1cccc(C(=O)O)c1C(=O)O ; CP(=O)(O)OCCOCCOCCNc1cccc(C(=O)O)c1C(=O)O ; Cc1ncsc1-c1ccc(CNC(=O)[C@@H]2C[C@@H](O)CN2C(=O)[C@@H](NC(=O)CCCCCOP(C)(=O)O)C(C)(C)C)cc1 ; Cc1ncsc1-c1ccc(CNC(=O)[C@@H]2C[C@@H](O)CN2C(=O)[C@@H](NC(=O)COCCOCCOP(C)(=O)O)C(C)(C)C)cc1]

35 sequences were synthesized in total, and they are shown in the table below.

| NO. | TARGET | NAME | SEQUENCE | SEQ ID NO |
|---|---|---|---|---|
| 1 | WT p53 Consensus | OP1-F-CP1 | 5′-CP1-CCG GAC ATG CCC GGG CAT GT-3′ | 383 |
| 2 | | OP1-F-CP2 | 5′-CP2-CCG GAC ATG CCC GGG CAT GT-3′ | 384 |
| 3 | | OP1-F-V1 | 5′-V1-CCG GAC ATG CCC GGG CAT GT-3′ | 385 |
| 4 | | OP1-F-V2 | 5′-V2-CCG GAC ATG CCC GGG CAT GT-3′ | 386 |
| 5 | | OP1-R | 5′-ACA TGC CCG GGC ATG TCC GG-3′ | 387 |
| 6 | Mut p53-CTNNB1 | OP2-F-CP1 | 5′-CP1-GCC CCC TCG CGC CCC GCC CCT TGT C-3′ | 388 |
| 7 | | OP2-F-CP2 | 5′-CP2-GCC CCC TCG CGC CCC GCC CCT TGT C-3′ | 389 |
| 8 | | OP2-F-V1 | 5′-V1-GCC CCC TCG CGC CCC GCC CCT TGT C-3′ | 390 |
| 9 | | OP2-F-V2 | 5′-V2-GCC CCC TCG CGC CCC GCC CCT TGT C-3′ | 391 |
| 10 | | OP2-R | 5′-GAC AAG GGG CGG GGC GCG AGG GGG C-3′ | 392 |
| 11 | Mut p53-Ctnnbl | OP3-F-CP1 | 5′-CP1-GCC CGC CCG CGC CCC GCC CCT CGT C-3′ | 393 |
| 12 | | OP3-F-CP2 | 5′-CP2-GCC CGC CCG CGC CCC GCC CCT CGT C-3′ | 394 |
| 13 | | OP3-F-V1 | 5′-V1-GCC CGC CCG CGC CCC GCC CCT CGT C-3′ | 395 |
| 14 | | OP3-F-V2 | 5′-V2-GCC CGC CCG CGC CCC GCC CCT CGT C-3′ | 396 |
| 15 | | OP3-R | 5-GAC GAG GGG CGG GGC GCG GGC GGG C-3′ | 397 |
| 16 | Mut p53-KAT6A | OP4-F-CP1 | 5′-CP1-CTC CCC TCG CCG CCC GCC CAG GCT C-3′ | 398 |
| 17 | | OP4-F-CP2 | 5′-CP2-CTC CCC TCG CCG CCC GCC CAG GCT C-3′ | 399 |
| 18 | | OP4-F-V1 | 5′-V1-CTC CCC TCG CCG CCC GCC CAG GCT C-3′ | 400 |
| 19 | | OP4-F-V2 | 5′-V2-CTC CCC TCG CCG CCC GCC CAG GCT C-3′ | 401 |
| 20 | | OP4-R | 5′-GAG CCT GGG CGG GCG GCG AGG GGA G-3′ | 402 |
| 21 | Mut p53-KMT2A | OP5-F-CP1 | 5′-CP1-GCG GCC TCG GGC CTC CGC CTC TGA C-3′ | 403 |
| 22 | | OP5-F-CP2 | 5′-CP2-GCG GCC TCG GGC CTC CGC CTC TGA C-3′ | 404 |
| 23 | | OP5-F-V1 | 5′-V1-GCG GCC TCG GGC CTC CGC CTC TGA C-3′ | 405 |
| 24 | | OP5-F-V2 | 5′-V2-GCG GCC TCG GGC CTC CGC CTC TGA C-3′ | 406 |
| 25 | | OP5-R | 5′-GTC AGA GGC GGA GGC CCG AGG CCG C-3′ | 407 |
| 26 | Mut p53-MCL1 | OP6-F-CP1 | 5′-CP1-CCC GGC CCC GGC CCC GCC CCG GCC C-3′ | 408 |
| 27 | | OP6-F-CP2 | 5′-CP2-CCC GGC CCC GGC CCC GCC CCG GCC C-3′ | 409 |
| 28 | | OP6-F-V1 | 5′-V1-CCC GGC CCC GGC CCC GCC CCG GCC C-3′ | 410 |
| 29 | | OP6-F-V2 | 5′-V2-CCC GGC CCC GGC CCC GCC CCG GCC C-3′ | 411 |
| 30 | | OP6-R | 5′-GGG CCG GGG CGG GGC CGG GGC CGG G-3′ | 412 |
| 31 | Mut p53-MED23 | OP7-F-CP1 | 5′-CP1-TCC GGC TTG CGG GCC GCC CTT CTC C-3′ | 413 |
| 32 | | OP7-F-CP2 | 5′-CP2-TCC GGC TTG CGG GCC GCC CTT CTC C-3′ | 414 |
| 33 | | OP7-F-V1 | 5′-V1-TCC GGC TTG CGG GCC GCC CTT CTC C-3′ | 415 |
| 34 | | OP7-F-V2 | 5′-V2-TCC GGC TTG CGG GCC GCC CTT CTC C-3′ | 416 |
| 35 | | OP7-R | 5′-GGA GAA GGG CGG CCC GCA AGC CGG A-3′ | 417 |

| Modifier | |
|---|---|
| CP1 | 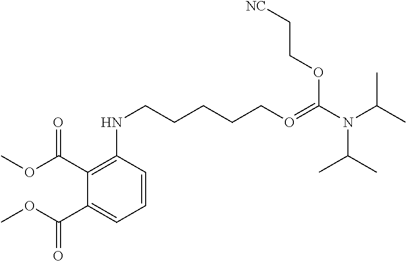 |
| CP2 | 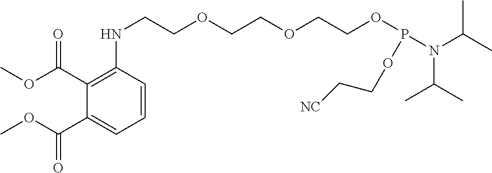 [SMILES: COC(=O)c1cccc(NCCOCCOCCOP(OCCC#N)N(C(C)C)C(C)C)c1C(=O)OC] |
| V1 | 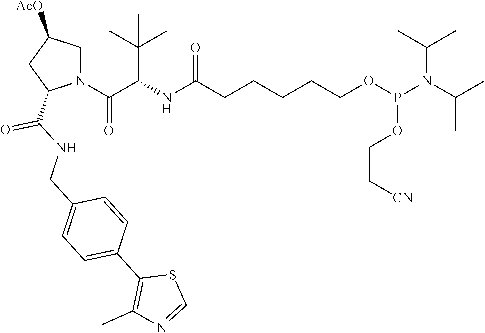 [SMILES: CC(=O)O[C@@H]1C[C@@H](C(=O)NCc2ccc(-c3scnc3C)cc2)N(C(=O)[C@@H](NC(=O)CCCCCOP(OCCC#N)N(C(C)C)C(C)C)C(C)(C)C)C1] |
| V2 | 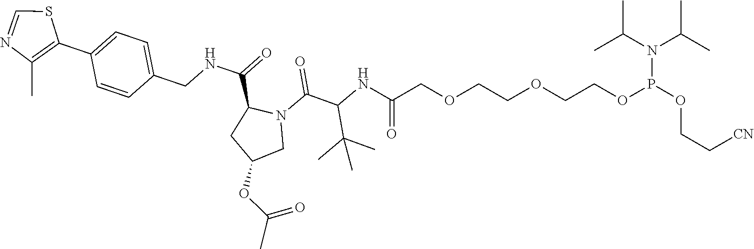 [SMILES: CC(=O)O[C@@H]1C[C@@H](C(=O)NCc2ccc(-c3scnc3C)cc2)N(C(=O)C(NC(=O)COCCOCCOP(OCCC#N)N(C(C)C)C(C)C)C(C)(C)C)C1] |

### Preparation of Lipid Nanoparticle

Ionizable lipid L319 (Chemicals, Cat #DC57006, 100 mg), distearoylphosphatidylcholine (DSPC; Avanti Polar Lipids, 850365C-25 mg), cholesterol (Sigma-Aldrich, C8667-500 mg), and PEG-DMG (Avanti Polar Lipids, 880151P-1g) were mixed at a molar ratio of 55:10:32.5:2.5 (L319: DSPC: cholesterol: PEG-DMG).

siRNA was diluted to ˜1 mg/mL in 10 mmol/L citrate buffer, pH 4.

The lipids were solubilized and mixed in the appropriate ratios in ethanol (e.g., 35% ethanol).

Syringe pumps were used to deliver the siRNA solution and lipid solution at 15 and 5 mL/min, respectively.

The syringes containing siRNA solution and lipid solution were connected to a union connector (0.05 in thru hole, #P-728; IDEX Health & Science, Oak Harbor, WA) with PEEK high-performance liquid chromatography tubing (0.02 in ID for siRNA solution and 0.01 in ID for lipid solution).

A length of PEEK high-performance liquid chromatography tubing (0.04 in ID) was connected to the outlet of the union connector and led to a collection tube.

The ethanol was then removed, and the external buffer was replaced with phosphate-buffered saline (155 mmol/L NaCl, 3 mmol/L Na₂HPO₄, 1 mmol/L KH₂PO₄, pH 7.5) by either dialysis or tangential flow diafiltration.

The LNPs were filtered through a 0.2 m sterile filter. Particle size was determined using a Malvern Zetasizer Nano Z S (Malvern, UK). siRNA content was determined by ultraviolet absorption at 260 nm and siRNA entrapment efficiency was determined by Quant-IT Ribogreen (Invitrogen, Carlsbad, CA) assay.

One or more of these sequences can be attached to any appropriate ligand. For example, one or more of these sequences can be attached to lenalidomide, pomalidomide, or thalidomide.

### Example 19: Exemplary Embodiments

Embodiment 1. A compound of Formula (IA):

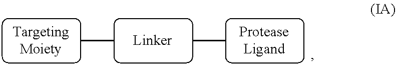

wherein the targeting moiety is an oligonucleotide capable of binding a target protein and the protease ligand is a ligand capable of binding a protease.

Embodiment 2. A pharmaceutical composition comprising the compound according to embodiment 1 and a pharmaceutically acceptable carrier.

Embodiment 3. A method for treating a disease or a disorder mediate by aberrant protein activity, wherein said method comprises administering an effective amount of the compound according to embodiment 1 or a pharmaceutical composition comprising the effective amount of the compound to a subject in need of a treatment for aberrant protein activity.

Embodiment 4. The compound, composition, or method according to any one of the preceding embodiments, wherein the targeting moiety is a double-stranded oligonucleotide.

Embodiment 5. The compound, composition, or method according to any one of the preceding embodiments, wherein the protease ligand is an E3 ligase ligand.

Embodiment 6. The compound, composition, or method according to any one of the preceding embodiments, wherein the target protein is selected from the group consisting of a transcription factor, a transcription co-regulator, a polymerase, a nuclease, a histone, and an RNA-binding protein.

Embodiment 7. The compound, composition, or method according to any one of the preceding embodiments, wherein aberrant protein activity of the target protein mediates a disease or a disorder.

Embodiment 8. The compound, composition, or method according to any one of the preceding embodiments, wherein aberrant protein activity of the target protein mediates a disease or a disorder selected from the group consisting of a cancer, an autoimmune disease, a central nervous system disease, a metabolic disease, and an infection.

Embodiment 9. The compound of embodiment 1, wherein the linker has formula:

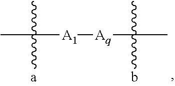

wherein a denotes a point of attachment of A₁ to the targeting moiety, b denotes a point of attachment of A to the protease ligand, and q is an integer from 1 to 20.

Embodiment 10. The compound of embodiment 9, wherein each A₁ and A_{q} are each independently selected from P(O)(OR^{L1})O, CR^{L1}R^{L2}, O, NR^{L3}, CONR^{L3}, C(O)O, C(S)O, CO, and heteroaryl optionally substituted with 0-6 R^{L1} R^{L2} groups, wherein R^{L1}, R^{L2} and R^{L3} are each independently selected from H, halo, C₁₋₈ alkyl, and OC₁₋₈ alkyl.

Embodiment 11. The compound of embodiment 9 or 10, wherein A₁ has formula:

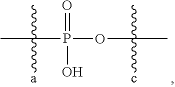

wherein c denotes a point of attachment to A.

Embodiment 12. The compound of embodiment 11, wherein the linker has formula:

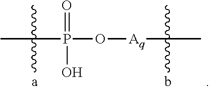

Embodiment 13. The compound of embodiment 10, wherein the heteroaryl has formula:

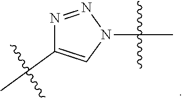 [SMILES: *c1cn(*)nn1]

Embodiment 14. The compound of embodiment 1, wherein the linker has any one of the following formula:

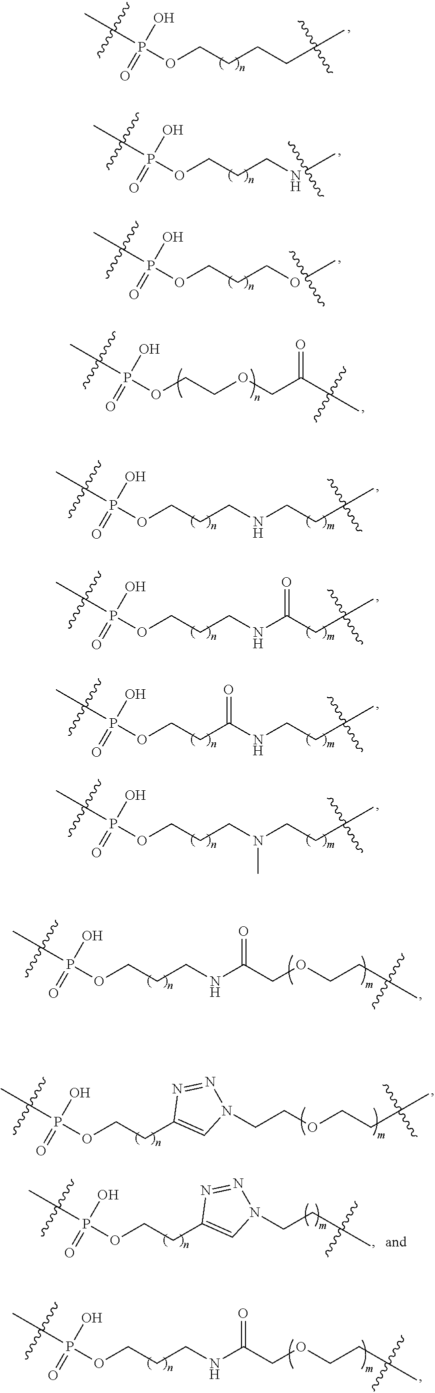

wherein each n and m is independently a number from 0 to 20.

Embodiment 15. The compound of embodiment 1, wherein the linker has any one of the following formula:

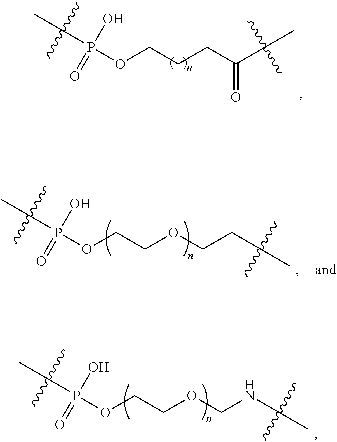

wherein each n is independently a number from 1 to 15.

Embodiment 16. The compound of any one of embodiments 1-15, wherein the protease ligand is selected from the group consisting of

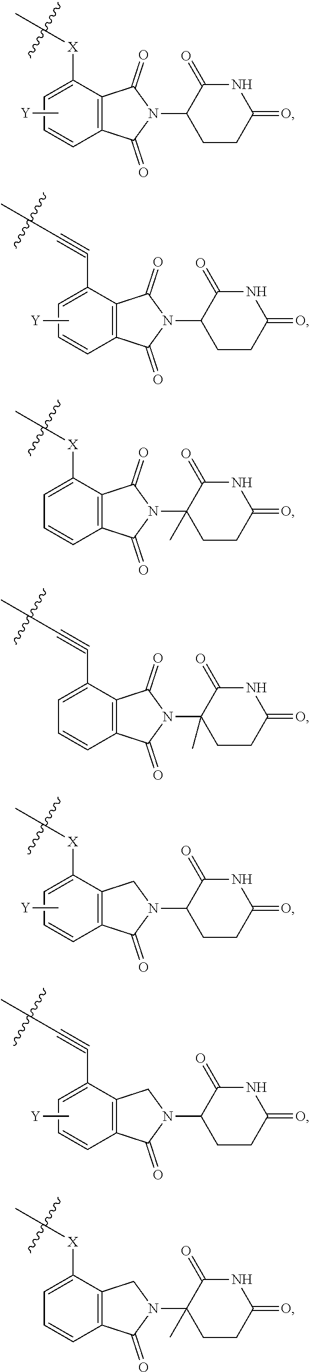

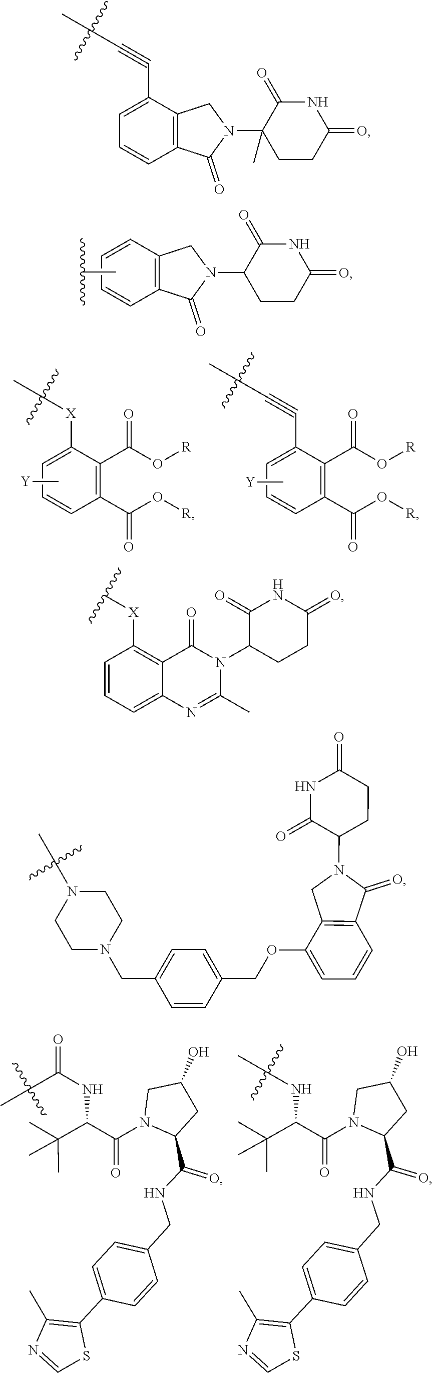

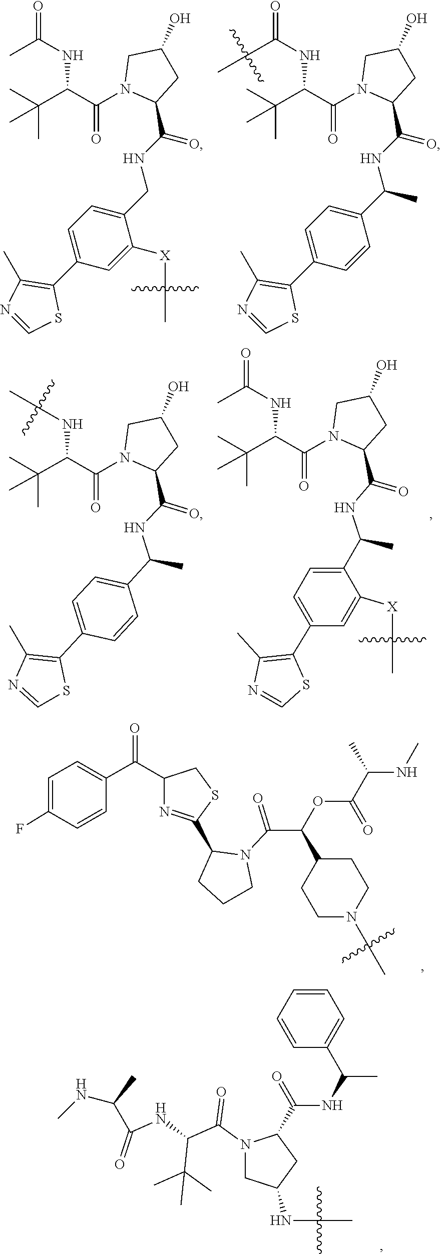

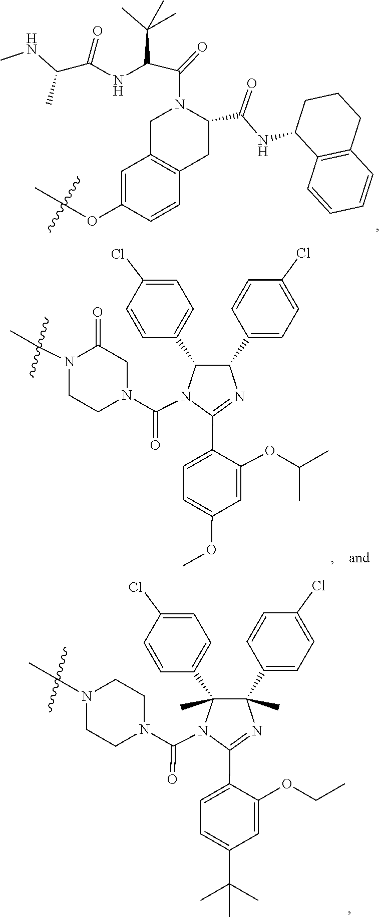

wherein:
each X is independently selected from a bond, NH, O and CH₂;
each Y is independently selected from halo, alkyl, CN, CF₃, OCF₃, and OCHF₂; and
each R is independently selected from H and C₁₋₈ alkyl.

Embodiment 17. The compound of any one of embodiments 1-16, wherein the protease ligand is selected from the group consisting of

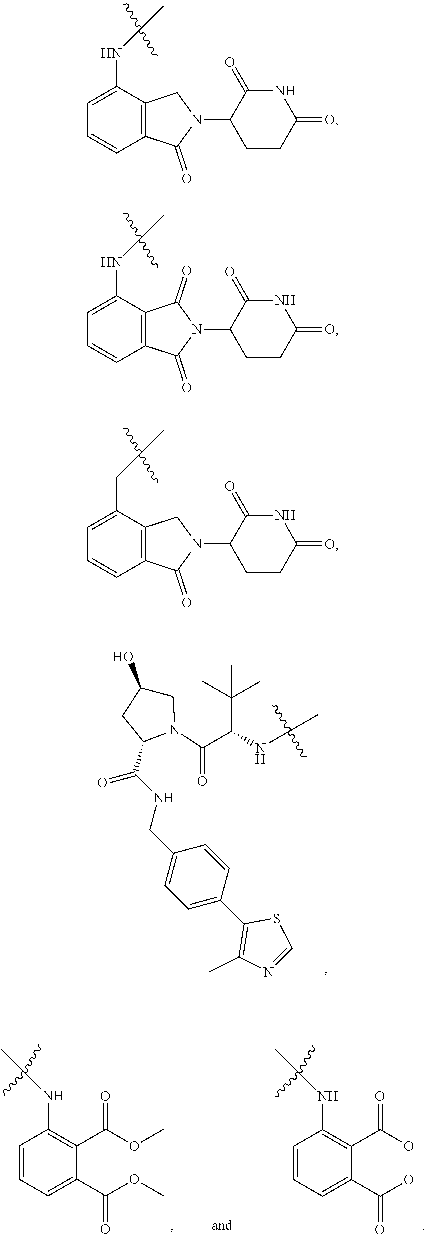 [SMILES: *Nc1cccc2c1CN(C1CCC(=O)NC1=O)C2=O ; *Nc1cccc2c1C(=O)N(C1CCC(=O)NC1=O)C2=O ; *Cc1cccc2c1CN(C1CCC(=O)NC1=O)C2=O ; *N[C@H](C(=O)N1C[C@H](O)C[C@H]1C(=O)NCc1ccc(-c2scnc2C)cc1)C(C)(C)C ; *Nc1cccc(C(=O)OC)c1C(=O)OC ; *Nc1cccc(C(=O)O)c1C(=O)O]

Embodiment 18. A compound of Formula (IB):

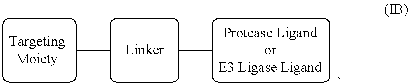

wherein the targeting moiety is an oligonucleotide capable of binding a target protein, and wherein said Protease Ligand or E3 Ligase Ligand component is an E3 ligase ligand.

Embodiment 19. A pharmaceutical composition comprising the compound according to embodiment 18 and a pharmaceutically acceptable carrier.

Embodiment 20. A method for treating a disease or a disorder mediate by aberrant protein activity, wherein said method comprises administering an effective amount of the compound according to embodiment 18 or a pharmaceutical composition comprising the effective amount of the compound to a subject in need of a treatment for aberrant protein activity.

Embodiment 21. The compound, composition, or method according to any one of embodiments 18-20, wherein the targeting moiety is a double-stranded oligonucleotide.

Embodiment 22. The compound, composition, or method according to any one of embodiments 18-21, wherein the target protein is selected from the group consisting of a transcription factor, a transcription co-regulator, a polymerase, a nuclease, a histone, and an RNA-binding protein.

Embodiment 23. The compound, composition, or method according to any one of embodiments 18-22, wherein aberrant protein activity of the target protein mediates a disease or a disorder.

Embodiment 24. The compound, composition, or method according to any one of embodiments 18-23, wherein aberrant protein activity of the target protein mediates a disease or a disorder selected from the group consisting of a cancer, an autoimmune disease, a central nervous system disease, a metabolic disease, and an infection.

Embodiment 25. The compound of embodiment 18, wherein the linker has formula:

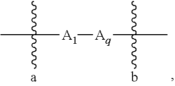

wherein a denotes a point of attachment of A₁ to the targeting moiety, b denotes a point of attachment of A to the E3 ligase ligand, and q is an integer from 1 to 20.

Embodiment 26. The compound of embodiment 25, wherein each A₁ and A_{q} are each independently selected from P(O)(OR^{L1})O, CR^{L1}R^{L2}, O, NR^{L3}, CONR^{L3}, C(O)O, C(S)O, CO, and heteroaryl optionally substituted with 0-6 R^{L1} R^{L2} groups, wherein R^{L1}, R^{L2} and R^{L3} are each independently selected from H, halo, C₁₋₈ alkyl, and OC₁₋₈ alkyl.

Embodiment 27. The compound of embodiment 25 or 26, wherein A₁ has formula:

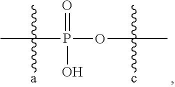

wherein c denotes a point of attachment to A.

Embodiment 28. The compound of embodiment 27, wherein the linker has formula:

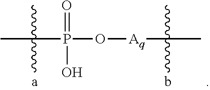

Embodiment 29. The compound of embodiment 26, wherein the heteroaryl has formula:

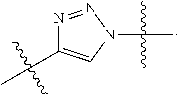

Embodiment 30. The compound of embodiment 18, wherein the linker has any one of the following formula:

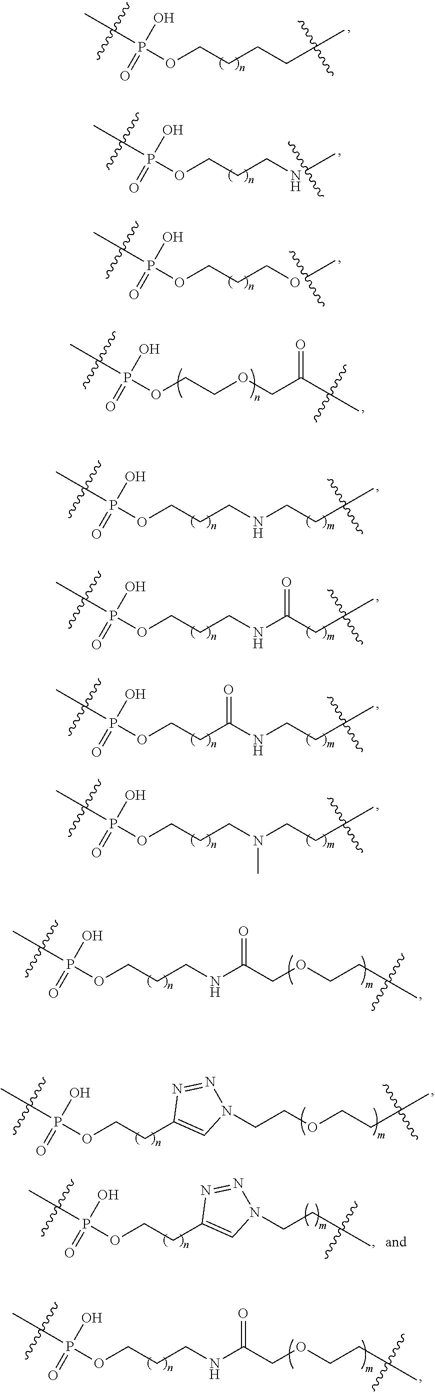

wherein each n and m is independently a number from 0 to 20.

Embodiment 31. The compound of embodiment 18, wherein the linker has any one of the following formula:

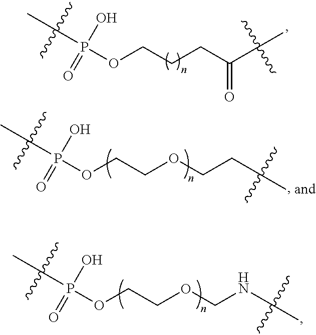

wherein each n is independently a number from 1 to 15.

Embodiment 32. The compound of any one of embodiments 18-31, wherein the E3 ligase ligand is selected from the group consisting of:

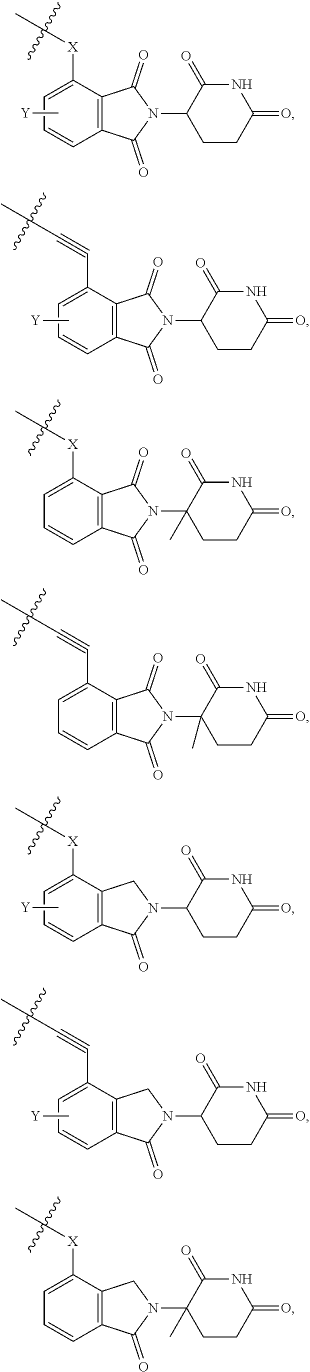

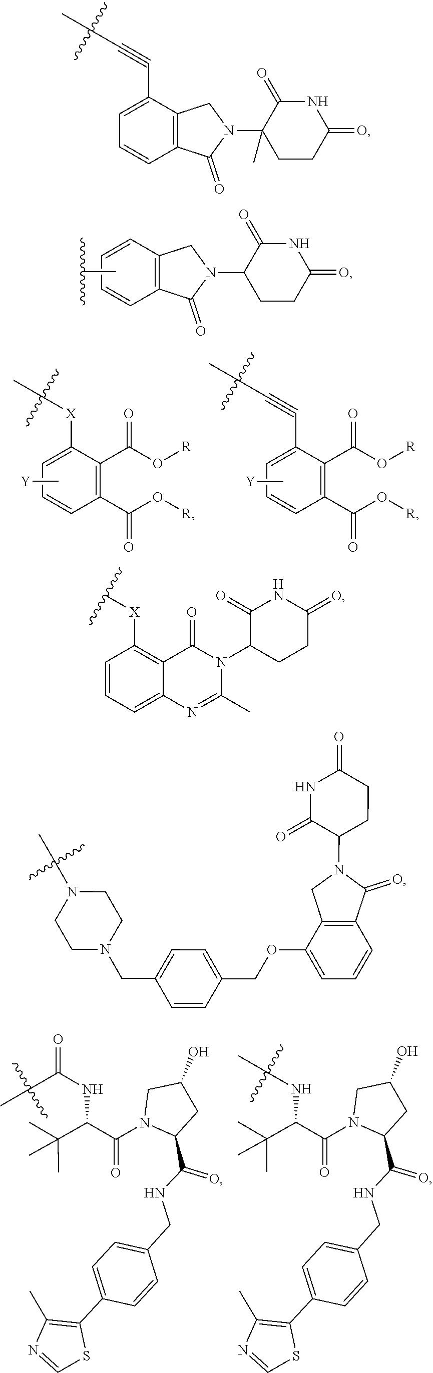

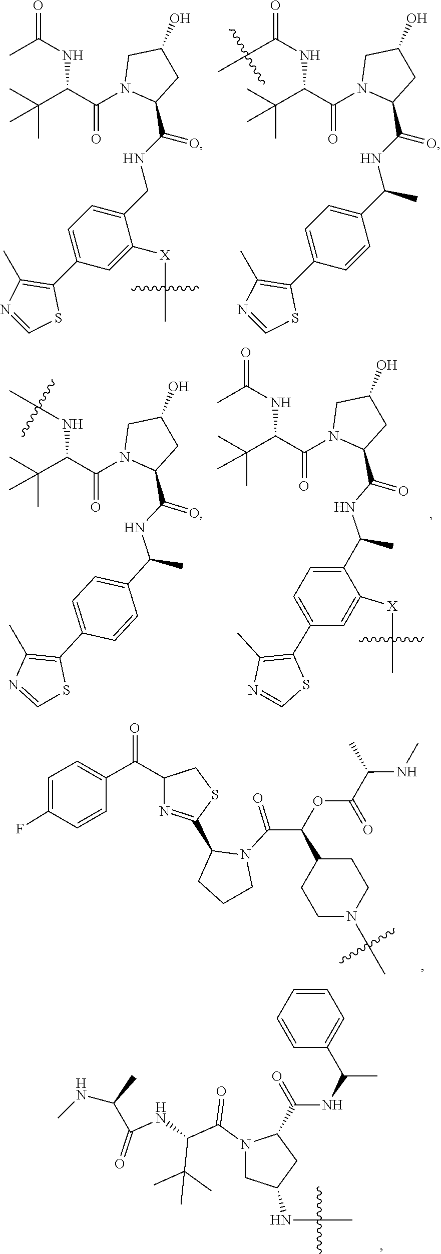

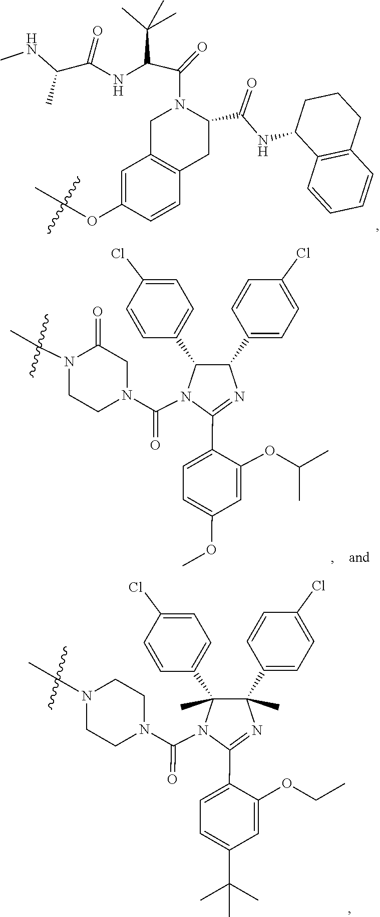

wherein:
each X is independently selected from a bond, NH, O and CH₂;
each Y is independently selected from halo, alkyl, CN, CF₃, OCF₃, and OCHF₂; and
each R is independently selected from H and C₁₋₈ alkyl.

Embodiment 33. The compound of any one of embodiments 18-32, wherein the E3 ligase ligand is selected from the group consisting of

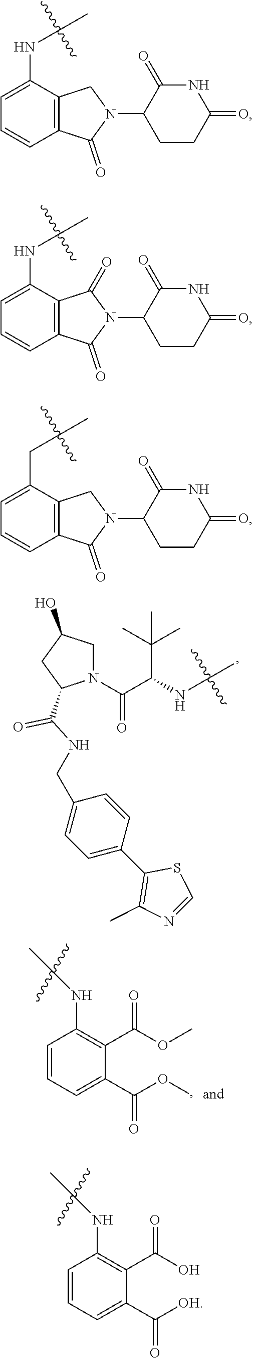

Embodiment 34. A compound of Formula (1B):

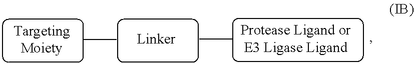

wherein the targeting moiety is capable of binding a target protein, wherein said Protease Ligand or E3 Ligase Ligand component is an E3 ligase ligand capable of binding an E3 ligase, and wherein the E3 ligase ligand is selected from the group consisting of

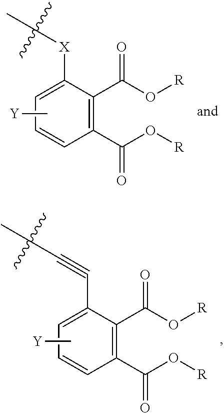

wherein each X is independently selected from a bond, NH, O and CH₂; wherein each Y is independently selected from halo, alkyl, CN, CF₃, OCF₃, and OCHF₂; and wherein each R is independently selected from H and C₁₋₈ alkyl.

Embodiment 35. The compound of embodiment 34, wherein the linker has formula:

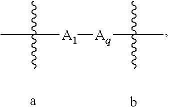

wherein a denotes a point of attachment of A₁ to the targeting moiety, b denotes a point of attachment of A to the E3 ligase ligand, and q is an integer from 1 to 20.

Embodiment 36. The compound of embodiment 35, wherein each A₁ and A_{q} are each independently selected from P(O)(OR^{L1})O, CR^{L1}R^{L2}, O, NR^{L3}, CONR^{L3}, C(O)O, C(S)O, CO, and heteroaryl optionally substituted with 0-6 R^{L1} R^{L2} groups, wherein R^{L1}, R^{L2} and R^{L3} are each independently selected from H, halo, C₁₋₈ alkyl, and OC₁₋₈ alkyl.

Embodiment 37. The compound of embodiment 35 or 36, wherein A₁ has formula:

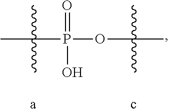

wherein c denotes a point of attachment to A.

Embodiment 38. The compound of embodiment 37, wherein the linker has formula:

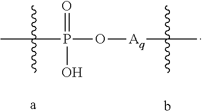

Embodiment 39. The compound of embodiment 35, wherein at least one of A₁ and A_{q} comprises the heteroaryl, and the heteroaryl has formula:

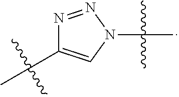

Embodiment 40. The compound of embodiment 34, wherein the linker has any one of the following formula:

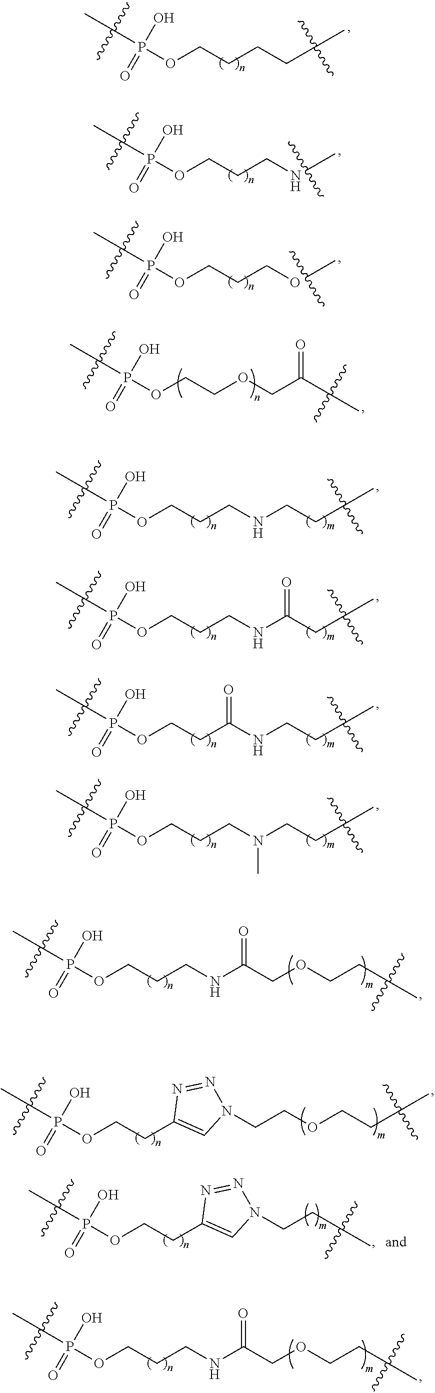

wherein each n and m is independently a number from 0 to 20.

Embodiment 41. The compound of embodiment 34, wherein the linker has any one of the following formula:

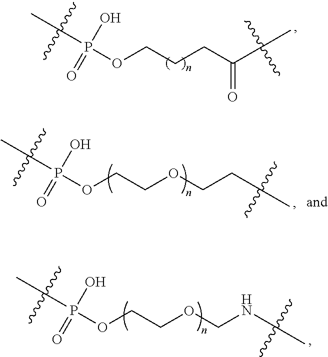

wherein each n is independently a number from 1 to 15.

Embodiment 42. The compound of any one of embodiments 18-41, wherein the targeting moiety comprises a double-stranded oligonucleotide.

Embodiment 43. The compound of embodiment 42, wherein the targeting moiety comprises at least one DNA strand or an analog thereof.

Embodiment 44. The compound of embodiment 42, wherein the targeting moiety comprises at least one RNA strand or an analog thereof.

Embodiment 45. The compound of embodiment 42, wherein the targeting moiety comprises at least one DNA strand or an analog thereof and at least one RNA strand or an analog thereof.

Embodiment 46. The compound of any one of embodiments 18-45, wherein the target protein is selected from the group consisting of a transcription factor, a transcription co-regulator, a polymerase, a nuclease, a histone, and an RNA-binding protein.

Embodiment 47. The compound of any one of embodiments 34-41, wherein the target protein is selected from the group consisting of a transcription factor, a transcription co-regulator, a polymerase, a nuclease, a histone, and an RNA-binding protein.

Embodiment 48. The compound of any one of embodiments 18-47, wherein the target protein is a transcription factor selected from the group consisting of androgen receptor (AR) polypeptide, ETS-related gene (ERG) polypeptide, forkhead box A1 (FOXA1) polypeptide, lymphoid enhancer-binding factor 1 (LEF1) polypeptide, estrogen receptor (ER) polypeptide, NF-κB polypeptide, E2 factor (E2F) polypeptide, transactivator of transcription (TAT) polypeptide, Jun proto-oncogene polypeptide, Fos proto-oncogene polypeptide, nuclear factor of activated T cell (NFAT) polypeptide, Runt-related transcription factor 1 (RUNX1/AML1) polypeptide, Myc proto-oncogene polypeptide, ETS proto-oncogene polypeptide, glioma-associated oncogene (GL1) polypeptide, ERG/FUS fusion polypeptide, T-cell leukemia homeobox 1 (TLX1) polypeptide, LIM domain only 1 (LMO1) polypeptide, LIM domain only 2 (LMO2) polypeptide, lymphoblastic leukemia associated hematopoiesis regulator 1 (LYL1/E2a heterodimer) polypeptide, MYB proto-oncogene (MYB) polypeptide, paired box 5 (PAX-5) polypeptide, SKI proto-oncogene (SKI) polypeptide, T-cell acute lymphocytic leukemia protein 1 (TAL1) polypeptide, T-cell acute lymphocytic leukemia protein 2 (TAL2) polypeptide, glucocorticoid receptor polypeptide, nuclear factor for IL-6 expression (NF-IL6) polypeptide, early growth response protein 1 (EGR-1) polypeptide, hypoxia-inducible factor 1-alpha (HIF-1a) polypeptide, signal transducer and activator of transcription 1 (STAT1) polypeptide, signal transducer and activator of transcription 3 (STAT3) polypeptide, signal transducer and activator of transcription 5 (STAT5) polypeptide, V-Maf avian musculoaponeurotic fibrosarcoma oncogene homolog-A (MAFA) polypeptide, SRY-box transcription factor 2 (SOX2) polypeptide, SRY-box transcription factor 9 (SOX9) polypeptide, CAAT/enhancer-binding protein alpha (CEBPA) polypeptide, CAAT/enhancer-binding protein beta (CEBPB) polypeptide, Globin transcription factor (GATA) polypeptide, myocyte enhancer factor 2 (MEF2) polypeptide, POU class 3 homeobox 2 (BRN2) polypeptide, zinc finger E-box binding homeobox 2 (ZEB2) polypeptide, nuclear receptor subfamily 4 group A member 1 (NR4A1) polypeptide, activating transcription factor 4 (ATF4) polypeptide, T-box transcription factor 21 (TBX21) polypeptide, RAR related orphan receptor C (RORC) polypeptide, X-box binding protein (XBP-1s) polypeptide, and tumor protein p53 (p53).

Embodiment 49. The compound of any one of embodiments 18-48, wherein the target protein is a mutated transcription factor, and wherein aberrant protein activity of the transcription factor mediates a disease.

Embodiment 50. The compound of embodiment 49, wherein the disease is selected from the group consisting of a cancer, an autoimmune disease, a central nervous system disease, a metabolic disease, and an infection.

Embodiment 51. The compound of any one of embodiments 49-50, wherein the mutated transcription factor is a mutated p53.

Embodiment 52. The compound of any one of embodiments 18-47, wherein the target protein is a transcription co-regulator.

Embodiment 53. The compound of embodiment 52, wherein the transcription co-regulator is selected from the group consisting of CBP, p300, SRC1 family polypeptides, SRC2 family polypeptides, SRC3 family polypeptides, BET polypeptides, TRIM family polypeptides, and CXXC-domain zinc finger polypeptides.

Embodiment 54. The compound of any one of embodiments 18-47, wherein the target protein is a polymerase.

Embodiment 55. The compound of embodiment 54, wherein the polymerase is selected from the group consisting of DNA polymerase and RNA polymerase.

Embodiment 56. The compound of any one of embodiments 18-47, wherein the target protein is a nuclease.

Embodiment 57. The compound of embodiment 56, wherein the nuclease is selected from the group consisting of DNA2 and FAN1.

Embodiment 58. The compound of any one of embodiments 18-47, wherein the target protein is a histone.

Embodiment 59. The compound of embodiment 58, wherein the histone is selected from the group consisting of H3, H4, H2A, H2B, and H1.

Embodiment 60. The compound of any one of embodiments 18-47, wherein the target protein is an RNA-binding protein.

Embodiment 61. The compound of embodiment 60, wherein the RNA-binding protein is selected from the group consisting of HIV protein TAT, HIV protein REV1, YTHDC1, YTHDC2, YTHDF1, YTHDF2, YTHDF3, HNRNPA2B1, HNRNPC, and HNRNPG.

Embodiment 62. A pharmaceutical composition comprising the compound of any one of embodiments 18-61, and a pharmaceutically acceptable carrier thereof.

Embodiment 63. A method for treating a disease or disorder mediated by aberrant protein activity, wherein said method comprises administering to a mammal in need of treatment for aberrant protein activity an effective amount of any one of the compounds of embodiments 18-61 or the pharmaceutical composition of embodiment 62 comprising a therapeutically effective amount of the compound, thereby treating said mammal having the disease or disorder mediated by aberrant protein activity.

Embodiment 64. The method of embodiment 63, wherein said mammal is a human.

Embodiment 65. A method of making a compound of Formula (B):

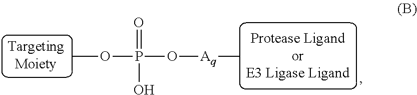

wherein:
the targeting moiety is an oligonucleotide capable of binding a target protein;
the protease ligand is ligand capable of binding a protease, and the E3 ligase ligand is a ligand capable of binding an E3 ligase;
each A is independently selected from P(O)(OR^{L1})O, CR^{L1}R^{L2}, O, NR^{L3}, CONR^{L3}, C(O)O, C(S)O, CO, and heteroaryl optionally substituted with 0-6 R^{L1} R^{L2} groups, wherein R^{L1}, R^{L2} and R^{L3} are each independently selected from H, halo, C₁₋₈ alkyl, and OC₁₋₈ alkyl;
q is an integer from 1 to 20;
wherein said method comprises reacting a compound of formula (i)

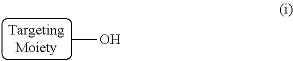

with a compound of formula (ii):

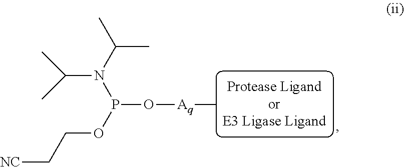

to obtain a compound of formula (iii):

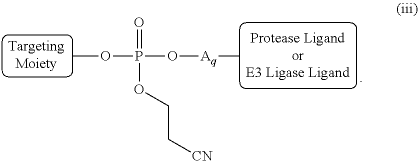

Embodiment 66. The method of embodiment 65, comprising deprotecting the compound of formula (iii) to obtain a compound of Formula (B).

Embodiment 67. The method of embodiment 66, wherein the compound of Formula (ii) is selected from any one of the following compounds:

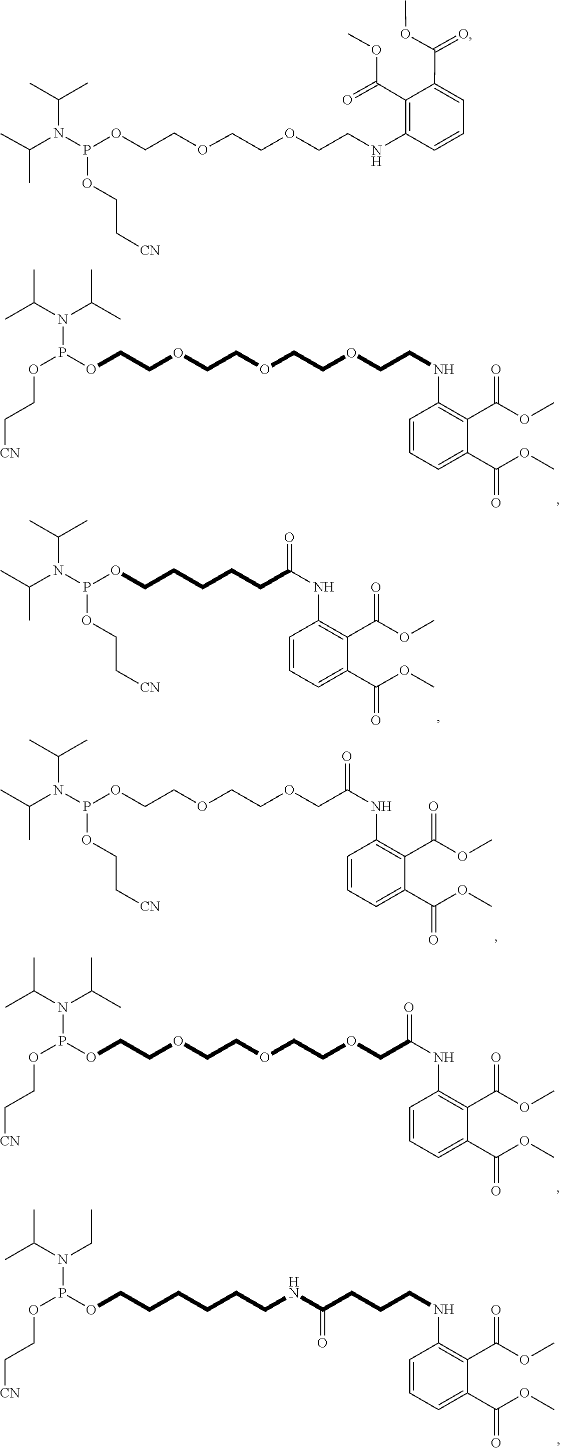

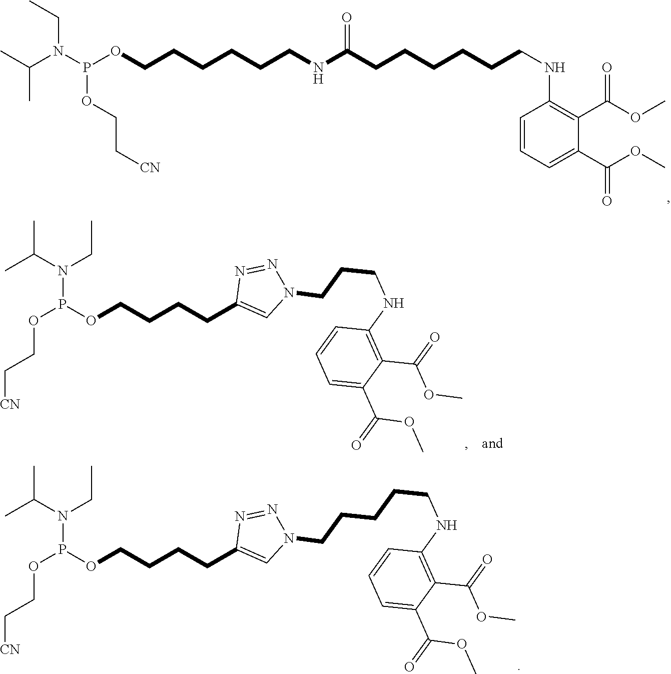

Embodiment 68. The method of embodiment 65, wherein the compound of formula (ii) is selected from any one of the following compounds:

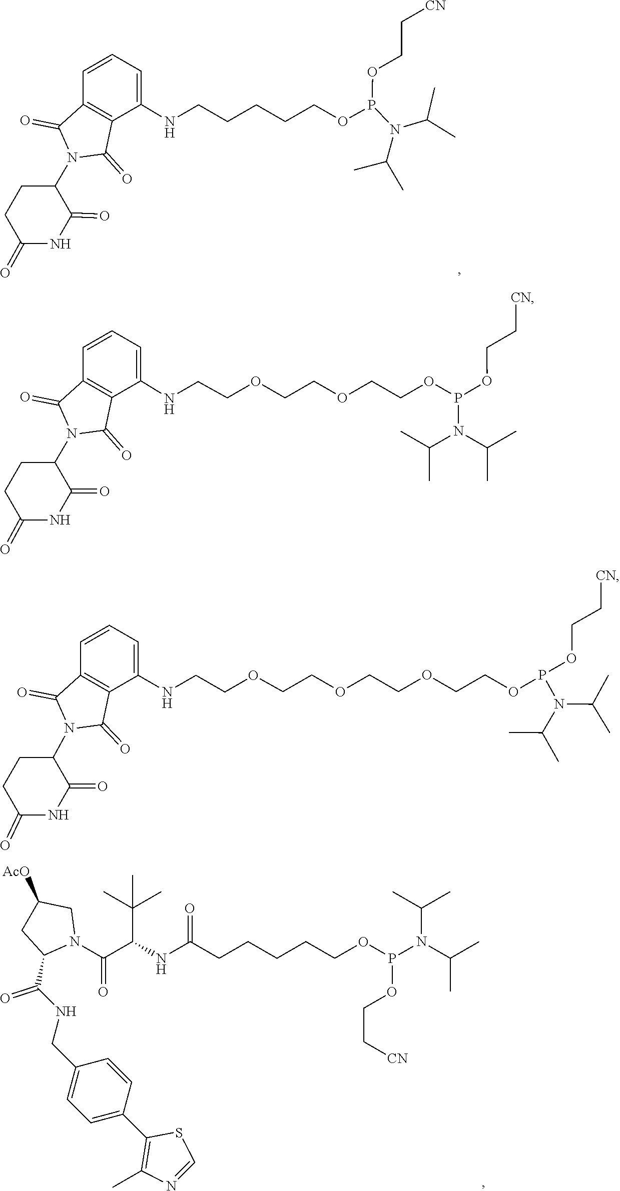

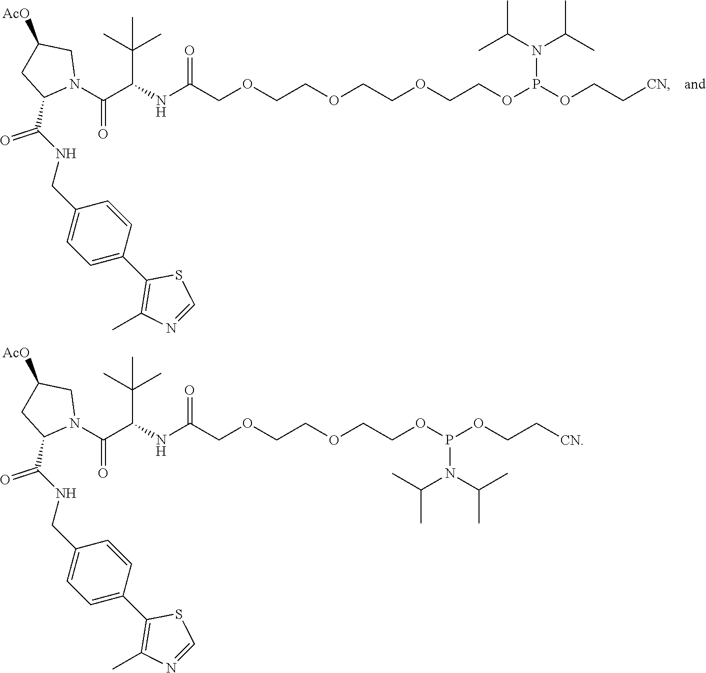

Embodiment 69. A method of making a compound of Formula (B):

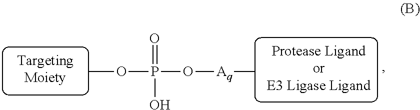

wherein:
the targeting moiety is an oligonucleotide capable of binding a target protein;
the protease ligand is ligand capable of binding a protease, and the E3 ligase ligand is a ligand capable of binding an E3 ligase;
each A is independently selected from P(O)(OR^{L1})O, CR^{L1}R^{L2}, O, NR^{L3}, CONR^{L3}, C(O)O, C(S)O, CO, and heteroaryl optionally substituted with 0-6 R^{L1} R^{L2} groups, wherein R^{L1}, R^{L2} and R^{L3} are each independently selected from H, halo, C₁₋₈ alkyl, and OC₁₋₈ alkyl; and
q is an integer from 1 to 20;
wherein said method comprises reacting a compound of formula (vi):

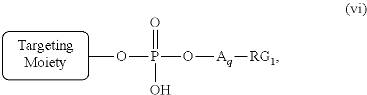

wherein RG₁ is a reactive group;
with a compound of formula (vii):

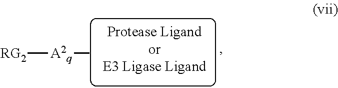

wherein A² is selected from a bond and an A; and wherein RG₂ is a chemical group capable of reacting with the reactive group RG₁ to form A;
to obtain the compound of Formula (B).

Embodiment 70. The method of embodiment 69, wherein RG₁ is an amino group, and RG₂ is an activated ester.

Embodiment 71. The method of embodiment 69, wherein RG₁ is an alkyne, and RG₂ is an azide.

Embodiment 72. The method of embodiment 69, wherein said method comprises deprotecting a compound of formula (v):

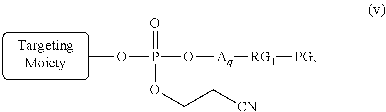

wherein
PG is a protecting group;
to obtain the compound of formula (vi).

Embodiment 73. The method of embodiment 69, wherein the reactive group is selected from an alkyne, an azide, a cycloalkyne, a cyclooctene, a tetrazine, an amino group, a hydroxyl group, and a carboxylic acid.

Embodiment 74. The method of embodiment 72, wherein the protecting group is selected from a hydroxyl protecting group, an amino protecting group, and a carboxylic acid protecting group.

Embodiment 75. The method of embodiment 72, wherein the reactive group is an amino group, and a protecting group is an amino-protecting group.

Embodiment 76. The method of embodiment 75, wherein the amino protecting group is selected from Fluorenylmethyloxycarbonyl (Fmoc), tert-butoxycarbonyl (Boc), benxyloxycarbonyl (Cbz), phthalimide, benzyl, acetyl, and trifluoroacetamide.

Embodiment 77. The method of embodiment 72, wherein the protecting group is a hydroxyl-protecting group.

Embodiment 78. The method of embodiment 77, wherein the hydroxyl-protecting group is selected from t-butyldimethylsilyl, diethylisopropylsilyl, triphenylsilyl, formate, methoxymethylcarbonate, t-butylcarbonate, 9-fluorenylmethylcarbonate, N-phenylcarbamate, 4,4′-dimethoxytrityl, monomethoxytrityl, trityl, and pixyl.

Embodiment 79. The method of embodiment 69, wherein said method comprises reacting a compound of formula (i):

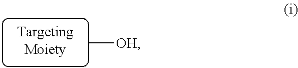

with a compound of formula (iv).

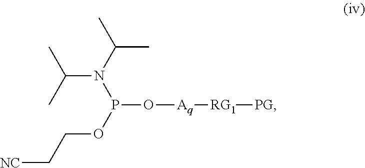

to obtain the compound of formula (v).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula (IB): 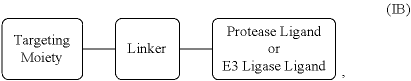
wherein the targeting moiety is an oligonucleotide capable of binding a target protein, and wherein said Protease Ligand or E3 Ligase Ligand component is an E3 ligase ligand.

2. The compound of claim 1, wherein the targeting moiety is a double-stranded oligonucleotide.

3. The compound of claim 1, wherein the target protein is selected from the group consisting of a transcription factor, a transcription co-regulator, a polymerase, a nuclease, a histone, and an RNA-binding protein.

4. The compound of claim 1, wherein the linker has formula: 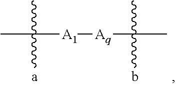
wherein a denotes a point of attachment of A₁ to the targeting moiety, b denotes a point of attachment of A to the E3 ligase ligand, and q is an integer from 1 to 20.

5. The compound of claim 4, wherein each A₁ and A_{q} are each independently selected from P(O)(OR^{L1})O, CR^{L1}R^{L2}, O, NR^{L3}, CONR^{L3}, C(O)O, C(S)O, CO, and heteroaryl optionally substituted with 0-6 R^{L1} R^{L2} groups, wherein R^{L1}, R^{L2} and R^{L3} are each independently selected from H, halo, C₁₋₈ alkyl, and OC₁₋₈ alkyl.

6. The compound of claim 4, wherein A₁ has formula: 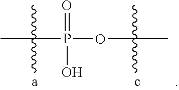
wherein c denotes a point of attachment to A.

7. The compound of claim 6, wherein the linker has formula:

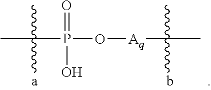

8. The compound of claim 5, wherein the heteroaryl has formula:

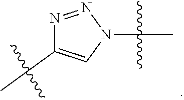 [SMILES: *c1cn(*)nn1]

9. The compound of claim 1, wherein the linker has any one of the following formula: 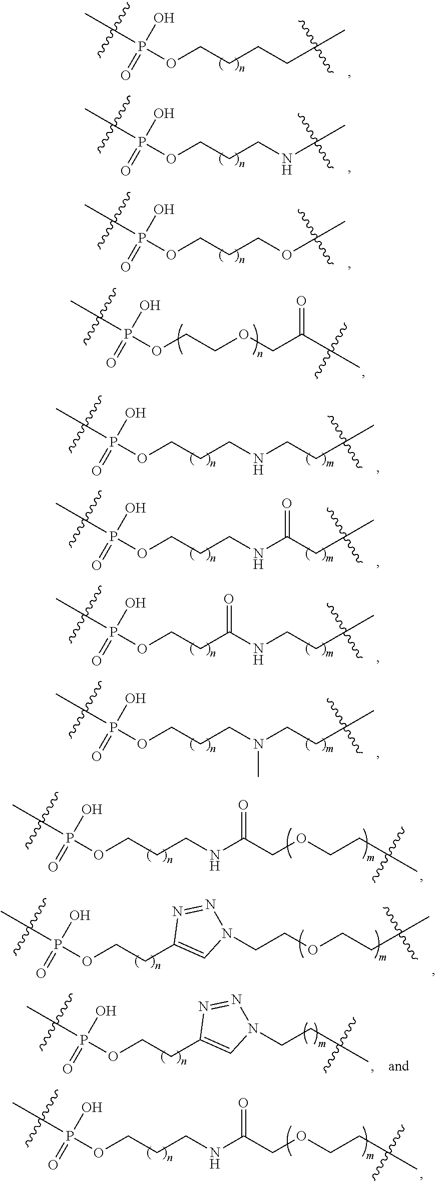
wherein each n and m is independently a number from 0 to 20.

10. The compound of claim 1, wherein the linker has any one of the following formula: 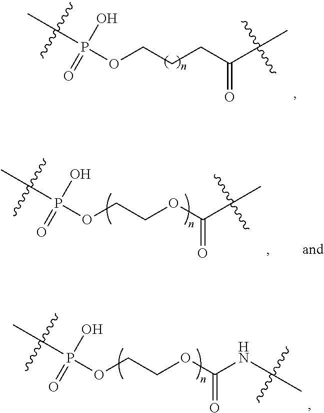
wherein each n is independently a number from 1 to 15.

11-12. (canceled)

13. A compound of Formula (1B): 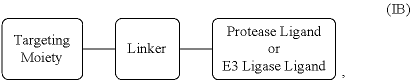
wherein the targeting moiety is capable of binding a target protein, wherein said Protease Ligand or E3 Ligase Ligand component is an E3 ligase ligand capable of binding an E3 ligase, and wherein the E3 ligase ligand is selected from the group consisting of 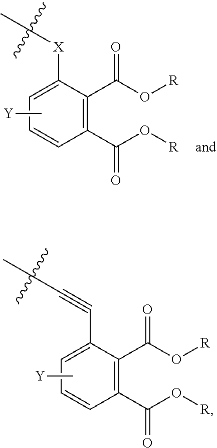
wherein each X is independently selected from a bond, NH, O and CH₂; wherein each Y is independently selected from halo, alkyl, CN, CF₃, OCF₃, and OCHF₂; and wherein each R is independently selected from H and C₁₋₈ alkyl.

14. The compound of claim 13, wherein the linker has formula: 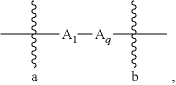
wherein a denotes a point of attachment of A₁ to the targeting moiety, b denotes a point of attachment of A to the E3 ligase ligand, and q is an integer from 1 to 20.

15. The compound of claim 14, wherein each A₁ and A_{q} are each independently selected from P(O)(OR^{L1})O, CR^{L1}R^{L2}, O, NR^{L3}, CONR^{L3}, C(O)O, C(S)O, CO, and heteroaryl optionally substituted with 0-6 R^{L1} R^{L2} groups, wherein R^{L1}, R^{L2} and R^{L3} are each independently selected from H, halo, C₁₋₈ alkyl, and OC₁₋₈ alkyl.

16. The compound of claim 14, wherein A₁ has formula: 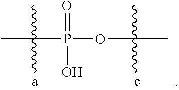
wherein c denotes a point of attachment to A.

17. The compound of claim 16, wherein the linker has formula:

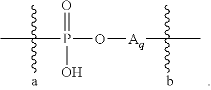

18. The compound of claim 14, wherein at least one of A₁ and A_{q} comprises the heteroaryl, and the heteroaryl has formula:

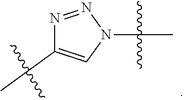 [SMILES: *c1cn(*)nn1]

19. The compound of claim 13, wherein the linker has any one of the following formula: 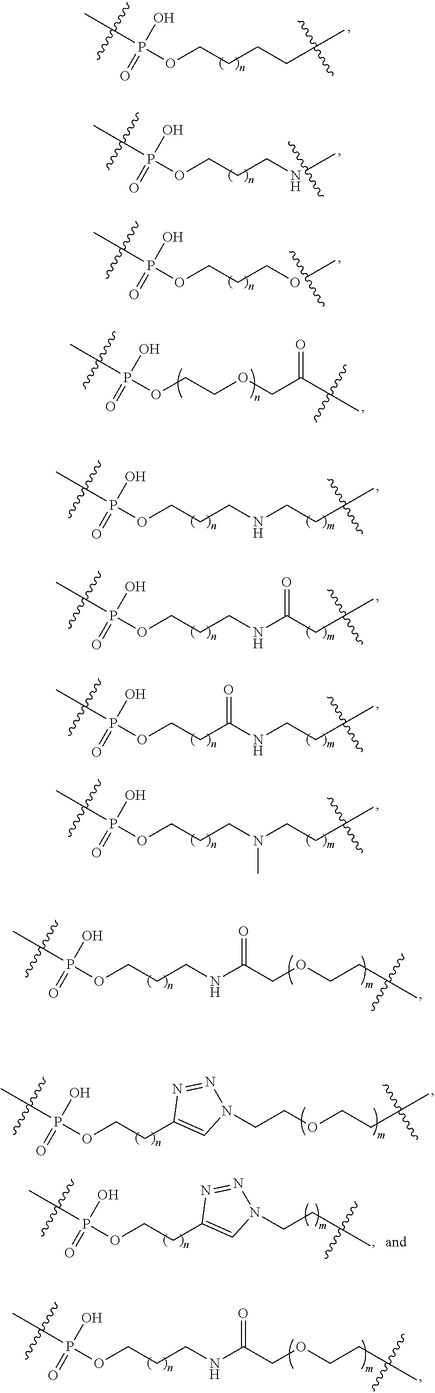
wherein each n and m is independently a number from 0 to 20.

20. The compound of claim 13, wherein the linker has any one of the following formula: 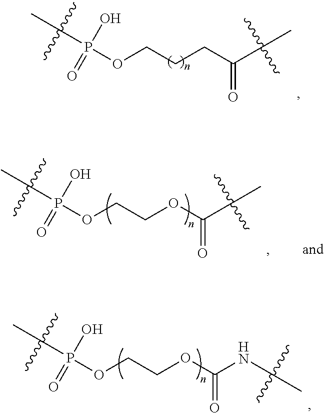
wherein each n is independently a number from 1 to 15.

21. The compound of claim 13, wherein the targeting moiety comprises a double-stranded oligonucleotide.

22-37. (canceled)

38. A compound of Formula (IB): 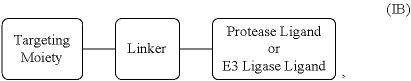
wherein the targeting moiety is an oligonucleotide capable of binding tumor protein p53 (p53), and wherein said Protease Ligand or E3 Ligase Ligand component is an E3 ligase ligand.

39. The compound of claim 38, wherein the targeting moiety is a double-stranded oligonucleotide.

40. The compound of claim 38, wherein said p53 is a mutant p53.

41. The compound of claim 40, wherein said mutant p53 is a gain of function mutant p53.

42-48. (canceled)

49. A method for treating a mammal having a cancer, wherein cancer cells of said cancer express a mutant p53, wherein said method comprises administering, to said mammal, a compound of Formula (IB): 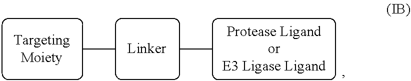
wherein the targeting moiety is an oligonucleotide capable of binding tumor protein p53 (p53), and wherein said Protease Ligand or E3 Ligase Ligand component is an E3 ligase ligand.

50. The method of claim 49, wherein said mammal is a human.
